(19) United States
(12) Patent Application Publication
(10) Pub. No.: US 2007/0048301 A1
(43) Pub. Date: Mar. 1, 2007

(54) Compositions and methods for the treatment of immune related diseases

(76) Inventors: Sarah C. Bodary-Winter, Menlo Park, CA (US); Hilary Clark, San Francisco, CA (US); Brisdell Hunte, San Francisco, CA (US); Jenet K. Jackman, Half Moon Bay, CA (US); Jill R. Schoenfeld, Ashland, OR (US); P. Mickey Williams, South Fran Francisco, CA (US)

Correspondence Address: GENENTECH, INC., 1 DNA WAY, SOUTH SAN FRANCISCO, CA, 94080, US

(21) Appl. No.: 10/533,520

(22) PCT Filed: Nov. 24, 2003
(86) PCT No.: PCT/US03/35971

§ 371 Date: Mar. 9, 2006

(30) Foreign Application Priority Data
Nov. 26, 2002 (US) 60429069

(51) Int. Cl. C12Q 1/68; G01N 33/53; C07H 21/04; C12P 21/06; A61K 39/395; C07K 14/705; C12N 15/74

(52) U.S. Cl. 424/143.1; 530/350; 530/388.22; 435/69.1; 435/252.33; 435/488; 536/23.5; 435/6; 435/7.1

(57) ABSTRACT

The present invention relates to compositions containing novel proteins and methods of using those compositions for the diagnosis and treatment of immune related diseases.

### FIELD OF THE INVENTION

The present invention relates to compositions and methods useful for the diagnosis and treatment of immune related diseases.

### BACKGROUND OF THE INVENTION

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect Therapeutic intervention can occur by either antagonism of a detrimental process/pathway or stimulation of a beneficial process/pathway.

Many immune related diseases are known and have been extensively studied. Such diseases include immune-mediated inflammatory diseases, non-immune-mediated inflammatory diseases, infectious diseases, immunodeficiency diseases, neoplasia, etc.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

Immune related diseases could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

CD4+ T cells are known to be important regulators of inflammation. Herein, CD4+ T cells were activated and the profile of genes differentially expressed upon activation was analyzed. As such, the activation specific genes may be potential therapeutic targets. In vivo co-stimulation is necessary for a productive immune proliferative response. The list of costimulatory molecules is quite extensive and it is still unclear just which co-stimulatory molecules play critical roles in different types and stages of inflammation. In this application the focus is on genes which are specifically upregulated or down-regulated by stimulation with anti-CD3/ICAM, or anti-CD3/anti-CD28 and may be useful in targeting inflammatory processes which are associated with these different molecules.

Despite the above identified advances in T cell research, there is a great need for additional diagnostic and therapeutic agents capable of detecting the presence of a T cell mediated disorders in a mammal and for effectively reducing these disorders. Accordingly, it is an objective of the present invention to identify polypeptides that are overexpressed in activated T cells as compared to resting T cells, and to use those polypeptides, and their encoding nucleic acids, to produce compositions of matter useful in the therapeutic treatment and diagnostic detection of T cell mediated disorders in mammals.

### SUMMARY OF THE INVENTION

### A. Embodiments

The present invention concerns compositions and methods useful for the diagnosis and treatment of immune related disease in mammals, including humans. The present invention is based on the identification of proteins (including agonist and antagonist antibodies) which are a result of stimulation of the immune response in mammals. Immune related diseases can be treated by suppressing or enhancing the immune response. Molecules that enhance the immune response stimulate or potentiate the immune response to an antigen. Molecules which stimulate the immune response can be used therapeutically where enhancement of the immune response would be beneficial. Alternatively, molecules that suppress the immune response attenuate or reduce the immune response to an antigen (e.g., neutralizing antibodies) can be used therapeutically where attenuation of the immune response would be beneficial (e.g., inflammation). Accordingly, the PRO polypeptides, agonists and antagonists thereof are also useful to prepare medicines and medicaments for the treatment of immune-related and inflammatory diseases. In a specific aspect, such medicines and medicaments comprise a therapeutically effective amount of a PRO polypeptide, agonist or antagonist thereof with a pharmaceutically acceptable carrier. Preferably, the admixture is sterile.

In a further embodiment, the invention concerns a method of identifying agonists or antagonists to a PRO polypeptide which comprises contacting the PRO polypeptide with a candidate molecule and monitoring a biological activity mediated by said PRO polypeptide. Preferably, the PRO polypeptide is a native sequence PRO polypeptide. In a specific aspect, the PRO agonist or antagonist is an anti-PRO antibody.

In another embodiment, the invention concerns a composition of matter comprising a PRO polypeptide or an agonist or antagonist antibody which binds the polypeptide in admixture with a carrier or excipient. In one aspect, the composition comprises a therapeutically effective amount of the polypeptide or antibody. In another aspect, when the composition comprises an immune stimulating molecule, the composition is useful for: (a) increasing infiltration of inflammatory cells into a tissue of a mammal in need thereof, (b) stimulating or enhancing an immune response in a mammal in need thereof, (c) increasing the proliferation of T-lymphocytes in a mammal in need thereof in response to an antigen, (d) stimulating the activity of T-lymphocytes or (e) increasing the vascular permeability. In a further aspect, when the composition comprises an immune inhibiting molecule, the composition is useful for: (a) decreasing infiltration of inflammatory cells into a tissue of a mammal in need thereof, (b) inhibiting or reducing an immune response in a mammal in need thereof, (c) decreasing the activity of T-lymphocytes or (d) decreasing the proliferation of T-lymphocytes in a mammal in need thereof in response to an antigen. In another aspect, the composition comprises a further active ingredient, which may, for example, be a further antibody or a cytotoxic or chemotherapeutic agent. Preferably, the composition is sterile.

In another embodiment, the invention concerns a method of treating an immune related disorder in a mammal in need thereof, comprising administering to the mammal an effective amount of a PRO polypeptide, an agonist thereof, or an antagonist thereto. In a preferred aspect, the immune related disorder is selected from the group consisting of: systemic lupus erythematosis, rheumatoid arthritis, osteoarthritis, juvenile chronic arthritis, spondyloarthropathies, systemic sclerosis, idiopathic inflammatory myopathies, Sjögren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In another embodiment, the invention provides an antibody which specifically binds to any of the above or below described polypeptides. Optionally, the antibody is a monoclonal antibody, humanized antibody, antibody fragment or single-chain antibody. In one aspect, the present invention concerns an isolated antibody which binds a PRO polypeptide. In another aspect, the antibody mimics the activity of a PRO polypeptide (an agonist antibody) or conversely the antibody inhibits or neutralizes the activity of a PRO polypeptide (an antagonist antibody). In another aspect, the antibody is a monoclonal antibody, which preferably has nonhuman complementarity determining region (CDR) residues and human framework region (FR) residues. The antibody may be labeled and may be immobilized on a solid support In a further aspect, the antibody is an antibody fragment, a monoclonal antibody, a single-chain antibody, or an anti-idiotypic antibody.

In yet another embodiment, the present invention provides a composition comprising an anti-PRO antibody in admixture with a pharmaceutically acceptable carrier. In one aspect, the composition comprises a therapeutically effective amount of the antibody. Preferably, the composition is sterile. The composition may be administered in the form of a liquid pharmaceutical formulation, which may be preserved to achieve extended storage stability. Alternatively, the antibody is a monoclonal antibody, an antibody fragment, a humanized antibody, or a single-chain antibody.

In a further embodiment, the invention concerns an article of manufacture, comprising:

(a) a composition of matter comprising a PRO polypeptide or agonist or antagonist thereof;

(b) a container containing said composition; and

(c) a label affixed to said container, or a package insert included in said container referring to the use of said PRO polypeptide or agonist or antagonist thereof in the treatment of an immune related disease. The composition may comprise a therapeutically effective amount of the PRO polypeptide or the agonist or antagonist thereof.

In yet another embodiment, the present invention concerns a method of diagnosing an immune related disease in a mammal, comprising detecting the level of expression of a gene encoding a PRO polypeptide (a) in a test sample of tissue cells obtained from the mammal, and (b) in a control sample of known normal tissue cells of the same cell type, wherein a higher or lower expression level in the test sample as compared to the control sample indicates the presence of immune related disease in the mammal from which the test tissue cells were obtained.

In another embodiment, the present invention concerns a method of diagnosing an immune disease in a mammal, comprising (a) contacting an anti-PRO antibody with a test sample of tissue cells obtained from the mammal, and (b) detecting the formation of a complex between the antibody and a PRO polypeptide, in the test sample; wherein the formation of said complex is indicative of the presence or absence of said disease. The detection may be qualitative or quantitative, and may be performed in comparison with monitoring the complex formation in a control sample of known normal tissue cells of the same cell type. A larger quantity of complexes formed in the test sample indicates the presence or absence of an immune disease in the mammal from which the test tissue cells were obtained. The antibody preferably carries a detectable label. Complex formation can be monitored, for example, by light microscopy, flow cytometry, fluorimetry, or other techniques known in the art. The test sample is usually obtained from an individual suspected of having a deficiency or abnormality of the immune system.

In another embodiment, the invention provides a method for determining the presence of a PRO polypeptide in a sample comprising exposing a test sample of cells suspected of containing the PRO polypeptide to an anti-PRO antibody and determining the binding of said antibody to said cell sample. In a specific aspect, the sample comprises a cell suspected of containing the PRO polypeptide and the antibody binds to the cell. The antibody is preferably detectably labeled and/or bound to a solid support.

In another embodiment, the present invention concerns an immune-related disease diagnostic kit, comprising an anti-PRO antibody and a carrier in suitable packaging. The kit preferably contains instructions for using the antibody to detect the presence of the PRO polypeptide. Preferably the carrier is pharmaceutically acceptable.

In another embodiment, the present invention concerns a diagnostic kit, containing an anti-PRO antibody in suitable packaging. The kit preferably contains instructions for using the antibody to detect the PRO polypeptide.

In another embodiment, the invention provides a method of diagnosing an immune-related disease in a mammal which comprises detecting the presence or absence or a PRO polypeptide in a test sample of tissue cells obtained from said mammal, wherein the presence or absence of the PRO polypeptide in said test sample is indicative of the presence of an immune-related disease in said mammal.

In another embodiment, the present invention concerns a method for identifying an agonist of a PRO polypeptide comprising:

(a) contacting cells and a test compound to be screened under conditions suitable for the induction of a cellular response normally induced by a PRO polypeptide; and

(b) determining the induction of said cellular response to determine if the test compound is an effective agonist, wherein the induction of said cellular response is indicative of said test compound being an effective agonist.

In another embodiment, the invention concerns a method for identifying a compound capable of inhibiting the activity of a PRO polypeptide comprising contacting a candidate compound with a PRO polypeptide under conditions and for a time sufficient to allow these two components to interact and determining whether the activity of the PRO polypeptide is inhibited. In a specific aspect, either the candidate compound or the PRO polypeptide is immobilized on a solid support. In another aspect, the non-immobilized component carries a detectable label. In a preferred aspect, this method comprises the steps of:

(a) contacting cells and a test compound to be screened in the presence of a PRO polypeptide under conditions suitable for the induction of a cellular response normally induced by a PRO polypeptide; and

(b) determining the induction of said cellular response to determine if the test compound is an effective antagonist.

In another embodiment, the invention provides a method for identifying a compound that inhibits the expression of a PRO polypeptide in cells that normally express the polypeptide, wherein the method comprises contacting the cells with a test compound and determining whether the expression of the PRO polypeptide is inhibited. In a preferred aspect, this method comprises the steps of.

(a) contacting cells and a test compound to be screened under conditions suitable for allowing expression of the PRO polypeptide; and

(b) determining the inhibition of expression of said polypeptide.

In yet another embodiment, the present invention concerns a method for treating an immune-related disorder in a mammal that suffers therefrom comprising administering to the mammal a nucleic acid molecule that codes for either (a) a PRO polypeptide, (b) an agonist of a PRO polypeptide or (c) an antagonist of a PRO polypeptide, wherein said agonist or antagonist may be an anti-PRO antibody. In a preferred embodiment, the mammal is human. In another preferred embodiment, the nucleic acid is administered via ex vivo gene therapy. In a further preferred embodiment, the nucleic acid is comprised within a vector, more preferably an adenoviral, adeno-associated viral, lentiviral or retroviral vector.

In yet another aspect, the invention provides a recombinant viral particle comprising a viral vector consisting essentially of a promoter, nucleic acid encoding (a) a PRO polypeptide, (b) an agonist polypeptide of a PRO polypeptide, or (c) an antagonist polypeptide of a PRO polypeptide, and a signal sequence for cellular secretion of the polypeptide, wherein the viral vector is in association with viral structural proteins. Preferably, the signal sequence is from a mammal, such as from a native PRO polypeptide.

In a still further embodiment, the invention concerns an ex vivo producer cell comprising a nucleic acid construct that expresses retroviral structural proteins and also comprises a retroviral vector consisting essentially of a promoter, nucleic acid encoding (a) a PRO polypeptide, (b) an agonist polypeptide of a PRO polypeptide or (c) an antagonist polypeptide of a PRO polypeptide, and a signal sequence for cellular secretion of the polypeptide, wherein said producer cell packages the retroviral vector in association with the structural proteins to produce recombinant retroviral particles.

In a still further embodiment, the invention provides a method of increasing the activity of T-lymphocytes in a mammal comprising administering to said mammal (a) a PRO polypeptide, (b) an agonist of a PRO polypeptide, or (c) an antagonist of a PRO polypeptide, wherein the activity of T-lymphocytes in the mammal is increased.

In a still further embodiment, the invention provides a method of decreasing the activity of T-lymphocytes in a mammal comprising administering to said mammal (a) a PRO polypeptide, (b) an agonist of a PRO polypeptide, or (c) an antagonist of a PRO polypeptide, wherein the activity of T-lymphocytes in the mammal is decreased.

In a still further embodiment, the invention provides a method of increasing the proliferation of T-lymphocytes in a mammal comprising administering to said mammal (a) a PRO polypeptide, (b) an agonist of a PRO polypeptide, or (c) an antagonist of a PRO polypeptide, wherein the proliferation of T-lymphocytes in the mammal is increased.

In a still further embodiment, the invention provides a method of decreasing the proliferation of T-lymphocytes in a mammal comprising administering to said mammal (a) a PRO polypeptide, (b) an agonist of a PRO polypeptide, or (c) an antagonist of a PRO polypeptide, wherein the proliferation of T-lymphocytes in the mammal is decreased.

### B. Additional Embodiments

In other embodiments of the present invention, the invention provides vectors comprising DNA encoding any of the herein described polypeptides. Host cell comprising any such vector are also provided. By way of example, the host cells may be CHO cells, E. coli, or yeast A process for producing any of the herein described polypeptides is further provided and comprises culturing host cells under conditions suitable for expression of the desired polypeptide and recovering the desired polypeptide from the cell culture.

In other embodiments, the invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

In another embodiment, the invention provides an antibody which specifically binds to any of the above or below described polypeptides. Optionally, the antibody is a monoclonal antibody, humanized antibody, antibody fragment or single-chain antibody.

In yet other embodiments, the invention provides oligonucleotide probes useful for isolating genomic and cDNA nucleotide sequences or as antisense probes, wherein those probes may be derived from any of the above or below described nucleotide sequences.

In other embodiments, the invention provides an isolated nucleic acid molecule comprising a nucleotide sequence that encodes a PRO polypeptide.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full-length PRO polypeptide cDNA as disclosed herein, the coding sequence of a PRO polypeptide lacking the signal peptide as disclosed herein, the coding sequence of an extracellular domain of a transmembrane PRO polypeptide, with or without the signal peptide, as disclosed herein or the coding sequence of any other specifically defined fragment of the full-length amino acid sequence as disclosed herein, or (b) the complement of the DNA molecule of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by any of the human protein cDNAs as disclosed herein, or (b) the complement of the DNA molecule of (a).

Another aspect the invention provides an isolated nucleic acid molecule comprising a nucleotide sequence encoding a PRO polypeptide which is either transmembrane domain-deleted or transmembrane domain-inactivated, or is complementary to such encoding nucleotide sequence, wherein the transmembrane domain(s) of such polypeptide are disclosed herein. Therefore, soluble extracellular domains of the herein described PRO polypeptides are contemplated.

Another embodiment is directed to fragments of a PRO polypeptide coding sequence, or the complement thereof, that may find use as, for example, hybridization probes, for encoding fragments of a PRO polypeptide that may optionally encode a polypeptide comprising a binding site for an anti-PRO antibody or as antisense oligonucleotide probes. Such nucleic acid fragments are usually at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length and alternatively at least about 1000 nucleotides in length, wherein in this context the term “about” means the referenced nucleotide sequence length plus or minus 10% of that referenced length. It is noted that novel fragments of a PRO polypeptide-encoding nucleotide sequence may be determined in a routine manner by aligning the PRO polypeptide-encoding nucleotide sequence with other known nucleotide sequences using any of a number of well known sequence alignment programs and determining which PRO polypeptide-encoding nucleotide sequence fragment(s) are novel. All of such PRO polypeptide-encoding nucleotide sequences are contemplated herein. Also contemplated are the PRO polypeptide fragments encoded by these nucleotide molecule fragments, preferably those PRO polypeptide fragments that comprise a binding site for an anti-PRO antibody.

In another embodiment, the invention provides isolated PRO polypeptide encoded by any of the isolated nucleic acid sequences herein above identified.

In a certain aspect, the invention concerns an isolated PRO polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein.

In a further aspect, the invention concerns an isolated PRO polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by any of the human protein cDNAs as disclosed herein.

In a specific aspect, the invention provides an isolated PRO polypeptide without the N-terminal signal sequence and/or the initiating methionine and is encoded by a nucleotide sequence that encodes such an amino acid sequence as herein before described. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

Another aspect the invention provides an isolated PRO polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

In yet another embodiment, the invention concerns agonists and antagonists of a native PRO polypeptide as defined herein. In a particular embodiment, the agonist or antagonist is an anti-PRO antibody or a small molecule.

In a further embodiment, the invention concerns a method of identifying agonists or antagonists to a PRO polypeptide which comprise contacting the PRO polypeptide with a candidate molecule and monitoring a biological activity mediated by said PRO polypeptide. Preferably, the PRO polypeptide is a native PRO polypeptide.

In a still further embodiment, the invention concerns a composition of matter comprising a PRO polypeptide, or an agonist or antagonist of a PRO polypeptide as herein described, or an anti-PRO antibody, in combination with a carrier. Optionally, the carrier is a pharmaceutically acceptable carrier.

Another embodiment of the present invention is directed to the use of a PRO polypeptide, or an agonist or antagonist thereof as herein before described, or an anti-PRO antibody, for the preparation of a medicament useful in the treatment of a condition which is responsive to the PRO polypeptide, an agonist or antagonist thereof or an anti-PRO antibody.

### LIST OF FIGURES

List of Figures

FIG. 1: DNA325395, NP_000973.2, 200012_x_at
FIG. 2: PRO81927
FIG. 3: DNA329897, NP_031401.1, 200020_at
FIG. 4: PRO69676
FIG. 5: DNA326769, NP_001000.2, 200024_at
FIG. 6: PRO83105
FIG. 7: DNA329898, NP_000979.1, 200025_s_at
FIG. 8: PRO10643
FIG. 9: DNA293451, NP_296374.1, 200026_at
FIG. 10: PRO70720
FIG. 11: DNA326466, NP_004530.1, 200027_at
FIG. 12: PRO60800
FIG. 13: DNA329899, NP_002785.1, 200039_s_at
FIG. 14: PRO69614
FIG. 15: DNA326953, HSPC117, 200042_at
FIG. 16: PRO83270
FIG. 17: DNA255084, NP_001081.1, 200045_at
FIG. 18: PRO50170
FIG. 19: DNA272614, NP_004506.1, 200052_s_at
FIG. 20: PRO60747
FIG. 21: DNA304680, HSPCB, 200064_at
FIG. 22: PRO71106
FIG. 23: DNA189703, NP_005539.1, 200079_s_at
FIG. 24: PRO22637
FIG. 25: DNA329900, NP_002905.1, 1053_at
FIG. 26: PRO81549
FIG. 27: DNA88189, NP_037362.1, 266_s_at
FIG. 28: PRO2690
FIG. 29: DNA272992, NP_055479.1, 32069_at
FIG. 30: PRO61064
FIG. 31A-B: DNA329901, BAA32291.2, 32091_at
FIG. 32: PRO85218
FIG. 33: DNA329902, NP_110419.2, 32502_at
FIG. 34: PRO85219
FIG. 35: DNA329903, NP_005596.2, 32541_at
FIG. 36: PRO85220
FIG. 37: DNA327521, NR_002192.2, 33304_at
FIG. 38: PRO58320
FIG. 39: DNA272223, NP_004444.1, 33494_at
FIG. 40: PRO60485
FIG. 41A-B: DNA329904, NP_066554.1, 33767_at
FIG. 42: PRO85221
FIG. 43: DNA210121, NP_001794.1, 34210_at
FIG. 44: PRO33667
FIG. 45: DNA269828, NP_006691.1, 35254_at
FIG. 46: PRO58230
FIG. 47: DNA88643, NP_000190.1, 35626_at
FIG. 48: PRO2455
FIG. 49: DNA331435, NP_006143.1, 35974_at
FIG. 50: PRO86495
FIG. 51A-B: DNA272022, NP_002607.1, 36829_at
FIG. 52: PRO60296
FIG. 53: DNA226967, NP_055145.2, 37028_at
FIG. 54: PRO37430
FIG. 55: DNA226043, NP_006424.2, 37145_at
FIG. 56: PRO36506
FIG. 57: DNA329906, MGC14258, 37577_at
FIG. 58: PRO85223
FIG. 59: DNA256295, NP_002310.1, 37796_at
FIG. 60: PRO51339
FIG. 61: DNA328354, AF237769, 37966_at
FIG. 62: PRO84215
FIG. 63A-B: DNA329907, NP_036423.1, 38158_at
FIG. 64: PRO85224
FIG. 65A-B: DNA329908, BAA13246.1, 38892_at
FIG. 66: PRO85225
FIG. 67: DNA328356, BC013566, 39248_at
FIG. 68: PRO38028
FIG. 69: DNA327523, NP_004916.1, 39249_at
FIG. 70: PRO38028
FIG. 71A-B: DNA328358, STK10, 40420_at
FIG. 72: PRO84218
FIG. 73: DNA329909, NP_077084.1, 40446_at
FIG. 74: PRO62251
FIG. 75: DNA329910, NP_003251.2, 40837_at
FIG. 76: PRO82891
FIG. 77A-B: DNA329093, NP_006631.1, 41220_at
FIG. 78: PRO84745
FIG. 79A-C: DNA331436, 7689629.6, 43427_at
FIG. 80: PRO86496
FIG. 81: DNA154653, DNA154653, 43511_s_at
FIG. 82: DNA262129, NP_079389.1, 44790_s_at
FIG. 83: PRO54740
FIG. 84: DNA326185, NP_073607.2, 45633_at
FIG. 85: PRO82602
FIG. 86: DNA329912, NP_004614.1, 46167_at
FIG. 87: PRO85227
FIG. 88: DNA329913, SSB-3, 46256_at
FIG. 89: PRO85228
FIG. 90: DNA324145, NP_060259.1, 46665_at
FIG. 91: PRO80846
FIG. 92: DNA329094, NP_077285.1, 48531_at
FIG. 93: PRO84746
FIG. 94: DNA329914, NP_079175.2, 52285_f_at
FIG. 95: PRO85229
FIG. 96: DNA328364, NP_068577.1, 52940_at
FIG. 97: PRO84223
FIG. 98: DNA329915, NP_065093.1, 56197_at
FIG. 99: PRO85230
FIG. 100A-B: DNA328966, AK024397, 57082_at
FIG. 101: PRO84670
FIG. 102A-B: DNA226870, NP_000782.1, 48808_at
FIG. 103: PRO37333
FIG. 104: DNA328366, NP_079233.1, 59375_at
FIG. 105: PRO84225
FIG. 106: DNA331437, 338326.15, 60084_at
FIG. 107: PRO86497
FIG. 108: DNA328367, NP_079108.2, 60471_at
FIG. 109: PRO84226
FIG. 110: DNA327876, NP_005081.1, 60528_at
FIG. 111: PRO83815
FIG. 112: DNA329917, NP_065174.1, 64486_at
FIG. 113: PRO85232
FIG. 114: DNA329918, BC008671, 65630_at
FIG. 115: PRO85233
FIG. 116A-B: DNA196428, BAA31649.1,
76897_s_at
FIG. 117: PRO71274
FIG. 118: DNA329919, C20orf67, 89948_at
FIG. 119: PRO85234
FIG. 120: DNA328369, BC007634, 90610_at
FIG. 121: DNA269410, NP_002725.1, 200605_s_at
FIG. 122: PRO57836
FIG. 123A-B: DNA326380, NP_004850.1, 200614_at
FIG. 124: PRO82774
FIG. 125A-B: DNA194778, NP_055545.1,
200616_s_at
FIG. 126: PRO24056
FIG. 127: DNA287245, NP_004175.1, 200628_s_at
FIG. 128: PRO69520
FIG. 129: DNA287245, WARS, 200629_at
FIG. 130: PRO69520
FIG. 131: DNA327532, GLUL, 200648_s_at
FIG. 132: PRO71134
FIG. 133: DNA97285, NP_005557.1, 200650_s_at
FIG. 134: PRO3632
FIG. 135: DNA226125, NP_003136.1, 200652_at
FIG. 136: PRO36588
FIG. 137: DNA325923, NP_008819.1, 200655_s_at
FIG. 138: PRO4904
FIG. 139: DNA227055, NP_002625.1, 200658_s_at
FIG. 140: PRO37518
FIG. 141: DNA275062, NP_006136.1, 200664_s_at
FIG. 142: PRO62782
FIG. 143: DNA275062, DNAJB1, 200666_s_at
FIG. 144: PRO62782
FIG. 145A-B: DNA328372, 105551.7, 200685_at
FIG. 146: PRO84229
FIG. 147A-B: DNA329920, NP_036558.1,
200687_s_at
FIG. 148: PRO85235
FIG. 149: DNA324633, BC000478, 200691_s_at
FIG. 150: PRO81277
FIG. 151: DNA324897, NP_006845.1, 200700_s_at
FIG. 152: PRO12468
FIG. 153: DNA275267, NP_003737.1, 200703_at
FIG. 154: PRO62952
FIG. 155: DNA328373, AB034747, 200704_at
FIG. 156: PRO84230
FIG. 157: DNA328374, NP_004853.1, 200706_s_at
FIG. 158: PRO84231
FIG. 159: DNA290260, NP_036555.1, 200715_x_at
FIG. 160: PRO70385
FIG. 161: DNA329921, 1315403.9, 200719_at
FIG. 162: PRO85236
FIG. 163: DNA329538, M11S1, 200722_s_at
FIG. 164: PRO85088
FIG. 165: DNA227618, HSGPIP137, 200723_s_at
FIG. 166: PRO38081
FIG. 167: DNA327114, NP_006004.1, 200725_x_at
FIG. 168: PRO62466
FIG. 169A-B: DNA327534, NP_003454.1,
200730_s_at
FIG. 170: PRO41180
FIG. 171A-B: DNA327534, PTP4A1, 200731_s_at
FIG. 172: PRO41180
FIG. 173: DNA331438, 402431.7, 200732_s_at
FIG. 174: PRO86498
FIG. 175: DNA327845, NP_000282.1, 200737_at
FIG. 176: PRO61271
FIG. 177: DNA327845, PGK1, 200738_s_at
FIG. 178: PRO61271
FIG. 179: DNA287207, NP_006316.1, 200750_s_at
FIG. 180: PRO39268
FIG. 181A-B: DNA274977, HSU97105, 200762_at
FIG. 182: PRO62709
FIG. 183: DNA324135, BC001854, 200768_s_at
FIG. 184: PRO80837
FIG. 185: DNA324135, NP_005902.1, 200769_s_at
FIG. 186: PRO80837
FIG. 187: DNA271608, NP_055485.1, 200777_s_at
FIG. 188: PRO59895
FIG. 189: DNA226262, NP_005554.1, 200783_s_at
FIG. 190: PRO36725
FIG. 191: DNA324060, NP_002530.1, 200790_at
FIG. 192: PRO80773
FIG. 193: DNA272928, NP_055579.1, 200794_x_at
FIG. 194: PRO61012
FIG. 195: DNA304668, NP_005336.2, 200799_at
FIG. 196: PRO71095
FIG. 197: DNA227607, NP_005337.1, 200800_s_at
FIG. 198: PRO38070
FIG. 199: DNA287211, NP_002147.1, 200806_s_at
FIG. 200: PRO69492
FIG. 201: DNA287211, HSPD1, 200807_s_at
FIG. 202: PRO69492
FIG. 203: DNA269874, NP_001271.1, 200810_s_at
FIG. 204: PRO58272
FIG. 205: DNA269874, CIRBP, 200811_at
FIG. 206: PRO58272
FIG. 207: DNA227795, NP_006420.1, 200812_at
FIG. 208: PRO38258
FIG. 209: DNA325596, NP_000356.1, 200822_x_at
FIG. 210: PRO69549
FIG. 211A-B: DNA328700, AF097514, 200831_s_at
FIG. 212: PRO84464
FIG. 213A-B: DNA328378, AB032261, 200832_s_at
FIG. 214: PRO84233
FIG. 215: DNA329922, CTSB, 200838_at
FIG. 216: PRO3344
FIG. 217: DNA88165, HUMCTSB, 200839_s_at
FIG. 218: PRO2678
FIG. 219: DNA329923, NP_057211.3, 200847_s_at
FIG. 220: PRO85237
FIG. 221: DNA324509, NP_002097.1, 200853_at
FIG. 222: PRO10297
FIG. 223A-C: DNA331439, NP_001447.1,
200859_x_at
FIG. 224: PRO86499
FIG. 225A-B: DNA228029, NP_055577.1, 200862_at
FIG. 226: PRO38492
FIG. 227: DNA226112, NP_002769.1, 200866_s_at
FIG. 228: PRO36575
FIG. 229: DNA226112, PSAP, 200871_s_at
FIG. 230: PRO36575
FIG. 231: DNA254537, NP_002957.1, 200872_at
FIG. 232: PRO49642
FIG. 233: DNA271030, NP_006383.1, 200875_s_at
FIG. 234: PRO59358
FIG. 235: DNA324107, NP_006421_1, 200877_at
FIG. 236: PRO80814
FIG. 237: DNA328379, BC015869, 200878_at
FIG. 238: PRO84234
FIG. 239: DNA329099, 1164406.9, 200880_at
FIG. 240: PRO60127
FIG. 241: DNA271847, NP_001530.1, 200881_s_at
FIG. 242: PRO60127
FIG. 243: DNA287187, NP_002620.1, 200886_s_at
FIG. 244: PRO69473
FIG. 245A-B: DNA327539, NP_009330.1,
200887_s_at
FIG. 246: PRO12211
FIG. 247: DNA326326, NP_000969.1, 200888_s_at
FIG. 248: PRO82724
FIG. 249: DNA325584, NP_002005.1, 200894_s_at
FIG. 250: PRO59262
FIG. 251: DNA325584, FKBP4, 200895_s_at
FIG. 252: PRO59262
FIG. 253: DNA328380, HSHLAEHCM, 200904_at
FIG. 254: DNA304665, NP_000995.1, 200909_s_at
FIG. 255: PRO71092
FIG. 256: DNA272695, NP_001722.1, 200920_s_at
FIG. 257: PRO60817
FIG. 258: DNA272695, BTG1, 200921_s_at
FIG. 259: PRO60817
FIG. 260: DNA227077, NP_005558.1, 200923_at
FIG. 261: PRO37540
FIG. 262: DNA327255, NP_002385.2, 200924_s_at
FIG. 263: PRO57298
FIG. 264: DNA225878, NP_004334.1, 200935_at
FIG. 265: PRO36341
FIG. 266: DNA329925, NP_001528.1, 200942_s_at
FIG. 267: PRO85239
FIG. 268A-B: DNA287217, NP_001750.1,
200951_s_at
FIG. 269: PRO36766
FIG. 270A-B: DNA287217, CCND2, 200952_s_at
FIG. 271: PRO36766
FIG. 272: DNA227491, NP_009027.1, 200960_x_at
FIG. 273: PRO37954
FIG. 274: DNA331440, NP_036380.2, 200961_at
FIG. 275: PRO86500
FIG. 276A-B: DNA331289, ABLIM1, 200965_s_at
FIG. 277: PRO86390
FIG. 278: DNA287355, NP_000025.1, 200966_x_at
FIG. 279: PRO69617
FIG. 280: DNA324110, NP_005908.1, 200978_at
FIG. 281: PRO4918
FIG. 282: DNA329928, ANXA6, 200982_s_at
FIG. 283: PRO85241
FIG. 284A-B: DNA325896, NP_001521.1, 200989_at
FIG. 285: PRO82352
FIG. 286: DNA329929, 400903.6, 200994_at
FIG. 287: PRO85242
FIG. 288: DNA325778, CKAP4, 200998_s_at
FIG. 289: PRO82248
FIG. 290: DNA331441, BC015436, 200999_s_at
FIG. 291: DNA275408, NP_001596.1, 201000_at
FIG. 292: PRO63068
FIG. 293: DNA304713, NP_006463.2, 201008_s_at
FIG. 294: PRO71139
FIG. 295: DNA304713, TXNIP, 201009_s_at
FIG. 296: PRO71139
FIG. 297: DNA304713, S73591, 201010_s_at
FIG. 298: PRO71139
FIG. 299: DNA89242, NP_000691.1, 201012_at
FIG. 300: PRO2907
FIG. 301: DNA328388, BC010273, 201013_s_at
FIG. 302: PRO84240
FIG. 303: DNA328388, NP_006443.1, 201014_s_at
FIG. 304: PRO84240
FIG. 305: DNA151697, DNA151697, 201016_at
FIG. 306: PRO11993
FIG. 307A-B: DNA329101, NP_056988.2,
201024_x_at
FIG. 308: PRO84751
FIG. 309A-B: DNA329101, IF2, 201027_s_at
FIG. 310: PRO84751
FIG. 311: DNA287372, NP_002618.1, 201037_at
FIG. 312: PRO69632
FIG. 313: DNA328391, NP_004408.1, 201041_s_at
FIG. 314: PRO84242
FIG. 315: DNA328391, DUSP1, 201044_x_at
FIG. 316: PRO84242
FIG. 317: DNA274743, NP_002850.1, 201087_at
FIG. 318: PRO62517
FIG. 319: DNA254725, NP_002257.1, 201088_at
FIG. 320: PRO49824
FIG. 321: DNA329930, ATP6V1B2, 201089_at
FIG. 322: PRO85243
FIG. 323: DNA287198, NP_006073.1, 201090_x_at
FIG. 324: PRO69484
FIG. 325A-B: DNA328395, NP_056198.1,
201104_x_at
FIG. 326: PRO84245
FIG. 327: DNA304719, NP_002296.1, 201105_at
FIG. 328: PRO71145
FIG. 329: DNA329931, AF053642, 201111_at
FIG. 330: DNA331442, NP_002783.1, 201114_x_at
FIG. 331: PRO83189
FIG. 332: DNA273865, NP_006221.1, 201115_at
FIG. 333: PRO61824
FIG. 334: DNA326273, NP_001961.1, 201123_s_at
FIG. 335: PRO82678
FIG. 336: DNA329255, NP_006267.1, 201129_at
FIG. 337: PRO84855
FIG. 338: DNA329103, NP_002112.2, 201137_s_at
FIG. 339: PRO84752
FIG. 340: DNA329104, NP_004085.1, 201144_s_at
FIG. 341: PRO69550
FIG. 342: DNA329105, NP_006109.2, 201145_at
FIG. 343: PRO84753
FIG. 344: DNA329015, NFE2L2, 201146_at
FIG. 345: PRO84691
FIG. 346: DNA329932, BC008801, 201160_s_at
FIG. 347: PRO85244
FIG. 348: DNA151802, NP_003661.1, 201169_s_at
FIG. 349: PRO12890
FIG. 350: DNA151802, BHLHB2, 201170_s_at
FIG. 351: PRO12890
FIG. 352: DNA273342, NP_005887.1, 201193_at
FIG. 353: PRO61345
FIG. 354: DNA331443, NP_003000.1, 201194_at
FIG. 355: PRO86501
FIG. 356A-B: DNA103453, HUME16GEN,
201195_s_at
FIG. 357: PRO4780
FIG. 358: DNA272251, NP_002798.1, 201198_s_at
FIG. 359: PRO60513
FIG. 360: DNA103488, NP_002583.1, 201202_at
FIG. 361: PRO4815
FIG. 362: DNA327544, NP_002865.1, 201222_s_at
FIG. 363: PRO70357
FIG. 364: DNA287173, ENO1, 201231_s_at
FIG. 365: PRO69463
FIG. 366: DNA287331, NP_002645.1, 201251_at
FIG. 367: PRO69595
FIG. 368: DNA274139, NP_006494.1, 201252_at
FIG. 369: PRO62075
FIG. 370: DNA270950, NP_003182.1, 201263_at
FIG. 371: PRO59281
FIG. 372A-B: DNA328404, NP_003321.1, 201266_at
FIG. 373: PRO84251
FIG. 374: DNA331444, NP_002781.1, 201274_at
FIG. 375: PRO60981
FIG. 376: DNA323936, NP_001995.1, 201275_at
FIG. 377: PRO80669
FIG. 378: DNA328405, NP_112556.1, 201277_s_at
FIG. 379: PRO84252
FIG. 380: DNA270526, NP_001166.1, 201288_at
FIG. 381: PRO58903
FIG. 382A-B: DNA327545, TOP2A, 201291_s_at
FIG. 383: PRO82731
FIG. 384: DNA327546, HSTOP2A10, 201292_at
FIG. 385: DNA328407, WSB1, 201294_s_at
FIG. 386: PRO84254
FIG. 387A-B: DNA226778, HSM800772,
201295_s_at
FIG. 388: PRO37241
FIG. 389: DNA327547, NP_060691.1, 201298_s_at
FIG. 390: PRO83583
FIG. 391: DNA287222, NP_055555.1, 201303_at
FIG. 392: PRO69501
FIG. 393: DNA324612, P311, 201310_s_at
FIG. 394: PRO81261
FIG. 395: DNA325595, NP_001966.1, 201313_at
FIG. 396: PRO38010
FIG. 397: DNA331445, NP_002778.1, 201317_s_at
FIG. 398: PRO71133
FIG. 399: DNA274745, NP_006815.1, 201323_at
FIG. 400: PRO62518
FIG. 401: DNA150781, NP_001414.1, 201324_at
FIG. 402: PRO12467
FIG. 403: DNA329002, AF385084, 201326_at
FIG. 404: PRO4912
FIG. 405: DNA329002, NP_001753.1, 201327_s_at
FIG. 406: PRO4912
FIG. 407: DNA269536, S80343, 201330_at
FIG. 408: PRO57952
FIG. 409: DNA273323, NP_004243.1, 201349_at
FIG. 410: PRO61330
FIG. 411: DNA103227, NP_004466.1, 201350_at
FIG. 412: PRO4557
FIG. 413: DNA329934, NP_000090.1, 201360_at
FIG. 414: PRO2721
FIG. 415: DNA329107, NP_008818.3, 201367_s_at
FIG. 416: PRO84754
FIG. 417A-B: DNA329108, 1383643.16, 201368_at
FIG. 418: PRO84755
FIG. 419: DNA329107, ZFP36L2, 201369_s_at
FIG. 420: PRO84754
FIG. 421A-E: DNA331446, NP_000436.1, 201373_at
FIG. 422: PRO86502
FIG. 423: DNA329109, NP_004957.1, 201376_s_at
FIG. 424: PRO81854
FIG. 425: DNA329111, NP_001349.1, 201385_at
FIG. 426: PRO84756
FIG. 427: DNA270979, NP_002800.1, 201388_at
FIG. 428: PRO59309
FIG. 429: DNA331447, NP_006614.1, 201397_at
FIG. 430: PRO85247
FIG. 431: DNA329937, NP_002786.2, 201400_at
FIG. 432: PRO61014
FIG. 433: DNA328412, NP_060428.1, 201411_s_at
FIG. 434: PRO84257
FIG. 435: DNA329938, 1394805.1, 201416_at
FIG. 436: PRO70544
FIG. 437: DNA329939, 1393503.1, 201417_at
FIG. 438: PRO85248
FIG. 439: DNA83109, NP_006323.1, 201422_at
FIG. 440: PRO2592
FIG. 441: DNA226600, NP_003371.1, 201426_s_at
FIG. 442: PRO37063
FIG. 443: DNA272286, NP_001743.1, 201432_at
FIG. 444: PRO60544
FIG. 445: DNA327550, NP_001959.1, 201437_s_at
FIG. 446: PRO81164
FIG. 447A-C: DNA88140, COL6A3, 201438_at
FIG. 448: PRO2670
FIG. 449: DNA150535, NP_004809.1, 201440_at
FIG. 450: PRO12078
FIG. 451: DNA325049, NP_005605.1, 201453_x_at
FIG. 452: PRO37938
FIG. 453: DNA326736, NP_006657.1, 201459_at
FIG. 454: PRO83076
FIG. 455: DNA226359, NP_002219.1, 201464_x_at
FIG. 456: PRO36822
FIG. 457: DNA226359, JUN, 201465_s_at
FIG. 458: PRO36822
FIG. 459: DNA226359, DNA226359, 201466_s_at
FIG. 460: PRO36822
FIG. 461: DNA328413, NP_004823.1, 201470_at
FIG. 462: PRO84258
FIG. 463: DNA103320, NP_002220.1, 201473_at
FIG. 464: PRO4650
FIG. 465: DNA325704, NP_004981.2, 201475_x_at
FIG. 466: PRO82188
FIG. 467: DNA327551, NP_001024.1, 201476_s_at
FIG. 468: PRO59289
FIG. 469: DNA327551, RRM1, 201477_s_at
FIG. 470: PRO59289
FIG. 471: DNA254783, NP_001354.1, 201478_s_at
FIG. 472: PRO49881
FIG. 473: DNA329940, NP_001805.1, 201487_at
FIG. 474: PRO2679
FIG. 475: DNA304459, BC005020, 201489_at
FIG. 476: PRO37073
FIG. 477: DNA304459, NP_005720.1, 201490_s_at
FIG. 478: PRO37073
FIG. 479: DNA325920, NP_036243.1, 201491_at
FIG. 480: PRO82373
FIG. 481: DNA328415, BC006997, 201503_at
FIG. 482: PRO60207
FIG. 483: DNA329941, NP_001543.1, 201508_at
FIG. 484: PRO85249
FIG. 485: DNA323741, NP_003123.1, 201516_at
FIG. 486: PRO80498
FIG. 487: DNA329942, NCBP2, 201521_s_at
FIG. 488: PRO85250
FIG. 489: DNA328418, NP_003398.1, 201531_at
FIG. 490: PRO84261
FIG. 491: DNA331292, NP_002779.1, 201532_at
FIG. 492: PRO84262
FIG. 493: DNA329943, NP_009037.1, 201534_s_at
FIG. 494: PRO85251
FIG. 495: DNA331448, UBL3, 201535_at
FIG. 496: PRO86503
FIG. 497: DNA272171, NP_002379.2, 201555_at
FIG. 498: PRO60438
FIG. 499: DNA226291, NP_055047.1, 201556_s_at
FIG. 500: PRO36754
FIG. 501: DNA226291, VAMP2, 201557_at
FIG. 502: PRO36754
FIG. 503A-B: DNA270995, NP_004721.1, 201574_at
FIG. 504: PRO59324
FIG. 505: DNA227071, NP_000260.1, 201577_at
FIG. 506: PRO37534
FIG. 507: DNA327199, NP_066979.1, 201580_s_at
FIG. 508: PRO83475
FIG. 509A-B: DNA329944, AB032988, 201581_at
FIG. 510: DNA329945, NP_006354.2, 201583_s_at
FIG. 511: PRO85252
FIG. 512A-B: DNA329946, D80000, 201589_at
FIG. 513: DNA290280, NP_004359.1, 201605_x_at
FIG. 514: PRO70425
FIG. 515: DNA329947, NP_536806.1, 201613_s_at
FIG. 516: PRO37674
FIG. 517: DNA272904, NP_006784.1, 201619_at
FIG. 518: PRO60991
FIG. 519: DNA255406, NP_005533.1, 201625_s_at
FIG. 520: PRO50473
FIG. 521: DNA255406, INSIG1, 201627_s_at
FIG. 522: PRO50473
FIG. 523: DNA329115, NP_434702.1, 201631_s_at
FIG. 524: PRO84760
FIG. 525: DNA287240, NP_004326.1, 201641_at
FIG. 526: PRO29371
FIG. 527: DNA327557, NP_004214.1, 201649_at
FIG. 528: PRO83588
FIG. 529A-B: DNA220748, NP_000201.1, 201656_at
FIG. 530: PRO34726
FIG. 531A-B: DNA328422, NP_004448.1,
201662_s_at
FIG. 532: PRO84263
FIG. 533A-B: DNA273732, NP_005487.2,
201663_s_at
FIG. 534: PRO61695
FIG. 535A-B: DNA273732, HSM801845, 201664_at
FIG. 536: PRO61695
FIG. 537: DNA273090, NP_002347.4, 201669_s_at
FIG. 538: PRO61148
FIG. 539: DNA273090, MARCKS, 201670_s_at
FIG. 540: PRO61148
FIG. 541: DNA290244, NP_000261.1, 201695_s_at
FIG. 542: PRO70353
FIG. 543: DNA329948, NP_002797.1, 201699_at
FIG. 544: PRO85253
FIG. 545: DNA324742, NP_001751.1, 201700_at
FIG. 546: PRO81367
FIG. 547: DNA270883, NP_001061.1, 201714_at
FIG. 548: PRO59218
FIG. 549A-B: DNA151806, NP_001422.1,
201719_s_at
FIG. 550: PRO12768
FIG. 551: DNA227461, NP_006753.1, 201720_s_at
FIG. 552: PRO37924
FIG. 553: DNA227461, LAPTM5, 201721_s_at
FIG. 554: PRO37924
FIG. 555: DNA329949, BC003376, 201726_at
FIG. 556: PRO85254
FIG. 557: DNA227576, NP_005618.1, 201739_at
FIG. 558: PRO38039
FIG. 559: DNA326373, NP_008855.1, 201742_x_at
FIG. 560: PRO82769
FIG. 561: DNA327559, NP_058432.1, 201752_s_at
FIG. 562: PRO83589
FIG. 563: DNA331294, ADD3, 201753_s_at
FIG. 564: PRO86393
FIG. 565: DNA227035, NP_006730.1, 201755_at
FIG. 566: PRO37498
FIG. 567: DNA329016, NP_006283.1, 201758_at
FIG. 568: PRO4887
FIG. 569: DNA328427, NP_061109.1, 201760_s_at
FIG. 570: PRO84265
FIG. 571: DNA287167, NP_006627.1, 201761_at
FIG. 572: PRO59136
FIG. 573: DNA287625, NP_002809.1, 201762_s_at
FIG. 574: PRO69491
FIG. 575: DNA329950, NP_076961.1, 201764_at
FIG. 576: PRO11558
FIG. 577A-B: DNA329951, NP_055680.1,
201774_s_at
FIG. 578: PRO85255
FIG. 579: DNA151017, NP_004835.1, 201810_s_at
FIG. 580: PRO12841
FIG. 581: DNA151017, SH3BP5, 201811_x_at
FIG. 582: PRO12841
FIG. 583: DNA227929, NP_061932.1, 201812_s_at
FIG. 584: PRO38392
FIG. 585: DNA324015, NP_006326.1, 201821_s_at
FIG. 586: PRO80735
FIG. 587: DNA329952, BC010285, 201829_at
FIG. 588: PRO85256
FIG. 589: DNA329952, NET1, 201830_s_at
FIG. 590: PRO85256
FIG. 591: DNA329954, NP_001518.1, 201833_at
FIG. 592: PRO85258
FIG. 593A-B: DNA329955, AB029551, 201845_s_at
FIG. 594: PRO85259
FIG. 595: DNA254350, NP_004043.2, 201848_s_at
FIG. 596: PRO49461
FIG. 597: DNA254350, BNIP3, 201849_at
FIG. 598: PRO49461
FIG. 599: DNA329118, NP_068660.1, 201853_s_at
FIG. 600: PRO83123
FIG. 601: DNA272066, NP_002931.1, 201872_s_at
FIG. 602: PRO60337
FIG. 603: DNA150805, NP_055703.1, 201889_at
FIG. 604: PRO11583
FIG. 605: DNA253582, DNA253582, 201890_at
FIG. 606: PRO49181
FIG. 607: DNA329956, NP_000875.1, 201892_s_at
FIG. 608: PRO85260
FIG. 609: DNA328431, NP_001817.1, 201897_s_at
FIG. 610: PRO45093
FIG. 611: DNA254978, NP_060625.1, 201917_s_at
FIG. 612: PRO50067
FIG. 613: DNA329057, NP_004116.2, 201921_at
FIG. 614: PRO84719
FIG. 615: DNA227112, NP_006397.1, 201923_at
FIG. 616: PRO37575
FIG. 617: DNA275240, NP_005906.2, 201930_at
FIG. 618: PRO62927
FIG. 619: DNA273014, NP_00117.1, 201931_at
FIG. 620: PRO61085
FIG. 621A-B: DNA329120, NP_002560.1, 201945_at
FIG. 622: PRO2752
FIG. 623: DNA274167, NP_006422.1, 201946_s_at
FIG. 624: PRO62097
FIG. 625: DNA274167, CCT2, 201947_s_at
FIG. 626: PRO62097
FIG. 627: DNA103481, NP_037417.1, 201948_at
FIG. 628: PRO4808
FIG. 629A-B: DNA327563, NP_066945.1, 201963_at
FIG. 630: PRO83592
FIG. 631: DNA275214, NP_002473.1, 201970_s_at
FIG. 632: PRO62908
FIG. 633A-B: DNA328433, ATP6V1A1,
201971_s_at
FIG. 634: PRO84268
FIG. 635A-B: DNA272191, NP_002947.1, 201975_at
FIG. 636: PRO60456
FIG. 637: DNA328809, PTPN12, 202006_at
FIG. 638: PRO4803
FIG. 639: DNA328437, AF083441, 202021_x_at
FIG. 640: PRO84271
FIG. 641: DNA329957, NP_005156.1, 202022_at
FIG. 642: PRO85261
FIG. 643A-B: DNA329958, NP_510880.1, 202039_at
FIG. 644: PRO85262
FIG. 645: DNA327017, NP_004586.2, 202043_s_at
FIG. 646: PRO61744
FIG. 647A-B: DNA227985, NP_055107.1,
202047_s_at
FIG. 648: PRO38448
FIG. 649A-B: DNA225991, NP_000518.1,
202067_s_at
FIG. 650: PRO36454
FIG. 651A-B: DNA225991, LDLR, 202068_s_at
FIG. 652: PRO36454
FIG. 653: DNA327567, NP_005521.1, 202069_s_at
FIG. 654: PRO83596
FIG. 655: DNA226116, NP_002990.1, 202071_at
FIG. 656: PRO36579
FIG. 657: DNA289522, NP_004994.1, 202077_at
FIG. 658: PRO70276
FIG. 659: DNA327568, NP_002453.1, 202086_at
FIG. 660: PRO57922
FIG. 661: DNA327569, CTSL, 202087_s_at
FIG. 662: PRO2683
FIG. 663: DNA329959, 251651.5, 202094_at
FIG. 664: PRO85263
FIG. 665: DNA129504, NP_001159.1, 202095_s_at
FIG. 666: PRO7143
FIG. 667: DNA328440, NP_004517.1, 202107_s_at
FIG. 668: PRO84274
FIG. 669: DNA329960, 1381890.1, 202136_at
FIG. 670: PRO85264
FIG. 671: DNA324895, NP_006294.2, 202138_x_at
FIG. 672: PRO81501
FIG. 673: DNA227150, NP_002337.1, 202145_at
FIG. 674: PRO37613
FIG. 675: DNA329020, NP_057637.1, 202153_s_at
FIG. 676: PRO84695
FIG. 677: DNA328442, NP_006078.2, 202154_x_at
FIG. 678: PRO84275
FIG. 679A-C: DNA331449, NP_004371.1, 202160_at
FIG. 680: PRO86504
FIG. 681: DNA327573, BC007655, 202165_at
FIG. 682: PRO59301
FIG. 683: DNA329962, AASDHPPT, 202170_s_at
FIG. 684: PRO85266
FIG. 685A-B: DNA329963, NP_060700.1,
202184_s_at
FIG. 686: PRO85267
FIG. 687: DNA254570, NP_055484.1, 202188_at
FIG. 688: PRO49673
FIG. 689A-B: DNA304479, BC016556, 202194_at
FIG. 690: PRO733
FIG. 691A-B: DNA329599, NP_003128.2,
202200_s_at
FIG. 692: PRO85131
FIG. 693A-B: DNA329964, 215949.9, 202206_at
FIG. 694: PRO85268
FIG. 695: DNA329965, BC001051, 202208_s_at
FIG. 696: PRO85269
FIG. 697: DNA325477, NP_004256.1, 202218_s_at
FIG. 698: PRO12878
FIG. 699: DNA328258, SLC16A1, 202236_s_at
FIG. 700: PRO84151
FIG. 701: DNA326133, NP_005021.2, 202240_at
FIG. 702: PRO82557
FIG. 703: DNA328444, MGC14458, 202246_s_at
FIG. 704: PRO84277
FIG. 705A-B: DNA227176, NP_056371.1,
202255_s_at
FIG. 706: PRO37639
FIG. 707: DNA326120, NP_006101.1, 202257_s_at
FIG. 708: PRO82546
FIG. 709: DNA150808, NP_002044.1, 202269_x_at
FIG. 710: PRO12478
FIG. 711: DNA150808, GBP1, 202270_at
FIG. 712: PRO12478
FIG. 713: DNA329966, NP_006295.1, 202276_at
FIG. 714: PRO22705
FIG. 715: DNA304716, NP_510867.1, 202284_s_at
FIG. 716: PRO71142
FIG. 717: DNA331450, NP_004381.1, 202295_s_at
FIG. 718: PRO2682
FIG. 719A-B: DNA329967, NP_003592.2,
202303_x_at
FIG. 720: PRO85270
FIG. 721: DNA329524, NP_000584.2, 202307_s_at
FIG. 722: PRO36996
FIG. 723A-B: DNA151108, NP_004167.3, 202308_at
FIG. 724: PRO12105
FIG. 725: DNA270142, NP_005947.2, 202309_at
FIG. 726: PRO58531
FIG. 727: DNA269842, NP_002708.1, 202313_at
FIG. 728: PRO58243
FIG. 729: DNA328448, NP_000777.1, 202314_at
FIG. 730: PRO62362
FIG. 731: DNA331451, UNG, 202330_s_at
FIG. 732: PRO86505
FIG. 733A-B: DNA329970, NP_000910.2,
202336_s_at
FIG. 734: PRO85272
FIG. 735: DNA255088, NP_003249.1, 202338_at
FIG. 736: PRO50174
FIG. 737: DNA325115, NP_001435.1, 202345_s_at
FIG. 738: PRO81689
FIG. 739: DNA270502, NP_002807.1, 202352_s_at
FIG. 740: PRO58880
FIG. 741A-B: DNA227353, NP_055637.1, 202375_at
FIG. 742: PRO37816
FIG. 743: DNA328449, NP_005462.1, 202382_s_at
FIG. 744: PRO60304
FIG. 745: DNA290234, NP_002914.1, 202388_at
FIG. 746: PRO70333
FIG. 747: DNA325417, NP_001742.1, 202402_s_at
FIG. 748: PRO69635
FIG. 749: DNA150989, NP_005523.1, 202411_at
FIG. 750: PRO12569
FIG. 751: DNA326563, NP_036421.2, 202417_at
FIG. 752: PRO82927
FIG. 753: DNA150514, NP_065203.1, 202418_at
FIG. 754: PRO12304
FIG. 755: DNA88332, NP_002026.1, 202419_at
FIG. 756: PRO2753
FIG. 757A-B: DNA329971, NP_075266.1,
202422_s_at
FIG. 758: PRO85273
FIG. 759: DNA227121, NP_066928.1, 202430_s_at
FIG. 760: PRO37584
FIG. 761: DNA66487, NP_002458.1, 202431_s_at
FIG. 762: PRO1213
FIG. 763: DNA103322, NP_005818.1, 202433_at
FIG. 764: PRO4652
FIG. 765: DNA68868, DNA68868, 202441_at
FIG. 766: PRO1460
FIG. 767: DNA227121, PLSCR1, 202446_s_at
FIG. 768: PRO37584
FIG. 769: DNA329972, BC004452, 202451_at
FIG. 770: PRO85274
FIG. 771A-B: DNA329973, NP_055461.1,
202459_s_at
FIG. 772: PRO82824
FIG. 773A-B: DNA269642, NP_004557.1,
202464_s_at
FIG. 774: PRO58054
FIG. 775: DNA227921, NP_003789.1, 202468_s_at
FIG. 776: PRO38384
FIG. 777A-B: DNA329122, NP_067675.1, 202478_at
FIG. 778: PRO84764
FIG. 779: DNA329123, NP_002873.1, 202483_s_at
FIG. 780: PRO84765
FIG. 781A-B: DNA103449, NP_008862.1,
202498_s_at
FIG. 782: PRO4776
FIG. 783: DNA329974, NP_055083.1, 202501_at
FIG. 784: PRO85275
FIG. 785: DNA234442, NP_055551.1, 202503_s_at
FIG. 786: PRO38852
FIG. 787A-B: DNA273879, NP_055753.1, 202519_at
FIG. 788: PRO61835
FIG. 789A-B: DNA277809, NP_055582.1,
202524_s_at
FIG. 790: PRO64556
FIG. 791: DNA328452, NP_000394.1, 202528_at
FIG. 792: PRO63289
FIG. 793A-B: DNA226870, DHFR, 202532_s_at
FIG. 794: PRO37333
FIG. 795: DNA331452, BC003584, 202533_s_at
FIG. 796: PRO86506
FIG. 797: DNA331453, NP_060993.1, 202534_x_at
FIG. 798: PRO69586
FIG. 799: DNA329976, NP_003815.1, 202535_at
FIG. 800: PRO4801
FIG. 801: DNA329977, BC001553, 202536_at
FIG. 802: PRO85276
FIG. 803A-B: DNA255105, NP_000850.1,
202539_s_at
FIG. 804: PRO50187
FIG. 805A-B: DNA255105, HMGCR, 202540_s_at
FIG. 806: PRO50187
FIG. 807A-B: DNA274852, NP_004115.1,
202543_s_at
FIG. 808: PRO62605
FIG. 809: DNA275244, DNA275244, 202557_at
FIG. 810A-C: DNA331454, NP_068506.1,
202565_s_at
FIG. 811: PRO86507
FIG. 812A-C: DNA329978, SVIL, 202566_s_at
FIG. 813: PRO85277
FIG. 814: DNA326939, NP_004166.1, 202567_at
FIG. 815: PRO83257
FIG. 816: DNA325587, NP_068772.1, 202580_x_at
FIG. 817: PRO82083
FIG. 818: DNA227607, HSPA1B, 202581_at
FIG. 819: PRO38070
FIG. 820: DNA328456, NP_000467.1, 202587_s_at
FIG. 821: PRO84283
FIG. 822: DNA329979, NP_001062.1, 202589_at
FIG. 823: PRO82821
FIG. 824: DNA329125, NP_056159.1, 202595_s_at
FIG. 825: PRO84767
FIG. 826A-C: DNA270287, NP_003480.1,
202599_s_at
FIG. 827: PRO58675
FIG. 828A-C: DNA270287, NRIP1, 202600_s_at
FIG. 829: PRO58675
FIG. 830A-C: DNA329268, NP_004220.1,
202610_s_at
FIG. 831: PRO84864
FIG. 832: DNA274881, NP_001896.1, 202613_at
FIG. 833: PRO62626
FIG. 834A-B: DNA329980, 1134366.16, 202615_at
FIG. 835: PRO85278
FIG. 836: DNA329126, NP_005025.1, 202635_s_at
FIG. 837: PRO84768
FIG. 838: DNA59763, NP_000192.1, 202637_s_at
FIG. 839: PRO160
FIG. 840: DNA59763, ICAM1, 202638_s_at
FIG. 841: PRO160
FIG. 842: DNA289528, NP_004302.1, 202641_at
FIG. 843: PRO70286
FIG. 844A-B: DNA151841, NP_006281.1,
202643_s_at
FIG. 845: PRO12904
FIG. 846A-B: DNA151841, TNFAIP3, 202644_s_at
FIG. 847: PRO12904
FIG. 848: DNA329981, NP_001155.1, 202652_at
FIG. 849: PRO49894
FIG. 850: DNA254129, NP_006001.1, 202655_at
FIG. 851: PRO49244
FIG. 852: DNA331455, NP_002792.1, 202659_at
FIG. 853: PRO58763
FIG. 854: DNA287424, NP_004292.1, 202666_s_at
FIG. 855: PRO69681
FIG. 856: DNA326896, NP_003672.1, 202671_s_at
FIG. 857: PRO69486
FIG. 858: DNA289526, ATF3, 202672_s_at
FIG. 859: PRO70282
FIG. 860: DNA84130, NP_003801.1, 202687_s_at
FIG. 861: PRO1096
FIG. 862: DNA84130, TNFSF10, 202688_at
FIG. 863: PRO1096
FIG. 864: DNA329982, NP_008937.1, 202697_at
FIG. 865: PRO85279
FIG. 866A-B: DNA150467, NP_055513.1,
202699_s_at
FIG. 867: PRO12272
FIG. 868A-B: DNA150467, KIAA0792, 202700_s_at
FIG. 869: PRO12272
FIG. 870: DNA326000, NP_004692.1, 202705_at
FIG. 871: PRO82442
FIG. 872: DNA273371, NP_000364.1, 202706_s_at
FIG. 873: PRO61373
FIG. 874: DNA329983, BC012595, 202710_at
FIG. 875: PRO85280
FIG. 876: DNA43010, NP_000588.1, 202718_at
FIG. 877: PRO36145
FIG. 878A-B: DNA270254, NP_002006.2,
202723_s_at
FIG. 879: PRO58642
FIG. 880: DNA150713, NP_006570.1, 202735_at
FIG. 881: PRO12082
FIG. 882: DNA58828, DNA58828, 202746_at
FIG. 883: PRO1189
FIG. 884: DNA327192, NP_004858.1, 202747_s_at
FIG. 885: PRO1189
FIG. 886: DNA227133, NP_004111.1, 202748_at
FIG. 887: PRO37596
FIG. 888: DNA329984, NP_004618.2, 202749_at
FIG. 889: PRO11656
FIG. 890A-C: DNA329129, NP_009134.1,
202760_s_at
FIG. 891: PRO84288
FIG. 892: DNA329008, NP_004337.2, 202763_at
FIG. 893: PRO12832
FIG. 894A-B: DNA328464, 977954.20, 202769_at
FIG. 895: PRO84290
FIG. 896: DNA226578, NP_004345.1, 202770_s_at
FIG. 897: PRO37041
FIG. 898: DNA273346, NP_055316.1, 202779_s_at
FIG. 899: PRO61349
FIG. 900: DNA329985, NP_002185.1, 202794_at
FIG. 901: PRO60589
FIG. 902: DNA88428, NP_000202.1, 202803_s_at
FIG. 903: PRO2787
FIG. 904: DNA329986, NP_006454.1, 202811_at
FIG. 905: PRO61895
FIG. 906A-B: DNA226364, NP_001612.1, 202820_at
FIG. 907: PRO36827
FIG. 908: DNA328465, NP_005639.1, 202823_at
FIG. 909: PRO84291
FIG. 910: DNA329987, NP_000286.2, 202833_s_at
FIG. 911: PRO85281
FIG. 912: DNA269828, FLN29, 202837_at
FIG. 913: PRO58230
FIG. 914: DNA329988, NP_036460.1, 202842_s_at
FIG. 915: PRO1471
FIG. 916: DNA329988, DNAJB9, 202843_at
FIG. 917: PRO1471
FIG. 918: DNA103394, NP_004198.1, 202855_s_at
FIG. 919: PRO4722
FIG. 920: DNA103394, SLC16A3, 202856_s_at
FIG. 921: PRO4722
FIG. 922A-B: DNA272022, PER1, 202861_at
FIG. 923: PRO60296
FIG. 924: DNA275144, NP_000128.1, 202862_at
FIG. 925: PRO62852
FIG. 926: DNA328467, SP100, 202864_s_at
FIG. 927: PRO84293
FIG. 928: DNA287289, NP_058132.1, 202869_at
FIG. 929: PRO69559
FIG. 930: DNA273060, NP_001246.1, 202870_s_at
FIG. 931: PRO61125
FIG. 932: DNA329130, NP_004286.2, 202871_at
FIG. 933: PRO20124
FIG. 934: DNA328469, NP_001686.1, 202874_s_at
FIG. 935: PRO84295
FIG. 936: DNA271881, PSCD1, 202880_s_at
FIG. 937: PRO60160
FIG. 938: DNA329989, HSPPP2R15, 202886_s_at
FIG. 939A-B: DNA225538, NP_002476.1,
202906_s_at
FIG. 940: PRO36001
FIG. 941A-B: DNA225538, NBS1, 202907_s_at
FIG. 942: PRO36001
FIG. 943: DNA328483, NP_061163.1, 202911_at
FIG. 944: PRO84309
FIG. 945: DNA327584, NP_002955.2, 202917_s_at
FIG. 946: PRO80649
FIG. 947A-B: DNA329132, NP_002648.1,
202925_s_at
FIG. 948: PRO83145
FIG. 949: DNA272979, NP_003841.1, 202930_s_at
FIG. 950: PRO61058
FIG. 951: DNA331456, BIN1, 202931_x_at
FIG. 952: PRO86508
FIG. 953: DNA327585, NP_056518.1, 202937_x_at
FIG. 954: PRO83605
FIG. 955: DNA328471, ZMPSTE24, 202939_at
FIG. 956: PRO84297
FIG. 957: DNA304681, NP_066552.1, 202941_at
FIG. 958: PRO71107
FIG. 959: DNA269481, NP_001976.1, 202942_at
FIG. 960: PRO57901
FIG. 961: DNA273320, NP_008950.1, 202954_at
FIG. 962: PRO61327
FIG. 963: DNA273334, NP_000246.1, 202960_s_at
FIG. 964: PRO61341
FIG. 965A-B: DNA328473, NP_006473.1,
202968_s_at
FIG. 966: PRO84299
FIG. 967A-B: DNA227293, NP_055698.1,
202972_s_at
FIG. 968: PRO37756
FIG. 969A-B: DNA227293, KIAA0914, 202973_x_at
FIG. 970: PRO37756
FIG. 971: DNA329135, NP_002913.2, 202988_s_at
FIG. 972: PRO58102
FIG. 973: DNA274034, NP_006388.1, 203022_at
FIG. 974: PRO61977
FIG. 975: DNA329136, NP_057475.1, 203023_at
FIG. 976: PRO84772
FIG. 977A-B: DNA271865, NP_055566.1,
203037_s_at
FIG. 978: PRO60145
FIG. 979A-B: DNA304464, NP_055733.1, 203044_at
FIG. 980: PRO71042
FIG. 981A-B: DNA329991, NP_003911.1,
203046_s_at
FIG. 982: PRO85284
FIG. 983: DNA331457, AF119894, 203047_at
FIG. 984: PRO86509
FIG. 985: DNA150976, NP_071503.1, 203054_s_at
FIG. 986: PRO12565
FIG. 987: DNA326693, NP_004697.2, 203055_s_at
FIG. 988: PRO83039
FIG. 989: DNA188357, NP_000651.1, 203085_s_at
FIG. 990: PRO21897
FIG. 991: DNA324133, NP_037379.1, 203089_s_at
FIG. 992: PRO80835
FIG. 993: DNA269984, NP_055443.1, 203094_at
FIG. 994: PRO58380
FIG. 995: DNA329992, NP_002399.1, 203102_s_at
FIG. 996: PRO59267
FIG. 997: DNA329993, NP_115754.1, 203113_s_at
FIG. 998: PRO85285
FIG. 999: DNA329994, PCSK7, 203118_at
FIG. 1000: PRO85286
FIG. 1001A-B: DNA150447, NP_004854.1,
203128_at
FIG. 1002: PRO12256
FIG. 1003: DNA254543, NP_006799.1, 203133_at
FIG. 1004: PRO49648
FIG. 1005: DNA269918, NP_003633.1, 203138_at
FIG. 1006: PRO58316
FIG. 1007: DNA329001, BCL6, 203140_at
FIG. 1008: PRO26296
FIG. 1009A-B: DNA329995, NP_006452.1,
203145_at
FIG. 1010: PRO85287
FIG. 1011A-B: DNA226330, NP_001461.1,
203146_s_at
FIG. 1012: PRO36793
FIG. 1013: DNA271624, NP_001539.1, 203153_at
FIG. 1014: PRO59911
FIG. 1015: DNA269660, NP_003192.1, 203177_x_at
FIG. 1016: PRO58071
FIG. 1017: DNA304720, NP_062427.1, 203186_s_at
FIG. 1018: PRO71146
FIG. 1019A-B: DNA271744, NP_055476.1,
203206_at
FIG. 1020: PRO60028
FIG. 1021: DNA329997, BC001866, 203209_at
FIG. 1022: PRO61115
FIG. 1023: DNA329997, NP_031396.1, 203210_s_at
FIG. 1024: PRO61115
FIG. 1025A-B: DNA328481, MTMR2, 203211_s_at
FIG. 1026: PRO84307
FIG. 1027: DNA331458, 995529.4, 203213_at
FIG. 1028: PRO86510
FIG. 1029: DNA331459, CDC2, 203214_x_at
FIG. 1030: PRO70806
FIG. 1031: DNA76514, NP_000409.1, 203233_at
FIG. 1032: PRO2540
FIG. 1033: DNA325507, NP_005842.1, 203252_at
FIG. 1034: PRO69461
FIG. 1035: DNA330000, NP_036277.1, 203270_at
FIG. 1036: PRO85289
FIG. 1037: DNA302020, NP_005564.1, 203276_at
FIG. 1038: PRO70993
FIG. 1039: DNA328486, NP_000149.1, 203282_at
FIG. 1040: PRO60119
FIG. 1041A-B: DNA330001, NP_036394.1,
203285_s_at
FIG. 1042: PRO85290
FIG. 1043: DNA225675, NP_005561.1, 203293_s_at
FIG. 1044: PRO36138
FIG. 1045: DNA330002, BC007195, 203315_at
FIG. 1046: PRO80853
FIG. 1047A-B: DNA330003, NP_005532.1,
203331_s_at
FIG. 1048: PRO85291
FIG. 1049: DNA330004, NP_055785.2, 203333_at
FIG. 1050: PRO85292
FIG. 1051: DNA330005, NP_003696.2, 203340_s_at
FIG. 1052: PRO85293
FIG. 1053: DNA271959, NP_002885.1, 203344_s_at
FIG. 1054: PRO60234
FIG. 1055: DNA330006, NP_031384.1, 203347_s_at
FIG. 1056: PRO85294
FIG. 1057: DNA330007, NP_055111.1, 203357_s_at
FIG. 1058: PRO85295
FIG. 1059: DNA330008, NP_004447.2, 203358_s_at
FIG. 1060: PRO85296
FIG. 1061: DNA272449, NP_036465.1, 203360_s_at
FIG. 1062: PRO60698
FIG. 1063: DNA324514, NP_002349.1, 203362_s_at
FIG. 1064: PRO81169
FIG. 1065: DNA325749, NP_003868.1, 203372_s_at
FIG. 1066: PRO12839
FIG. 1067: DNA325749, STATI2, 203373_at
FIG. 1068: PRO12839
FIG. 1069: DNA274960, NP_008856.1, 203380_x_at
FIG. 1070: PRO62694
FIG. 1071: DNA151022, NP_001336.1, 203385_at
FIG. 1072: PRO12096
FIG. 1073: DNA331460, NP_002780.1, 203396_at
FIG. 1074: PRO60499
FIG. 1075: DNA326892, NP_003711.1, 203405_at
FIG. 1076: PRO83213
FIG. 1077: DNA274778, NP_005917.1, 203406_at
FIG. 1078: PRO62545
FIG. 1079: DNA270134, NP_000098.1, 203409_at
FIG. 1080: PRO58523
FIG. 1081: DNA28759, NP_006150.1, 203413_at
FIG. 1082: PRO2520
FIG. 1083: DNA287267, NP_001228.1, 203418_at
FIG. 1084: PRO37015
FIG. 1085A-B: DNA256807, NP_057339.1,
203420_at
FIG. 1086: PRO51738
FIG. 1087: DNA326745, NP_002682.1, 203422_at
FIG. 1088: PRO83083
FIG. 1089: DNA330009, NP_054753.1, 203428_s_at
FIG. 1090: PRO85297
FIG. 1091A-B: DNA275186, DNA275186,
203432_at
FIG. 1092A-B: DNA330010, NP_005721.2,
203445_s_at
FIG. 1093: PRO85298
FIG. 1094: DNA273410, NP_004036.1, 203454_s_at
FIG. 1095: PRO61409
FIG. 1096: DNA328495, NP_055578.1, 203465_at
FIG. 1097: PRO58967
FIG. 1098A-C: DNA331461, NP_005493.2,
203504_s_at
FIG. 1099: PRO86511
FIG. 1100A-B: DNA331462, NP_003096.1,
203509_at
FIG. 1101: PRO86512
FIG. 1102: DNA272911, NP_006545.1, 203517_at
FIG. 1103: PRO60997
FIG. 1104A-D: DNA331463, NP_000072.1,
203518_at
FIG. 1105: PRO86513
FIG. 1106A-C: DNA331464, NP_055160.1,
203520_s_at
FIG. 1107: PRO86514
FIG. 1108A-C: DNA330014, HRIHFB2436,
203521_s_at
FIG. 1109: PRO85302
FIG. 1110: DNA325404, NP_002330.1, 203523_at
FIG. 1111: PRO81936
FIG. 1112: DNA323910, NP_002956.1, 203535_at
FIG. 1113: PRO80648
FIG. 1114A-B: DNA272399, NP_001197.1,
203542_s_at
FIG. 1115: PRO60653
FIG. 1116A-B: DNA272399, BTEB1, 203543_s_at
FIG. 1117: PRO60653
FIG. 1118: DNA324684, NP_004210.1, 203554_x_at
FIG. 1119: PRO81319
FIG. 1120: DNA330015, NP_004620.1, 203564_at
FIG. 1121: PRO58704
FIG. 1122: DNA330016, NP_006346.1, 203567_s_at
FIG. 1123: PRO85303
FIG. 1124A-B: DNA150765, NP_003974.1,
203579_s_at
FIG. 1125: PRO12458
FIG. 1126: DNA273676, NP_055488.1, 203584_at
FIG. 1127: PRO61644
FIG. 1128: DNA271003, NP_003720.1, 203594_at
FIG. 1129: PRO59332
FIG. 1130A-B: DNA270323, NP_036552.1,
203595_s_at
FIG. 1131: PRO58710
FIG. 1132A-B: DNA270323, RI58, 203596_s_at
FIG. 1133: PRO58710
FIG. 1134: DNA330017, NP_009118.1, 203597_s_at
FIG. 1135: PRO60916
FIG. 1136: DNA329604, NP_003127.1, 203605_at
FIG. 1137: PRO85134
FIG. 1138: DNA287246, NP_004044.2, 203612_at
FIG. 1139: PRO69521
FIG. 1140: DNA330018, NP_064528.1, 203622_s_at
FIG. 1141: PRO85304
FIG. 1142: DNA331465, SKP2, 203625_x_at
FIG. 1143: PRO81225
FIG. 1144A-B: DNA327596, 345314.2, 203628_at
FIG. 1145: PRO1920
FIG. 1146A-B: DNA331466, BCL2, 203685_at
FIG. 1147: PRO86515
FIG. 1148A-B: DNA330021, NP_001940.1,
203693_s_at
FIG. 1149: PRO85306
FIG. 1150: DNA329900, RFC2, 203696_s_at
FIG. 1151: PRO81549
FIG. 1152A-C: DNA331467, NP_002213.1,
203710_at
FIG. 1153: PRO86516
FIG. 1154: DNA329144, KIAA0020, 203712_at
FIG. 1155: PRO84779
FIG. 1156: DNA326402, NP_004515.1, 203713_s_at
FIG. 1157: PRO82793
FIG. 1158: DNA324183, DPP4, 203716_s_at
FIG. 1159: PRO80881
FIG. 1160: DNA150784, NP_001974.1, 203720_s_at
FIG. 1161: PRO12800
FIG. 1162A-B: DNA269573, NP_002212.1,
203723_at
FIG. 1163: PRO57986
FIG. 1164: DNA330023, NP_001915.1, 203725_at
FIG. 1165: PRO85308
FIG. 1166: DNA227020, NP_001416.1, 203729_at
FIG. 1167: PRO37483
FIG. 1168A-B: DNA325369, NP_055877.2,
203737_s_at
FIG. 1169: PRO81905
FIG. 1170A-B: DNA150748, NP_001105.1,
203741_s_at
FIG. 1171: PRO12446
FIG. 1172: DNA327523, AQP3, 203747_at
FIG. 1173: PRO38028
FIG. 1174: DNA330024, NP_058521.1, 203748_x_at
FIG. 1175: PRO85309
FIG. 1176: DNA97279, NP_005345.2, 203751_x_at
FIG. 1177: PRO3628
FIG. 1178A-B: DNA325972, BUB1B, 203755_at
FIG. 1179: PRO82417
FIG. 1180: DNA330025, NP_055565.2, 203764_at
FIG. 1181: PRO85310
FIG. 1182: DNA330026, NP_005899.1, 203778_at
FIG. 1183: PRO85311
FIG. 1184: DNA330027, NP_036578.1, 203787_at
FIG. 1185: PRO85312
FIG. 1186A-B: DNA150954, NP_055695.1,
203799_at
FIG. 1187: PRO12558
FIG. 1188: DNA331468, DGUOK, 203816_at
FIG. 1189: PRO86517
FIG. 1190: DNA274125, NP_071739.1, 203830_at
FIG. 1191: PRO62061
FIG. 1192A-B: DNA331469, 094680.4, 203845_at
FIG. 1193: PRO86518
FIG. 1194A-B: DNA325529, GAB2, 203853_s_at
FIG. 1195: PRO82037
FIG. 1196A-B: DNA275079, NP_056648.1,
203865_s_at
FIG. 1197: PRO62797
FIG. 1198: DNA275339, NP_005685.1, 203880_at
FIG. 1199: PRO63012
FIG. 1200: DNA329034, NP_006075.2, 203882_at
FIG. 1201: PRO84701
FIG. 1202A-B: DNA288692, NP_055719.1,
203884_s_at
FIG. 1203: PRO70078
FIG. 1204: DNA328513, TAF9, 203893_at
FIG. 1205: PRO37815
FIG. 1206A-B: DNA330030, NP_055684.1,
203907_s_at
FIG. 1207: PRO85315
FIG. 1208: DNA82376, NP_002407.1, 203915_at
FIG. 1209: PRO1723
FIG. 1210: DNA271676, NP_002052.1, 203925_at
FIG. 1211: PRO59961
FIG. 1212: DNA288249, NP_002940.1, 203931_s_at
FIG. 1213: PRO69507
FIG. 1214: DNA330031, NP_057210.1, 203960_s_at
FIG. 1215: PRO85316
FIG. 1216: DNA275012, NP_004679.1, 203964_at
FIG. 1217: PRO62740
FIG. 1218: DNA272338, NP_001245.1, 203967_at
FIG. 1219: PRO60595
FIG. 1220: DNA272338, CDC6, 203968_s_at
FIG. 1221: PRO60595
FIG. 1222: DNA227232, NP_001850.1, 203971_at
FIG. 1223: PRO37695
FIG. 1224: DNA271374, NP_005474.1, 203976_s_at
FIG. 1225: PRO59673
FIG. 1226: DNA226133, NP_001983.1, 203989_x_at
FIG. 1227: PRO36596
FIG. 1228: DNA225915, NP_000561.1, 204006_s_at
FIG. 1229: PRO36378
FIG. 1230: DNA330032, HUMGCRFC, 204007_at
FIG. 1231: PRO85317
FIG. 1232: DNA329145, DUSP4, 204014_at
FIG. 1233: PRO84780
FIG. 1234: DNA331470, HSU48807, 204015_s_at
FIG. 1235: PRO86519
FIG. 1236: DNA326089, NP_000508.1, 204018_x_at
FIG. 1237: PRO3629
FIG. 1238: DNA330033, NP_056492.1, 204019_s_at
FIG. 1239: PRO85318
FIG. 1240: DNA330034, NP_002907.1, 204023_at
FIG. 1241: PRO85319
FIG. 1242: DNA328271, NP_008988.2, 204026_s_at
FIG. 1243: PRO81868
FIG. 1244: DNA330035, NP_004228.1, 204033_at
FIG. 1245: PRO85320
FIG. 1246: DNA325181, CLTA, 204050_s_at
FIG. 1247: PRO81742
FIG. 1248: DNA226342, NP_000305.1, 204054_at
FIG. 1249: PRO36805
FIG. 1250A-B: DNA331471, NP_055498.1,
204063_s_at
FIG. 1251: PRO61468
FIG. 1252: DNA274783, NP_006272.1, 204068_at
FIG. 1253: PRO62549
FIG. 1254A-C: DNA331472, NP_075463.1,
204072_s_at
FIG. 1255: PRO86520
FIG. 1256: DNA270476, NP_003591.1, 204092_s_at
FIG. 1257: PRO58855
FIG. 1258: DNA216689, NP_002975.1, 204103_at
FIG. 1259: PRO34276
FIG. 1260: DNA328522, NP_001769.2, 204118_at
FIG. 1261: PRO2696
FIG. 1262: DNA150529, NP_003323.1, 204122_at
FIG. 1263: PRO12313
FIG. 1264: DNA328524, NP_057097.1, 204125_at
FIG. 1265: PRO84336
FIG. 1266: DNA304489, NP_003495.1, 204126_s_at
FIG. 1267: PRO71058
FIG. 1268: DNA330037, BC000149, 204127_at
FIG. 1269: PRO82290
FIG. 1270: DNA325824, NP_002906.1, 204128_s_at
FIG. 1271: PRO82290
FIG. 1272: DNA328525, BC021224, 204131_s_at
FIG. 1273: PRO84337
FIG. 1274: DNA103532, NP_003263.1, 204137_at
FIG. 1275: PRO4859
FIG. 1276: DNA330038, BC016330, 204146_at
FIG. 1277: PRO85322
FIG. 1278: DNA330039, NP_002396.2, 204152_s_at
FIG. 1279: PRO85323
FIG. 1280: DNA330039, MFNG, 204153_s_at
FIG. 1281: PRO85323
FIG. 1282: DNA330040, NP_523240.1, 204159_at
FIG. 1283: PRO59546
FIG. 1284: DNA273694, NP_006092.1, 204162_at
FIG. 1285: PRO61661
FIG. 1286: DNA227116, NP_006738.1, 204164_at
FIG. 1287: PRO37579
FIG. 1288A-B: DNA254376, NP_055778.1,
204166_at
FIG. 1289: PRO49486
FIG. 1290: DNA272655, NP_001818.1, 204170_s_at
FIG. 1291: PRO60781
FIG. 1292: DNA330041, NP_000088.2, 204172_at
FIG. 1293: PRO85324
FIG. 1294: DNA328528, MLC1SA, 204173_at
FIG. 1295: PRO60636
FIG. 1296: DNA329148, NP_056955.1, 204175_at
FIG. 1297: PRO84782
FIG. 1298: DNA226380, NP_001765.1, 204192_at
FIG. 1299: PRO4695
FIG. 1300: DNA271778, NP_068594.1, 204205_at
FIG. 1301: PRO60062
FIG. 1302: DNA330042, HSU16307, 204221_x_at
FIG. 1303: PRO85325
FIG. 1304: DNA150812, NP_006842.1, 204222_s_at
FIG. 1305: PRO12481
FIG. 1306: DNA227514, NP_000152.1, 204224_s_at
FIG. 1307: PRO37977
FIG. 1308: DNA88308, NP_004097.1, 204232_at
FIG. 1309: PRO2739
FIG. 1310: DNA226881, NP_002008.2, 204236_at
FIG. 1311: PRO37344
FIG. 1312: DNA270434, NP_006434.1, 204238_s_at
FIG. 1313: PRO58814
FIG. 1314A-B: DNA287273, NP_006435.1,
204240_s_at
FIG. 1315: PRO69545
FIG. 1316: DNA330043, NP_001789.2, 204252_at
FIG. 1317: PRO85326
FIG. 1318A-B: DNA103527, NP_000367.1,
204253_s_at
FIG. 1319: PRO4854
FIG. 1320A-B: DNA103527, VDR, 204254_s_at
FIG. 1321: PRO4854
FIG. 1322A-B: DNA103527, HUMVDR,
204255_s_at
FIG. 1323: PRO4854
FIG. 1324: DNA228132, NP_076995.1, 204256_at
FIG. 1325: PRO38595
FIG. 1326: DNA226577, NP_071390.1, 204265_s_at
FIG. 1327: PRO37040
FIG. 1328: DNA88643, SGSH, 204293_at
FIG. 1329: PRO2455
FIG. 1330: DNA330044, GTSE1, 204318_s_at
FIG. 1331: PRO85327
FIG. 1332: DNA330045, NP_005943.1, 204326_x_at
FIG. 1333: PRO82583
FIG. 1334: DNA328530, NP_009198.2, 204328_at
FIG. 1335: PRO24118
FIG. 1336: DNA330046, 987987.10, 204334_at
FIG. 1337: PRO85328
FIG. 1338: DNA328531, NP_037542.1, 204348_s_at
FIG. 1339: PRO84338
FIG. 1340: DNA330047, BC005250, 204349_at
FIG. 1341: PRO37777
FIG. 1342A-B: DNA193847, NP_055518.1,
204377_s_at
FIG. 1343: PRO23272
FIG. 1344: DNA328533, NP_003647.1, 204392_at
FIG. 1345: PRO84340
FIG. 1346: DNA226462, NP_002241.1, 204401_at
FIG. 1347: PRO36925
FIG. 1348A-B: DNA330048, AF080255, 204407_at
FIG. 1349: PRO85329
FIG. 1350: DNA327616, NP_075011.1, 204415_at
FIG. 1351: PRO83624
FIG. 1352: DNA331473, NP_000839.1, 204418_x_at
FIG. 1353: PRO60552
FIG. 1354: DNA226286, NP_001657.1, 204425_at
FIG. 1355: PRO36749
FIG. 1356: DNA327617, NP_006811.1, 204439_at
FIG. 1357: PRO83625
FIG. 1358A-B: DNA330049, NP_004514.2,
204444_at
FIG. 1359: PRO85330
FIG. 1360: DNA329150, NP_000689.1, 204446_s_at
FIG. 1361: PRO84783
FIG. 1362: DNA270496, NP_001316.1, 204459_at
FIG. 1363: PRO58875
FIG. 1364: DNA330050, NP_056289.1, 204502_at
FIG. 1365: PRO85331
FIG. 1366: DNA273612, HSU79274, 204521_at
FIG. 1367: PRO61586
FIG. 1368: DNA330051, NP_003431.1, 204523_at
FIG. 1369: PRO85332
FIG. 1370A-B: DNA330052, NP_009227.1,
204531_s_at
FIG. 1371: PRO25103
FIG. 1372: DNA82362, NP_001556.1, 204533_at
FIG. 1373: PRO1718
FIG. 1374A-B: DNA331474, 357276.11, 204552_at
FIG. 1375: PRO86521
FIG. 1376A-B: DNA329036, NP_002451.1,
204562_at
FIG. 1377: PRO84703
FIG. 1378: DNA287284, NP_060943.1, 204565_at
FIG. 1379: PRO59915
FIG. 1380: DNA151910, NP_004906.2, 204567_s_at
FIG. 1381: PRO12754
FIG. 1382A-B: DNA273627, NP_055739.1,
204568_at
FIG. 1383: PRO61599
FIG. 1384: DNA272992, N4BP1, 204601_at
FIG. 1385: PRO61064
FIG. 1386: DNA254157, NP_005245.2, 204618_s_at
FIG. 1387: PRO49271
FIG. 1388: DNA151048, NP_006177.1, 204621_s_at
FIG. 1389: PRO12850
FIG. 1390: DNA151048, NR4A2, 204622_x_at
FIG. 1391: PRO12850
FIG. 1392A-B: DNA330054, NP_004746.1,
204633_s_at
FIG. 1393: PRO85334
FIG. 1394: DNA254470, NP_002488.1, 204641_at
FIG. 1395: PRO49578
FIG. 1396: DNA226182, EDG1, 204642_at
FIG. 1397: PRO36645
FIG. 1398: DNA210121, CDW52, 204661_at
FIG. 1399: PRO33667
FIG. 1400: DNA103526, LRMP, 204674_at
FIG. 1401: PRO4853
FIG. 1402: DNA225974, NP_000864.1, 204683_at
FIG. 1403: PRO36437
FIG. 1404: DNA256295, LRN, 204692_at
FIG. 1405: PRO51339
FIG. 1406: DNA227573, NP_001780.1, 204696_s_at
FIG. 1407: PRO38036
FIG. 1408: DNA329151, NP_004280.3, 204702_s_at
FIG. 1409: PRO84784
FIG. 1410: DNA331475, KNSL5, 204709_s_at
FIG. 1411: PRO86522
FIG. 1412A-B: DNA331476, NP_000121.1,
204713_s_at
FIG. 1413: PRO86523
FIG. 1414A-B: DNA225911, F5, 204714_s_at
FIG. 1415: PRO36374
FIG. 1416A-B: DNA218283, NP_004436.1,
204718_at
FIG. 1417: PRO34335
FIG. 1418A-B: DNA256461, NP_009017.1,
204728_s_at
FIG. 1419: PRO51498
FIG. 1420A-C: DNA274487, NP_055562.1,
204730_at
FIG. 1421: PRO62389
FIG. 1422A-B: DNA83176, NP_003234.1, 204731_at
FIG. 1423: PRO2620
FIG. 1424A-B: DNA325192, NP_038203.1,
204744_s_at
FIG. 1425: PRO81753
FIG. 1426: DNA330057, NP_005941.1, 204745_x_at
FIG. 1427: PRO85337
FIG. 1428: DNA287178, NP_001540.1, 204747_at
FIG. 1429: PRO69467
FIG. 1430: DNA330058, NP_004529.2, 204749_at
FIG. 1431: PRO85338
FIG. 1432: DNA329153, NP_001259.1, 204759_at
FIG. 1433: PRO84786
FIG. 1434: DNA330059, NP_068370.1, 204760_s_at
FIG. 1435: PRO85339
FIG. 1436: DNA330060, NP_002443.2, 204766_s_at
FIG. 1437: PRO85340
FIG. 1438: DNA329154, BC000323, 204767_s_at
FIG. 1439: PRO69568
FIG. 1440: DNA325479, NP_004102.1, 204768_s_at
FIG. 1441: PRO69568
FIG. 1442: DNA328541, NP_004503.1, 204773_at
FIG. 1443: PRO4843
FIG. 1444: DNA329155, NP_000034.1, 204780_s_at
FIG. 1445: PRO1207
FIG. 1446: DNA329155, TNFRSF6, 204781_s_at
FIG. 1447: PRO1207
FIG. 1448: DNA272121, NP_005895.1, 204790_at
FIG. 1449: PRO60391
FIG. 1450A-B: DNA330061, NP_055525.1,
204793_at
FIG. 1451: PRO85341
FIG. 1452: DNA103269, NP_005366.1, 204798_at
FIG. 1453: PRO4599
FIG. 1454: DNA287168, NP_003132.2, 204804_at
FIG. 1455: PRO69460
FIG. 1456: DNA330062, NP_006017.1, 204805_s_at
FIG. 1457: PRO85342
FIG. 1458A-B: DNA329907, ESPL1, 204817_at
FIG. 1459: PRO85224
FIG. 1460: DNA331477, NP_003309.1, 204822_at
FIG. 1461: PRO58276
FIG. 1462: DNA255289, NP_055606.1, 204825_at
FIG. 1463: PRO50363
FIG. 1464A-B: DNA226387, NP_001752.1,
204826_at
FIG. 1465: PRO36850
FIG. 1466: DNA328544, NP_006673.1, 204834_at
FIG. 1467: PRO84347
FIG. 1468A-B: DNA270446, NP_058633.1,
204835_at
FIG. 1469: PRO58825
FIG. 1470: DNA330063, HUMLPTPASE,
204852_s_at
FIG. 1471: PRO85343
FIG. 1472: DNA150598, NP_003541.1, 204857_at
FIG. 1473: PRO12142
FIG. 1474: DNA225661, NP_001944.1, 204858_s_at
FIG. 1475: PRO36124
FIG. 1476A-B: DNA330064, 332518.2, 204886_at
FIG. 1477: PRO85344
FIG. 1478: DNA330065, NP_055079.2, 204887_s_at
FIG. 1479: PRO85345
FIG. 1480: DNA103444, LCK, 204890_s_at
FIG. 1481: PRO4771
FIG. 1482: DNA331478, BC013200, 204891_s_at
FIG. 1483: PRO86524
FIG. 1484: DNA194139, DNA194139, 204897_at
FIG. 1485: PRO23533
FIG. 1486: DNA255326, NP_003855.1, 204900_x_at
FIG. 1487: PRO50396
FIG. 1488: DNA329157, NP_004271.1, 204905_s_at
FIG. 1489: PRO62861
FIG. 1490: DNA329011, NP_005169.1, 204908_s_at
FIG. 1491: PRO4785
FIG. 1492A-B: DNA76503, NP_001549.1, 204912_at
FIG. 1493: PRO2536
FIG. 1494: DNA330066, NP_004520.1, 204917_s_at
FIG. 1495: PRO85346
FIG. 1496: DNA228014, NP_002153.1, 204949_at
FIG. 1497: PRO38477
FIG. 1498: DNA271093, NP_004064.1, 204958_at
FIG. 1499: PRO59417
FIG. 1500: DNA103283, NP_002423.1, 204959_at
FIG. 1501: PRO4613
FIG. 1502: DNA330067, NP_001800.1, 204962_s_at
FIG. 1503: PRO60368
FIG. 1504: DNA287399, NP_058197.1, 204972_at
FIG. 1505: PRO69656
FIG. 1506: DNA269665, NP_002454.1, 204994_at
FIG. 1507: PRO58076
FIG. 1508: DNA331479, 411441.5, 204995_at
FIG. 1509: PRO86525
FIG. 1510: DNA272427, NP_004799.1, 205005_s_at
FIG. 1511: PRO60679
FIG. 1512: DNA272427, NMT2, 205006_s_at
FIG. 1513: PRO60679
FIG. 1514: DNA329534, NP_004615.2, 205019_s_at
FIG. 1515: PRO2904
FIG. 1516: DNA331480, RAD51, 205024_s_at
FIG. 1517: PRO86526
FIG. 1518: DNA329159, NP_005195.2, 205027_s_at
FIG. 1519: PRO4660
FIG. 1520: DNA325061, NP_005208.1, 205033_s_at
FIG. 1521: PRO9980
FIG. 1522: DNA328297, NP_477097.1, 205034_at
FIG. 1523: PRO59418
FIG. 1524A-C: DNA331481, NP_001804.1,
205046_at
FIG. 1525: PRO86527
FIG. 1526: DNA324991, ASNS, 205047_s_at
FIG. 1527: PRO81585
FIG. 1528: DNA271461, NP_000937.1, 205053_at
FIG. 1529: PRO59757
FIG. 1530A-B: DNA220750, NP_002199.2,
205055_at
FIG. 1531: PRO34728
FIG. 1532: DNA330071, NP_003607.1, 205063_at
FIG. 1533: PRO85350
FIG. 1534: DNA330072, NP_071801.1, 205072_s_at
FIG. 1535: PRO85351
FIG. 1536: DNA304705, NP_002634.1, 205078_at
FIG. 1537: PRO71131
FIG. 1538: DNA327632, NP_001302.1, 205081_at
FIG. 1539: PRO83635
FIG. 1540: DNA255336, NP_061332.1, 205084_at
FIG. 1541: PRO50406
FIG. 1542: DNA330073, NP_004144.1, 205085_at
FIG. 1543: PRO85352
FIG. 1544: DNA330074, HUMHM145, 205098_at
FIG. 1545: PRO85353
FIG. 1546: DNA226177, NP_001286.1, 205099_s_at
FIG. 1547: PRO36640
FIG. 1548: DNA192060, NP_002974.1, 205114_s_at
FIG. 1549: PRO21960
FIG. 1550: DNA299899, NP_002148.1, 205133_s_at
FIG. 1551: PRO62760
FIG. 1552: DNA331482, NP_001241.1, 205153_s_at
FIG. 1553: PRO34457
FIG. 1554: DNA330075, CDC25C, 205167_s_at
FIG. 1555: PRO85354
FIG. 1556: DNA330076, NP_005410.1, 205170_at
FIG. 1557: PRO85355
FIG. 1558: DNA328810, NP_001770.1, 205173_x_at
FIG. 1559: PRO2557
FIG. 1560: DNA330077, ITGB3BP, 205176_s_at
FIG. 1561: PRO85356
FIG. 1562: DNA151804, NP_006500.1, 205205_at
FIG. 1563: PRO12188
FIG. 1564: DNA272443, NP_055531.1, 205213_at
FIG. 1565: PRO60693
FIG. 1566: DNA273535, NP_004217.1, 205214_at
FIG. 1567: PRO61515
FIG. 1568: DNA325255, NP_001994.2, 205237_at
FIG. 1569: PRO1910
FIG. 1570: DNA330078, NP_001648.1, 205239_at
FIG. 1571: PRO46
FIG. 1572: DNA327634, NP_005129.1, 205241_at
FIG. 1573: PRO83636
FIG. 1574: DNA188333, NP_006410.1, 205242_at
FIG. 1575: PRO21708
FIG. 1576: DNA227081, NP_000390.2, 205249_at
FIG. 1577: PRO37544
FIG. 1578: DNA227447, NP_003193.1, 205254_x_at
FIG. 1579: PRO37910
FIG. 1580: DNA227447, TCF7, 205255_x_at
FIG. 1581: PRO37910
FIG. 1582A-B: DNA226483, NP_000892.1,
205259_at
FIG. 1583: PRO36946
FIG. 1584A-B: DNA330079, 341358.1, 205263_at
FIG. 1585: PRO1162
FIG. 1586A-B: DNA188301, NP_002300.1,
205266_at
FIG. 1587: PRO21834
FIG. 1588: DNA227173, NP_001456.1, 205285_s_at
FIG. 1589: PRO37636
FIG. 1590A-B: DNA331483, CDC14A, 205288_at
FIG. 1591: PRO86528
FIG. 1592A-B: DNA331484, NP_000869.1,
205291_at
FIG. 1593: PRO3276
FIG. 1594: DNA88119, NP_000617.1, 205297_s_at
FIG. 1595: PRO2663
FIG. 1596A-B: DNA330081, NP_003026.1,
205339_at
FIG. 1597: PRO85358
FIG. 1598: DNA256854, NP_000456.1, 205345_at
FIG. 1599: PRO51785
FIG. 1600: DNA270415, NP_002059.1, 205349_at
FIG. 1601: PRO58796
FIG. 1602: DNA325568, NP_001265.1, 205393_s_at
FIG. 1603: PRO12187
FIG. 1604: DNA325568, CHEK1, 205394_at
FIG. 1605: PRO12187
FIG. 1606: DNA330082, NP_005582.1, 205395_s_at
FIG. 1607: PRO60497
FIG. 1608: DNA328561, NP_004624.1, 205403_at
FIG. 1609: PRO2019
FIG. 1610: DNA329010, NP_004942.1, 205419_at
FIG. 1611: PRO23370
FIG. 1612A-B: DNA210654, NP_055726.1,
205434_s_at
FIG. 1613: PRO54603
FIG. 1614: DNA287337, NP_002096.1, 205436_s_at
FIG. 1615: PRO69600
FIG. 1616: DNA330083, NP_003073.1, 205443_at
FIG. 1617: PRO69499
FIG. 1618: DNA272221, NP_037431.1, 205449_at
FIG. 1619: PRO60483
FIG. 1620: DNA88194, NP_000724.1, 205456_at
FIG. 1621: PRO2220
FIG. 1622: DNA188355, NP_004582.1, 205476_at
FIG. 1623: PRO21885
FIG. 1624: DNA287224, NP_005092.1, 205483_s_at
FIG. 1625: PRO69503
FIG. 1626: DNA330084, NP_055265.1, 205484_at
FIG. 1627: PRO9895
FIG. 1628: DNA225959, NP_006135.1, 205488_at
FIG. 1629: PRO36422
FIG. 1630: DNA331485, GNLY, 205495_s_at
FIG. 1631: PRO86529
FIG. 1632: DNA328566, NP_060446.1, 205510_s_at
FIG. 1633: PRO84363
FIG. 1634: DNA327639, NP_001053.2, 205513_at
FIG. 1635: PRO83640
FIG. 1636: DNA330085, D86324, 205518_s_at
FIG. 1637: PRO85359
FIG. 1638: DNA330086, NP_079184.1, 205519_at
FIG. 1639: PRO85360
FIG. 1640: DNA254810, NP_056536.1, 205527_s_at
FIG. 1641: PRO49906
FIG. 1642: DNA331486, OAS1, 205552_s_at
FIG. 1643: PRO69559
FIG. 1644: DNA330087, PCSK5, 205559_s_at
FIG. 1645: PRO85361
FIG. 1646: DNA256257, NP_055213.1, 205569_at
FIG. 1647: PRO51301
FIG. 1648A-B: DNA327643, NP_055712.1,
205594_at
FIG. 1649: PRO83644
FIG. 1650: DNA329013, NP_005649.1, 205599_at
FIG. 1651: PRO20128
FIG. 1652: DNA324324, NP_000679.1, 205633_s_at
FIG. 1653: PRO81000
FIG. 1654: DNA330088, NP_003087.1, 205644_s_at
FIG. 1655: PRO61962
FIG. 1656: DNA287317, NP_003724.1, 205660_at
FIG. 1657: PRO69582
FIG. 1658: DNA328570, NP_004040.1, 205681_at
FIG. 1659: PRO37843
FIG. 1660: DNA330089, NP_004200.2, 205691_at
FIG. 1661: PRO12507
FIG. 1662: DNA226234, NP_001766.1, 205692_s_at
FIG. 1663: PRO36697
FIG. 1664: DNA330090, NP_002749.2, 205698_s_at
FIG. 1665: PRO62976
FIG. 1666: DNA220761, NP_000880.1, 205718_at
FIG. 1667: PRO34739
FIG. 1668A-B: DNA271762, NP_000048.1,
205733_at
FIG. 1669: PRO60046
FIG. 1670: DNA331318, NP_003636.1, 205768_s_at
FIG. 1671: PRO51139
FIG. 1672: DNA331318, SLC27A2, 205769_at
FIG. 1673: PRO51139
FIG. 1674: DNA330091, NP_057461.1, 205771_s_at
FIG. 1675: PRO85362
FIG. 1676: DNA330092, NP_004904.1, 205781_at
FIG. 1677: PRO85363
FIG. 1678A-B: DNA220752, NP_000623.1,
205786_s_at
FIG. 1679: PRO34730
FIG. 1680: DNA330093, NP_003717.2, 205790_at
FIG. 1681: PRO85364
FIG. 1682: DNA76517, NP_002176.1, 205798_at
FIG. 1683: PRO2541
FIG. 1684A-B: DNA271915, NP_056191.1,
205801_s_at
FIG. 1685: PRO60192
FIG. 1686: DNA194766, NP_079504.1, 205804_s_at
FIG. 1687: PRO24046
FIG. 1688A-B: DNA328574, NP_004963.1,
205841_at
FIG. 1689: PRO84368
FIG. 1690A-B: DNA328574, JAK2, 205842_s_at
FIG. 1691: PRO84368
FIG. 1692: DNA330094, TREX1, 205875_s_at
FIG. 1693: PRO85365
FIG. 1694: DNA331320, HSU37122, 205882_x_at
FIG. 1695: PRO86409
FIG. 1696A-B: DNA220746, NP_000876.1,
205884_at
FIG. 1697: PRO34724
FIG. 1698A-B: DNA220746, ITGA4, 205885_s_at
FIG. 1699: PRO34724
FIG. 1700: DNA329540, NP_006389.1, 205890_s_at
FIG. 1701: PRO85090
FIG. 1702: DNA330095, NP_004732.1, 205895_s_at
FIG. 1703: PRO85366
FIG. 1704: DNA328576, HSU20350, 205898_at
FIG. 1705: PRO4940
FIG. 1706: DNA287318, NP_002683.1, 205909_at
FIG. 1707: PRO69583
FIG. 1708: DNA75525, NP_005805.1, 205929_at
FIG. 1709: PRO2524
FIG. 1710: DNA76516, NP_000556.1, 205945_at
FIG. 1711: PRO2022
FIG. 1712: DNA329047, NP_006390.1, 205965_at
FIG. 1713: PRO58425
FIG. 1714: DNA273487, NP_004785.1, 206039_at
FIG. 1715: PRO61470
FIG. 1716A-B: DNA290265, NP_003421.1,
206059_at
FIG. 1717: PRO70395
FIG. 1718: DNA330096, NP_057051.1, 206060_s_at
FIG. 1719: PRO37163
FIG. 1720: DNA271992, NP_006665.1, 206082_at
FIG. 1721: PRO60267
FIG. 1722: DNA270851, NP_006617.1, 206098_at
FIG. 1723: PRO59189
FIG. 1724: DNA226105, NP_002925.1, 206111_at
FIG. 1725: PRO36568
FIG. 1726: DNA83063, NP_004429.1, 206114_at
FIG. 1727: PRO2068
FIG. 1728A-B: DNA151420, NP_004421.1,
206115_at
FIG. 1729: PRO12876
FIG. 1730: DNA287306, NP_059993.1, 206133_at
FIG. 1731: PRO69572
FIG. 1732: DNA330097, NP_001233.1, 206150_at
FIG. 1733: PRO2024
FIG. 1734: DNA331487, GABPB2, 206173_x_at
FIG. 1735: PRO86530
FIG. 1736: DNA329005, NP_003028.1, 206181_at
FIG. 1737: PRO12612
FIG. 1738: DNA330098, NP_073619.1, 206205_at
FIG. 1739: PRO85367
FIG. 1740: DNA329168, CLC, 206207_at
FIG. 1741: PRO84794
FIG. 1742: DNA281446, NP_031394.1, 206220_s_at
FIG. 1743: PRO66285
FIG. 1744: DNA281446, GAP1IP4BP, 206221_at
FIG. 1745: PRO66285
FIG. 1746A-B: DNA331488, NP_055523.1,
206316_s_at
FIG. 1747: PRO86531
FIG. 1748: DNA327661, NP_005522.1, 206332_s_at
FIG. 1749: PRO83652
FIG. 1750: DNA218278, NP_000408.1, 206341_at
FIG. 1751: PRO34330
FIG. 1752: DNA269870, NP_005382.1, 206348_s_at
FIG. 1753: PRO58270
FIG. 1754A-B: DNA330100, NP_055690.1,
206364_at
FIG. 1755: PRO85369
FIG. 1756: DNA329169, NP_002986.1, 206366_x_at
FIG. 1757: PRO1610
FIG. 1758: DNA271310, NP_004411.1, 206374_at
FIG. 1759: PRO59617
FIG. 1760A-E: DNA331489, NP_066267.1,
206385_s_at
FIG. 1761: PRO86532
FIG. 1762: DNA326727, NP_001527.1, 206445_s_at
FIG. 1763: PRO83069
FIG. 1764A-B: DNA271891, NP_055766.1,
206448_at
FIG. 1765: PRO60170
FIG. 1766: DNA153751, NP_005942.1, 206461_x_at
FIG. 1767: PRO12925
FIG. 1768: DNA88203, NP_055022.1, 206485_at
FIG. 1769: PRO2698
FIG. 1770: DNA288243, NP_002277.3, 206486_at
FIG. 1771: PRO36451
FIG. 1772: DNA269850, NP_002003.1, 206492_at
FIG. 1773: PRO58251
FIG. 1774: DNA270444, NP_004824.1, 206513_at
FIG. 1775: PRO58823
FIG. 1776A-B: DNA188192, NP_006130.1,
206545_at
FIG. 1777: PRO21704
FIG. 1778A-B: DNA330102, NP_004289.1,
206550_s_at
FIG. 1779: PRO85371
FIG. 1780: DNA331490, OAS2, 206553_at
FIG. 1781: PRO69656
FIG. 1782: DNA227540, NP_003036.1, 206566_at
FIG. 1783: PRO38003
FIG. 1784: DNA330103, NP_056179.1, 206584_at
FIG. 1785: PRO19671
FIG. 1786: DNA329172, NP_005254.1, 206589_at
FIG. 1787: PRO84796
FIG. 1788: DNA103451, NP_003846.1, 206618_at
FIG. 1789: PRO4778
FIG. 1790: DNA227709, NP_000947.1, 206631_at
FIG. 1791: PRO38172
FIG. 1792: DNA331491, NP_004891.2, 206632_s_at
FIG. 1793: PRO62308
FIG. 1794: DNA331492, BCL2L1, 206665_s_at
FIG. 1795: PRO83141
FIG. 1796: DNA88374, NP_002095.1, 206666_at
FIG. 1797: PRO2768
FIG. 1798: DNA330105, HUMNCAX, 206676_at
FIG. 1799: PRO85372
FIG. 1800: DNA328590, C6orf32, 206707_x_at
FIG. 1801: PRO84375
FIG. 1802: DNA330106, NP_003646.1, 206724_at
FIG. 1803: PRO85373
FIG. 1804A-B: DNA88191, NP_001234.1, 206729_at
FIG. 1805: PRO2691
FIG. 1806A-B: DNA88650, NP_005807.1, 206761_at
FIG. 1807: PRO2460
FIG. 1808: DNA226427, NP_002251.1, 206785_s_at
FIG. 1809: PRO36890
FIG. 1810: DNA88195, NP_000064.1, 206804_at
FIG. 1811: PRO2693
FIG. 1812: DNA256561, NP_062550.1, 206914_at
FIG. 1813: PRO51592
FIG. 1814: DNA93439, NP_006555.1, 206974_at
FIG. 1815: PRO4515
FIG. 1816: DNA35629, NP_000586.2, 206975_at
FIG. 1817: PRO7
FIG. 1818: DNA328591, NP_006635.1, 206976_s_at
FIG. 1819: PRO84376
FIG. 1820: DNA331493, CCR2, 206978_at
FIG. 1821: PRO84690
FIG. 1822: DNA188346, NP_001450.1, 206980_s_at
FIG. 1823: PRO21766
FIG. 1824A-B: DNA227659, NP_000570.1,
206991_s_at
FIG. 1825: PRO38122
FIG. 1826A-B: DNA227750, NP_001550.1,
206999_at
FIG. 1827: PRO38213
FIG. 1828: DNA329903, PPP3CC, 207000_s_at
FIG. 1829: PRO85220
FIG. 1830: DNA330108, NP_004080.1, 207001_x_at
FIG. 1831: PRO85374
FIG. 1832: DNA331494, PLAGL1, 207002_s_at
FIG. 1833: PRO62736
FIG. 1834: DNA331495, HUMBCL2B, 207005_s_at
FIG. 1835: PRO86533
FIG. 1836: DNA330110, HUMK10A, 207023_x_at
FIG. 1837: PRO85375
FIG. 1838: DNA225550, NP_003844.1, 207072_at
FIG. 1839: PRO36013
FIG. 1840: DNA273159, NP_005457.1, 207078_at
FIG. 1841: PRO61201
FIG. 1842: DNA227481, VAMP1, 207100_s_at
FIG. 1843: PRO37944
FIG. 1844: DNA218655, NP_000585.1, 207113_s_at
FIG. 1845: PRO34451
FIG. 1846: DNA330111, NP_002615.2, 207132_x_at
FIG. 1847: PRO85376
FIG. 1848: DNA330112, NP_444504.1, 207153_s_at
FIG. 1849: PRO61610
FIG. 1850: DNA103418, NP_036616.1, 207165_at
FIG. 1851: PRO4746
FIG. 1852: DNA330113, NP_203124.1, 207181_s_at
FIG. 1853: PRO85377
FIG. 1854: DNA330114, NP_006134.1, 207183_at
FIG. 1855: PRO4946
FIG. 1856: DNA331496, RBMS1, 207266_x_at
FIG. 1857: PRO86534
FIG. 1858: DNA83048, NP_001916.1, 207269_at
FIG. 1859: PRO2057
FIG. 1860A-B: DNA330115, NP_077739.1,
207324_s_at
FIG. 1861: PRO85378
FIG. 1862A-B: DNA226536, NP_003225.1,
207332_s_at
FIG. 1863: PRO36999
FIG. 1864: DNA331497, LTB, 207339_s_at
FIG. 1865: PRO11604
FIG. 1866: DNA330117, NP_003966.1, 207351_s_at
FIG. 1867: PRO85379
FIG. 1868: DNA330118, NP_036389.2, 207361_at
FIG. 1869: PRO85380
FIG. 1870: DNA226396, NP_002180.1, 207375_s_at
FIG. 1871: PRO36859
FIG. 1872: DNA227668, NP_000158.1, 207387_s_at
FIG. 1873: PRO38131
FIG. 1874A-B: DNA329093, MSF, 207425_s_at
FIG. 1875: PRO84745
FIG. 1876: DNA36718, NP_000563.1, 207433_at
FIG. 1877: PRO73
FIG. 1878A-B: DNA330119, NP_060189.2,
207474_at
FIG. 1879: PRO85381
FIG. 1880: DNA328597, NP_001680.1, 207507_s_at
FIG. 1881: PRO84381
FIG. 1882: DNA328597, ATP5G3, 207508_at
FIG. 1883: PRO84381
FIG. 1884A-B: DNA256059, NP_005164.1,
207521_s_at
FIG. 1885: PRO51107
FIG. 1886A-B: DNA256059, ATP2A3, 207522_s_at
FIG. 1887: PRO51107
FIG. 1888: DNA304473, NP_001552.2, 207536_s_at
FIG. 1889: PRO2023
FIG. 1890: DNA325454, NP_003637.1, 207556_s_at
FIG. 1891: PRO81977
FIG. 1892: DNA328601, NP_056490.1, 207574_s_at
FIG. 1893: PRO84384
FIG. 1894A-B: DNA330120, FLJ10971, 207606_s_at
FIG. 1895: PRO85382
FIG. 1896: DNA255271, NP_038475.1, 207610_s_at
FIG. 1897: PRO50348
FIG. 1898: DNA331498, TANK, 207616_s_at
FIG. 1899: PRO86535
FIG. 1900: DNA226337, NP_005683.2, 207622_s_at
FIG. 1901: PRO36800
FIG. 1902: DNA227606, NP_001872.2, 207630_s_at
FIG. 1903: PRO38069
FIG. 1904: DNA196426, NP_037440.1, 207651_at
FIG. 1905: PRO24924
FIG. 1906: DNA328554, NP_038202.1, 207677_s_at
FIG. 1907: PRO84354
FIG. 1908A-B: DNA226405, NP_006525.1,
207700_s_at
FIG. 1909: PRO36868
FIG. 1910: DNA329064, NP_060301.1, 207735_at
FIG. 1911: PRO84724
FIG. 1912: DNA329020, NUP62, 207740_s_at
FIG. 1913: PRO84695
FIG. 1914: DNA325654, NP_054752.1, 207761_s_at
FIG. 1915: PRO4348
FIG. 1916A-B: DNA329179, NP_056958.1,
207785_s_at
FIG. 1917: PRO84802
FIG. 1918: DNA227494, NP_002158.1, 207826_s_at
FIG. 1919: PRO37957
FIG. 1920A-C: DNA331499, NP_057427.2,
207828_s_at
FIG. 1921: PRO86536
FIG. 1922: DNA329182, HPIP, 207838_x_at
FIG. 1923: PRO84805
FIG. 1924: DNA330123, NP_008984.1, 207840_at
FIG. 1925: PRO35080
FIG. 1926: DNA227175, NP_006857.1, 207857_at
FIG. 1927: PRO37638
FIG. 1928: DNA330124, NP_002981.2, 207861_at
FIG. 1929: PRO34107
FIG. 1930: DNA217245, NP_000579.1, 207906_at
FIG. 1931: PRO34287
FIG. 1932: DNA218651, NP_003798.1, 207907_at
FIG. 1933: PRO34447
FIG. 1934: DNA330125, NP_002729.2, 207957_s_at
FIG. 1935: PRO85385
FIG. 1936A-B: DNA226290, NP_036333.1,
207966_s_at
FIG. 1937: PRO36753
FIG. 1938: DNA329183, NP_055962.1, 207971_s_at
FIG. 1939: PRO84806
FIG. 1940A-B: DNA330126, NP_008912.1,
207978_s_at
FIG. 1941: PRO85386
FIG. 1942: DNA329184, CITED2, 207980_s_at
FIG. 1943: PRO84807
FIG. 1944A-C: DNA254145, NP_004329.1,
207996_s_at
FIG. 1945: PRO49260
FIG. 1946: DNA275286, NP_009205.1, 208002_s_at
FIG. 1947: PRO62967
FIG. 1948: DNA288217, NP_002101.1, 208018_s_at
FIG. 1949: PRO69990
FIG. 1950: DNA227224, NP_060877.1, 208029_s_at
FIG. 1951: PRO37687
FIG. 1952A-B: DNA188492, NAB1, 208047_s_at
FIG. 1953: PRO22070
FIG. 1954: DNA330127, NP_006442.2, 208051_s_at
FIG. 1955: PRO85387
FIG. 1956A-B: DNA328607, NP_003639.1,
208072_s_at
FIG. 1957: PRO84390
FIG. 1958A-C: DNA331500, NP_003307.2,
208073_x_at
FIG. 1959: PRO86537
FIG. 1960A-B: DNA328312, NP_110378.1,
208078_s_at
FIG. 1961: PRO84180
FIG. 1962: DNA331501, STK6, 208079_s_at
FIG. 1963: PRO58855
FIG. 1964: DNA323896, NP_112182.1, 208103_s_at
FIG. 1965: PRO80638
FIG. 1966: DNA330129, NP_112495.1, 208119_s_at
FIG. 1967: PRO85389
FIG. 1968: DNA325329, NP_004719.1, 208152_s_at
FIG. 1969: PRO81872
FIG. 1970: DNA36717, NP_000581.1, 208193_at
FIG. 1971: PRO72
FIG. 1972A-E: DNA330130, HSTITIN, 208195_at
FIG. 1973: DNA328611, RASGRP2, 208206_s_at
FIG. 1974: PRO84393
FIG. 1975: DNA328612, NP_000166.2, 208308_s_at
FIG. 1976: PRO84394
FIG. 1977A-D: DNA331502, NP_000050.1,
208368_s_at
FIG. 1978: PRO86538
FIG. 1979: DNA324250, NP_536349.1, 208392_x_at
FIG. 1980: PRO80934
FIG. 1981A-B: DNA331503, RAD50, 208393_s_at
FIG. 1982: PRO86539
FIG. 1983: DNA327690, NP_004022.1, 208436_s_at
FIG. 1984: PRO83673
FIG. 1985: DNA103427, NP_005239.1, 208438_s_at
FIG. 1986: PRO4755
FIG. 1987A-C: DNA331504, ATM, 208442_s_at
FIG. 1988: PRO86540
FIG. 1989A-B: DNA330134, BAZ1B, 208445_s_at
FIG. 1990: PRO85394
FIG. 1991A-C: DNA331505, NP_000642.2,
208488_s_at
FIG. 1992: PRO86541
FIG. 1993: DNA330136, NP_002441.1, 208581_x_at
FIG. 1994: PRO82583
FIG. 1995A-C: DNA331506, NP_001448.1,
208614_s_at
FIG. 1996: PRO86542
FIG. 1997A-B: DNA330138, PTP4A2, 208617_s_at
FIG. 1998: PRO85397
FIG. 1999A-B: DNA273567, NP_004944.1,
208624_s_at
FIG. 2000: PRO61545
FIG. 2001A-B: DNA273567, EIF4G1, 208625_s_at
FIG. 2002: PRO61545
FIG. 2003: DNA325912, NP_001093.1, 208636_at
FIG. 2004: PRO82367
FIG. 2005: DNA325912, ACTN1, 208637_x_at
FIG. 2006: PRO82367
FIG. 2007: DNA329188, BC012142, 208638_at
FIG. 2008: PRO84810
FIG. 2009: DNA324641, NP_005608.1, 208646_at
FIG. 2010: PRO10849
FIG. 2011: DNA271268, NP_009057.1, 208649_s_at
FIG. 2012: PRO59579
FIG. 2013: DNA328617, AF299343, 208653_s_at
FIG. 2014: PRO84399
FIG. 2015: DNA330139, AK022493, 208657_s_at
FIG. 2016: PRO85398
FIG. 2017A-C: DNA151898, TTC3, 208661_s_at
FIG. 2018: PRO12135
FIG. 2019A-C: DNA151898, D84294, 208662_s_at
FIG. 2020: PRO12135
FIG. 2021A-C: DNA331507, D83327, 208663_s_at
FIG. 2022: DNA304686, NP_002565.1, 208680_at
FIG. 2023: PRO71112
FIG. 2024A-B: DNA328619, BC001188, 208691_at
FIG. 2025: PRO84401
FIG. 2026: DNA287189, NP_002038.1, 208693_s_at
FIG. 2027: PRO69475
FIG. 2028: DNA330140, AF275798, 208696_at
FIG. 2029: PRO85399
FIG. 2030A-C: DNA331508, 198777.9, 208707_at
FIG. 2031: PRO86543
FIG. 2032: DNA97298, NP_003899.1, 208726_s_at
FIG. 2033: PRO3645
FIG. 2034: DNA330142, BC003564, 208737_at
FIG. 2035: PRO85401
FIG. 2036: DNA331509, 1138554.23, 208740_at
FIG. 2037: PRO86544
FIG. 2038: DNA328591, HSP105B, 208744_x_at
FIG. 2039: PRO84376
FIG. 2040: DNA287285, NP_005794.1, 208748_s_at
FIG. 2041: PRO69556
FIG. 2042: DNA324217, ATIC, 208758_at
FIG. 2043: PRO80908
FIG. 2044: DNA327696, AF228339, 208763_s_at
FIG. 2045: PRO83679
FIG. 2046A-B: DNA331510, 1298307.1, 208776_at
FIG. 2047: PRO86545
FIG. 2048: DNA287427, NP_002806.1, 208777_s_at
FIG. 2049: PRO69684
FIG. 2050: DNA287219, NP_110379.1, 208778_s_at
FIG. 2051: PRO69498
FIG. 2052: DNA329189, NP_009139.1, 208787_at
FIG. 2053: PRO4911
FIG. 2054: DNA238565, NP_005907.2, 208795_s_at
FIG. 2055: PRO39210
FIG. 2056: DNA330145, NP_002788.1, 208799_at
FIG. 2057: PRO84403
FIG. 2058: DNA331511, HSMPIO, 208805_at
FIG. 2059A-C: DNA331512, 1397486.26, 208806_at
FIG. 2060: PRO86547
FIG. 2061A-B: DNA330147, HSU91543,
208807_s_at
FIG. 2062: PRO85405
FIG. 2063: DNA324531, NP_002120.1, 208808_s_at
FIG. 2064: PRO81185
FIG. 2065: DNA273521, NP_002070.1, 208813_at
FIG. 2066: PRO61502
FIG. 2067A-B: DNA330148, AB020636, 208838_at
FIG. 2068A-B: DNA330149, HSM801778,
208839_s_at
FIG. 2069: PRO82209
FIG. 2070: DNA227874, NP_003320.1, 208864_s_at
FIG. 2071: PRO38337
FIG. 2072: DNA328624, BC003562, 208891_at
FIG. 2073: PRO59076
FIG. 2074: DNA331513, DUSP6, 208892_s_at
FIG. 2075: PRO84404
FIG. 2076: DNA331330, BC005047, 208893_s_at
FIG. 2077: PRO82215
FIG. 2078: DNA329221, NP_061984.1, 208894_at
FIG. 2079: PRO4555
FIG. 2080A-B: DNA329007, NP_003277.1,
208900_s_at
FIG. 2081: PRO37029
FIG. 2082A-B: DNA329007, TOP1, 208901_s_at
FIG. 2083: PRO37029
FIG. 2084: DNA327700, BC015130, 208905_at
FIG. 2085: PRO83683
FIG. 2086: DNA327701, NP_001203.1, 208910_s_at
FIG. 2087: PRO82667
FIG. 2088: DNA281442, NP_149124.1, 208912_s_at
FIG. 2089: PRO66281
FIG. 2090A-B: DNA330151, AB029003, 208914_at
FIG. 2091: DNA325473, NP_006353.2, 208922_s_at
FIG. 2092: PRO81996
FIG. 2093: DNA329552, NP_063948.1, 208925_at
FIG. 2094: PRO85097
FIG. 2095: DNA326233, NP_000968.2, 208929_x_at
FIG. 2096: PRO82645
FIG. 2097: DNA327702, NP_006490.2, 208934_s_at
FIG. 2098: PRO83684
FIG. 2099: DNA330152, NP_001939.1, 208956_x_at
FIG. 2100: PRO85406
FIG. 2101: DNA290261, NP_001291.2, 208960_s_at
FIG. 2102: PRO70387
FIG. 2103A-B: DNA325478, NP_037534.2,
208962_s_at
FIG. 2104: PRO81999
FIG. 2105: DNA327661, IFI16, 208965_s_at
FIG. 2106: PRO83652
FIG. 2107A-B: DNA270277, AF208043,
208966_x_at
FIG. 2108: PRO58665
FIG. 2109: DNA326343, KPNB1, 208974_x_at
FIG. 2110: PRO82739
FIG. 2111A-B: DNA330153, HUMIMP90A,
208975_s_at
FIG. 2112: PRO82739
FIG. 2113: DNA328629, NP_006079.1, 208977_x_at
FIG. 2114: PRO84407
FIG. 2115: DNA330154, HUMPECAM27,
208981_at
FIG. 2116: DNA330155, 7692317.2, 208982_at
FIG. 2117: PRO85407
FIG. 2118: DNA330156, NP_003749.1, 208985_s_at
FIG. 2119: PRO85408
FIG. 2120: DNA331514, STAT3, 208992_s_at
FIG. 2121: PRO86548
FIG. 2122: DNA227552, NP_003346.2, 208997_s_at
FIG. 2123: PRO38015
FIG. 2124: DNA227552, UCP2, 208998_at
FIG. 2125: PRO38015
FIG. 2126: DNA328630, NP_036293.1, 209004_s_at
FIG. 2127: PRO84408
FIG. 2128: DNA331515, FBXL5, 209005_at
FIG. 2129: PRO86549
FIG. 2130: DNA328631, AK027318, 209006_s_at
FIG. 2131: PRO84409
FIG. 2132: DNA331516, DNAJB6, 209015_s_at
FIG. 2133: PRO83680
FIG. 2134: DNA328633, NP_004784.2, 209017_s_at
FIG. 2135: PRO84411
FIG. 2136: DNA330158, NP_057554.4, 209020_at
FIG. 2137: PRO85410
FIG. 2138: DNA327851, NP_006363.2, 209024_s_at
FIG. 2139: PRO83795
FIG. 2140: DNA328635, BC020946, 209026_x_at
FIG. 2141: PRO84413
FIG. 2142: DNA331517, NP_004150.1, 209040_s_at
FIG. 2143: PRO69506
FIG. 2144A-C: DNA328637, HSA7042, 209052_s_at
FIG. 2145: PRO81109
FIG. 2146A-B: DNA331518, AF330040,
209053_s_at
FIG. 2147: PRO86550
FIG. 2148A-B: DNA226405, NCOA3, 209060_x_at
FIG. 2149: PRO36868
FIG. 2150: DNA330159, HSM801885, 209064_x_at
FIG. 2151: PRO85411
FIG. 2152: DNA330160, NP_006285.1, 209066_x_at
FIG. 2153: PRO85412
FIG. 2154: DNA329194, NP_112740.1, 209068_at
FIG. 2155: PRO84814
FIG. 2156A-B: DNA330161, NP_085059.1,
209081_s_at
FIG. 2157: PRO85413
FIG. 2158: DNA330162, NP_057093.1, 209091_s_at
FIG. 2159: PRO85414
FIG. 2160: DNA330163, NP_060308.1, 209104_s_at
FIG. 2161: PRO85415
FIG. 2162: DNA330164, NP_004752.1, 209110_s_at
FIG. 2163: PRO85416
FIG. 2164: DNA327709, NP_000509.1, 209116_x_at
FIG. 2165: PRO83690
FIG. 2166: DNA288254, NP_006000.2, 209118_s_at
FIG. 2167: PRO69536
FIG. 2168: DNA325163, NP_001113.1, 209122_at
FIG. 2169: PRO81730
FIG. 2170: DNA330165, BC015833, 209138_x_at
FIG. 2171: PRO85417
FIG. 2172: DNA327713, BC010653, 209146_at
FIG. 2173: PRO37975
FIG. 2174: DNA325285, AKR1C3, 209160_at
FIG. 2175: PRO81832
FIG. 2176: DNA330166, BC001588, 209161_at
FIG. 2177: PRO85418
FIG. 2178: DNA271722, NP_004688.1, 209162_s_at
FIG. 2179: PRO60006
FIG. 2180: DNA330167, CAB43224.1, 209177_at
FIG. 2181: PRO85419
FIG. 2182A-B: DNA328642, AF073310,
209184_s_at
FIG. 2183: PRO84418
FIG. 2184: DNA331331, AF161416, 209185_s_at
FIG. 2185A-B: DNA328643, HUMHK1A,
209186_at
FIG. 2186: PRO84419
FIG. 2187: DNA189700, NP_005243.1, 209189_at
FIG. 2188: PRO25619
FIG. 2189: DNA324766, NP_005443.2, 209196_at
FIG. 2190: PRO81387
FIG. 2191: DNA226176, NP_003458.1, 209201_x_at
FIG. 2192: PRO36639
FIG. 2193: DNA326267, NP_004861.1, 209208_at
FIG. 2194: PRO82674
FIG. 2195: DNA326891, NP_001748.1, 209213_at
FIG. 2196: PRO83212
FIG. 2197: DNA227483, NP_003120.1, 209218_at
FIG. 2198: PRO37946
FIG. 2199: DNA330168, NP_006322.1, 209233_at
FIG. 2200: PRO85420
FIG. 2201: DNA328649, NP_116093.1, 209251_x_at
FIG. 2202: PRO84424
FIG. 2203: DNA255255, NP_071437.1, 209267_s_at
FIG. 2204: PRO50332
FIG. 2205A-B: DNA188492, AF045451, 209272_at
FIG. 2206: PRO22070
FIG. 2207A-B: DNA226827, NP_001673.1,
209281_s_at
FIG. 2208: PRO37290
FIG. 2209: DNA328601, GADD45B, 209304_x_at
FIG. 2210: PRO84384
FIG. 2211: DNA328651, AF087853, 209305_s_at
FIG. 2212: PRO82889
FIG. 2213: DNA151780, NP_006611.1, 209314_s_at
FIG. 2214: PRO12057
FIG. 2215: DNA330169, NP_006709.1, 209318_x_at
FIG. 2216: PRO62736
FIG. 2217: DNA275106, HSU76248, 209339_at
FIG. 2218: PRO62821
FIG. 2219: DNA269630, NP_003281.1, 209344_at
FIG. 2220: PRO58042
FIG. 2221A-B: DNA328658, AF055376,
209348_s_at
FIG. 2222: PRO84432
FIG. 2223: DNA330170, AF109161, 209357_at
FIG. 2224: PRO84807
FIG. 2225: DNA327720, NP_001970.1, 209368_at
FIG. 2226: PRO83699
FIG. 2227: DNA330171, CAA34971.1, 209374_s_at
FIG. 2228: PRO85421
FIG. 2229: DNA330172, BC009529, 209377_s_at
FIG. 2230: PRO85422
FIG. 2231: DNA330173, HUMAUTOTAX,
209392_at
FIG. 2232: PRO85423
FIG. 2233: DNA330174, AK027512, 209404_s_at
FIG. 2234: PRO85424
FIG. 2235: DNA330175, NP_006836.1, 209408_at
FIG. 2236: PRO59681
FIG. 2237A-B: DNA271241, HSU61500, 209412_at
FIG. 2238: PRO59556
FIG. 2239: DNA330176, AAB61703.1, 209417_s_at
FIG. 2240: PRO85425
FIG. 2241: DNA225767, NP_000534.1, 209420_s_at
FIG. 2242: PRO36230
FIG. 2243: DNA330177, BC001743, 209446_s_at
FIG. 2244: PRO10283
FIG. 2245: DNA273076, HSU59863, 209451_at
FIG. 2246: PRO61137
FIG. 2247: DNA326089, HBA2, 209458_x_at
FIG. 2248: PRO3629
FIG. 2249: DNA287304, AAH00040.1, 209461_x_at
FIG. 2250: PRO69571
FIG. 2251: DNA297388, NP_004208.1, 209464_at
FIG. 2252: PRO70812
FIG. 2253: DNA330178, HSTM2CEA, 209498_at
FIG. 2254: PRO85426
FIG. 2255: DNA330179, NP_067023.1, 209504_s_at
FIG. 2256: PRO85427
FIG. 2257: DNA324899, NP_002938.1, 209507_at
FIG. 2258: PRO81503
FIG. 2259: DNA330180, NP_009149.2, 209510_at
FIG. 2260: PRO85428
FIG. 2261: DNA274027, RAB27A, 209514_s_at
FIG. 2262: PRO61971
FIG. 2263: DNA274027, HSU38654, 209515_s_at
FIG. 2264: PRO61971
FIG. 2265: DNA272213, NP_002477.1, 209520_s_at
FIG. 2266: PRO60475
FIG. 2267: DNA330181, HSM802358, 209523_at
FIG. 2268: DNA328663, NP_057157.1, 209524_at
FIG. 2269: PRO36183
FIG. 2270: DNA330182, PLAA, 209533_s_at
FIG. 2271: PRO85430
FIG. 2272: DNA330183, AF181265, 209536_s_at
FIG. 2273: DNA327724, AF323542S7, 209546_s_at
FIG. 2274: DNA257501, NP_115688.1, 209551_at
FIG. 2275: PRO52073
FIG. 2276: DNA330184, BC022475, 209566_at
FIG. 2277: PRO85432
FIG. 2278: DNA290251, NP_055207.1, 209569_x_at
FIG. 2279: PRO70367
FIG. 2280: DNA329202, BC001745, 209570_s_at
FIG. 2281: PRO70367
FIG. 2282: DNA329203, NP_003788.1, 209572_s_at
FIG. 2283: PRO84819
FIG. 2284: DNA304797, NP_005935.3, 209583_s_at
FIG. 2285: PRO71209
FIG. 2286: DNA328666, AF084943, 209585_s_at
FIG. 2287: PRO1917
FIG. 2288: DNA270689, NP_002042.1, 209604_s_at
FIG. 2289: PRO59053
FIG. 2290: DNA271823, NP_004279.2, 209606_at
FIG. 2291: PRO60104
FIG. 2292A-B: DNA328670, BC001618,
209610_s_at
FIG. 2293: PRO70011
FIG. 2294: DNA330185, NP_071415.1, 209624_s_at
FIG. 2295: PRO85433
FIG. 2296: DNA328599, HSNFKBS, 209636_at
FIG. 2297: PRO84382
FIG. 2298: DNA330186, NP_004327.1, 209642_at
FIG. 2299: PRO85434
FIG. 2300A-B: DNA330187, HSM801454,
209649_at
FIG. 2301: PRO85435
FIG. 2302: DNA330188, NP_004356.1, 209662_at
FIG. 2303: PRO85436
FIG. 2304: DNA323856, PAI-RBP1, 209669_s_at
FIG. 2305: PRO80599
FIG. 2306: DNA330189, BC000712, 209680_s_at
FIG. 2307: PRO85437
FIG. 2308: DNA193881, AAF15129.1, 209681_at
FIG. 2309: PRO23299
FIG. 2310A-B: DNA272671, HSU26710, 209682_at
FIG. 2311: PRO60796
FIG. 2312: DNA330125, HUMPKB, 209685_s_at
FIG. 2313: PRO85385
FIG. 2314: DNA331519, HMMR, 209709_s_at
FIG. 2315: PRO86551
FIG. 2316: DNA328264, NP_005183.2, 209714_s_at
FIG. 2317: PRO12087
FIG. 2318: DNA330191, NP_036249.1, 209715_at
FIG. 2319: PRO85439
FIG. 2320A-C: DNA254412, AF008915, 209717_at
FIG. 2321: PRO49522
FIG. 2322A-B: DNA330192, 234780.1, 209733_at
FIG. 2323: PRO85440
FIG. 2324: DNA330193, BC015929, 209750_at
FIG. 2325: DNA330194, HSU09087, 209754_s_at
FIG. 2326: PRO85442
FIG. 2327: DNA275195, NP_001025.1, 209773_s_at
FIG. 2328: PRO62893
FIG. 2329: DNA329205, NP_001343.1, 209782_s_at
FIG. 2330: PRO84821
FIG. 2331: DNA226436, NP_001772.1, 209795_at
FIG. 2332: PRO36899
FIG. 2333: DNA327731, NP_003302.1, 209803_s_at
FIG. 2334: PRO83707
FIG. 2335A-B: DNA271368, HUMKIAAI,
209804_at
FIG. 2336: PRO59668
FIG. 2337: DNA329206, AF151103, 209813_x_at
FIG. 2338: PRO84822
FIG. 2339A-C: DNA331520, 1096711.1, 209815_at
FIG. 2340: PRO86552
FIG. 2341: DNA327732, UMPK, 209825_s_at
FIG. 2342: PRO61801
FIG. 2343: DNA328676, IL16, 209827_s_at
FIG. 2344: PRO84448
FIG. 2345A-B: DNA196499, AB002384, 209829_at
FIG. 2346: PRO24988
FIG. 2347: DNA330197, NP_112190.1, 209832_s_at
FIG. 2348: PRO85445
FIG. 2349: DNA328677, AF060511, 209836_x_at
FIG. 2350: PRO84449
FIG. 2351: DNA270180, NP_478123.1, 209849_s_at
FIG. 2352: PRO58569
FIG. 2353: DNA331521, BC018951, 209868_s_at
FIG. 2354: PRO58719
FIG. 2355A-B: DNA329536, NP_005494.2,
209870_s_at
FIG. 2356: PRO22775
FIG. 2357: DNA330198, AB014719, 209871_s_at
FIG. 2358: PRO85446
FIG. 2359: DNA324184, NP_065726.1, 209891_at
FIG. 2360: PRO80882
FIG. 2361: DNA328258, HSM802616, 209900_s_at
FIG. 2362: PRO84151
FIG. 2363: DNA330152, DUT, 209932_s_at
FIG. 2364: PRO85406
FIG. 2365: DNA150133, AAD01646.1, 209933_s_at
FIG. 2366: PRO12219
FIG. 2367: DNA329208, CFLAR, 209939_x_at
FIG. 2368: PRO84823
FIG. 2369: DNA330199, BC004357, 209944_at
FIG. 2370: PRO85447
FIG. 2371A-B: DNA329065, HSU12767, 209959_at
FIG. 2372: PRO84725
FIG. 2373: DNA154921, DNA154921, 209967_s_at
FIG. 2374: DNA327736, BC002704, 209969_s_at
FIG. 2375: PRO83711
FIG. 2376: DNA324895, JTV1, 209971_x_at
FIG. 2377: PRO81501
FIG. 2378: DNA226658, NP_003736.1, 209999_x_at
FIG. 2379: PRO37121
FIG. 2380: DNA226658, SSI-1, 210001_s_at
FIG. 2381: PRO37121
FIG. 2382: DNA330200, NP_056222.1, 210006_at
FIG. 2383: PRO85448
FIG. 2384: DNA269534, NP_002155.1, 210029_at
FIG. 2385: PRO57950
FIG. 2386: DNA326054, NP_002159.1, 210046_s_at
FIG. 2387: PRO82489
FIG. 2388: DNA326809, NP_036244.2, 210052_s_at
FIG. 2389: PRO83142
FIG. 2390: DNA150551, AAB97010.1, 210054_at
FIG. 2391: PRO12778
FIG. 2392: DNA274960, SFRS5, 210077_s_at
FIG. 2393: PRO62694
FIG. 2394: DNA324922, BC018962, 210095_s_at
FIG. 2395: PRO119
FIG. 2396A-B: DNA328685, NP_127497.1,
210113_s_at
FIG. 2397: PRO34751
FIG. 2398: DNA330201, NP_003774.1, 210121_at
FIG. 2399: PRO50625
FIG. 2400: DNA330202, NP_005400.1, 210163_at
FIG. 2401: PRO19838
FIG. 2402: DNA287620, NP_004122.1, 210164_at
FIG. 2403: PRO2081
FIG. 2404: DNA270196, HUMZFM1B, 210172_at
FIG. 2405: PRO58584
FIG. 2406: DNA330203, NP_003755.1, 210190_at
FIG. 2407: PRO85449
FIG. 2408: DNA331335, AF070576, 210201_x_at
FIG. 2409: DNA331522, AF068918, 210202_s_at
FIG. 2410: PRO86553
FIG. 2411: DNA331523, RAD1, 210216_x_at
FIG. 2412: PRO61690
FIG. 2413: DNA328467, AF056322, 210218_s_at
FIG. 2414: PRO84293
FIG. 2415: DNA217253, NP_000749.1, 210229_s_at
FIG. 2416: PRO34295
FIG. 2417: DNA331084, BC008487, 210254_at
FIG. 2418: PRO81984
FIG. 2419A-B: DNA270015, NP_003444.1,
210281_s_at
FIG. 2420: PRO58410
FIG. 2421: DNA330206, NP_005801.2, 210288_at
FIG. 2422: PRO85450
FIG. 2423: DNA329945, SEC23B, 210293_s_at
FIG. 2424: PRO85252
FIG. 2425: DNA218653, NP_003799.1, 210314_x_at
FIG. 2426: PRO34449
FIG. 2427: DNA326239, NP_006752.1, 210317_s_at
FIG. 2428: PRO39530
FIG. 2429: DNA329213, NP_219491.1, 210321_at
FIG. 2430: PRO2313
FIG. 2431A-B: DNA329214, NP_001087.1,
210337_s_at
FIG. 2432: PRO84826
FIG. 2433: DNA225528, NP_000610.1, 210354_at
FIG. 2434: PRO35991
FIG. 2435: DNA196621, HUMLY9, 210370_s_at
FIG. 2436: DNA330207, BC001131, 210387_at
FIG. 2437: PRO85451
FIG. 2438: DNA226229, NP_002432.1, 210410_s_at
FIG. 2439: PRO36692
FIG. 2440A-B: DNA330208, AF164622,
210425_x_at
FIG. 2441: PRO85452
FIG. 2442: DNA329215, NP_036224.1, 210439_at
FIG. 2443: PRO7424
FIG. 2444: DNA226394, NP_002552.1, 210448_s_at
FIG. 2445: PRO36857
FIG. 2446: DNA331524, BC003388, 210458_s_at
FIG. 2447: PRO86554
FIG. 2448: DNA331525, BC002448, 210461_s_at
FIG. 2449: PRO86555
FIG. 2450: DNA329216, AF022375, 210512_s_at
FIG. 2451: PRO84827
FIG. 2452: DNA227633, NP_001156.1, 210538_s_at
FIG. 2453: PRO38096
FIG. 2454: DNA330209, BC000585, 210542_s_at
FIG. 2455: PRO85453
FIG. 2456: DNA331526, BC014563, 210559_s_at
FIG. 2457: PRO58324
FIG. 2458: DNA331527, BC001602, 210563_x_at
FIG. 2459: PRO86556
FIG. 2460: DNA331528, AF00619, 210564_x_at
FIG. 2461: PRO86557
FIG. 2462: DNA329217, BC003406, 210571_s_at
FIG. 2463: PRO84828
FIG. 2464: DNA330210, HSU03858, 210607_at
FIG. 2465: PRO126
FIG. 2466: DNA330211, NP_009092.1, 210629_x_at
FIG. 2467: PRO85454
FIG. 2468: DNA330212, HUMKRT10A,
210633_x_at
FIG. 2469: PRO85455
FIG. 2470: DNA331529, LAIR1, 210644_s_at
FIG. 2471: PRO86558
FIG. 2472A-C: DNA330214, HUMTPRD,
210645_s_at
FIG. 2473: PRO12135
FIG. 2474: DNA329218, NP_055227.1, 210691_s_at
FIG. 2475: PRO84829
FIG. 2476: DNA330215, DKFZp762A227Homo,
210692_s_at
FIG. 2477: DNA331530, AF064103, 210742_at
FIG. 2478: PRO86559
FIG. 2479: DNA237817, NP_001307.1, 210766_s_at
FIG. 2480: PRO38923
FIG. 2481A-B: DNA330216, NP_006445.1,
210778_s_at
FIG. 2482: PRO85457
FIG. 2483: DNA226881, FLI1, 210786_s_at
FIG. 2484: PRO37344
FIG. 2485: DNA255402, NP_055288.1, 210802_s_at
FIG. 2486: PRO50469
FIG. 2487: DNA330027, SSBP2, 210829_s_at
FIG. 2488: PRO85312
FIG. 2489: DNA329219, BC000385, 210844_x_at
FIG. 2490: PRO81278
FIG. 2491A-B: DNA331107, HSU26455,
210858_x_at
FIG. 2492: PRO86255
FIG. 2493: DNA188234, NP_000630.1, 210865_at
FIG. 2494: PRO21942
FIG. 2495: DNA331531, PFDN5, 210908_s_at
FIG. 2496: PRO86560
FIG. 2497: DNA330217, AF043183, 210915_x_at
FIG. 2498: PRO85458
FIG. 2499: DNA274326, NP_003079.1, 210933_s_at
FIG. 2500: PRO62244
FIG. 2501: DNA329317, NP_057353.1, 210948_s_at
FIG. 2502: PRO81157
FIG. 2503: DNA331532, AF125393, 210951_x_at
FIG. 2504: PRO86561
FIG. 2505: DNA330218, HUMTCAXA,
210972_x_at
FIG. 2506: DNA273236, NP_004306.1, 210980_s_at
FIG. 2507: PRO61263
FIG. 2508: DNA269888, NP_002073.1, 210981_s_at
FIG. 2509: PRO58286
FIG. 2510: DNA329221, HLA-DRA, 210982_s_at
FIG. 2511: PRO4555
FIG. 2512: DNA238565, MCM7, 210983_s_at
FIG. 2513: PRO39210
FIG. 2514: DNA326239, YWHAE, 210996_s_at
FIG. 2515: PRO39530
FIG. 2516A-B: DNA330219, NP_150241.1,
211013_x_at
FIG. 2517: PRO85459
FIG. 2518: DNA327699, AB023420, 211015_s_at
FIG. 2519: PRO83682
FIG. 2520: DNA288254, TUBA3, 211058_x_at
FIG. 2521: PRO69536
FIG. 2522: DNA329992, MGAT2, 211061_s_at
FIG. 2523: PRO59267
FIG. 2524: DNA324171, NP_065438.1, 211070_x_at
FIG. 2525: PRO60753
FIG. 2526: DNA330220, NP_006809.1, 211071_s_at
FIG. 2527: PRO60769
FIG. 2528: DNA287198, K-ALPHA-1, 211072_x_at
FIG. 2529: PRO69484
FIG. 2530: DNA254470, NEK2, 211080_s_at
FIG. 2531: PRO49578
FIG. 2532: DNA196432, AF064804, 211106_at
FIG. 2533: PRO24928
FIG. 2534: DNA330202, CXCL11, 211122_s_at
FIG. 2535: PRO19838
FIG. 2536: DNA304765, HUMTCRGAD,
211144_x_at
FIG. 2537: PRO71178
FIG. 2538: DNA327752, HSDHACTYL,
211150_s_at
FIG. 2539A-B: DNA328700, SCD, 211162_x_at
FIG. 2540: PRO84464
FIG. 2541: DNA330221, NP_056071.1, 211207_s_at
FIG. 2542: PRO85460
FIG. 2543: DNA330222, NP_003848.1, 211226_at
FIG. 2544: PRO45958
FIG. 2545: DNA218278, IL2RA, 211269_s_at
FIG. 2546: PRO34330
FIG. 2547: DNA151022, DGKA, 211272_s_at
FIG. 2548: PRO12096
FIG. 2549: DNA330223, NP_001790.1, 211297_s_at
FIG. 2550: PRO49730
FIG. 2551A-C: DNA328811, ITPR1, 211323_s_at
FIG. 2552: PRO84551
FIG. 2553: DNA188234, TNFSF6, 211333_s_at
FIG. 2554: PRO21942
FIG. 2555: DNA103395, HSU80737, 211352_s_at
FIG. 2556: PRO4723
FIG. 2557A-B: DNA275066, NP_000170.1,
211450_s_at
FIG. 2558: PRO62786
FIG. 2559: DNA327755, NP_115957.1, 211458_s_at
FIG. 2560: PRO83725
FIG. 2561: DNA93439, CXCR6, 211469_s_at
FIG. 2562: PRO4515
FIG. 2563: DNA330175, KNSL6, 211519_s_at
FIG. 2564: PRO59681
FIG. 2565: DNA327756, NP_068814.2, 211538_s_at
FIG. 2566: PRO83726
FIG. 2567: DNA269888, GPRK6, 211543_s_at
FIG. 2568: PRO58286
FIG. 2569: DNA226255, NP_003047.1, 211576_s_at
FIG. 2570: PRO36718
FIG. 2571: DNA330211, LST1, 211581_x_at
FIG. 2572: PRO85454
FIG. 2573: DNA330224, HUMNCA, 211657_at
FIG. 2574: PRO85461
FIG. 2575: DNA327709, HBB, 211696_x_at
FIG. 2576: PRO83690
FIG. 2577: DNA331533, PPARG, 211699_x_at
FIG. 2578: PRO86562
FIG. 2579: DNA331534, AF116616, 211708_s_at
FIG. 2580: DNA226342, PTEN, 211711_s_at
FIG. 2581: PRO36805
FIG. 2582: DNA328706, BC021909, 211714_x_at
FIG. 2583: PRO10347
FIG. 2584: DNA88307, NP_001992.1, 211734_s_at
FIG. 2585: PRO2280
FIG. 2586: DNA329225, EVI2B, 211742_s_at
FIG. 2587: PRO84833
FIG. 2588: DNA331535, AF105974, 211745_x_at
FIG. 2589: PRO3629
FIG. 2590: DNA328649, TUBA6, 211750_x_at
FIG. 2591: PRO84424
FIG. 2592: DNA254725, KPNA2, 211762_s_at
FIG. 2593: PRO49824
FIG. 2594: DNA330225, NP_115712.1, 211767_at
FIG. 2595: PRO85462
FIG. 2596A-B: DNA329226, BC006181,
211784_s_at
FIG. 2597: PRO60388
FIG. 2598: DNA304473, BC006196, 211786_at
FIG. 2599: PRO2023
FIG. 2600: DNA330226, AF198052, 211794_at
FIG. 2601: PRO85463
FIG. 2602: DNA227173, FYB, 211795_s_at
FIG. 2603: PRO37636
FIG. 2604: DNA331536, AAA60662.1, 211796_s_at
FIG. 2605: PRO86563
FIG. 2606: DNA331537, CCNE2, 211814_s_at
FIG. 2607: PRO59418
FIG. 2608A-B: DNA331342, DEFCAP, 211822_s_at
FIG. 2609: PRO86422
FIG. 2610: DNA331343, AK026398, 211824_x_at
FIG. 2611: PRO86423
FIG. 2612: DNA331538, AF327066, 211825_s_at
FIG. 2613: PRO86564
FIG. 2614A-B: DNA331539, BRCA1, 211851_x_at
FIG. 2615: PRO86565
FIG. 2616A-B: DNA188192, CD28, 211856_x_at
FIG. 2617: PRO21704
FIG. 2618: DNA331540, AF222343, 211861_x_at
FIG. 2619: PRO86566
FIG. 2620: DNA330228, HUMTCRAZ, 211902_x_at
FIG. 2621: PRO85465
FIG. 2622: DNA226176, CXCR4, 211919_s_at
FIG. 2623: PRO36639
FIG. 2624: DNA272286, CAT, 211922_s_at
FIG. 2625: PRO60544
FIG. 2626: DNA330229, BC011915, 211926_s_at
FIG. 2627: PRO85466
FIG. 2628: DNA226254, NP_001408.1, 211937_at
FIG. 2629: PRO36717
FIG. 2630: DNA330230, NP_060977.1, 211938_at
FIG. 2631: PRO85467
FIG. 2632A-B: DNA325306, ITGB1, 211945_s_at
FIG. 2633: PRO81851
FIG. 2634A-B: DNA272195, HUMORFGA,
211951_at
FIG. 2635: DNA328437, NP_005792.1, 211956_s_at
FIG. 2636: PRO84271
FIG. 2637: DNA325941, NP_005339.1, 211969_at
FIG. 2638: PRO82388
FIG. 2639: DNA287194, AAA60258.1, 211974_x_at
FIG. 2640: PRO69480
FIG. 2641A-C: DNA331541, 1390535.1, 211986_at
FIG. 2642: PRO86567
FIG. 2643: DNA330232, NP_291032.1, 211991_s_at
FIG. 2644: PRO85469
FIG. 2645: DNA330233, AF218029, 211999_at
FIG. 2646: PRO11403
FIG. 2647: DNA287433, NP_006810.1, 212009_s_at
FIG. 2648: PRO69690
FIG. 2649A-D: DNA103461, MKI67, 212020_s_at
FIG. 2650: PRO4788
FIG. 2651A-D: DNA226463, HSMKI67A,
212021_s_at
FIG. 2652: PRO36926
FIG. 2653A-D: DNA103461, HSMKI67,
212022_s_at
FIG. 2654: PRO4788
FIG. 2655A-D: DNA226463, DNA226463,
212023_s_at
FIG. 2656: PRO36926
FIG. 2657: DNA275447, HSMEMA, 212037_at
FIG. 2658: PRO63095
FIG. 2659: DNA103380, NP_003365.1, 212038_s_at
FIG. 2660: PRO4710
FIG. 2661A-B: DNA330234, 215138.24, 212045_at
FIG. 2662: PRO85470
FIG. 2663: DNA328709, BC004151, 212048_s_at
FIG. 2664: PRO37676
FIG. 2665A-B: DNA330235, BAA20790.1,
212061_at
FIG. 2666: PRO85471
FIG. 2667: DNA330236, 228447.20, 212071_s_at
FIG. 2668: PRO85472
FIG. 2669: DNA154139, DNA154139, 212099_at
FIG. 2670A-B: DNA331542, BAA74910.1,
212108_at
FIG. 2671: PRO86568
FIG. 2672A-B: DNA150956, BAA06685.1,
212110_at
FIG. 2673: PRO12560
FIG. 2674: DNA328711, AK023154, 212115_at
FIG. 2675: PRO84468
FIG. 2676: DNA219225, NP_002874.1, 212125_at
FIG. 2677: PRO34531
FIG. 2678: DNA330238, BC019676, 212127_at
FIG. 2679: DNA328713, AF100737, 212130_x_at
FIG. 2680: PRO84470
FIG. 2681: DNA330239, AK027643, 212131_at
FIG. 2682: PRO85474
FIG. 2683: DNA330240, CAA52801.1, 212141_at
FIG. 2684: PRO85475
FIG. 2685: DNA330240, HSP1CDC21, 212142_at
FIG. 2686A-B: DNA150829, AB014568, 212144_at
FIG. 2687: DNA329602, AK2, 212175_s_at
FIG. 2688: PRO85133
FIG. 2689: DNA330241, AF314185, 212176_at
FIG. 2690: DNA328716, HSM800707, 212179_at
FIG. 2691: DNA330242, BC007034, 212185_x_at
FIG. 2692: PRO85477
FIG. 2693: DNA330243, BC015663, 212190_at
FIG. 2694: PRO2584
FIG. 2695: DNA326233, RPL13, 212191_x_at
FIG. 2696: PRO82645
FIG. 2697A-C: DNA330244, 253946.17, 212196_at
FIG. 2698: PRO85478
FIG. 2699A-B: DNA330245, 230497.7, 212206_s_at
FIG. 2700: PRO85479
FIG. 2701: DNA331543, BC008710, 212227_x_at
FIG. 2702: PRO84271
FIG. 2703: DNA327770, 1384008.4, 212239_at
FIG. 2704: PRO83736
FIG. 2705: DNA151120, DNA151120, 212240_s_at
FIG. 2706: PRO12179
FIG. 2707: DNA330246, AF326773, 212241_at
FIG. 2708A-B: DNA329229, 1345070.7, 212249_at
FIG. 2709: PRO84835
FIG. 2710: DNA329182, BC016852, 212259_s_at
FIG. 2711: PRO84805
FIG. 2712: DNA331544, BC018823, 212266_s_at
FIG. 2713: PRO86569
FIG. 2714: DNA327771, NP_109591.1, 212268_at
FIG. 2715: PRO83737
FIG. 2716: DNA326463, NP_000976.1, 212270_x_at
FIG. 2717: PRO82846
FIG. 2718: DNA150980, HUMMAC30X, 212279_at
FIG. 2719: DNA150980, DNA150980, 212281_s_at
FIG. 2720: PRO12566
FIG. 2721: DNA253017, DNA253017, 212282_at
FIG. 2722: PRO48926
FIG. 2723: DNA328719, BC012895, 212295_s_at
FIG. 2724: PRO84475
FIG. 2725: DNA271103, NP_005796.1, 212296_at
FIG. 2726: PRO59425
FIG. 2727: DNA207620, DNA207620, 212300_at
FIG. 2728: DNA330247, BC019110, 212313_at
FIG. 2729: PRO85481
FIG. 2730: DNA330248, BC019924, 212320_at
FIG. 2731: PRO10347
FIG. 2732A-B: DNA124122, HSP130K, 212331_at
FIG. 2733: PRO6323
FIG. 2734A-B: DNA124122, NP_005602.2,
212332_at
FIG. 2735: PRO6323
FIG. 2736: DNA287190, CAB43217.1, 212333_at
FIG. 2737: PRO69476
FIG. 2738A-B: DNA330216, MAD4, 212347_x_at
FIG. 2739: PRO85457
FIG. 2740A-B: DNA327773, BAA25456.1,
212366_at
FIG. 2741: PRO83739
FIG. 2742A-C: DNA330249, AAA99177.1,
212372_at
FIG. 2743: PRO85482
FIG. 2744: DNA329231, NP_000810.1, 212378_at
FIG. 2745: PRO84837
FIG. 2746: DNA329231, GART, 212379_at
FIG. 2747: PRO84837
FIG. 2748A-B: DNA150950, HUMKIAAH,
21239_s_at
FIG. 2749A-B: DNA328549, NP_002897.1,
212397_at
FIG. 2750: PRO84350
FIG. 2751A-B: DNA328549, RDX, 212398_at
FIG. 2752: PRO84350
FIG. 2753: DNA151330, DNA151330, 212400_at
FIG. 2754: PRO11708
FIG. 2755A-B: DNA330250, NP_060727.1,
212406_s_at
FIG. 2756: PRO85483
FIG. 2757: DNA254828, AK023204, 212408_at
FIG. 2758: PRO49923
FIG. 2759: DNA330251, NP_059965.1, 212430_at
FIG. 2760: PRO85484
FIG. 2761: DNA327774, BC016808, 212460_at
FIG. 2762: PRO83740
FIG. 2763A-B: DNA330252, NP_055447.1,
212473_s_at
FIG. 2764: PRO85485
FIG. 2765: DNA269630, TPM4, 212481_s_at
FIG. 2766: PRO58042
FIG. 2767: DNA330253, BC007665, 212493_s_at
FIG. 2768: PRO85486
FIG. 2769: DNA330254, AK024029, 212508_at
FIG. 2770: PRO85487
FIG. 2771A-B: DNA254192, BAA07648.1,
212510_at
FIG. 2772: PRO49304
FIG. 2773: DNA329233, 383512.16, 212527_at
FIG. 2774: PRO84839
FIG. 2775: DNA226041, NP_005555.1, 212531_at
FIG. 2776: PRO36504
FIG. 2777: DNA269882, HSWEE1HU, 212533_at
FIG. 2778: PRO58280
FIG. 2779A-D: DNA328737, 148650.1, 212560_at
FIG. 2780: PRO84490
FIG. 2781A-B: DNA330255, AK025499, 212561_at
FIG. 2782: PRO85488
FIG. 2783: DNA225632, NP_002037.2, 212581_x_at
FIG. 2784: PRO36095
FIG. 2785A-C: DNA329236, AF392452, 212582_at
FIG. 2786: PRO84841
FIG. 2787A-B: DNA331545, AB040884, 212585_at
FIG. 2788: DNA275100, DNA275100, 212589_at
FIG. 2789: DNA330256, BC020889, 212592_at
FIG. 2790: PRO81145
FIG. 2791: DNA330257, 441179.4, 212605_s_at
FIG. 2792: PRO85489
FIG. 2793A-B: DNA330258, BAA22955.2,
212619_at
FIG. 2794: PRO85490
FIG. 2795A-B: DNA330258, AB006624, 212621_at
FIG. 2796: DNA330259, NP_008944.1, 212638_s_at
FIG. 2797: PRO49366
FIG. 2798: DNA331357, BC010494, 212639_x_at
FIG. 2799: PRO38556
FIG. 2800A-D: DNA327777, HSIL1RECA,
212657_s_at
FIG. 2801A-B: DNA327778, AB011154,
21267_s_at
FIG. 2802: DNA273465, DNA273465, 212677_s_at
FIG. 2803: DNA328744, AF318364, 212680_x_at
FIG. 2804: PRO84496
FIG. 2805A-B: DNA329901, AB007915, 212683_at
FIG. 2806A-B: DNA269508, AB011110, 212706_at
FIG. 2807A-B: DNA331546, 332730.12, 212714_at
FIG. 2808: PRO86570
FIG. 2809: DNA331547, BC010994, 212734_x_at
FIG. 2810: PRO82645
FIG. 2811: DNA329906, BC007848, 212738_at
FIG. 2812: PRO85223
FIG. 2813: DNA330261, NP_110383.1, 212762_s_at
FIG. 2814: PRO85492
FIG. 2815: DNA330262, NP_006409.2, 212768_s_at
FIG. 2816: PRO85493
FIG. 2817A-B: DNA254149, BAA06224.1,
212789_at
FIG. 2818: PRO49264
FIG. 2819: DNA331548, BC017356, 212827_at
FIG. 2820: PRO86571
FIG. 2821A-B: DNA150452, BAA32470.1,
212830_at
FIG. 2822: PRO12260
FIG. 2823A-B: DNA331549, BAA07892.2,
212832_s_at
FIG. 2824: PRO86572
FIG. 2825: DNA271714, BAA05039.1, 212836_at
FIG. 2826: PRO59998
FIG. 2827: DNA331550, AAA59587.1, 212859_x_at
FIG. 2828: PRO6386
FIG. 2829A-B: DNA328753, BAA13212.1,
212873_at
FIG. 2830: PRO84502
FIG. 2831: DNA330265, NP_056436.1, 212886_at
FIG. 2832: PRO85495
FIG. 2833A-B: DNA271215, BAA24380.1,
212892_at
FIG. 2834: PRO59530
FIG. 2835A-B: DNA330266, CAA10334.1,
212902_at
FIG. 2836: PRO85496
FIG. 2837A-B: DNA271137, AB014589, 212905_at
FIG. 2838: DNA271630, DNA271630, 212907_at
FIG. 2839: DNA330267, 235076.14, 212914_at
FIG. 2840: PRO85497
FIG. 2841: DNA330268, BC009116, 212928_at
FIG. 2842: PRO85498
FIG. 2843: DNA331551, BC013078, 212933_x_at
FIG. 2844: PRO82645
FIG. 2845A-B: DNA330269, BC020584, 212936_at
FIG. 2846: PRO23868
FIG. 2847: DNA330270, HUMORF007, 212949_at
FIG. 2848A-B: DNA331552, PAM, 212958_x_at
FIG. 2849: PRO86573
FIG. 2850: DNA273283, HUMCYSTRNA,
212971_at
FIG. 2851: DNA330271, 399773.20, 212980_at
FIG. 2852: PRO85500
FIG. 2853: DNA330272, AF119896, 212981_s_at
FIG. 2854: DNA330273, AK027564, 213007_at
FIG. 2855: PRO85502
FIG. 2856: DNA254940, BAA91770.1, 213008_at
FIG. 2857: PRO50030
FIG. 2858: DNA325596, TPI1, 213011_s_at
FIG. 2859: PRO69549
FIG. 2860: DNA331553, 228519.3, 213021_at
FIG. 2861: PRO86574
FIG. 2862A-B: DNA253815, BAA20833.2,
213035_at
FIG. 2863: PRO49218
FIG. 2864A-B: DNA329240, NP_056133.1,
213039_at
FIG. 2865: PRO84845
FIG. 2866A-B: DNA330275, BAA25487.1,
213045_at
FIG. 2867: PRO85504
FIG. 2868A-B: DNA329242, BAA76857.1,
213056_at
FIG. 2869: PRO84847
FIG. 2870: DNA323869, NP_000960.2, 213080_x_at
FIG. 2871: PRO80612
FIG. 2872: DNA270466, HUMG6PD, 213093_at
FIG. 2873: DNA330276, NP_001614.3, 213095_x_at
FIG. 2874: PRO85505
FIG. 2875: DNA331554, AF118070, 213113_s_at
FIG. 2876: PRO86575
FIG. 2877: DNA287230, AAA36325.1, 213138_at
FIG. 2878: PRO69509
FIG. 2879: DNA330277, CAB45152.1, 213142_x_at
FIG. 2880: PRO85506
FIG. 2881: DNA228053, DNA228053, 213156_at
FIG. 2882: DNA151370, DNA151370, 213158_at
FIG. 2883: PRO11747
FIG. 2884: DNA106374, DNA106374, 213164_at
FIG. 2885A-B: DNA330278, BAA13216.1,
213174_at
FIG. 2886: PRO85507
FIG. 2887: DNA330279, AF043182, 213193_x_at
FIG. 2888: PRO85508
FIG. 2889: DNA227909, NP_005024.1, 213226_at
FIG. 2890: PRO38372
FIG. 2891A-B: DNA330280, BAA83045.2,
213254_at
FIG. 2892: PRO85509
FIG. 2893A-B: DNA328761, BAA82991.1,
213280_at
FIG. 2894: PRO84509
FIG. 2895: DNA331555, BC009874, 213281_at
FIG. 2896A-B: DNA274945, HSACKI10,
213287_s_at
FIG. 2897: DNA260974, NP_006065.1, 213293_s_at
FIG. 2898: PRO54720
FIG. 2899: DNA328357, 1452321.2, 213295_at
FIG. 2900: PRO84217
FIG. 2901A-B: DNA329248, BAA20816.1,
213302_at
FIG. 2902: PRO84850
FIG. 2903A-B: DNA255273, BAA83044.1,
213309_at
FIG. 2904: PRO50349
FIG. 2905: DNA155418, DNA155418, 213326_at
FIG. 2906A-B: DNA331355, AAG24545.1,
213330_s_at
FIG. 2907: PRO86431
FIG. 2908A-B: DNA330281, AB058688, 213341_at
FIG. 2909: DNA327789, 1449824.5, 213348_at
FIG. 2910: PRO83753
FIG. 2911: DNA287176, AB025254, 213361_at
FIG. 2912: DNA327790, 1448999.3, 213364_s_at
FIG. 2913: PRO83754
FIG. 2914A-B: DNA330282, 217860.13, 213376_at
FIG. 2915: PRO85510
FIG. 2916: DNA330283, BC020225, 213408_s_at
FIG. 2917: PRO85511
FIG. 2918: DNA330284, 235806.16, 213434_at
FIG. 2919: PRO85512
FIG. 2920: DNA225632, GAPD, 213453_x_at
FIG. 2921: PRO36095
FIG. 2922A-B: DNA330285, 241020.1, 213469_at
FIG. 2923: PRO85513
FIG. 2924: DNA328766, NP_006077.1, 213476_x_at
FIG. 2925: PRO84514
FIG. 2926: DNA330286, BC018130, 213506_at
FIG. 2927: PRO85514
FIG. 2928: DNA326639, NP_001229.1, 213523_at
FIG. 2929: PRO82992
FIG. 2930A-C: DNA330287, AF380180, 213538_at
FIG. 2931: PRO85515
FIG. 2932: DNA227483, SQLE, 213562_s_at
FIG. 2933: PRO37946
FIG. 2934: DNA330288, NP_005606.1, 213566_at
FIG. 2935: PRO2869
FIG. 2936: DNA330289, 197444.1, 213567_at
FIG. 2937: PRO85516
FIG. 2938: DNA159560, DNA159560, 213577_at
FIG. 2939: DNA330290, 1398807.8, 213581_at
FIG. 2940: PRO85517
FIG. 2941: DNA327799, HSRP26AA, 213587_s_at
FIG. 2942: PRO40011
FIG. 2943: DNA273753, AAC39561.1, 213599_at
FIG. 2944: PRO61716
FIG. 2945: DNA330291, 1500175.9, 213616_at
FIG. 2946: PRO85518
FIG. 2947A-C: DNA330292, NP_056045.2,
213618_at
FIG. 2948: PRO85519
FIG. 2949: DNA225974, ICAM2, 213620_s_at
FIG. 2950: PRO36437
FIG. 2951: DNA331556, BC009513, 213646_x_at
FIG. 2952: PRO38556
FIG. 2953A-B: DNA273985, BAA07647.1,
213647_at
FIG. 2954: PRO61932
FIG. 2955: DNA270758, HSU54778, 213655_at
FIG. 2956: PRO59117
FIG. 2957: DNA330293, BC011922, 213666_at
FIG. 2958: PRO85520
FIG. 2959: DNA325704, MARS, 213671_s_at
FIG. 2960: PRO82188
FIG. 2961: DNA304796, NP_443109.1, 213696_s_at
FIG. 2962: PRO71208
FIG. 2963: DNA273236, ASAH1, 213702_x_at
FIG. 2964: PRO61263
FIG. 2965: DNA255913, DNA255913, 213725_x_at
FIG. 2966: DNA328629, TUBB2, 213726_x_at
FIG. 2967: PRO84407
FIG. 2968: DNA328771, HSMYOSIE, 213733_at
FIG. 2969A-C: DNA151167, FLNA, 213746_s_at
FIG. 2970: PRO12867
FIG. 2971: DNA326273, BC001832, 213757_at
FIG. 2972: PRO82678
FIG. 2973A-B: DNA274483, NP_000126.1,
213778_x_at
FIG. 2974: PRO62385
FIG. 2975: DNA328774, NP_004263.1, 213793_s_at
FIG. 2976: PRO60536
FIG. 2977: DNA327804, AF442151, 213797_at
FIG. 2978: PRO69493
FIG. 2979A-B: DNA329967, SMARCA5,
213859_x_at
FIG. 2980: PRO85270
FIG. 2981: DNA151041, HSAMYB2, 213906_at
FIG. 2982: DNA330294, 426625.1, 213908_at
FIG. 2983: PRO85521
FIG. 2984: DNA330295, NP_037515.1, 213951_s_at
FIG. 2985: PRO85522
FIG. 2986: DNA327807, NP_115613.1, 213975_s_at
FIG. 2987: PRO83768
FIG. 2988: DNA327808, NP_002961.1, 213988_s_at
FIG. 2989: PRO83769
FIG. 2990: DNA329136, HSPC111, 214011_s_at
FIG. 2991: PRO84772
FIG. 2992: DNA196110, DNA196110, 214016_s_at
FIG. 2993: PRO24635
FIG. 2994: DNA227224, LC27, 214039_s_at
FIG. 2995: PRO37687
FIG. 2996: DNA330296, 206955.3, 214054_at
FIG. 2997: PRO85523
FIG. 2998: DNA273696, DNA273696, 214060_at
FIG. 2999A-B: DNA330297, AF378753, 214081_at
FIG. 3000: PRO85524
FIG. 3001: DNA227091, NP_000256.1, 214084_x_at
FIG. 3002: PRO37554
FIG. 3003: DNA331557, BC016778, 214085_x_at
FIG. 3004: PRO86576
FIG. 3005: DNA254686, NP_005475.1, 214086_s_t
FIG. 3006: PRO49786
FIG. 3007: DNA330298, BC011911, 214095_at
FIG. 3008: PRO83772
FIG. 3009: DNA329254, BC004215, 214096_s_at
FIG. 3010: PRO84854
FIG. 3011: DNA330299, AK023737, 214102_at
FIG. 3012: PRO85525
FIG. 3013: DNA331360, AK022497, 214177_s_at
FIG. 3014: PRO86435
FIG. 3015A-B: DNA269826, NP_003195.1,
214179_s_at
FIG. 3016: PRO58228
FIG. 3017: DNA331558, AF000424, 214181_x_at
FIG. 3018: PRO86577
FIG. 3019: DNA290295, NP_055203.1, 214193_s_at
FIG. 3020: PRO70455
FIG. 3021: DNA327701, C1QBP, 214214_s_at
FIG. 3022: PRO82667
FIG. 3023: DNA331361, NP_003318.1, 214228_x_at
FIG. 3024: PRO2398
FIG. 3025: DNA154914, DNA154914, 214230_at
FIG. 3026: DNA330300, NP_004883.1, 214257_s_at
FIG. 3027: PRO41086
FIG. 3028: DNA273940, DNA273940, 214272_at
FIG. 3029: DNA97279, JUND, 214326_x_at
FIG. 3030: PRO3628
FIG. 3031: DNA84130, HSU37518, 214329_x_at
FIG. 3032: PRO1096
FIG. 3033: DNA272928, DAZAP2, 214334_x_at
FIG. 3034: PRO61012
FIG. 3035: DNA331362, AF275719, 214359_s_at
FIG. 3036: PRO86436
FIG. 3037: DNA331559, AF043723, 214368_at
FIG. 3038: PRO85114
FIG. 3039: DNA328611, AF043722, 214369_s_at
FIG. 3040: PRO84393
FIG. 3041: DNA273138, NP_005495.1, 214390_s_at
FIG. 3042: PRO61182
FIG. 3043: DNA273174, NP_001951.1, 214394_x_at
FIG. 3044: PRO61211
FIG. 3045: DNA328782, 337794.1, 214405_at
FIG. 3046: PRO84528
FIG. 3047: DNA326090, NP_000549.1, 214414_x_at
FIG. 3048: PRO3629
FIG. 3049: DNA271374, CHAF1A, 214426_x_at
FIG. 3050: PRO59673
FIG. 3051: DNA287630, NP_000160.1, 214430_at
FIG. 3052: PRO2154
FIG. 3053: DNA327811, SHMT2, 214437_s_at
FIG. 3054: PRO83772
FIG. 3055: DNA331363, AF001383, 214439_x_at
FIG. 3056: PRO86437
FIG. 3057: DNA331560, NP_001326.1, 214450_at
FIG. 3058: PRO85081
FIG. 3059: DNA273138, BCAT1, 214452_at
FIG. 3060: PRO61182
FIG. 3061: DNA327812, NP_006408.2, 214453_s_at
FIG. 3062: PRO83773
FIG. 3063: DNA150971, NP_002249.1, 214470_at
FIG. 3064: PRO12564
FIG. 3065: DNA330301, NP_008908.1, 214482_at
FIG. 3066: PRO85526
FIG. 3067: DNA325246, RRP4, 214507_s_at
FIG. 3068: PRO81800
FIG. 3069: DNA331561, CREM, 214508_x_at
FIG. 3070: PRO86578
FIG. 3071: DNA331562, NP_003090.1, 214531_s_at
FIG. 3072: PRO58654
FIG. 3073: DNA216515, NP_003166.1, 214567_s_at
FIG. 3074: PRO34267
FIG. 3075: DNA331223, HUMPRF1M, 214617_at
FIG. 3076: DNA331563, BC004101, 214643_x_at
FIG. 3077: PRO86579
FIG. 3078: DNA150552, AAB97011.1, 214661_s_at
FIG. 3079: PRO12326
FIG. 3080: DNA330303, BAA05499.1, 214662_at
FIG. 3081: PRO85528
FIG. 3082: DNA330304, HSIGVL026, 214677_x_at
FIG. 3083: PRO85529
FIG. 3084: DNA287355, ALDOA, 214687_x_at
FIG. 3085: PRO69617
FIG. 3086: DNA330305, HSU79263, 214700_x_at
FIG. 3087: DNA288259, NP_114172.1, 214710_s_at
FIG. 3088: PRO4676
FIG. 3089: DNA324984, NP_115540.1, 214714_at
FIG. 3090: PRO81578
FIG. 3091: DNA330306, 407311.1, 214743_at
FIG. 3092: PRO85531
FIG. 3093: DNA331564, BC014654, 214752_x_at
FIG. 3094: PRO86580
FIG. 3095: DNA254338, AAA60119.1, 214765_s_at
FIG. 3096: PRO49449
FIG. 3097: DNA275473, DNA275473, 214787_at
FIG. 3098A-B: DNA272353, AB007958, 214833_at
FIG. 3099: DNA226577, C6orf9, 214847_s_at
FIG. 3100: PRO37040
FIG. 3101A-B: DNA331565, BAA34472.1,
214945_at
FIG. 3102: PRO86581
FIG. 3103: DNA328530, LAK-4P, 214958_s_at
FIG. 3104: PRO24118
FIG. 3105A-B: DNA271654, AB020704,
214978_s_at
FIG. 3106A-B: DNA329261, HSM802517,
215001_s_at
FIG. 3107: PRO84859
FIG. 3108: DNA330308, 307914.1, 215029_at
FIG. 3109: PRO85533
FIG. 3110: DNA196372, HSBCLXL, 215037_s_at
FIG. 3111: PRO24874
FIG. 3112: DNA331566, AIF1, 215051_x_at
FIG. 3113: PRO86582
FIG. 3114: DNA330309, NP_003503.1, 215071_s_at
FIG. 3115: PRO85534
FIG. 3116A-B: DNA330307, AB018295,
215133_s_at
FIG. 3117: DNA331567, 333089.1, 215147_at
FIG. 3118: PRO86583
FIG. 3119: DNA273371, UMPS, 215165_x_at
FIG. 3120: PRO61373
FIG. 3121A-B: DNA150496, AB023212, 215175_at
FIG. 3122A-B: DNA220748, ITGA6, 215177_s_at
FIG. 3123: PRO34726
FIG. 3124: DNA330311, 405318.1, 215221_at
FIG. 3125: PRO85536
FIG. 3126: DNA227597, NP_000627.1, 215223_s_at
FIG. 3127: PRO38060
FIG. 3128A-B: DNA330312, 406407.1, 215262_at
FIG. 3129: PRO85537
FIG. 3130: DNA331568, TADA3L, 215273_s_at
FIG. 3131: PRO80953
FIG. 3132: DNA330314, 026641.5, 215275_at
FIG. 3133: PRO85538
FIG. 3134: DNA330315, 1500205.1, 215283_at
FIG. 3135: PRO85539
FIG. 3136: DNA330316, 1448781.1, 215284_at
FIG. 3137: PRO85540
FIG. 3138: DNA330317, 228133.1, 215330_at
FIG. 3139: PRO85541
FIG. 3140: DNA331569, NP_000552.1, 215333_x_at
FIG. 3141: PRO85542
FIG. 3142: DNA327831, NP_076956.1, 215380_s_at
FIG. 3143: PRO83783
FIG. 3144: DNA328801, 407831.1, 215392_at
FIG. 3145: PRO84543
FIG. 3146: DNA325174, NP_038470.1, 215416_s_at
FIG. 3147: PRO9819
FIG. 3148: DNA275385, NP_002085.1, 215438_x_at
FIG. 3149: PRO63048
FIG. 3150: DNA331570, BC015794, 215440_s_at
FIG. 3151: PRO84545
FIG. 3152: DNA330319, AF247727, 215483_at
FIG. 3153: DNA331571, MAP2K3, 215498_s_at
FIG. 3154: PRO86584
FIG. 3155: DNA330186, BUB1, 215509_s_at
FIG. 3156: PRO85434
FIG. 3157: DNA330321, 315726.1, 215605_at
FIG. 3158: PRO85545
FIG. 3159: DNA331572, AF000426, 215633_x_at
FIG. 3160: PRO86585
FIG. 3161: DNA330031, LOC51668, 215691_x_at
FIG. 3162: PRO85316
FIG. 3163: DNA331573, HSAPT1, 215719_x_at
FIG. 3164A-B: DNA254376, KIAA0963,
215760_s_at
FIG. 3165: PRO49486
FIG. 3166A-B: DNA328805, BAA86482.1,
215785_s_at
FIG. 3167: PRO84547
FIG. 3168: DNA331574, HUMTCGCH, 215806_x_at
FIG. 3169: DNA330322, 234025.21, 215855_s_at
FIG. 3170: PRO85546
FIG. 3171: DNA330323, 335053.1, 215908_at
FIG. 3172: PRO85547
FIG. 3173: DNA330324, HHEX, 215933_s_at
FIG. 3174: PRO58034
FIG. 3175: DNA331575, AF223408, 215942_s_at
FIG. 3176: PRO86587
FIG. 3177: DNA330325, NP_055057.1, 215948_x_at
FIG. 3178: PRO85548
FIG. 3179: DNA227668, GK, 215966_x_at
FIG. 3180: PRO38131
FIG. 3181: DNA330326, AY007142, 215967_s_at
FIG. 3182: PRO85549
FIG. 3183: DNA331576, HSRNAGLK, 215977_x_at
FIG. 3184: DNA188736, U00115, 215990_s_at
FIG. 3185: PRO26296
FIG. 3186: DNA331577, 208045.1, 216109_at
FIG. 3187: PRO86588
FIG. 3188: DNA331578, HSTCELD, 216133_at
FIG. 3189: PRO86589
FIG. 3190: DNA254783, DKC1, 216212_s_at
FIG. 3191: PRO49881
FIG. 3192A-B: DNA255273, AB029015,
216218_s_at
FIG. 3193A-B: DNA256461, AND-1, 216228_s_at
FIG. 3194: PRO51498
FIG. 3195: DNA329266, BC000142, 216237_s_at
FIG. 3196: PRO12845
FIG. 3197: DNA151048, HSNOT, 216248_s_at
FIG. 3198: PRO12850
FIG. 3199: DNA329155, BC012479, 216252_x_at
FIG. 3200: PRO1207
FIG. 3201: DNA331579, HSCLCA, 216295_s_at
FIG. 3202: DNA88189, CD24, 216379_x_at
FIG. 3203: PRO2690
FIG. 3204: DNA330329, IR1875335, 216483_s_at
FIG. 3205: DNA287243, NP_004452.1, 216602_s_at
FIG. 3206: PRO69518
FIG. 3207: DNA331580, 1099517.2, 216607_s_at
FIG. 3208: PRO86590
FIG. 3209: DNA227874, TXN, 216609_at
FIG. 3210: PRO38337
FIG. 3211: DNA88296, NP_005733.1, 216640_s_at
FIG. 3212: PRO2274
FIG. 3213: DNA269692, S59049, 216834_at
FIG. 3214: PRO58102
FIG. 3215: DNA227597, SOD2, 216841_s_at
FIG. 3216: PRO38060
FIG. 3217A-B: DNA330331, BAA86451.1,
216873_s_at
FIG. 3218: PRO85554
FIG. 3219: DNA188275, NP_002181.1, 216876_s_at
FIG. 3220: PRO21800
FIG. 3221A-B: DNA150987, NP_006051.1,
216901_s_at
FIG. 3222: PRO12163
FIG. 3223: DNA329267, HUMTCRGAAC,
216920_s_at
FIG. 3224A-C: DNA328811, HUMINSP3R1,
216944_s_at
FIG. 3225: PRO84551
FIG. 3226A-B: DNA151027, AAA80979.1,
216952_s_at
FIG. 3227: PRO12843
FIG. 3228A-E: DNA269650, PLEC1, 216971_s_at
FIG. 3229A-B: PRO58061
FIG. 3230: DNA328812, BAA86575.1, 216997_x_at
FIG. 3231: PRO84552
FIG. 3232: DNA331581, AAB59396.1, 217022_s_at
FIG. 3233: PRO86591
FIG. 3234: DNA331366, HUMGPCR, 217028_at
FIG. 3235: PRO4516
FIG. 3236A-B: DNA227293, AB020721,
217047_s_at
FIG. 3237: PRO37756
FIG. 3238A-B: DNA329269, BAA32292.2,
217122_s_at
FIG. 3239: PRO84865
FIG. 3240: DNA331582, AAA59588.1, 217165_x_at
FIG. 3241: PRO86592
FIG. 3242: DNA331583, S70123, 217173_s_at
FIG. 3243: DNA331584, AF105973, 217232_x_at
FIG. 3244: PRO86593
FIG. 3245: DNA330334, NP_114402.1, 217286_s_at
FIG. 3246: PRO85557
FIG. 3247: DNA331369, HSU88968, 217294_s_at
FIG. 3248A-B: DNA331585, AF051334,
217299_s_at
FIG. 3249: PRO86594
FIG. 3250: DNA331586, S81916, 217356_s_at
FIG. 3251: DNA331587, HSNGMRNA, 217398_x_at
FIG. 3252: PRO86595
FIG. 3253: DNA330335, NP_054765.1, 217408_at
FIG. 3254: PRO62166
FIG. 3255: DNA331588, AF097635, 217414_x_at
FIG. 3256: PRO3629
FIG. 3257: DNA329539, HLA-DMA, 217478_s_at
FIG. 3258: PRO85089
FIG. 3259: DNA331589, 243999.2, 217502_at
FIG. 3260: PRO86596
FIG. 3261: DNA329271, 406848.1, 217591_at
FIG. 3262: PRO84867
FIG. 3263: DNA330337, 1447003.1, 217616_at
FIG. 3264: PRO85559
FIG. 3265: DNA331590, 368556.1, 217655_at
FIG. 3266: PRO86597
FIG. 3267: DNA330339, HSA012375, 217672_x_at
FIG. 3268: DNA323856, HSM800628, 217725_x_at
FIG. 3269: PRO80599
FIG. 3270: DNA326523, NP_001121.2, 217729_s_at
FIG. 3271: PRO71126
FIG. 3272: DNA325832, NP_068839.1, 217731_s_at
FIG. 3273: PRO1869
FIG. 3274A-B: DNA327847, 142131.14, 217738_at
FIG. 3275: PRO2834
FIG. 3276: DNA88541, NP_005737.1, 217739_s_at
FIG. 3277: PRO2834
FIG. 3278: DNA327935, NP_079422.1, 217745_s_at
FIG. 3279: PRO83866
FIG. 3280: DNA327849, NP_057269.1, 217755_at
FIG. 3281: PRO83794
FIG. 3282A-B: DNA274131, AF183421,
217762_s_at
FIG. 3283: PRO62067
FIG. 3284: DNA330340, NP_006859.1, 217763_s_at
FIG. 3285: PRO85562
FIG. 3286A-B: DNA274131, DNA274131,
217764_s_at
FIG. 3287: PRO62067
FIG. 3288: DNA325821, NP_057016.1, 217769_s_at
FIG. 3289: PRO82287
FIG. 3290: DNA325910, AF167438, 217776_at
FIG. 3291: PRO82365
FIG. 3292: DNA227358, NP_057479.1, 217777_s_at
FIG. 3293: PRO37821
FIG. 3294: DNA328819, NP_057145.1, 217783_s_at
FIG. 3295: PRO84557
FIG. 3296: DNA325873, SKB1, 217786_at
FIG. 3297: PRO82331
FIG. 3298: DNA331591, NP_055241.1, 217792_at
FIG. 3299: PRO69560
FIG. 3300: DNA328303, NP_056525.1, 217807_s_at
FIG. 3301: PRO84173
FIG. 3302: DNA227223, NP_064583.1, 217814_at
FIG. 3303: PRO37686
FIG. 3304: DNA327851, NSAP1, 217834_s_at
FIG. 3305: PRO83795
FIG. 3306: DNA328823, NP_057421.1, 217838_s_at
FIG. 3307: PRO84561
FIG. 3308: DNA330341, NP_006061.2, 217839_at
FIG. 3309: PRO85563
FIG. 3310: DNA254773, NP_057231.1, 217841_s_at
FIG. 3311: PRO49871
FIG. 3312: DNA330342, NP_067021.1, 217844_at
FIG. 3313: PRO85564
FIG. 3314: DNA329272, NP_055181.1, 217850_at
FIG. 3315: PRO84868
FIG. 3316A-B: DNA330343, AF403012,
217857_s_at
FIG. 3317: DNA330344, NP_057392.1, 217870_s_at
FIG. 3318: PRO85565
FIG. 3319: DNA326937, NP_002406.1, 217871_s_at
FIG. 3320: PRO83255
FIG. 3321: DNA330345, NP_055130.1, 217906_at
FIG. 3322: PRO85566
FIG. 3323: DNA330346, NP_054880.2, 217907_at
FIG. 3324: PRO85567
FIG. 3325: DNA325780, NP_060371.1, 217914_at
FIG. 3326: PRO82250
FIG. 3327: DNA327853, NP_054769.1, 217919_s_at
FIG. 3328: PRO82223
FIG. 3329: DNA330347, 255559.4, 217922_at
FIG. 3330: PRO85568
FIG. 3331: DNA330348, NP_079150.1, 217929_s_at
FIG. 3332: PRO85569
FIG. 3333: DNA330349, BC022093, 217931_at
FIG. 3334: DNA287241, NP_056991.1, 217933_s_at
FIG. 3335: PRO69516
FIG. 3336A-B: DNA225648, NP_061165.1,
217941_s_at
FIG. 3337: PRO36111
FIG. 3338: DNA326730, NP_057037.1, 217950_at
FIG. 3339: PRO83072
FIG. 3340: DNA329273, NP_037374.1, 217957_at
FIG. 3341: PRO84869
FIG. 3342: DNA328829, NP_057230.1, 217959_s_at
FIG. 3343: PRO84566
FIG. 3344: DNA328830, NP_061118.1, 217962_at
FIG. 3345: PRO84567
FIG. 3346: DNA329274, NP_055195.1, 217963_s_at
FIG. 3347: PRO84870
FIG. 3348: DNA325496, NP_037397.2, 217969_at
FIG. 3349: PRO82013
FIG. 3350: DNA327855, NP_057387.1, 217975_at
FIG. 3351: PRO83367
FIG. 3352: DNA227218, NP_003721.2, 217983_s_at
FIG. 3353: PRO37681
FIG. 3354: DNA227218, RNASE6PL, 217984_at
FIG. 3355: PRO37681
FIG. 3356A-B: DNA227238, NP_038476.1,
217985_s_at
FIG. 3357: PRO37701
FIG. 3358A-B: DNA227238, BAZ1A, 217986_s_at
FIG. 3359: PRO37701
FIG. 3360: DNA328831, NP_057329.1, 217989_at
FIG. 3361: PRO233
FIG. 3362: DNA328832, NP_067022.1, 217995_at
FIG. 3363: PRO84568
FIG. 3364: DNA328833, BC018929, 217996_at
FIG. 3365: PRO84569
FIG. 3366: DNA328834, AF220656, 217997_at
FIG. 3367: DNA287364, NP_031376.1, 218000_s_at
FIG. 3368: PRO69625
FIG. 3369: DNA273008, NP_003972.1, 218009_s_at
FIG. 3370: PRO61079
FIG. 3371: DNA330350, NP_006108.1, 218025_s_at
FIG. 3372: PRO85570
FIG. 3373: DNA328836, NP_054894.1, 218027_at
FIG. 3374: PRO84572
FIG. 3375: DNA329275, AF070673, 218032_at
FIG. 3376: PRO12342
FIG. 3377: DNA331592, ANKT, 218039_at
FIG. 3378: PRO82424
FIG. 3379: DNA328838, NP_054797.2, 218049_s_at
FIG. 3380: PRO70319
FIG. 3381: DNA330352, NP_075059.1, 218051_s_at
FIG. 3382: PRO85571
FIG. 3383: DNA329276, NP_077001.1, 218069_at
FIG. 3384: PRO12104
FIG. 3385: DNA328841, NP_060557.2, 218073_s_at
FIG. 3386: PRO84575
FIG. 3387: DNA329277, NP_054883.3, 218084_x_at
FIG. 3388: PRO6241
FIG. 3389: DNA330353, BC020796, 218085_at
FIG. 3390: PRO69464
FIG. 3391: DNA329278, NP_004495.1, 218092_s_at
FIG. 3392: PRO84871
FIG. 3393: DNA227313, NP_060945.1, 218095_s_at
FIG. 3394: PRO37776
FIG. 3395: DNA331593, MRPL4, 218105_s_at
FIG. 3396: PRO86598
FIG. 3397: DNA326596, NP_060624.1, 218115_at
FIG. 3398: PRO82954
FIG. 3399: DNA330355, NP_055063.1, 218117_at
FIG. 3400: PRO83289
FIG. 3401: DNA330356, NP_006318.1, 218118_s_at
FIG. 3402: PRO85572
FIG. 3403: DNA330357, NP_078786.2, 218130_at
FIG. 3404: PRO85573
FIG. 3405: DNA227155, NP_057654.1, 218135_at
FIG. 3406: PRO37618
FIG. 3407: DNA254496, NP_060076.1, 218149_s_at
FIG. 3408: PRO49604
FIG. 3409: DNA330358, NP_079012.1, 218154_at
FIG. 3410: PRO85574
FIG. 3411: DNA254739, NP_068766.1, 218156_s_at
FIG. 3412: PRO49837
FIG. 3413: DNA304470, PRO2577, 218172_s_at
FIG. 3414: PRO2577
FIG. 3415: DNA330359, NP_065145.1, 218178_s_at
FIG. 3416: PRO85575
FIG. 3417: DNA304495, NP_057156.1, 218193_s_at
FIG. 3418: PRO793
FIG. 3419A-C: DNA330360, NP_078789.1,
218204_s_at
FIG. 3420: PRO85576
FIG. 3421: DNA327858, NP_036473.1, 218238_at
FIG. 3422: PRO83800
FIG. 3423: DNA327858, CRFG, 218239_s_at
FIG. 3424: PRO83800
FIG. 3425A-B: DNA330361, CKAP2, 218252_at
FIG. 3426: PRO85577
FIG. 3427: DNA328850, NP_057187.1, 218254_s_at
FIG. 3428: PRO84581
FIG. 3429: DNA331594, MRPL24, 218270_at
FIG. 3430: PRO11652
FIG. 3431: DNA273230, NP_060914.1, 218273_s_at
FIG. 3432: PRO61257
FIG. 3433: DNA324444, NP_006333.1, 218308_at
FIG. 3434: PRO81108
FIG. 3435: DNA330363, NP_060252.1, 218331_s_at
FIG. 3436: PRO85578
FIG. 3437: DNA329281, NP_036526.2, 218336_at
FIG. 3438: PRO84874
FIG. 3439A-B: DNA330364, NP_004417.1,
218338_at
FIG. 3440: PRO85579
FIG. 3441: DNA272918, NP_055269.1, 218346_s_at
FIG. 3442: PRO61003
FIG. 3443: DNA327862, NP_060445.1, 218349_s_at
FIG. 3444: PRO83803
FIG. 3445: DNA328854, NP_056979.1, 218350_s_at
FIG. 3446: PRO84585
FIG. 3447A-B: DNA273415, KIF4A, 218355_at
FIG. 3448: PRO61414
FIG. 3449: DNA324890, NP_037525.1, 218356_at
FIG. 3450: PRO81496
FIG. 3451: DNA330365, NP_036591.1, 218357_s_at
FIG. 3452: PRO85580
FIG. 3453A-B: DNA331595, NP_073602.2,
218376_s_at
FIG. 3454: PRO86599
FIG. 3455: DNA330367, NP_057174.1, 218379_at
FIG. 3456: PRO85582
FIG. 3457: DNA328856, NP_068376.1, 218380_at
FIG. 3458: PRO84586
FIG. 3459: DNA227248, NP_006287.1, 218397_at
FIG. 3460: PRO37711
FIG. 3461A-B: DNA287192, NP_006178.1,
218400_at
FIG. 3462: PRO69478
FIG. 3463: DNA329912, TTC4, 218442_at
FIG. 3464: PRO85227
FIG. 3465: DNA150661, NP_057162.1, 218446_s_at
FIG. 3466: PRO12398
FIG. 3467: DNA304781, NP_057385.2, 218461_at
FIG. 3468: PRO71191
FIG. 3469: DNA328861, NP_057030.2, 218472_s_at
FIG. 3470: PRO84589
FIG. 3471: DNA330368, NP_064446.1, 218494_s_at
FIG. 3472: PRO85583
FIG. 3473: DNA150648, NP_037464.1, 218507_at
FIG. 3474: PRO11576
FIG. 3475: DNA328864, NP_060726.2, 218512_at
FIG. 3476: PRO84592
FIG. 3477: DNA330369, NP_060822.1, 218513_at
FIG. 3478: PRO85584
FIG. 3479: DNA330370, NP_060415.1, 218519_at
FIG. 3480: PRO190
FIG. 3481: DNA327867, NP_061873.2, 218532_s_at
FIG. 3482: PRO83808
FIG. 3483: DNA330371, NP_060813.1, 218535_s_at
FIG. 3484: PRO85585
FIG. 3485: DNA327868, NP_060601.2, 218542_at
FIG. 3486: PRO83809
FIG. 3487: DNA255113, NP_073587.1, 218543_s_at
FIG. 3488: PRO50195
FIG. 3489: DNA330372, NP_057117.1, 218549_s_at
FIG. 3490: PRO85586
FIG. 3491: DNA330373, NP_060751.1, 218552_at
FIG. 3492: PRO85587
FIG. 3493: DNA330374, NP_054901.1, 218556_at
FIG. 3494: PRO23321
FIG. 3495: DNA330375, NP_059142.1, 218558_s_at
FIG. 3496: PRO85588
FIG. 3497: DNA329587, NP_036256.1, 218566_s_at
FIG. 3498: PRO85121
FIG. 3499: DNA329286, NP_005691.2, 218567_x_at
FIG. 3500: PRO69644
FIG. 3501: DNA329054, NP_078805.2, 218578_at
FIG. 3502: PRO84716
FIG. 3503A-B: DNA273435, NP_057532.1,
218585_s_at
FIG. 3504: PRO61430
FIG. 3505: DNA227327, NP_060547.1, 218593_at
FIG. 3506: PRO37790
FIG. 3507: DNA328628, NP_060542.2, 218594_at
FIG. 3508: PRO84406
FIG. 3509: DNA287642, NP_060934.1, 218597_s_at
FIG. 3510: PRO9902
FIG. 3511A-B: DNA254789, NP_057301.1,
218603_at
FIG. 3512: PRO49887
FIG. 3513: DNA330376, NP_076962.1, 218622_at
FIG. 3514: PRO85589
FIG. 3515: DNA327869, NP_057672.1, 218625_at
FIG. 3516: PRO1898
FIG. 3517: DNA330377, NP_036577.1, 218638_s_at
FIG. 3518: PRO85590
FIG. 3519: DNA330378, NP_071741.2, 218662_s_at
FIG. 3520: PRO81126
FIG. 3521: DNA330378, HCAP-G, 218663_at
FIG. 3522: PRO81126
FIG. 3523: DNA287291, NP_067036.1, 218676_s_at
FIG. 3524: PRO69561
FIG. 3525: DNA304835, NP_071327.1, 218681_s_at
FIG. 3526: PRO71242
FIG. 3527: DNA330379, NP_073562.1, 218689_at
FIG. 3528: PRO85591
FIG. 3529: DNA329288, NP_061910.1, 218695_at
FIG. 3530: PRO84880
FIG. 3531: DNA287378, NP_060898.1, 218715_at
FIG. 3532: PRO69637
FIG. 3533: DNA327202, NP_057289.1, 218718_at
FIG. 3534: PRO200
FIG. 3535: DNA330380, NP_078937.2, 218722_s_at
FIG. 3536: PRO85592
FIG. 3537: DNA324251, NP_060880.2, 218726_at
FIG. 3538: PRO80935
FIG. 3539: DNA227617, NP_057161.1, 218732_at
FIG. 3540: PRO38080
FIG. 3541: DNA330381, NP_076958.1, 218741_at
FIG. 3542: PRO38668
FIG. 3543: DNA330382, NP_005724.1, 218755_at
FIG. 3544: PRO61907
FIG. 3545: DNA330383, NP_054828.1, 218782_s_at
FIG. 3546: PRO85593
FIG. 3547: DNA330384, NP_060388.1, 218802_at
FIG. 3548: PRO51129
FIG. 3549: DNA88315, NP_004098.1, 218831_s_at
FIG. 3550: PRO2743
FIG. 3551: DNA330385, NP_057733.2, 218859_s_at
FIG. 3552: PRO85594
FIG. 3553: DNA330386, NP_057394.1, 218866_s_at
FIG. 3554: PRO85595
FIG. 3555: DNA330387, NP_036309.1, 218875_s_at
FIG. 3556: PRO85596
FIG. 3557: DNA327874, BC022791, 218880_at
FIG. 3558: PRO4805
FIG. 3559A-B: DNA271829, NP_006775.1,
218882_s_at
FIG. 3560: PRO60109
FIG. 3561: DNA330388, NP_078905.1, 218883_s_at
FIG. 3562: PRO85597
FIG. 3563: DNA226633, NP_060376.1, 218886_at
FIG. 3564: PRO37096
FIG. 3565: DNA304780, NP_060562.2, 218888_s_at
FIG. 3566: PRO69889
FIG. 3567: DNA256762, AK022882, 218889_at
FIG. 3568: PRO51695
FIG. 3569: DNA328881, NP_057706.2, 218890_x_at
FIG. 3570: PRO49469
FIG. 3571: DNA325622, NP_060518.1, 218894_s_at
FIG. 3572: PRO82113
FIG. 3573: DNA225694, NP_060087.1, 218902_at
FIG. 3574: PRO36157
FIG. 3575: DNA325690, NP_076973.1, 218903_s_at
FIG. 3576: PRO82179
FIG. 3577: DNA328364, SIGIRR, 218921_at
FIG. 3578: PRO84223
FIG. 3579: DNA287166, NP_055129.1, 218943_s_at
FIG. 3580: PRO69459
FIG. 3581: DNA330389, NP_079221.1, 218979_at
FIG. 3582: PRO85598
FIG. 3583: DNA329050, NP_057053.1, 218982_s_at
FIG. 3584: PRO84712
FIG. 3585: DNA330390, NP_057630.1, 218983_at
FIG. 3586: PRO85599
FIG. 3587: DNA288277, NP_061915.1, 218984_at
FIG. 3588: PRO70034
FIG. 3589: DNA256265, NP_060101.1, 218986_s_at
FIG. 3590: PRO51309
FIG. 3591: DNA227194, FLJ11000, 218999_at
FIG. 3592: PRO37657
FIG. 3593: DNA330391, NP_076999.1, 219000_s_at
FIG. 3594: PRO34008
FIG. 3595: DNA330392, NP_078923.2, 219007_at
FIG. 3596: PRO85600
FIG. 3597: DNA328885, NP_061108.2, 219017_at
FIG. 3598: PRO50294
FIG. 3599: DNA330393, NP_067635.1, 219024_at
FIG. 3600: PRO85601
FIG. 3601: DNA329292, NP_057185.1, 219031_s_at
FIG. 3602: PRO84882
FIG. 3603: DNA330394, NP_079402.1, 219035_s_at
FIG. 3604: PRO85602
FIG. 3605: DNA329293, NP_057136.1, 219037_at
FIG. 3606: PRO84883
FIG. 3607: DNA328886, NP_078811.1, 219040_at
FIG. 3608: PRO84610
FIG. 3609: DNA331596, NP_060841.1, 219049_at
FIG. 3610: PRO84884
FIG. 3611: DNA330395, NP_060212.1, 219062_s_at
FIG. 3612: PRO85603
FIG. 3613: DNA330396, NP_077303.1, 219088_s_at
FIG. 3614: PRO85604
FIG. 3615: DNA330397, NP_054873.1, 219094_at
FIG. 3616: PRO85605
FIG. 3617: DNA331597, PLA2G4B, 219095_at
FIG. 3618: PRO86600
FIG. 3619: DNA330398, NP_060367.1, 219133_at
FIG. 3620: PRO85606
FIG. 3621: DNA297191, NP_060962.2, 219148_at
FIG. 3622: PRO70808
FIG. 3623: DNA329295, NP_036549.1, 219155_at
FIG. 3624: PRO84885
FIG. 3625A-B: DNA329438, NP_476516.1,
219158_s_at
FIG. 3626: PRO85008
FIG. 3627: DNA328892, NP_067643.2, 219165_at
FIG. 3628: PRO84616
FIG. 3629: DNA330399, NP_060609.1, 219166_at
FIG. 3630: PRO85607
FIG. 3631: DNA330400, NP_078796.1, 219176_at
FIG. 3632: PRO85608
FIG. 3633: DNA271455, NP_057735.1, 219179_at
FIG. 3634: PRO59751
FIG. 3635: DNA330401, NP_057377.1, 219191_s_at
FIG. 3636: PRO85609
FIG. 3637: DNA330402, NP_076996.1, 219200_at
FIG. 3638: PRO85610
FIG. 3639: DNA287235, NP_060598.1, 219204_s_at
FIG. 3640: PRO69514
FIG. 3641: DNA327879, NP_071451.1, 219209_at
FIG. 3642: PRO83818
FIG. 3643: DNA330403, NP_059110.1, 219211_at
FIG. 3644: PRO85611
FIG. 3645: DNA330404, ZNF361, 219228_at
FIG. 3646: PRO85612
FIG. 3647: DNA225594, NP_037404.1, 219229_at
FIG. 3648: PRO36057
FIG. 3649: DNA328894, NP_060796.1, 219243_at
FIG. 3650: PRO84617
FIG. 3651: DNA329296, NP_060328.1, 219258_at
FIG. 3652: PRO84886
FIG. 3653: DNA304461, NP_054877.1, 219283_at
FIG. 3654: PRO71039
FIG. 3655: DNA330405, RBM15, 219286_s_at
FIG. 3656: PRO85613
FIG. 3657A-B: DNA329076, NP_064627.1,
219306_at
FIG. 3658: PRO84733
FIG. 3659: DNA329914, FLJ12542, 219311_at
FIG. 3660: PRO85229
FIG. 3661: DNA255939, NP_078876.1, 219315_s_at
FIG. 3662: PRO50991
FIG. 3663: DNA287404, NP_073748.1, 219334_s_at
FIG. 3664: PRO69661
FIG. 3665: DNA254710, NP_060382.1, 219352_at
FIG. 3666: PRO49810
FIG. 3667: DNA325169, HSPC177, 219356_s_at
FIG. 3668: PRO81734
FIG. 3669: DNA330406, NP_079368.1, 219359_at
FIG. 3670: PRO85614
FIG. 3671: DNA330407, NP_057026.2, 219363_s_at
FIG. 3672: PRO85615
FIG. 3673: DNA330408, NP_077024.1, 219364_at
FIG. 3674: PRO85616
FIG. 3675: DNA254518, NP_057354.1, 219371_s_at
FIG. 3676: PRO49625
FIG. 3677: DNA327886, NP_060832.1, 219399_at
FIG. 3678: PRO41077
FIG. 3679: DNA256417, NP_077271.1, 219402_s_at
FIG. 3680: PRO51457
FIG. 3681A-B: DNA327887, NP_006656.1,
219403_s_at
FIG. 3682: PRO83823
FIG. 3683: DNA271811, NP_036514.1, 219421_at
FIG. 3684: PRO60092
FIG. 3685: DNA329014, NP_005746.2, 219424_at
FIG. 3686: PRO9998
FIG. 3687: DNA328901, FLJ20533, 219449_s_at
FIG. 3688: PRO84622
FIG. 3689: DNA328902, NP_071750.1, 219452_at
FIG. 3690: PRO84623
FIG. 3691: DNA328367, RIN3, 219456_s_at
FIG. 3692: PRO84226
FIG. 3693: DNA331598, AK026092, 219457_s_at
FIG. 3694: PRO86601
FIG. 3695: DNA327890, NP_079021.1, 219493_at
FIG. 3696: PRO83826
FIG. 3697A-B: DNA227179, NP_059120.1,
219505_at
FIG. 3698: PRO37642
FIG. 3699A-C: DNA331599, BCL11B, 219528_s_at
FIG. 3700: PRO86602
FIG. 3701: DNA329300, GEMIN6, 219539_at
FIG. 3702: PRO84889
FIG. 3703: DNA328908, 7691567.2, 219540_at
FIG. 3704: PRO84629
FIG. 3705: DNA256737, NP_060276.1, 219541_at
FIG. 3706: PRO51671
FIG. 3707: DNA330410, NP_060925.1, 219555_s_at
FIG. 3708: PRO85618
FIG. 3709A-B: DNA331600, NP_061985.1,
219577_s_at
FIG. 3710: PRO86603
FIG. 3711: DNA325053, NP_060230.2, 219588_s_at
FIG. 3712: PRO81637
FIG. 3713: DNA330412, NP_057617.1, 219594_at
FIG. 3714: PRO23600
FIG. 3715: DNA331601, NP_071915.1, 219628_at
FIG. 3716: PRO85620
FIG. 3717: DNA330414, NP_057615.1, 219657_s_at
FIG. 3718: PRO81138
FIG. 3719A-B: DNA274044, HSM801565,
219671_at
FIG. 3720: PRO61987
FIG. 3721: DNA293243, RCP, 219681_s_at
FIG. 3722: PRO70699
FIG. 3723: DNA255161, NP_071430.1, 219684_at
FIG. 3724: PRO50241
FIG. 3725: DNA287206, NP_060124.1, 219691_at
FIG. 3726: PRO69488
FIG. 3727A-B: DNA330297, NP_065138.2,
219700_at
FIG. 3728: PRO85524
FIG. 3729: DNA330416, TDP1, 219715_s_at
FIG. 3730: PRO85622
FIG. 3731: DNA330417, NP_085144.1, 219716_at
FIG. 3732: PRO21341
FIG. 3733A-B: DNA227255, NP_036579.1,
219753_at
FIG. 3734: PRO37718
FIG. 3735: DNA328919, NP_078987.1, 219777_at
FIG. 3736: PRO84637
FIG. 3737A-B: DNA331602, NP_060568.3,
219787_s_at
FIG. 3738: PRO86604
FIG. 3739: DNA255822, NP_036346.1, 219797_at
FIG. 3740: PRO50877
FIG. 3741: DNA227305, NP_064564.1, 219806_s_at
FIG. 3742: PRO37768
FIG. 3743: DNA329303, NP_054737.1, 219819_s_at
FIG. 3744: PRO84892
FIG. 3745: DNA287295, NP_078784.1, 219836_at
FIG. 3746: PRO69564
FIG. 3747: DNA287234, NP_114174.1, 219862_s_at
FIG. 3748: PRO69513
FIG. 3749: DNA287221, NP_057407.1, 219863_at
FIG. 3750: PRO69500
FIG. 3751: DNA330419, NP_038469.1, 219864_s_at
FIG. 3752: PRO85624
FIG. 3753: DNA255255, LOC64116, 219869_s_at
FIG. 3754: PRO50332
FIG. 3755: DNA330420, NP_078890.1, 219871_at
FIG. 3756: PRO85625
FIG. 3757: DNA256325, NP_005470.1, 219889_at
FIG. 3758: PRO51367
FIG. 3759: DNA330421, NP_057438.2, 219911_s_at
FIG. 3760: PRO85626
FIG. 3761A-B: DNA330422, NP_057736.2,
219913_s_at
FIG. 3762: PRO85627
FIG. 3763: DNA227787, NP_060606.1, 219918_s_at
FIG. 3764: PRO38250
FIG. 3765: DNA330423, NP_037466.2, 219920_s_at
FIG. 3766: PRO85628
FIG. 3767A-B: DNA330424, LTBP3, 219922_s_at
FIG. 3768: PRO85629
FIG. 3769: DNA328924, NP_057150.2, 219933_at
FIG. 3770: PRO84641
FIG. 3771: DNA218280, NP_068570.1, 219971_at
FIG. 3772: PRO34332
FIG. 3773: DNA325979, NP_060924.4, 219978_s_at
FIG. 3774: PRO82424
FIG. 3775: DNA330425, NP_078956.1, 219990_at
FIG. 3776: PRO85630
FIG. 3777A-B: DNA330426, SGKL, 220038_at
FIG. 3778: PRO85631
FIG. 3779: DNA328926, NP_064703.1, 220046_s_at
FIG. 3780: PRO84643
FIG. 3781A-B: DNA218680, NP_071731.1,
220048_at
FIG. 3782: PRO21724
FIG. 3783: DNA330427, NP_036593.1, 220052_s_at
FIG. 3784: PRO85632
FIG. 3785: DNA330428, NP_060385.1, 220060_s_at
FIG. 3786: PRO85633
FIG. 3787: DNA330537, NP_060533.2, 220085_at
FIG. 3788: PRO81892
FIG. 3789: DNA256091, NP_071385.1, 220094_s_at
FIG. 3790: PRO51141
FIG. 3791: DNA330430, NP_078945.1, 220112_at
FIG. 3792: PRO85634
FIG. 3793: DNA330431, NP_055198.1, 220118_at
FIG. 3794: PRO85635
FIG. 3795: DNA227302, NP_037401.1, 220132_s_at
FIG. 3796: PRO37765
FIG. 3797: DNA330432, NP_079219.1, 220169_at
FIG. 3798: PRO85636
FIG. 3799: DNA331603, TMPRSS3, 220177_s_at
FIG. 3800: PRO83482
FIG. 3801: DNA256291, NP_079182.1, 220232_at
FIG. 3802: PRO51335
FIG. 3803: DNA330434, NP_060842.1, 220235_s_at
FIG. 3804: PRO85637
FIG. 3805: DNA330435, NP_060179.1, 220306_at
FIG. 3806: PRO85638
FIG. 3807: DNA330436, NP_037394.1, 220319_s_at
FIG. 3808: PRO85639
FIG. 3809: DNA327904, NP_071419.2, 220330_s_at
FIG. 3810: PRO83839
FIG. 3811: DNA287186, NP_061134.1, 220358_at
FIG. 3812: PRO69472
FIG. 3813A-B: DNA330437, NP_079366.1,
220370_s_at
FIG. 3814: PRO85640
FIG. 3815: DNA330438, NP_061026.1, 220485_s_at
FIG. 3816: PRO50795
FIG. 3817: DNA327214, NP_078991.2, 220495_s_at
FIG. 3818: PRO83483
FIG. 3819: DNA324252, NP_060444.1, 220521_s_at
FIG. 3820: PRO80936
FIG. 3821: DNA331604, PHEMX, 220558_x_at
FIG. 3822: PRO86605
FIG. 3823: DNA256363, NP_057686.1, 220565_at
FIG. 3824: PRO51405
FIG. 3825: DNA255798, NP_079265.1, 220576_at
FIG. 3826: PRO50853
FIG. 3827: DNA330440, NP_079098.1, 220591_s_at
FIG. 3828: PRO85642
FIG. 3829: DNA255734, NP_057607.1, 220646_s_at
FIG. 3830: PRO50791
FIG. 3831A-B: DNA327908, MCM10, 220651_s_at
FIG. 3832: PRO83843
FIG. 3833: DNA329306, NP_079149.2, 220655_at
FIG. 3834: PRO84895
FIG. 3835A-B: DNA327909, ARNTL2, 220658_s_at
FIG. 3836: PRO83844
FIG. 3837: DNA329307, NP_037483.1, 220684_at
FIG. 3838: PRO84896
FIG. 3839: DNA323756, NP_057267.2, 220688_s_at
FIG. 3840: PRO80512
FIG. 3841: DNA331380, DKFZp566O084Homo,
220690_s_at
FIG. 3842: DNA330442, NP_054866.1, 220692_at
FIG. 3843: PRO85643
FIG. 3844: DNA330443, NP_061086.1, 220702_at
FIG. 3845: PRO85644
FIG. 3846: DNA288247, NP_478059.1, 220892_s_at
FIG. 3847: PRO70011
FIG. 3848: DNA327916, NP_079466.1, 220940_at
FIG. 3849: PRO83851
FIG. 3850: DNA327953, NP_055182.2, 220942_x_at
FIG. 3851: PRO83878
FIG. 3852: DNA327917, NP_112240.1, 220966_x_at
FIG. 3853: PRO83852
FIG. 3854: DNA329078, VMP1, 220990_s_at
FIG. 3855: PRO23253
FIG. 3856A-B: DNA254516, NP_112196.1,
220992_s_at
FIG. 3857: PRO49623
FIG. 3858: DNA330444, NP_110405.1, 220999_s_at
FIG. 3859: PRO85645
FIG. 3860: DNA324246, NP_112188.1, 221004_s_at
FIG. 3861: PRO80930
FIG. 3862: DNA330445, NP_112174.1, 221012_s_at
FIG. 3863: PRO85646
FIG. 3864A-B: DNA254816, NP_110444.1,
221031_s_at
FIG. 3865: PRO49912
FIG. 3866: DNA330446, NP_054889.1, 221046_s_at
FIG. 3867: PRO85647
FIG. 3868: DNA330447, NP_079174.1, 221080_s_at
FIG. 3869: PRO85648
FIG. 3870: DNA226227, NP_060872.1, 221111_at
FIG. 3871: PRO36690
FIG. 3872: DNA227267, NP_061130.1, 221123_x_at
FIG. 3873: PRO37730
FIG. 3874: DNA217256, NP_065386.1, 221165_s_at
FIG. 3875: PRO34298
FIG. 3876: DNA329310, AK027224, 221185_s_at
FIG. 3877: PRO84899
FIG. 3878: DNA324408, NP_060493.2, 221203_s_at
FIG. 3879: PRO81072
FIG. 3880A-B: DNA330448, NP_059111.1,
221221_s_at
FIG. 3881: PRO85649
FIG. 3882: DNA331605, CISH, 221223_x_at
FIG. 3883: PRO86458
FIG. 3884: DNA330450, AK025947, 221235_s_at
FIG. 3885: PRO85651
FIG. 3886: DNA330451, NP_110429.1, 221249_s_at
FIG. 3887: PRO85652
FIG. 3888: DNA330452, NP_112494.2, 221258_s_at
FIG. 3889: PRO85653
FIG. 3890: DNA295327, NP_068575.1, 221271_at
FIG. 3891: PRO70773
FIG. 3892: DNA330453, NP_112597.1, 221277_s_at
FIG. 3893: PRO85654
FIG. 3894: DNA329312, NP_005205.2, 221331_x_at
FIG. 3895: PRO84901
FIG. 3896: DNA288250, NP_112487.1, 221434_s_at
FIG. 3897: PRO70013
FIG. 3898: DNA330454, NP_112589.1, 221436_s_at
FIG. 3899: PRO85655
FIG. 3900: DNA330455, 1097190.16, 221477_s_at
FIG. 3901: PRO85656
FIG. 3902: DNA150865, NP_057005.1, 221488_s_at
FIG. 3903: PRO11587
FIG. 3904: DNA272972, NP_057356.1, 221496_s_at
FIG. 3905: PRO61052
FIG. 3906A-B: DNA329316, AF158555,
221510_s_at
FIG. 3907: PRO84904
FIG. 3908: DNA330456, NP_060571.1, 221520_s_at
FIG. 3909: PRO85657
FIG. 3910: DNA326221, NP_057179.1, 221521_s_at
FIG. 3911: PRO82634
FIG. 3912: DNA328953, NP_004086.1, 221539_at
FIG. 3913: PRO70296
FIG. 3914: DNA329317, AF288571, 221558_s_at
FIG. 3915: PRO81157
FIG. 3916: DNA330457, NP_076944.1, 221559_s_at
FIG. 3917: PRO85658
FIG. 3918: DNA329319, BC006401, 221601_s_at
FIG. 3919: PRO1607
FIG. 3920: DNA329319, NP_005440.1, 221602_s_at
FIG. 3921: PRO1607
FIG. 3922: DNA254308, NP_060950.1, 221622_s_at
FIG. 3923: PRO49419
FIG. 3924: DNA287254, NP_077004.1, 221637_s_at
FIG. 3925: PRO69528
FIG. 3926: DNA330458, NP_060634.1, 221652_s_at
FIG. 3927: PRO85659
FIG. 3928: DNA218280, IL21R, 221658_s_at
FIG. 3929: PRO34332
FIG. 3930: DNA327927, NP_037390.2, 221666_s_at
FIG. 3931: PRO57311
FIG. 3932: DNA254777, NP_055140.1, 221676_s_at
FIG. 3933: PRO49875
FIG. 3934: DNA330459, NP_060083.1, 221677_s_at
FIG. 3935: PRO50083
FIG. 3936: DNA330460, NP_060255.2, 221685_s_at
FIG. 3937: PRO85660
FIG. 3938: DNA273185, DNA273185, 221727_at
FIG. 3939: DNA330461, BC005104, 221732_at
FIG. 3940: DNA328961, NP_443112.1, 221756_at
FIG. 3941: PRO84667
FIG. 3942: DNA328961, MGC17330, 221757_at
FIG. 3943: PRO84667
FIG. 3944: DNA330462, NP_060103.1, 221766_s_at
FIG. 3945: PRO85661
FIG. 3946: DNA193901, DNA193901, 221768_at
FIG. 3947: PRO23319
FIG. 3948: DNA328964, AK056028, 221770_at
FIG. 3949: PRO84669
FIG. 3950: DNA330463, HSM801191, 221790_s_at
FIG. 3951A-B: DNA151745, DNA151745,
221805_at
FIG. 3952: PRO12033
FIG. 3953: DNA274058, NP_057203.1, 221816_s_at
FIG. 3954: PRO61999
FIG. 3955: DNA325039, NP_004902.1, 221824_s_at
FIG. 3956: PRO2733
FIG. 3957: DNA273311, NP_003022.1, 221833_at
FIG. 3958: PRO61319
FIG. 3959: DNA272419, AF105036, 221841_s_at
FIG. 3960: PRO60672
FIG. 3961: DNA330464, NP_067082.1, 221882_s_at
FIG. 3962: PRO85663
FIG. 3963A-B: DNA330465, 253695.2, 221916_at
FIG. 3964: PRO85664
FIG. 3965A-B: DNA330466, AB018304, 221922_at
FIG. 3966: DNA329321, NP_112493.1, 221931_s_at
FIG. 3967: PRO84906
FIG. 3968: DNA330467, NP_060114.1, 221986_s_at
FIG. 3969: PRO85665
FIG. 3970: DNA287235, FLJ10534, 221987_s_at
FIG. 3971: PRO69514
FIG. 3972: DNA327114, RPL10, 221989_at
FIG. 3973: PRO62466
FIG. 3974: DNA331606, BC018529, 222017_x_at
FIG. 3975: PRO86606
FIG. 3976: DNA257797, DNA257797, 222036_s_at
FIG. 3977: DNA257798, DNA257798, 222037_at
FIG. 3978: DNA330468, 1454101.4, 222044_at
FIG. 3979: PRO85666
FIG. 3980: DNA329919, BC013365, 222045_s_at
FIG. 3981: PRO85234
FIG. 3982: DNA304466, NP_004834.1, 222062_at
FIG. 3983: PRO34336
FIG. 3984: DNA325648, NP_037409.2, 222077_s_at
FIG. 3985: PRO82139
FIG. 3986: DNA331386, HST000012, 222150_s_at
FIG. 3987: DNA287209, NP_056350.1, 222154_s_at
FIG. 3988: PRO69490
FIG. 3989: DNA256784, NP_075069.1, 222209_s_at
FIG. 3990: PRO51716
FIG. 3991: DNA328977, NP_071344.1, 222216_s_at
FIG. 3992: PRO84678
FIG. 3993: DNA330469, NP_056249.1, 222250_s_at
FIG. 3994: PRO85667
FIG. 3995: DNA328885, EKI1, 222262_s_at
FIG. 3996: PRO50294
FIG. 3997: DNA330470, 096828.1, 222307_at
FIG. 3998: PRO85668
FIG. 3999: DNA330471, 027307.1, 222309_at
FIG. 4000: PRO85669
FIG. 4001: DNA330472, 128864.1, 222326_at
FIG. 4002: PRO85670
FIG. 4003: DNA330473, NP_060676.2, 222387_s_at
FIG. 4004: PRO85671
FIG. 4005: DNA330474, AF186382, 222388_s_at
FIG. 4006: PRO85672
FIG. 4007: DNA331607, HSA251830, 222392_x_at
FIG. 4008: PRO86607
FIG. 4009A-B: DNA270901, NP_004238.1,
222398_s_at
FIG. 4010: PRO59235
FIG. 4011: DNA325821, BC014334, 222402_at
FIG. 4012: PRO82287
FIG. 4013: DNA227358, HSPC121, 222404_x_at
FIG. 4014: PRO37821
FIG. 4015: DNA330476, AK027421, 222405_at
FIG. 4016: PRO85674
FIG. 4017: DNA328819, CGI-127, 222408_s_at
FIG. 4018: PRO84557
FIG. 4019: DNA331608, SNX5, 222417_s_at
FIG. 4020: PRO69560
FIG. 4021: DNA326307, NP_056399.1, 222425_s_at
FIG. 4022: PRO82707
FIG. 4023: DNA227223, GK001, 222432_s_at
FIG. 4024: PRO37686
FIG. 4025A-B: DNA329326, NP_005110.1,
222439_s_at
FIG. 4026: PRO84910
FIG. 4027: DNA327939, NP_060654.1, 222442_s_at
FIG. 4028: PRO83869
FIG. 4029: DNA329327, AF198620, 222443_s_at
FIG. 4030: PRO84911
FIG. 4031A-B: DNA256489, NP_079110.1,
222464_s_at
FIG. 4032: PRO51526
FIG. 4033A-B: DNA225648, ERBB2IP, 222473_s_at
FIG. 4034: PRO36111
FIG. 4035: DNA304460, BC003048, 222500_at
FIG. 4036: PRO4984
FIG. 4037: DNA330477, NP_036227.1, 222516_at
FIG. 4038: PRO37979
FIG. 4039: DNA329328, NP_067026.2, 222532_at
FIG. 4040: PRO84912
FIG. 4041: DNA16435, DNA16435, 222543_at
FIG. 4042: PRO276
FIG. 4043: DNA330478, NP_056978.2, 222557_at
FIG. 4044: PRO85675
FIG. 4045A-B: DNA330479, 900264.1, 222572_at
FIG. 4046: PRO85676
FIG. 4047: DNA330480, NP_060697.2, 222600_s_at
FIG. 4048: PRO85677
FIG. 4049A-B: DNA330481, AB058718, 222603_at
FIG. 4050: DNA330482, AK027468, 222606_at
FIG. 4051: PRO85678
FIG. 4052: DNA330483, AK001472, 222608_s_at
FIG. 4053: PRO85679
FIG. 4054: DNA329330, NP_057130.1, 222609_s_at
FIG. 4055: PRO84914
FIG. 4056A-B: DNA331609, 402471.3, 222613_at
FIG. 4057: PRO86608
FIG. 4058: DNA330485, NP_057415.1, 222624_s_at
FIG. 4059: PRO85681
FIG. 4060: DNA327942, NP_060596.1, 222642_s_at
FIG. 4061: PRO83870
FIG. 4062: DNA327943, NP_055399.1, 222646_s_at
FIG. 4063: PRO865
FIG. 4064A-B: DNA273435, RAMP, 222680_s_at
FIG. 4065: PRO61430
FIG. 4066: DNA330486, HSM802473, 222692_s_at
FIG. 4067: DNA330487, AB052751, 222696_at
FIG. 4068: DNA330488, AK025568, 222704_at
FIG. 4069: PRO85682
FIG. 4070: DNA272874, NP_057111.1, 222714_s_at
FIG. 4071: PRO60967
FIG. 4072: DNA275116, DNA275116, 222726_s_at
FIG. 4073: DNA330489, BC019909, 222740_at
FIG. 4074: PRO85683
FIG. 4075: DNA330490, 399171.38, 222754_at
FIG. 4076: PRO85684
FIG. 4077: DNA330491, BC002522, 222759_at
FIG. 4078: PRO85685
FIG. 4079A-B: DNA330492, FLJ11294, 222763_s_at
FIG. 4080: PRO85686
FIG. 4081: DNA329332, LOC51605, 222768_s_at
FIG. 4082: PRO84916
FIG. 4083: DNA330493, AK025248, 222770_s_at
FIG. 4084: PRO85687
FIG. 4085: DNA304780, NETO2, 222774_s_at
FIG. 4086: PRO69889
FIG. 4087: DNA330494, BC020651, 222775_s_at
FIG. 4088: PRO85688
FIG. 4089: DNA330495, NP_060468.1, 222781_s_at
FIG. 4090: PRO85689
FIG. 4091: DNA330496, HSM802366, 222793_at
FIG. 4092: DNA330395, FLJ20281, 222816_s_at
FIG. 4093: PRO85603
FIG. 4094A-B: DNA331610, TBDN100,
222837_s_at
FIG. 4095: PRO86609
FIG. 4096: DNA330881, AB027233, 222838_at
FIG. 4097: PRO1138
FIG. 4098: DNA329335, AK023411, 222843_at
FIG. 4099: PRO84919
FIG. 4100: DNA273489, NP_055210.1, 222858_s_at
FIG. 4101: PRO61472
FIG. 4102: DNA273489, DAPP1, 222859_s_at
FIG. 4103: PRO61472
FIG. 4104: DNA330498, NP_036225.1, 222862_s_at
FIG. 4105: PRO85691
FIG. 4106: DNA329336, NP_057144.1, 222867_s_at
FIG. 4107: PRO84920
FIG. 4108: DNA287404, FLJ22833, 222872_x_at
FIG. 4109: PRO69661
FIG. 4110: DNA330499, AK026944, 222875_at
FIG. 4111: PRO85692
FIG. 4112: DNA330500, AK022872, 222889_at
FIG. 4113: PRO85693
FIG. 4114A-C: DNA330409, HSA404614,
222895_s_at
FIG. 4115: PRO85617
FIG. 4116: DNA330501, AK022792, 222958_s_at
FIG. 4117: PRO85694
FIG. 4118A-B: DNA330502, AB042719,
222962_s_at
FIG. 4119: PRO85695
FIG. 4120: DNA329337, AF279437, 222974_at
FIG. 4121: PRO10096
FIG. 4122: DNA329338, 459502.10, 222977_at
FIG. 4123: PRO84921
FIG. 4124A-B: DNA329339, 459502.5, 222978_at
FIG. 4125: PRO84922
FIG. 4126: DNA329340, AF078866, 222979_s_at
FIG. 4127: PRO81805
FIG. 4128: DNA152786, NP_057215.1, 222980_at
FIG. 4129: PRO10928
FIG. 4130: DNA152786, RAB10, 222981_s_at
FIG. 4131: PRO10928
FIG. 4132A-B: DNA287236, AB024334, 222985_at
FIG. 4133: PRO10607
FIG. 4134: DNA330503, NP_038466.2, 222989_s_at
FIG. 4135: PRO85696
FIG. 4136: DNA331611, UBQLN1, 222991_s_at
FIG. 4137: PRO86610
FIG. 4138: DNA330504, NP_057575.2, 222993_at
FIG. 4139: PRO84923
FIG. 4140: DNA329571, NP_057547.3, 222996_s_at
FIG. 4141: PRO51662
FIG. 4142: DNA326195, NP_054781.1, 223018_at
FIG. 4143: PRO82611
FIG. 4144: DNA330505, BC005937, 223021_x_at
FIG. 4145: PRO85697
FIG. 4146A-B: DNA329342, AF172847, 223027_at
FIG. 4147: PRO84924
FIG. 4148: DNA329344, FRSB, 223035_s_at
FIG. 4149: PRO84926
FIG. 4150: DNA330506, NP_067061.1, 223038_s_at
FIG. 4151: PRO82123
FIG. 4152: DNA330507, AK054681, 223039_at
FIG. 4153: PRO85698
FIG. 4154: DNA287260, NP_057184.1, 223040_at
FIG. 4155: PRO69532
FIG. 4156: DNA324198, HSM801908, 223044_at
FIG. 4157: PRO37675
FIG. 4158: DNA330508, AF116694, 223047_at
FIG. 4159: PRO85699
FIG. 4160: DNA189412, NP_057390.1, 223054_at
FIG. 4161: PRO25349
FIG. 4162A-B: DNA256347, AF298880,
223055_s_at
FIG. 4163: PRO51389
FIG. 4164A-B: DNA329345, AB033117,
223056_s_at
FIG. 4165: DNA327948, NP_060394.1, 223060_at
FIG. 4166: PRO69660
FIG. 4167: DNA288247, PSA, 223062_s_at
FIG. 4168: PRO70011
FIG. 4169: DNA330509, AK024555, 223066_at
FIG. 4170: PRO80652
FIG. 4171: DNA227294, NP_060225.1, 223076_s_at
FIG. 4172: PRO37757
FIG. 4173: DNA331612, AF097492, 223079_s_at
FIG. 4174: PRO86611
FIG. 4175: DNA326258, NP_077273.1, 223081_at
FIG. 4176: PRO82665
FIG. 4177: DNA329346, AK027070, 223085_at
FIG. 4178: PRO84928
FIG. 4179: DNA327949, NP_057581.2, 223086_x_at
FIG. 4180: PRO83874
FIG. 4181: DNA331613, 238178.17, 223087_at
FIG. 4182: PRO86612
FIG. 4183: DNA329347, NP_060949.1, 223088_x_at
FIG. 4184: PRO84929
FIG. 4185: DNA330511, AK001338, 223090_x_at
FIG. 4186: PRO85701
FIG. 4187: DNA324209, NP_057018.1, 223096_at
FIG. 4188: PRO80902
FIG. 4189: DNA329349, NP_054861.1, 223100_s_at
FIG. 4190: PRO84931
FIG. 4191: DNA327917, MGC3038, 223101_s_at
FIG. 4192: PRO83852
FIG. 4193: DNA330512, NP_056494.1, 223109_at
FIG. 4194: PRO85702
FIG. 4195: DNA330436, MIR, 223129_x_at
FIG. 4196: PRO85639
FIG. 4197: DNA330513, AF212221, 223130_s_at
FIG. 4198: PRO85703
FIG. 4199A-: DNA330514, DDX36, 223138_s_at
FIG. 4200: PRO85704
FIG. 4201A-: DNA330514, AF217190, 223139_s_at
FIG. 4202: PRO85704
FIG. 4203: DNA325557, NP_115675.1, 223151_at
FIG. 4204: PRO82060
FIG. 4205: DNA329352, NP_057154.2, 223156_at
FIG. 4206: PRO84932
FIG. 4207: DNA329353, NP_113665.1, 223179_at
FIG. 4208: PRO84933
FIG. 4209: DNA254276, NP_054896.1, 223180_s_at
FIG. 4210: PRO49387
FIG. 4211: DNA327953, E2IG5, 223193_x_at
FIG. 4212: PRO83878
FIG. 4213A-B: DNA281444, NP_064544.1,
223197_s_at
FIG. 4214: PRO66283
FIG. 4215: DNA304467, NP_115703.1, 223212_at
FIG. 4216: PRO71043
FIG. 4217: DNA227267, LOC55893, 223216_x_at
FIG. 4218: PRO37730
FIG. 4219: DNA327954, NP_113646.1, 223220_s_at
FIG. 4220: PRO83879
FIG. 4221: DNA330515, NP_004580.1, 223221_at
FIG. 4222: PRO85705
FIG. 4223: DNA329321, SEC13L, 223225_s_at
FIG. 4224: PRO84906
FIG. 4225: DNA247474, NP_054895.1, 223229_at
FIG. 4226: PRO44999
FIG. 4227: DNA330516, AK000796, 223239_at
FIG. 4228: PRO85706
FIG. 4229: DNA287171, NP_036312.1, 223240_at
FIG. 4230: PRO69462
FIG. 4231: DNA324046, NP_115700.1, 223272_s_at
FIG. 4232: PRO80763
FIG. 4233: DNA330517, NP_115879.1, 223273_at
FIG. 4234: PRO85707
FIG. 4235: DNA330518, BC002493, 223274_at
FIG. 4236: PRO85708
FIG. 4237: DNA330519, NP_060607.1, 223275_at
FIG. 4238: PRO85709
FIG. 4239: DNA330520, NP_005777.2, 223283_s_at
FIG. 4240: PRO85710
FIG. 4241: DNA330521, BC002762, 223286_at
FIG. 4242: PRO85711
FIG. 4243A-B: DNA330522, AF250920,
223287_s_at
FIG. 4244: PRO85712
FIG. 4245: DNA330523, BC001220, 223294_at
FIG. 4246: PRO85713
FIG. 4247: DNA330524, MGC4268, 223297_at
FIG. 4248: PRO85714
FIG. 4249: DNA329356, NP_115671.1, 223304_at
FIG. 4250: PRO84935
FIG. 4251: DNA330454, BC002551, 223307_at
FIG. 4252: PRO85655
FIG. 4253: DNA330526, NP_115682.1, 223318_s_at
FIG. 4254: PRO34564
FIG. 4255A-B: DNA330527, AF272663, 223319_at
FIG. 4256: PRO85716
FIG. 4257: DNA329358, NP_115649.1, 223334_at
FIG. 4258: PRO84937
FIG. 4259: DNA330528, AF151063, 223335_at
FIG. 4260: PRO50764
FIG. 4261: DNA330529, 241399.1, 223343_at
FIG. 4262: PRO85717
FIG. 4263A-B: DNA255756, HUMPDE7A,
223358_s_at
FIG. 4264: PRO50812
FIG. 4265: DNA227125, AF132297, 223377_x_at
FIG. 4266: PRO37588
FIG. 4267: DNA331614, CDCA1, 223381_at
FIG. 4268: PRO38881
FIG. 4269A-B: DNA329360, NP_115644.1,
223382_s_at
FIG. 4270: PRO84939
FIG. 4271A-B: DNA329360, NIN283, 223383_at
FIG. 4272: PRO84939
FIG. 4273: DNA330531, NP_037508.1, 223394_at
FIG. 4274: PRO85718
FIG. 4275: DNA329361, AF161528, 223397_s_at
FIG. 4276: PRO84940
FIG. 4277: DNA324156, NP_115588.1, 223403_s_at
FIG. 4278: PRO80856
FIG. 4279A-B: DNA254516, C1orf25, 223404_s_at
FIG. 4280: PRO49623
FIG. 4281: DNA256407, NP_055188.1, 223423_at
FIG. 4282: PRO51448
FIG. 4283: DNA255676, HSM801648, 223434_at
FIG. 4284: PRO50738
FIG. 4285: DNA330532, NP_078804.1, 223439_at
FIG. 4286: PRO85719
FIG. 4287: DNA330533, NP_058647.1, 223451_s_at
FIG. 4288: PRO772
FIG. 4289: DNA329365, CAB56010.1, 223452_s_at
FIG. 4290: PRO84944
FIG. 4291: DNA327958, NP_115789.1, 223484_at
FIG. 4292: PRO23554
FIG. 4293: DNA329456, NP_057126.1, 223489_x_at
FIG. 4294: PRO85023
FIG. 4295: DNA329456, RRP40, 223490_s_at
FIG. 4296: PRO85023
FIG. 4297: DNA330534, AF307332, 223494_at
FIG. 4298: PRO85720
FIG. 4299: DNA304784, NP_006564.1, 223502_s_at
FIG. 4300: PRO738
FIG. 4301: DNA330535, NP_115883.1, 223506_at
FIG. 4302: PRO85721
FIG. 4303: DNA330536, NP_115666.1, 223542_at
FIG. 4304: PRO85722
FIG. 4305: DNA330537, AF155827, 223556_at
FIG. 4306: PRO81892
FIG. 4307A-B: DNA327908, HSM801808,
223570_at
FIG. 4308: PRO83843
FIG. 4309: DNA330538, AF262027, 223598_at
FIG. 4310: PRO85723
FIG. 4311: DNA330539, NP_055411.1, 223639_s_at
FIG. 4312: PRO85724
FIG. 4313: DNA330540, NP_055081.1, 223640_at
FIG. 4314: PRO85725
FIG. 4315: DNA330541, AF277625, 223675_s_at
FIG. 4316: PRO85726
FIG. 4317: DNA330542, NP_115493.1, 223700_at
FIG. 4318: PRO85727
FIG. 4319: DNA330543, NAG73, 223725_at
FIG. 4320: PRO85728
FIG. 4321: DNA329367, TTYH2, 223741_s_at
FIG. 4322: PRO84946
FIG. 4323: DNA331615, AB049635, 223743_s_at
FIG. 4324: PRO62669
FIG. 4325: DNA188735, NP_001506.1, 223758_s_at
FIG. 4326: PRO26224
FIG. 4327: DNA287253, LOC85028, 223774_at
FIG. 4328: PRO69527
FIG. 4329: DNA331616, BC004277, 223785_at
FIG. 4330: PRO86613
FIG. 4331: DNA330544, NP_277049.1, 223800_s_at
FIG. 4332: PRO85729
FIG. 4333: DNA256005, NP_004842.1, 223806_s_at
FIG. 4334: PRO51056
FIG. 4335: DNA330545, AF233516, 223834_at
FIG. 4336: PRO70906
FIG. 4337: DNA327200, NP_114156.1, 223836_at
FIG. 4338: PRO1065
FIG. 4339: DNA330546, AF132203, 223839_s_at
FIG. 4340: PRO85730
FIG. 4341: DNA330547, NP_066014.1, 223849_s_at
FIG. 4342: PRO85731
FIG. 4343: DNA331392, NP_004186.1, 223851_s_at
FIG. 4344: PRO364
FIG. 4345: DNA330548, NP_115590.1, 223880_x_at
FIG. 4346: PRO85732
FIG. 4347A-B: DNA330522, FOXP1, 223936_s_at
FIG. 4348: PRO85712
FIG. 4349A-B: DNA330550, HSM801744,
223946_at
FIG. 4350: PRO85734
FIG. 4351: DNA331393, D83532, 223961_s_at
FIG. 4352: PRO86458
FIG. 4353: DNA324248, SP110, 223980_s_at
FIG. 4354: PRO80932
FIG. 4355: DNA330551, BC009946, 223983_s_at
FIG. 4356: PRO85735
FIG. 4357: DNA330552, BC001104, 223984_s_at
FIG. 4358: PRO85736
FIG. 4359: DNA328847, NP_056338.1, 223989_s_at
FIG. 4360: PRO84579
FIG. 4361: DNA331617, AF332652, 224046_s_at
FIG. 4362: PRO86614
FIG. 4363: DNA329369, AF293026, 224130_s_at
FIG. 4364: PRO84948
FIG. 4365: DNA330553, AF116653, 224148_at
FIG. 4366: DNA331618, AF231339, 224204_x_at
FIG. 4367: PRO86615
FIG. 4368: DNA330554, AF277993, 224211_at
FIG. 4369: PRO85737
FIG. 4370A-C: DNA227619, NP_054831.1,
224218_s_at
FIG. 4371: PRO38082
FIG. 4372: DNA324707, NP_037369.1, 224232_s_at
FIG. 4373: PRO81339
FIG. 4374: DNA323935, NP_060586.1, 224233_s_at
FIG. 4375: PRO80668
FIG. 4376: DNA329370, NP_060611.2, 224247_s_at
FIG. 4377: PRO84949
FIG. 4378A-B: DNA330555, HSM801768,
224308_s_at
FIG. 4379: PRO85738
FIG. 4380: DNA330556, NP_061881.2, 224319_s_at
FIG. 4381: PRO85739
FIG. 4382: DNA330557, C20orf154, 224320_s_at
FIG. 4383: PRO85740
FIG. 4384: DNA330558, NP_057588.1, 224330_s_at
FIG. 4385: PRO84950
FIG. 4386: DNA327949, MRP64, 224334_s_at
FIG. 4387: PRO83874
FIG. 4388A-B: DNA330559, BAB21791.1,
224336_s_at
FIG. 4389: PRO85741
FIG. 4390: DNA331619, BC010896, 224345_x_at
FIG. 4391: PRO86616
FIG. 4392: DNA331620, NDRG3, 224368_s_at
FIG. 4393: PRO86617
FIG. 4394: DNA272626, RIP5, 224376_s_at
FIG. 4395: PRO60759
FIG. 4396: DNA330560, NP_510882.1, 224413_s_at
FIG. 4397: PRO85742
FIG. 4398: DNA330561, AF321617, 224416_s_at
FIG. 4399: PRO85743
FIG. 4400: DNA328323, NP_114148.2, 224428_s_at
FIG. 4401: PRO69531
FIG. 4402: DNA331621, AF060225, 224437_s_at
FIG. 4403: PRO86618
FIG. 4404: DNA330562, NP_115716.1, 224448_s_at
FIG. 4405: PRO85744
FIG. 4406: DNA330563, NP_113668.1, 224450_s_at
FIG. 4407: PRO85745
FIG. 4408: DNA330564, NP_115885.1, 224451_x_at
FIG. 4409: PRO85746
FIG. 4410: DNA330565, BC006111, 224454_at
FIG. 4411: PRO85747
FIG. 4412: DNA330566, NP_115720.1, 224464_s_at
FIG. 4413: PRO85748
FIG. 4414: DNA329373, NP_15722.1, 224467_s_at
FIG. 4415: PRO84952
FIG. 4416: DNA330567, NP_116114.1, 224504_s_at
FIG. 4417: PRO85749
FIG. 4418: DNA327976, NP_116120.1, 224511_s_at
FIG. 4419: PRO69574
FIG. 4420: DNA330568, BC006428, 224516_s_at
FIG. 4421: PRO85750
FIG. 4422: DNA329374, NP_115735.1, 224523_s_at
FIG. 4423: PRO84953
FIG. 4424: DNA331622, TNFRSF18, 224553_s_at
FIG. 4425: PRO86619
FIG. 4426: DNA330569, BC020516, 224572_s_at
FIG. 4427A-B: DNA330570, AB040903, 224578_at
FIG. 4428: DNA330571, AK027320, 224607_s_at
FIG. 4429: PRO85752
FIG. 4430: DNA327980, BC008959, 224615_x_at
FIG. 4431: PRO83900
FIG. 4432: DNA329376, BAA91036.1, 224632_at
FIG. 4433: PRO84954
FIG. 4434: DNA330572, CAB82324.1, 224648_at
FIG. 4435: PRO85753
FIG. 4436A-B: DNA327981, 344095.3, 224654_at
FIG. 4437: PRO83901
FIG. 4438: DNA330573, C20orf108, 224690_at
FIG. 4439: PRO85754
FIG. 4440A-B: DNA330574, AB033054, 224698_at
FIG. 4441: DNA330575, AK022542, 224701_at
FIG. 4442: PRO85756
FIG. 4443: DNA331623, BC014138, 224711_at
FIG. 4444: PRO86620
FIG. 4445: DNA329378, BC022990, 224713_at
FIG. 4446: PRO84956
FIG. 4447: DNA324173, NP_115766.2, 224714_at
FIG. 4448: PRO80871
FIG. 4449: DNA330577, NP_443076.1, 224715_at
FIG. 4450: PRO85758
FIG. 4451A-B: DNA330578, 1353105.1, 224718_at
FIG. 4452: PRO85759
FIG. 4453: DNA330579, BC009925, 224719_s_at
FIG. 4454: PRO85760
FIG. 4455: DNA287382, 1383817.3, 224738_x_at
FIG. 4456: PRO69641
FIG. 4457: DNA257352, DNA257352, 224739_at
FIG. 4458: PRO51940
FIG. 4459: DNA331624, BC014242, 224740_at
FIG. 4460: PRO86621
FIG. 4461: DNA330581, MGC16386, 224753_at
FIG. 4462: PRO82014
FIG. 4463: DNA330582, 1454377.6, 224755_at
FIG. 4464: PRO85762
FIG. 4465: DNA330583, BC020522, 224759_s_at
FIG. 4466: PRO85763
FIG. 4467A-B: DNA287330, BAA86479.1,
224799_at
FIG. 4468: PRO69594
FIG. 4469A-B: DNA330584, FENS-1, 224800_at
FIG. 4470: PRO85764
FIG. 4471: DNA331397, AK001723, 224802_at
FIG. 4472: PRO23259
FIG. 4473: DNA330585, 206983.10, 224806_at
FIG. 4474: PRO85765
FIG. 4475: DNA330586, NP_443183.1, 224825_at
FIG. 4476: PRO85766
FIG. 4477A-B: DNA330559, AB051487, 224832_at
FIG. 4478A-B: DNA330809, 336997.1, 224838_at
FIG. 4479: PRO85973
FIG. 4480A-B: DNA330587, 1045521.4,
224839_s_at
FIG. 4481: PRO85767
FIG. 4482: DNA329380, BC014868, 224855_at
FIG. 4483: PRO80743
FIG. 4484: DNA330588, BC019034, 224871_at
FIG. 4485: PRO85768
FIG. 4486: DNA196374, DNA196374, 224880_at
FIG. 4487A-B: DNA331625, 411236.19, 224897_at
FIG. 4488: PRO86622
FIG. 4489: DNA329382, BC009072, 224913_s_at
FIG. 4490: DNA330590, CIP29, 224914_s_at
FIG. 4491: PRO85770
FIG. 4492A-B: DNA169523, DNA169523,
224917_at
FIG. 4493: PRO23253
FIG. 4494: DNA330591, NP_115865.1, 224919_at
FIG. 4495: PRO85771
FIG. 4496: DNA330592, AB014733, 224953_at
FIG. 4497: DNA287258, C20orf52, 224972_at
FIG. 4498: PRO52174
FIG. 4499: DNA151170, DNA151170, 224989_at
FIG. 4500: PRO12626
FIG. 4501: DNA330593, HS126A53, 225005_at
FIG. 4502A-B: DNA329385, 330826.1, 225010_at
FIG. 4503: PRO84961
FIG. 4504: DNA161646, DNA161646, 225036_at
FIG. 4505: DNA330594, BC005148, 225039_at
FIG. 4506: DNA331626, 412293.2, 225047_at
FIG. 4507: PRO86623
FIG. 4508: DNA195755, DNA195755, 225051_at
FIG. 4509A-B: DNA330596, 998535.1, 225070_at
FIG. 4510: PRO85774
FIG. 4511A-C: DNA271612, BAA92642.1,
225076_s_at
FIG. 4512: PRO59899
FIG. 4513: DNA330597, NP_057291.1, 225082_at
FIG. 4514: PRO85775
FIG. 4515: DNA330598, 1384569.2, 225086_at
FIG. 4516: PRO85776
FIG. 4517: DNA330599, 898528.3, 225095_at
FIG. 4518: PRO85777
FIG. 4519: DNA330600, HSA272196, 225096_at
FIG. 4520: PRO85778
FIG. 4521: DNA330601, 1322727.6, 225098_at
FIG. 4522: PRO85779
FIG. 4523: DNA331627, BC013920, 225105_at
FIG. 4524: PRO86624
FIG. 4525: DNA225597, NP_060703.1, 225106_s_at
FIG. 4526: PRO36060
FIG. 4527A-B: DNA331628, 245994.3, 225113_at
FIG. 4528: PRO86625
FIG. 4529A-B: DNA327993, 898436.7, 225133_at
FIG. 4530: PRO81138
FIG. 4531: DNA155396, DNA155396, 225143_at
FIG. 4532: DNA330603, 235138.16, 225157_at
FIG. 4533: PRO85781
FIG. 4534: DNA329393, NP_079272.4, 225158_at
FIG. 4535: PRO84969
FIG. 4536: DNA329393, EFG1, 225161_at
FIG. 4537: PRO84969
FIG. 4538: DNA330604, NP_277050.1, 225171_at
FIG. 4539: PRO85782
FIG. 4540: DNA327996, BC010181, 225195_at
FIG. 4541: PRO83915
FIG. 4542: DNA199601, DNA199601, 225199_at
FIG. 4543: DNA329394, BC010416, 225201_s_at
FIG. 4544: DNA329396, NP_060866.1, 225253_s_at
FIG. 4545: PRO84972
FIG. 4546: DNA329397, NP_114109.1, 225260_s_at
FIG. 4547: PRO84973
FIG. 4548A-B: DNA329398, 411135.13, 225262_at
FIG. 4549: PRO4805
FIG. 4550A-B: DNA258863, DNA258863,
225266_at
FIG. 4551A-B: DNA331629, 233102.7, 225269_s_at
FIG. 4552: PRO86626
FIG. 4553A-B: DNA330606, 475590.1, 225290_at
FIG. 4554: PRO85784
FIG. 4555: DNA329400, BC005986, 225291_at
FIG. 4556: DNA326458, BC014003, 225297_at
FIG. 4557: PRO82841
FIG. 4558: DNA330607, 167391.12, 225300_at
FIG. 4559: PRO85785
FIG. 4560: DNA330608, BC016880, 225323_at
FIG. 4561: PRO85786
FIG. 4562A-B: DNA169918, DNA169918,
225340_s_at
FIG. 4563: PRO23256
FIG. 4564: DNA330609, AF419331, 225348_at
FIG. 4565: PRO22196
FIG. 4566: DNA327965, NP_060760.1, 225367_at
FIG. 4567: PRO83888
FIG. 4568: DNA273635, HSM801117, 225371_at
FIG. 4569A-B: DNA330610, BAB15739.1,
225372_at
FIG. 4570: PRO85787
FIG. 4571: DNA331630, BC020568, 225373_at
FIG. 4572: PRO86627
FIG. 4573A-B: DNA331631, 1383781.5,
225385_s_at
FIG. 4574: PRO86628
FIG. 4575: DNA329401, BC017480, 225386_s_at
FIG. 4576: PRO84976
FIG. 4577: DNA329402, 198708.7, 225387_at
FIG. 4578: PRO4845
FIG. 4579: DNA329403, AF288394, 225399_at
FIG. 4580: DNA331632, BC022030, 225400_at
FIG. 4581: PRO86629
FIG. 4582: DNA330612, C20orf64, 225402_at
FIG. 4583: PRO85789
FIG. 4584A-B: DNA328893, BC020490, 225406_at
FIG. 4585: PRO9914
FIG. 4586: DNA330613, BC019355, 225414_at
FIG. 4587A-B: DNA331633, 1449606.5, 225433_at
FIG. 4588: PRO86630
FIG. 4589: DNA304802, AAH00967.1, 225439_at
FIG. 4590: PRO71212
FIG. 4591: DNA328005, BC004413, 225440_at
FIG. 4592: DNA330615, NP_115732.1, 225441_x_at
FIG. 4593: PRO85791
FIG. 4594: DNA330616, 429555.1, 225443_at
FIG. 4595: PRO85792
FIG. 4596A-B: DNA330617, 336147.2, 225447_at
FIG. 4597: PRO59923
FIG. 4598: DNA329404, BC013949, 225454_at
FIG. 4599: PRO82972
FIG. 4600: DNA330618, CAB55990.1, 225457_s_at
FIG. 4601: PRO85793
FIG. 4602: DNA330618, HSM801081, 225458_at
FIG. 4603: DNA196561, DNA196561, 225470_at
FIG. 4604: DNA329405, HSM800962, 225520_at
FIG. 4605A-B: DNA330619, BC013128, 225527_at
FIG. 4606: PRO12186
FIG. 4607A-B: DNA330620, CAB55950.1,
225533_at
FIG. 4608: PRO85794
FIG. 4609: DNA330621, AF116628, 225535_s_at
FIG. 4610: DNA328008, 240051.4, 225541_at
FIG. 4611: PRO83926
FIG. 4612A-B: DNA330622, 233388.8, 225543_at
FIG. 4613: PRO85796
FIG. 4614: DNA330623, 1502854.5, 225549_at
FIG. 4615: PRO85797
FIG. 4616: DNA329406, 1503139.10, 225562_at
FIG. 4617: PRO84979
FIG. 4618: DNA330624, AK000500, 225580_at
FIG. 4619: PRO85798
FIG. 4620: DNA330625, AK025643, 225581_s_at
FIG. 4621: PRO85799
FIG. 4622: DNA304469, NP_149078.1, 225621_at
FIG. 4623: PRO71045
FIG. 4624: DNA151667, DNA151667, 225634_at
FIG. 4625: PRO11970
FIG. 4626: DNA330626, 1398905.1, 225638_at
FIG. 4627: PRO85800
FIG. 4628: DNA331634, CTSC, 225647_s_at
FIG. 4629: PRO86631
FIG. 4630A-B: DNA288261, NP_037414.2,
225655_at
FIG. 4631: PRO70021
FIG. 4632: DNA329408, NP_056235.2, 225676_s_at
FIG. 4633: PRO38893
FIG. 4634A-B: DNA330627, 987725.3, 225679_at
FIG. 4635: PRO85801
FIG. 4636: DNA329409, BC017248, 225682_s_at
FIG. 4637: PRO84981
FIG. 4638: DNA325272, NP_054891.1, 225683_x_at
FIG. 4639: PRO81822
FIG. 4640: DNA328012, BC017873, 225686_at
FIG. 4641: PRO83930
FIG. 4642: DNA328013, AAH01068.1, 225687_at
FIG. 4643: PRO83931
FIG. 4644A-C: DNA330628, 1400234.13, 225690_at
FIG. 4645: PRO85802
FIG. 4646A-B: DNA330629, BAA74856.2,
225692_at
FIG. 4647: PRO50227
FIG. 4543: DNA329394, BC010416, 225201_s_at
FIG. 4544: DNA329396, NP_060866.1, 225253_s_at
FIG. 4545: PRO84972
FIG. 4546: DNA329397, NP_114109.1, 225260_s_at
FIG. 4547: PRO84973
FIG. 4548A-B: DNA329398, 411135.13, 225262_at
FIG. 4549: PRO4805
FIG. 4550A-B: DNA258863, DNA258863,
225266_at
FIG. 4551A-B: DNA331629, 233102.7, 225269_s_at
FIG. 4552: PRO86626
FIG. 4553A-B: DNA330606, 475590.1, 225290_at
FIG. 4554: PRO85784
FIG. 4555: DNA329400, BC005986, 225291_at
FIG. 4556: DNA326458, BC014003, 225297_at
FIG. 4557: PRO82841
FIG. 4558: DNA330607, 167391.12, 225300_at
FIG. 4559: PRO85785
FIG. 4560: DNA330608, BC016880, 225323_at
FIG. 4561: PRO85786
FIG. 4562A-B: DNA169918, DNA169918,
225340_s_at
FIG. 4563: PRO23256
FIG. 4564: DNA330609, AF419331, 225348_at
FIG. 4565: PRO22196
FIG. 4566: DNA327965, NP_060760.1, 225367_at
FIG. 4567: PRO83888
FIG. 4568: DNA273635, HSM801117, 225371_at
FIG. 4569A-B: DNA330610, BAB15739.1,
225372_at
FIG. 4570: PRO85787
FIG. 4571: DNA331630, BC020568, 225373_at
FIG. 4572: PRO86627
FIG. 4573A-B: DNA331631, 1383781.5,
225385_s_at
FIG. 4574: PRO86628
FIG. 4575: DNA329401, BC017480, 225386_s_at
FIG. 4576: PRO84976
FIG. 4577: DNA329402, 198708.7, 225387_at
FIG. 4578: PRO4845
FIG. 4579: DNA329403, AF288394, 225399_at
FIG. 4580: DNA331632, BC022030, 225400_at
FIG. 4581: PRO86629
FIG. 4582: DNA330612, C20orf64, 225402_at
FIG. 4583: PRO85789
FIG. 4584A-B: DNA328893, BC020490, 225406_at
FIG. 4585: PRO9914
FIG. 4586: DNA330613, BC019355, 225414_at
FIG. 4587A-B: DNA331633, 1449606.5, 225433_at
FIG. 4588: PRO86630
FIG. 4589: DNA304802, AAH00967.1, 225439_at
FIG. 4590: PRO71212
FIG. 4591: DNA328005, BC004413, 225440_at
FIG. 4592: DNA330615, NP_115732.1, 225441_x_at
FIG. 4593: PRO85791
FIG. 4594: DNA330616, 429555.1, 225443_at
FIG. 4595: PRO85792
FIG. 4596A-B: DNA330617, 336147.2, 225447_at
FIG. 4597: PRO59923
FIG. 4598: DNA329404, BC013949, 225454_at
FIG. 4599: PRO82972
FIG. 4600: DNA330618, CAB55990.1, 225457_s_at
FIG. 4601: PRO85793
FIG. 4602: DNA330618, HSM801081, 225458_at
FIG. 4603: DNA196561, DNA196561, 225470_at
FIG. 4604: DNA329405, HSM800962, 225520_at
FIG. 4605A-B: DNA330619, BC013128, 225527_at
FIG. 4606: PRO12186
FIG. 4607A-B: DNA330620, CAB55950.1,
225533_at
FIG. 4608: PRO85794
FIG. 4609: DNA330621, AF116628, 225535_s_at
FIG. 4610: DNA328008, 240051.4, 225541_at
FIG. 4611: PRO83926
FIG. 4612A-B: DNA330622, 233388.8, 225543_at
FIG. 4613: PRO85796
FIG. 4614: DNA330623, 1502854.5, 225549_at
FIG. 4615: PRO85797
FIG. 4616: DNA329406, 1503139.10, 225562_at
FIG. 4617: PRO84979
FIG. 4618: DNA330624, AK000500, 225580_at
FIG. 4619: PRO85798
FIG. 4620: DNA330625, AK025643, 225581_s_at
FIG. 4621: PRO85799
FIG. 4622: DNA304469, NP_149078.1, 225621_at
FIG. 4623: PRO71045
FIG. 4624: DNA151667, DNA151667, 225634_at
FIG. 4625: PRO11970
FIG. 4626: DNA330626, 1398905.1, 225638_at
FIG. 4627: PRO85800
FIG. 4628: DNA331634, CTSC, 225647_s_at
FIG. 4629: PRO86631
FIG. 4630A-B: DNA288261, NP_037414.2,
225655_at
FIG. 4631: PRO70021
FIG. 4632: DNA329408, NP_056235.2, 225676_s_at
FIG. 4633: PRO38893
FIG. 4634A-B: DNA330627, 987725.3, 225679_at
FIG. 4635: PRO85801
FIG. 4636: DNA329409, BC017248, 225682_s_at
FIG. 4637: PRO84981
FIG. 4638: DNA325272, NP_054891.1, 225683_x_at
FIG. 4639: PRO81822
FIG. 4640: DNA328012, BC017873, 225686_at
FIG. 4641: PRO83930
FIG. 4642: DNA328013, AAH01068.1, 225687_at
FIG. 4643: PRO83931
FIG. 4644A-C: DNA330628, 1400234.13, 225690_at
FIG. 4645: PRO85802
FIG. 4646A-B: DNA330629, BAA74856.2,
225692_at
FIG. 4647: PRO50227
FIG. 4648: DNA273623, AY037153, 225693_s_at
FIG. 4649: PRO61596
FIG. 4650: DNA330630, TIGA1, 225698_at
FIG. 4651: PRO85803
FIG. 4652A-B: DNA331635, BAB13371.1,
225704_at
FIG. 4653: PRO86632
FIG. 4654: DNA331636, 221395.1, 225716_at
FIG. 4655: PRO86633
FIG. 4656: DNA330633, BC003515, 225723_at
FIG. 4657A-B: DNA330634, 243208.1, 225725_at
FIG. 4658: PRO85806
FIG. 4659A-B: DNA330635, 233691.4, 225736_at
FIG. 4660: PRO85807
FIG. 4661: DNA324266, NP_056268.1, 225741_at
FIG. 4662: PRO80949
FIG. 4663: DNA323970, MGC21854, 225763_at
FIG. 4664: PRO80699
FIG. 4665: DNA331637, 7693984.1, 225768_at
FIG. 4666: PRO86634
FIG. 4667: DNA330636, NP_201575.2, 225794_s_at
FIG. 4668: PRO85808
FIG. 4669: DNA330636, LOC91689, 225795_at
FIG. 4670: PRO85808
FIG. 4671: DNA329414, MGC4677, 225799_at
FIG. 4672: PRO84986
FIG. 4673: DNA330637, NP_478136.1, 225803_at
FIG. 4674: PRO85809
FIG. 4675A-C: DNA330638, CAB63749.1,
225814_at
FIG. 4676: PRO85810
FIG. 4677: DNA330639, 420605.8, 225834_at
FIG. 4678: PRO85811
FIG. 4679: DNA329417, 411336.1, 225842_at
FIG. 4680: PRO84989
FIG. 4681: DNA287622, AF041429, 225849_s_at
FIG. 4682: DNA329418, BC018969, 225850_at
FIG. 4683: PRO19906
FIG. 4684: DNA287370, BAB14983.1, 225866_at
FIG. 4685: PRO69630
FIG. 4686A-B: DNA331638, 1097910.3, 225886_at
FIG. 4687: PRO86635
FIG. 4688A-B: DNA331639, 1391157.25, 225888_at
FIG. 4689: PRO86636
FIG. 4690: DNA330642, NP_115494.1, 225898_at
FIG. 4691: PRO85814
FIG. 4692A-B: DNA331640, 481415.9, 225927_at
FIG. 4693: PRO86637
FIG. 4694A-B: DNA255887, BAB13380.1,
225929_s_at
FIG. 4695: PRO50940
FIG. 4696A-B: DNA255887, AB046774,
225931_s_at
FIG. 4697A-B: DNA330644, 236657.4, 225935_at
FIG. 4698: PRO85816
FIG. 4699: DNA331641, AK027752, 225959_s_at
FIG. 4700: PRO86638
FIG. 4701A-B: DNA329360, AF378524, 225962_at
FIG. 4702: PRO84939
FIG. 4703: DNA329420, BC018014, 225970_at
FIG. 4704A-B: DNA330645, 350385.2, 225973_at
FIG. 4705: PRO85817
FIG. 4706A-B: DNA329421, 343552.1, 225974_at
FIG. 4707: PRO84992
FIG. 4708A-B: DNA331642, BAB15719.1,
225979_at
FIG. 4709: PRO86639
FIG. 4710: DNA330647, AK002174, 226001_at
FIG. 4711: PRO85819
FIG. 4712: DNA330648, 1399123.1, 226005_at
FIG. 4713: PRO85820
FIG. 4714: DNA330649, AK056957, 226008_at
FIG. 4715: PRO85821
FIG. 4716A-B: DNA331643, 246054.6, 226021_at
FIG. 4717: PRO86640
FIG. 4718: DNA331644, 027830.2, 226034_at
FIG. 4719: PRO86641
FIG. 4720: DNA328021, BC004538, 226038_at
FIG. 4721: DNA273736, DNA273736, 226040_at
FIG. 4722: DNA330652, CLONE24945, 226055_at
FIG. 4723: PRO85824
FIG. 4724: DNA330653, 7687670.2, 226068_at
FIG. 4725: PRO85825
FIG. 4726: DNA304795, AK056513, 226077_at
FIG. 4727: PRO71207
FIG. 4728A-B: DNA331645, AAD09327.1,
226082_s_at
FIG. 4729: PRO86642
FIG. 4730: DNA287271, NP_116188.2, 226088_at
FIG. 4731: PRO69542
FIG. 4732: DNA330655, AF114264, 226103_at
FIG. 4733: PRO85827
FIG. 4734A-B: DNA330656, AK023825, 226109_at
FIG. 4735: PRO85828
FIG. 4736: DNA329425, BC008294, 226117_at
FIG. 4737: DNA330657, 198409.1, 226140_s_at
FIG. 4738: PRO85829
FIG. 4739: DNA330658, 204262.3, 226157_at
FIG. 4740: PRO85830
FIG. 4741: DNA330659, AF289605, 226175_at
FIG. 4742: PRO85831
FIG. 4743A-B: DNA330660, 979126.7, 226178_at
FIG. 4744: PRO85832
FIG. 4745A-B: DNA259025, DNA259025,
226180_at
FIG. 4746: PRO52958
FIG. 4747: DNA56350, DNA56350, 226181_at
FIG. 4748: PRO956
FIG. 4749: DNA330661, BAB47431.1, 226194_at
FIG. 4750: PRO85833
FIG. 4751A-B: DNA329428, 1446144.8, 226218_at
FIG. 4752: PRO84999
FIG. 4753: DNA195822, DNA195822, 226241_s_at
FIG. 4754A-B: DNA330662, 334156.1, 226252_at
FIG. 4755: PRO85834
FIG. 4756: DNA331646, 956845.3, 226261_at
FIG. 4757: PRO86643
FIG. 4758A-B: DNA330664, 400637.4, 226265_at
FIG. 4759: PRO85836
FIG. 4760A-B: DNA330665, 233070.3, 226270_at
FIG. 4761: PRO85837
FIG. 4762: DNA330666, 199829.14, 226272_at
FIG. 4763: PRO85838
FIG. 4764: DNA193896, DNA193896, 226276_at
FIG. 4765: PRO23314
FIG. 4766: DNA330667, AF301222, 226287_at
FIG. 4767: DNA330668, BC010176, 226308_at
FIG. 4768: PRO85840
FIG. 4769: DNA328028, NP_005773.1, 226319_s_at
FIG. 4770: PRO83945
FIG. 4771: DNA328028, ALY, 226320_at
FIG. 4772: PRO83945
FIG. 4773: DNA330669, 236903.4, 226321_at
FIG. 4774: PRO85841
FIG. 4775: DNA330670, BC018453, 226329_s_at
FIG. 4776: PRO85842
FIG. 4777A-B: DNA330671, 228447.18, 226342_at
FIG. 4778: PRO85843
FIG. 4779: DNA330672, 255309.4, 226347_at
FIG. 4780: PRO85844
FIG. 4781: DNA330673, 236879.2, 226348_at
FIG. 4782: PRO85845
FIG. 4783: DNA329430, NP_116191.2, 226353_at
FIG. 4784: PRO38524
FIG. 4785: DNA331647, 236137.11, 226354_at
FIG. 4786: PRO86644
FIG. 4787A-B: DNA330675, 177663.2, 226368_at
FIG. 4788: PRO85847
FIG. 4789: DNA330676, CAA11393.1, 226388_at
FIG. 4790: PRO85848
FIG. 4791: DNA330677, 1384190.6, 226390_at
FIG. 4792: PRO85849
FIG. 4793: DNA151740, DNA151740, 226419_s_at
FIG. 4794: PRO12029
FIG. 4795: DNA329433, NP_115937.1, 226442_at
FIG. 4796: PRO85003
FIG. 4797: DNA330678, 401430.1, 226444_at
FIG. 4798: PRO85850
FIG. 4799: DNA287657, BC009447, 226448_at
FIG. 4800: PRO69688
FIG. 4801: DNA330679, BC013040, 226456_at
FIG. 4802A-B: DNA330680, BC022792, 226477_at
FIG. 4803: PRO85852
FIG. 4804: DNA326066, NP_291022.1, 226488_at
FIG. 4805: PRO82501
FIG. 4806A-B: DNA330681, 971066.5, 226503_at
FIG. 4807: PRO85853
FIG. 4808: DNA330682, HSM801031, 226510_at
FIG. 4809: DNA304794, NP_115521.2, 226541_at
FIG. 4810: PRO71206
FIG. 4811: DNA330683, 1446727.8, 226546_at
FIG. 4812: PRO85854
FIG. 4813: DNA330684, 984114.1, 226548_at
FIG. 4814: PRO85855
FIG. 4815A-B: DNA328031, 331264.1, 226587_at
FIG. 4816: PRO83948
FIG. 4817: DNA330685, BAB13430.1, 226588_at
FIG. 4818: PRO85856
FIG. 4819A-B: DNA330686, 1502531.18,
226602_s_at
FIG. 4820: PRO85857
FIG. 4821: DNA328033, 1446419.1, 226625_at
FIG. 4822: PRO83949
FIG. 4823: DNA330687, 215158.5, 226650_at
FIG. 4824: PRO85858
FIG. 4825: DNA258913, DNA258913, 226661_at
FIG. 4826: PRO52846
FIG. 4827A-C: DNA328462, HSA303079,
226694_at
FIG. 4828: PRO84288
FIG. 4829: DNA328037, BC016969, 226702_at
FIG. 4830: DNA330688, 240121.1, 226725_at
FIG. 4831: PRO85859
FIG. 4832A-C: DNA330689, 978733.6, 226732_at
FIG. 4833: PRO85860
FIG. 4834: DNA257914, DNA257914, 226743_at
FIG. 4835: PRO52447
FIG. 4836: DNA330690, 245065.1, 226745_at
FIG. 4837: PRO85861
FIG. 4838: DNA330691, BC022075, 226748_at
FIG. 4839: PRO85862
FIG. 4840: DNA329435, 347092.10, 226750_at
FIG. 4841: PRO85005
FIG. 4842: DNA331648, 243999.3, 226757_at
FIG. 4843: PRO86645
FIG. 4844: DNA330692, 1446140.1, 226758_at
FIG. 4845: PRO85863
FIG. 4846A-B: DNA330331, AB032963, 226771_at
FIG. 4847: DNA330693, HSBRN1H12, 226773_at
FIG. 4848A-B: DNA330694, 481455.4, 226810_at
FIG. 4849: PRO85865
FIG. 4850: DNA330695, 404167.9, 226818_at
FIG. 4851: PRO85866
FIG. 4852A-C: DNA330696, 404167.10, 226841_at
FIG. 4853: PRO85867
FIG. 4854: DNA330697, BC011808, 226858_at
FIG. 4855: PRO85868
FIG. 4856A-B: DNA329436, 236863.1, 226869_at
FIG. 4857: PRO85006
FIG. 4858: DNA330698, BC020852, 226896_at
FIG. 4859: PRO85869
FIG. 4860: DNA329437, 156503.10, 226901_at
FIG. 4861: PRO85007
FIG. 4862: DNA330699, BC014203, 226905_at
FIG. 4863: DNA330564, ARHGAP9, 226906_s_at
FIG. 4864: PRO85746
FIG. 4865: DNA327917, BC000798, 226915_s_at
FIG. 4866: PRO83852
FIG. 4867A-C: DNA331649, 201042.4, 226921_at
FIG. 4868: PRO86646
FIG. 4869: DNA328044, 039170.3, 226936_at
FIG. 4870: PRO83958
FIG. 4871: DNA151713, DNA151713, 226943_at
FIG. 4872: PRO12003
FIG. 4873: DNA330701, NP_115652.1, 226945_at
FIG. 4874: PRO85872
FIG. 4875: DNA330702, 023085.2, 226965_at
FIG. 4876: PRO85873
FIG. 4877: DNA330703, 201413.1, 226970_at
FIG. 4878: PRO85874
FIG. 4879: DNA154627, DNA154627, 226976_at
FIG. 4880: DNA330704, BC019075, 226980_at
FIG. 4881: PRO85875
FIG. 4882A-B: DNA331650, HSA314788,
226998_at
FIG. 4883: PRO85008
FIG. 4884: DNA329439, HSM802614, 227014_at
FIG. 4885A-B: DNA330705, 198782.1, 227020_at
FIG. 4886: PRO85876
FIG. 4887A-B: DNA330706, AF445027, 227027_at
FIG. 4888: PRO85877
FIG. 4889: DNA330707, 028375.3, 227044_at
FIG. 4890: PRO85878
FIG. 4891: DNA330708, 1101317.1, 227066_at
FIG. 4892: PRO85879
FIG. 4893: DNA329440, 7691797.1, 227068_at
FIG. 4894: PRO85009
FIG. 4895: DNA330709, 7692923.1, 227117_at
FIG. 4896: PRO85880
FIG. 4897: DNA323785, NP_116261.1, 227134_at
FIG. 4898: PRO80537
FIG. 4899: DNA330710, 331040.11, 227135_at
FIG. 4900: PRO85881
FIG. 4901A-B: DNA330711, 425448.18, 227150_at
FIG. 4902: PRO85882
FIG. 4903: DNA330712, 1452648.12, 227167_s_at
FIG. 4904: PRO85883
FIG. 4905: DNA328281, BC000282, 227172_at
FIG. 4906: DNA330713, 334485.4, 227187_at
FIG. 4907: PRO85884
FIG. 4908: DNA330714, 034544.1, 227198_at
FIG. 4909: PRO85885
FIG. 4910: DNA330715, BC022374, 227211_at
FIG. 4911: PRO85886
FIG. 4912A-B: DNA330620, HSM800990,
227212_s_at
FIG. 4913: DNA330716, BC021675, 227236_at
FIG. 4914: PRO85887
FIG. 4915: DNA251633, AK023151, 227245_at
FIG. 4916: PRO47694
FIG. 4917: DNA327206, AY037161, 227262_at
FIG. 4918: PRO271
FIG. 4919A-B: DNA329442, AH007300S2,
227265_at
FIG. 4920A-B: DNA331651, 099572.12,
227266_s_at
FIG. 4921: PRO86647
FIG. 4922: DNA330717, 232831.10, 227290_at
FIG. 4923: PRO85888
FIG. 4924: DNA329445, 001839.3, 227291_s_at
FIG. 4925: PRO85013
FIG. 4926: DNA330718, 025465.3, 227295_at
FIG. 4927: PRO85889
FIG. 4928: DNA330719, 7697121.1, 227307_at
FIG. 4929: PRO85890
FIG. 4930: DNA330720, 186766.12, 227337_at
FIG. 4931: PRO85891
FIG. 4932: DNA35664, DNA35664, 227345_at
FIG. 4933: PRO34697
FIG. 4934A-B: DNA330721, 198680.1, 227350_at
FIG. 4935: PRO85892
FIG. 4936: DNA226872, NP_001955.1, 227404_s_at
FIG. 4937: PRO37335
FIG. 4938A-B: DNA330722, AB058729, 227418_at
FIG. 4939: DNA330723, AB040960, 227438_at
FIG. 4940: DNA329447, BC016981, 227449_at
FIG. 4941: PRO85015
FIG. 4942: DNA330724, AK056677, 227450_at
FIG. 4943: PRO1575
FIG. 4944A-B: DNA328054, 233014.1, 227458_at
FIG. 4945: PRO83968
FIG. 4946A-B: DNA258781, DNA258781,
227466_at
FIG. 4947: PRO52715
FIG. 4948: DNA329448, BC012948, 227477_at
FIG. 4949: PRO85016
FIG. 4950: DNA329053, LOC51105, 227523_s_at
FIG. 4951: PRO84715
FIG. 4952: DNA330725, 337360.7, 227545_at
FIG. 4953: PRO85894
FIG. 4954: DNA330726, BC014967, 227558_at
FIG. 4955: PRO85895
FIG. 4956A-B: DNA331652, 1447357.3, 227572_at
FIG. 4957: PRO86648
FIG. 4958: DNA330728, HSM801012, 227580_s_at
FIG. 4959: PRO85897
FIG. 4960: DNA330729, 031130.2, 227600_at
FIG. 4961: PRO85898
FIG. 4962A-B: DNA287193, AB037794,
227606_s_at
FIG. 4963: DNA330730, BC010846, 227607_at
FIG. 4964: PRO85899
FIG. 4965: DNA257714, NP_150280.1, 227609_at
FIG. 4966: PRO52268
FIG. 4967: DNA330731, BC012337, 227614_at
FIG. 4968: DNA196237, DNA196237, 227616_at
FIG. 4969A-B: DNA330426, AF085233, 227627_at
FIG. 4970: PRO85631
FIG. 4971A-B: DNA330733, BAA92631.1,
227653_at
FIG. 4972: PRO85902
FIG. 4973: DNA329449, 979180.1, 227682_at
FIG. 4974: PRO85017
FIG. 4975: DNA330734, BC008322, 227686_at
FIG. 4976: PRO85903
FIG. 4977: DNA273987, DNA273987, 227708_at
FIG. 4978: DNA330735, 1400830.3, 227722_at
FIG. 4979: PRO85904
FIG. 4980: DNA329450, BC017226, 227726_at
FIG. 4981: PRO85018
FIG. 4982A-B: DNA330736, BAA86532.1,
227732_at
FIG. 4983: PRO85905
FIG. 4984A-B: DNA330737, 331100.8, 227766_at
FIG. 4985: PRO85906
FIG. 4986: DNA331653, 212641.1, 227792_at
FIG. 4987: PRO86649
FIG. 4988: DNA151733, DNA151733, 227807_at
FIG. 4989: PRO12022
FIG. 4990A-B: DNA330739, AK000004, 227811_at
FIG. 4991: DNA329454, BC022534, 227856_at
FIG. 4992: PRO85022
FIG. 4993: DNA260485, DNA260485, 227867_at
FIG. 4994: PRO54411
FIG. 4995: DNA327214, AK056512, 227873_at
FIG. 4996: PRO83483
FIG. 4997: DNA151503, DNA151503, 227877_at
FIG. 4998: PRO11849
FIG. 4999: DNA329481, NP_057234.2, 227915_at
FIG. 5000: PRO60949
FIG. 5001: DNA329456, AF151860, 227916_x_at
FIG. 5002: PRO85023
FIG. 5003A-B: DNA330740, AY028320S2,
227928_at
FIG. 5004: DNA151580, DNA151580, 227930_at
FIG. 5005: PRO11901
FIG. 5006: DNA330741, 350868.1, 227952_at
FIG. 5007: PRO85909
FIG. 5008A-B: DNA331654, 476805.1, 228006_at
FIG. 5009: PRO86650
FIG. 5010: DNA330539, ZNRD1, 228009_x_at
FIG. 5011: PRO85724
FIG. 5012: DNA150660, NP_057151.1, 228019_s_at
FIG. 5013: PRO12397
FIG. 5014A-B: DNA328432, NP_005768.1,
228030_at
FIG. 5015: PRO61793
FIG. 5016: DNA331655, 1449874.3, 228053_s_at
FIG. 5017: PRO86651
FIG. 5018: DNA330743, AK054689, 228062_at
FIG. 5019: PRO85911
FIG. 5020: DNA331656, 244771.1, 228063_s_at
FIG. 5021: PRO86652
FIG. 5022: DNA331657, BLR1, 228065_at
FIG. 5023: PRO23970
FIG. 5024: DNA329459, 230998.1, 228066_at
FIG. 5025: PRO85026
FIG. 5026: DNA330745, BC011716, 228069_at
FIG. 5027: PRO85913
FIG. 5028: DNA196216, DNA196216, 228071_at
FIG. 5029: DNA329460, BC017117, 228092_at
FIG. 5030: PRO85027
FIG. 5031: DNA330746, 346395.7, 228097_at
FIG. 5032: PRO85914
FIG. 5033: DNA330436, AF187016, 228098_s_at
FIG. 5034: PRO85639
FIG. 5035A-C: DNA331658, 200650.1, 228109_at
FIG. 5036: PRO86653
FIG. 5037: DNA329461, BC016615, 228113_at
FIG. 5038: PRO85028
FIG. 5039: DNA330748, 224725.3, 228159_at
FIG. 5040: PRO85916
FIG. 5041: DNA330749, 337382.1, 228174_at
FIG. 5042: PRO85917
FIG. 5043: DNA330750, 984920.1, 228180_at
FIG. 5044: PRO85918
FIG. 5045: DNA153924, DNA153924, 228188_at
FIG. 5046: DNA330751, 334282.2, 228189_at
FIG. 5047: PRO85919
FIG. 5048: DNA330752, 7694335.3, 228191_at
FIG. 5049: PRO85920
FIG. 5050A-B: DNA331659, 198497.1, 228201_at
FIG. 5051: PRO86654
FIG. 5052A-C: DNA328072, AB051556, 228230_at
FIG. 5053: DNA330754, 349978.1, 228242_at
FIG. 5054: PRO85922
FIG. 5055: DNA328663, CGI-142, 228266_s_at
FIG. 5056: PRO36183
FIG. 5057: DNA260948, DNA260948, 228273_at
FIG. 5058: PRO54700
FIG. 5059: DNA330755, BC020784, 228280_at
FIG. 5060: PRO85923
FIG. 5061: DNA331660, 230589.4, 228281_at
FIG. 5062: PRO86655
FIG. 5063A-B: DNA330757, AB046790, 228323_at
FIG. 5064: DNA304814, BC016879, 228330_at
FIG. 5065: PRO52650
FIG. 5066: DNA194202, DNA194202, 228370_at
FIG. 5067: PRO23594
FIG. 5068: DNA330758, 238545.7, 228381_at
FIG. 5069: PRO85925
FIG. 5070: DNA330759, 337444.1, 228390_at
FIG. 5071: PRO85926
FIG. 5072A-B: DNA330760, 330900.8, 228401_at
FIG. 5073: PRO85927
FIG. 5074: DNA228118, DNA228118, 228456_s_at
FIG. 5075: DNA297188, NP_116233.1, 228468_at
FIG. 5076: PRO70805
FIG. 5077A-C: DNA331661, 388991.1, 228487_s_at
FIG. 5078: PRO86656
FIG. 5079: DNA330762, BC010269, 228499_at
FIG. 5080: PRO80989
FIG. 5081: DNA195938, DNA195938, 228531_at
FIG. 5082: DNA329463, 412954.5, 228532_at
FIG. 5083: PRO85030
FIG. 5084: DNA330763, 1306177.32, 228549_at
FIG. 5085: PRO85929
FIG. 5086: DNA331662, 1450017.11, 228559_at
FIG. 5087: PRO86657
FIG. 5088A-C: DNA331663, 475198.1, 228562_at
FIG. 5089: PRO86658
FIG. 5090: DNA330766, 977419.7, 228597_at
FIG. 5091: PRO85932
FIG. 5092A-B: DNA331664, 201954.14, 228603_at
FIG. 5093: PRO86659
FIG. 5094: DNA328079, 239903.1, 228617_at
FIG. 5095: PRO83991
FIG. 5096: DNA330768, NP_003681.1, 228620_at
FIG. 5097: PRO60565
FIG. 5098A-B: DNA271477, NP_055774.1,
228641_at
FIG. 5099: PRO59770
FIG. 5100: DNA330769, 230457.1, 228664_at
FIG. 5101: PRO85934
FIG. 5102: DNA330770, 1447329.13, 228702_at
FIG. 5103: PRO85935
FIG. 5104: DNA330771, 1447928.4, 228710_at
FIG. 5105: PRO85936
FIG. 5106: DNA330772, 286623.2, 228729_at
FIG. 5107: PRO85937
FIG. 5108: DNA330773, 027401.1, 228758_at
FIG. 5109: PRO85938
FIG. 5110: DNA330774, 024160.1, 228760_at
FIG. 5111: PRO85939
FIG. 5112: DNA273232, DNA273232, 228785_at
FIG. 5113: DNA330775, 407523.3, 228806_at
FIG. 5114: PRO85940
FIG. 5115: DNA330776, NP_005740.1, 228834_at
FIG. 5116: PRO58014
FIG. 5117: DNA256483, HSM802155, 228859_at
FIG. 5118: PRO51520
FIG. 5119: DNA331665, 330848.1, 228869_at
FIG. 5120: PRO86660
FIG. 5121: DNA330778, 998621.10, 228891_at
FIG. 5122: PRO85942
FIG. 5123: DNA328084, 236591.7, 228905_at
FIG. 5124: PRO83996
FIG. 5125: DNA330779, 244243.1, 228953_at
FIG. 5126: PRO85943
FIG. 5127: DNA330780, 335374.1, 228955_at
FIG. 5128: PRO85944
FIG. 5129: DNA330781, 289775.9, 228960_at
FIG. 5130: PRO85945
FIG. 5131A-B: DNA331666, 7684887.1, 228964_at
FIG. 5132: PRO86661
FIG. 5133: DNA330783, 239601.22, 228987_at
FIG. 5134: PRO85947
FIG. 5135: DNA330784, 233595.21, 228990_at
FIG. 5136: PRO85948
FIG. 5137: DNA330785, 475283.24, 228999_at
FIG. 5138: PRO85949
FIG. 5139: DNA330786, 233085.1, 229029_at
FIG. 5140: PRO85950
FIG. 5141: DNA330787, 349981.7, 229040_at
FIG. 5142: PRO85951
FIG. 5143: DNA330788, AF305195, 229060_at
FIG. 5144: PRO85952
FIG. 5145: DNA330789, 199829.13, 229064_s_at
FIG. 5146: PRO85953
FIG. 5147: DNA330790, NP_116133.1, 229070_at
FIG. 5148: PRO85954
FIG. 5149: DNA330791, 7697349.2, 229072_at
FIG. 5150: PRO85955
FIG. 5151: DNA330792, 983946.2, 229097_at
FIG. 5152: PRO85956
FIG. 5153: DNA155281, DNA155281, 229111_at
FIG. 5154: DNA330793, 215114.2, 229145_at
FIG. 5155: PRO85957
FIG. 5156: DNA330794, 481414.8, 229202_at
FIG. 5157: PRO85958
FIG. 5158: DNA330795, BC017339, 229253_at
FIG. 5159: PRO85959
FIG. 5160: DNA265865, DNA265865, 229274_at
FIG. 5161: DNA151375, DNA151375, 229327_s_at
FIG. 5162: PRO11752
FIG. 5163: DNA328919, FLJ22690, 229367_s_at
FIG. 5164: PRO84637
FIG. 5165: DNA257575, DNA257575, 229374_at
FIG. 5166: DNA268708, DNA268708, 229391_s_at
FIG. 5167A-C: DNA331667, 198342.3, 229394_s_at
FIG. 5168: PRO86662
FIG. 5169: DNA287421, 234832.1, 229437_at
FIG. 5170: PRO69678
FIG. 5171: DNA330797, 211332.1, 229442_at
FIG. 5172: PRO85961
FIG. 5173: DNA328090, 007911.2, 229450_at
FIG. 5174: PRO84001
FIG. 5175: DNA330798, 984597.1, 229483_at
FIG. 5176: PRO85962
FIG. 5177: DNA330799, 481875.1, 229551_x_at
FIG. 5178: PRO85963
FIG. 5179: DNA330800, AK056271, 229595_at
FIG. 5180: PRO85964
FIG. 5181: DNA330801, 199864.1, 229610_at
FIG. 5182: PRO85965
FIG. 5183: DNA327205, NP_443174.1, 229625_at
FIG. 5184: PRO83478
FIG. 5185A-B: DNA226321, NP_006021.1,
229636_at
FIG. 5186: PRO36784
FIG. 5187A-B: DNA330802, 7694410.1, 229686_at
FIG. 5188: PRO85966
FIG. 5189: DNA331668, 403448.4, 229699_at
FIG. 5190: PRO86663
FIG. 5191A-C: DNA330804, NP_055847.1,
229704_at
FIG. 5192: PRO85968
FIG. 5193: DNA331669, 1466538.1, 229718_at
FIG. 5194: PRO86664
FIG. 5195A-B: DNA227985, CBX6, 229733_s_at
FIG. 5196: PRO38448
FIG. 5197: DNA330806, 200298.1, 229809_at
FIG. 5198: PRO85970
FIG. 5199: DNA330807, 334422.1, 229814_at
FIG. 5200: PRO85971
FIG. 5201: DNA328092, NP_002598.2, 229830_at
FIG. 5202: PRO84003
FIG. 5203: DNA330808, 1397087.3, 229838_at
FIG. 5204: PRO85972
FIG. 5205: DNA328972, BC009950, 229872_s_at
FIG. 5206A-C: DNA330810, AF330041, 229881_at
FIG. 5207: PRO85974
FIG. 5208: DNA287290, AK001793, 229980_s_at
FIG. 5209: PRO69560
FIG. 5210: DNA330811, 1382987.2, 230000_at
FIG. 5211: PRO85975
FIG. 5212A-B: DNA330812, 000264.19, 230021_at
FIG. 5213: PRO85976
FIG. 5214: DNA330813, 246201.1, 230036_at
FIG. 5215: PRO85977
FIG. 5216: DNA258657, DNA258657, 230060_at
FIG. 5217: PRO52596
FIG. 5218: DNA330814, 309641.1, 230097_at
FIG. 5219: PRO85978
FIG. 5220: DNA329467, 029236.1, 230110_at
FIG. 5221: PRO85033
FIG. 5222: DNA330815, AK057940, 230165_at
FIG. 5223: PRO85979
FIG. 5224: DNA329468, BC011589, 230170_at
FIG. 5225: PRO88
FIG. 5226: DNA330816, 980409.1, 230192_at
FIG. 5227: PRO85980
FIG. 5228A-B: DNA194784, DNA194784,
230218_at
FIG. 5229: PRO24061
FIG. 5230: DNA331670, 373719.30, 230257_s_at
FIG. 5231: PRO86665
FIG. 5232A-C: DNA330817, AB020335, 230265_at
FIG. 5233: PRO85981
FIG. 5234: DNA330818, 212282.1, 230304_at
FIG. 5235: PRO85982
FIG. 5236: DNA330819, 982802.1, 230337_at
FIG. 5237: PRO85983
FIG. 5238: DNA330820, 230585.2, 230345_at
FIG. 5239: PRO85984
FIG. 5240: DNA329470, NP_002756.1, 230352_at
FIG. 5241: PRO85035
FIG. 5242: DNA331671, 277648.15, 230375_at
FIG. 5243: PRO86666
FIG. 5244: DNA331672, 332195.1, 230391_at
FIG. 5245: PRO86667
FIG. 5246: DNA257756, DNA257756, 230405_at
FIG. 5247: DNA330823, 010867.1, 230449_x_at
FIG. 5248: PRO85987
FIG. 5249A-B: DNA331673, 333480.5, 230489_at
FIG. 5250: PRO86668
FIG. 5251A-B: DNA330825, 406864.4, 230526_at
FIG. 5252: PRO85989
FIG. 5253: DNA331674, 059446.1, 230529_at
FIG. 5254: PRO86669
FIG. 5255: DNA330827, NP_079521.1, 230536_at
FIG. 5256: PRO85991
FIG. 5257: DNA330828, 233615.1, 230566_at
FIG. 5258: PRO85992
FIG. 5259: DNA330829, 007717.1, 230580_at
FIG. 5260: PRO85993
FIG. 5261: DNA257789, NP_116219.1, 230656_s_at
FIG. 5262: PRO52338
FIG. 5263: DNA330830, 216899.1, 230703_at
FIG. 5264: PRO85994
FIG. 5265A-B: DNA328499, SORL1, 230707_at
FIG. 5266: PRO84321
FIG. 5267A-B: DNA328099, 335889.1, 230779_at
FIG. 5268: PRO84009
FIG. 5269: DNA194391, HSM800477, 230848_s_at
FIG. 5270: DNA330831, 208876.1, 230913_at
FIG. 5271: PRO85995
FIG. 5272: DNA330832, 253831.5, 230930_at
FIG. 5273: PRO85996
FIG. 5274: DNA304827, AF293462, 230966_at
FIG. 5275: PRO1265
FIG. 5276: DNA330833, 984179.1, 230970_at
FIG. 5277: PRO85997
FIG. 5278: DNA331675, BC017477, 231094_s_at
FIG. 5279: PRO86670
FIG. 5280: DNA331676, 980781.1, 231109_at
FIG. 5281: PRO86671
FIG. 5282: DNA329473, 370473.13, 231124_x_at
FIG. 5283: PRO85038
FIG. 5284: DNA330835, 399441.1, 231166_at
FIG. 5285: PRO85999
FIG. 5286A-B: DNA330836, 242968.17, 231169_at
FIG. 5287: PRO86000
FIG. 5288: DNA330837, 429490.1, 231182_at
FIG. 5289: PRO86001
FIG. 5290: DNA150808, HUMGBP1, 231577_s_at
FIG. 5291: PRO12478
FIG. 5292: DNA155700, DNA155700, 231579_s_at
FIG. 5293: DNA330838, NP_037460.2, 231715_s_at
FIG. 5294: PRO80743
FIG. 5295: DNA330839, NP_060908.1, 231769_at
FIG. 5296: PRO86002
FIG. 5297: DNA330840, BC015355, 231772_x_at
FIG. 5298: PRO86003
FIG. 5299: DNA208647, DNA208647, 231775_at
FIG. 5300: PRO1206
FIG. 5301: DNA330841, 983019.1, 231776_at
FIG. 5302: PRO86004
FIG. 5303: DNA329474, AK001874, 231784_s_at
FIG. 5304: PRO38893
FIG. 5305: DNA330842, 242234.14, 231793_s_at
FIG. 5306: PRO86005
FIG. 5307: DNA329312, AF414120, 231794_at
FIG. 5308: PRO84901
FIG. 5309: DNA330843, 201388.1, 231832_at
FIG. 5310: PRO86006
FIG. 5311A-B: DNA330844, 243553.2, 231852_at
FIG. 5312: PRO86007
FIG. 5313: DNA330845, BC009777, 231863_at
FIG. 5314: PRO86008
FIG. 5315A-B: DNA330846, BC005847, 231876_at
FIG. 5316: DNA331677, 981573.1, 231890_at
FIG. 5317: PRO86672
FIG. 5318: DNA330848, 1447268.2, 231904_at
FIG. 5319: PRO86011
FIG. 5320: DNA330849, EFG2, 231918_s_at
FIG. 5321: PRO86012
FIG. 5322A-B: DNA256267, BAB13444.1,
231956_at
FIG. 5323: PRO51311
FIG. 5324A-B: DNA271768, AB037834, 231996_at
FIG. 5325A-C: DNA330850, 152462.1, 232044_at
FIG. 5326: PRO86013
FIG. 5327: DNA330851, 337679.3, 232081_at
FIG. 5328: PRO86014
FIG. 5329: DNA330852, 1383611.1, 232138_at
FIG. 5330: PRO86015
FIG. 5331: DNA330853, 255956.19, 232141_at
FIG. 5332: PRO86016
FIG. 5333: DNA328113, 218535.1, 232150_at
FIG. 5334: PRO84020
FIG. 5335: DNA330854, AK023113, 232155_at,
FIG. 5336: PRO86017
FIG. 5337: DNA331678, ABIN-2, 232160_s_at
FIG. 5338: PRO86673
FIG. 5339A-E: DNA331679, NP_112598.1,
232164_s_at
FIG. 5340: PRO86674
FIG. 5341: DNA330856, HSM802268, 232165_at
FIG. 5342: DNA330857, 271071.1, 232175_at
FIG. 5343: PRO86020
FIG. 5344: DNA330858, 252659.1, 232213_at
FIG. 5345: PRO86021
FIG. 5346: DNA330859, 016890.1, 232216_at
FIG. 5347: PRO86022
FIG. 5348: DNA331680, 393520.1, 232238_at
FIG. 5349: PRO86675
FIG. 5350: DNA330861, AK000490, 232278_s_at
FIG. 5351: PRO86024
FIG. 5352: DNA287658, 199168.2, 232291_at
FIG. 5353: PRO69902
FIG. 5354: DNA331681, 339154.9, 232304_at
FIG. 5355: PRO86676
FIG. 5356: DNA330863, 295041.1, 232365_at
FIG. 5357: PRO86026
FIG. 5358: DNA331682, 1384413.5, 232369_at
FIG. 5359: PRO86677
FIG. 5360: DNA287182, 424693.25, 232375_at
FIG. 5361: PRO69470
FIG. 5362: DNA330865, 066613.1, 232412_at
FIG. 5363: PRO86028
FIG. 5364: DNA331683, 422960.1, 232504_at
FIG. 5365: PRO86678
FIG. 5366: DNA330867, 333565.1, 232527_at
FIG. 5367: PRO86030
FIG. 5368: DNA331684, AF161339, 232543_x_at
FIG. 5369: DNA330868, 337037.1, 232584_at
FIG. 5370: PRO86031
FIG. 5371: DNA330869, 406591.1, 232687_at
FIG. 5372: PRO86032
FIG. 5373: DNA330870, 227719.1, 232883_at
FIG. 5374: PRO86033
FIG. 5375: DNA330871, 419923.1, 233019_at
FIG. 5376: PRO86034
FIG. 5377: DNA287404, AK026486, 233085_s_at
FIG. 5378: PRO69661
FIG. 5379: DNA330872, 056107.1, 233127_at
FIG. 5380: PRO86035
FIG. 5381A-B: DNA329422, BAA92605.1,
233208_x_at
FIG. 5382: PRO84993
FIG. 5383A-B: DNA330873, AB040885, 233458_at
FIG. 5384: DNA330874, NP_057528.1, 233461_x_at
FIG. 5385: PRO86037
FIG. 5386: DNA331423, AF176071, 233467_s_at
FIG. 5387: DNA330875, 006221.2, 233506_at
FIG. 5388: PRO86038
FIG. 5389: DNA330876, AK055587, 233528_s_at
FIG. 5390: PRO86039
FIG. 5391: DNA328812, AB033087, 233575_s_at
FIG. 5392: DNA330877, NP_055075.1, 233588_x_at
FIG. 5393: PRO86040
FIG. 5394A-C: DNA330638, HSM801490,
233632_s_at
FIG. 5395: DNA329287, NP_057484.2, 233746_x_at
FIG. 5396: PRO84879
FIG. 5397: DNA329481, ASB2, 233857_s_at
FIG. 5398: PRO60949
FIG. 5399: DNA326800, XRN2, 233878_s_at
FIG. 5400: PRO83133
FIG. 5401: DNA330547, MOV10, 233917_s_at
FIG. 5402: PRO85731
FIG. 5403: DNA330878, NP_079111.1, 233937_at
FIG. 5404: PRO86041
FIG. 5405: DNA329571, HSPC195, 233955_x_at
FIG. 5406: PRO51662
FIG. 5407: DNA329332, BC001262, 233970_s_at
FIG. 5408: PRO84916
FIG. 5409: DNA331685, AK026111, 233986_s_at
FIG. 5410: PRO86680
FIG. 5411: DNA331686, HSA271091, 234000_s_at
FIG. 5412: PRO86681
FIG. 5413: DNA331687, HUMVA25A, 234013_at
FIG. 5414: PRO86682
FIG. 5415: DNA330880, NP_150283.1, 234284_at
FIG. 5416: PRO86043
FIG. 5417: DNA330881, CRACC, 234306_s_at
FIG. 5418: PRO1138
FIG. 5419: DNA329312, CTLA4, 234362_s_at
FIG. 5420: PRO84901
FIG. 5421: DNA329483, NP_443104.1, 234408_at
FIG. 5422: PRO20110
FIG. 5423: DNA287425, NP_060979.1, 234464_s_at
FIG. 5424: PRO69682
FIG. 5425: DNA330387, FBXO5, 234863_x_at
FIG. 5426: PRO85596
FIG. 5427: DNA304813, NP_277053.1, 234973_at
FIG. 5428: PRO71222
FIG. 5429: DNA330882, 406739.1, 234974_at
FIG. 5430: PRO86044
FIG. 5431: DNA330883, 1384547.1, 234986_at
FIG. 5432: PRO86045
FIG. 5433A-B: DNA330884, 267153.16, 234987_at
FIG. 5434: PRO86046
FIG. 5435: DNA257389, NP_116248.1, 234993_at
FIG. 5436: PRO51974
FIG. 5437: DNA330885, AK055618, 235022_at
FIG. 5438: PRO86047
FIG. 5439: DNA331688, 404157.1, 235052_at
FIG. 5440: PRO86683
FIG. 5441: DNA331689, 996962.6, 235056_at
FIG. 5442: PRO86684
FIG. 5443: DNA328143, AK054678, 235061_at
FIG. 5444: PRO84048
FIG. 5445: DNA330888, 7687712.2, 235088_at
FIG. 5447: DNA330889, AK055762, 235096_at
FIG. 5448: PRO86050
FIG. 5449: DNA193891, DNA193891, 235099_at
FIG. 5450: PRO23309
FIG. 5451A-B: DNA330890, AB058722, 235106_at
FIG. 5452: DNA330891, AK027315, 235113_at
FIG. 5453: PRO86052
FIG. 5454: DNA328 146, BC019239, 235117_at
FIG. 5455: PRO84051
FIG. 5456: DNA330892, 198067.4, 235136_at
FIG. 5457: PRO86053
FIG. 5458: DNA330893, 337392.1, 235157_at
FIG. 5459: PRO86054
FIG. 5460: DNA329486, 1058246.1, 235175_at
FIG. 5461: PRO85047
FIG. 5462: DNA330894, AF455817, 235177_at
FIG. 5463: PRO86055
FIG. 5464: DNA331690, 200228.1, 235199_at
FIG. 5465: PRO86685
FIG. 5466: DNA330896, 250896.1, 235213_at
FIG. 5467: PRO86057
FIG. 5468: DNA329488, 1501300.6, 235244_at
FIG. 5469: PRO85049
FIG. 5470A-C: DNA330897, 332999.23, 235252_at
FIG. 5471: PRO86058
FIG. 5472: DNA324093, BC019263, 235256_s_at
FIG. 5473: PRO80802
FIG. 5474: DNA260946, NP_115741.1, 235266_at
FIG. 5475: PRO54699
FIG. 5476A-C: DNA329379, 010205.2, 235287_at
FIG. 5477: PRO84957
FIG. 5478: DNA330898, 227608.1, 235299_at
FIG. 5479: PRO86059
FIG. 5480A-B: DNA330899, 7690822.1, 235306_at
FIG. 5481: PRO86060
FIG. 5482A-B: DNA330900, 199492.10,
235331_x_at
FIG. 5483: PRO86061
FIG. 5484: DNA330901, 400258.1, 235360_at
FIG. 5485: PRO86062
FIG. 5486: DNA330902, 481462.4, 235389_at
FIG. 5487: PRO86063
FIG. 5488: DNA331691, 405045.1, 235412_at
FIG. 5489: PRO86686
FIG. 5490: DNA330904, 1446121.1, 235415_at
FIG. 5491: PRO86065
FIG. 5492: DNA331692, 979330.2, 235425_at
FIG. 5493: PRO86687
FIG. 5494: DNA257872, DNA257872, 235457_at
FIG. 5495: DNA330906, NP_116171.2, 235458_at
FIG. 5496: PRO86067
FIG. 5497: DNA257302, DNA257302, 235463_s_at
FIG. 5498: DNA330907, 7692322.1, 235469_at
FIG. 5499: PRO86068
FIG. 5500: DNA331693, 203586.1, 235508_at
FIG. 5501: PRO86688
FIG. 5502: DNA330909, 229234.17, 235523_at
FIG. 5503: PRO86070
FIG. 5504: DNA304793, NP_443173.1, 235574_at
FIG. 5505: PRO71205
FIG. 5506: DNA330910, 032253.1, 235581_at
FIG. 5507: PRO86071
FIG. 5508: DNA330911, 1446080.1, 235607_at
FIG. 5509: PRO86072
FIG. 5510: DNA330912, 984873.1, 235609_at
FIG. 5511: PRO86073
FIG. 5512: DNA331694, 222666.9, 235643_at
FIG. 5513: PRO86689
FIG. 5514: DNA331695, 350462.1, 235652_at
FIG. 5515: PRO86690
FIG. 5516: DNA330915, 238456.7, 235662_at
FIG. 5517: PRO86076
FIG. 5518: DNA330916, 234580.1, 235670_at
FIG. 5519: PRO86077
FIG. 5520: DNA330917, 238496.1, 235696_at
FIG. 5521: PRO86078
FIG. 5522: DNA330918, 250552.1, 235699_at
FIG. 5523: PRO86079
FIG. 5524: DNA330919, 423261.6, 235739_at
FIG. 5525: PRO86080
FIG. 5526: DNA330920, 249518.17, 235783_at
FIG. 5527: PRO86081
FIG. 5528: DNA330921, 246858.18, 235816_s_at
FIG. 5529: PRO86082
FIG. 5530: DNA330922, 1447139.1, 235907_at
FIG. 5531: PRO86083
FIG. 5532: DNA330923, 981520.1, 235940_at
FIG. 5533: PRO86084
FIG. 5534: DNA330924, 237828.3, 235984_at
FIG. 5535: PRO86085
FIG. 5536: DNA330925, 257787.1, 235985_at
FIG. 5537: PRO86086
FIG. 5538: DNA330926, 1446421.2, 236079_at
FIG. 5539: PRO86087
FIG. 5540: DNA330927, 1499766.1, 236156_at
FIG. 5541: PRO86088
FIG. 5542A-E: DNA328165, 327340.35, 236172_at
FIG. 5543: PRO38220
FIG. 5544: DNA330928, 227714.1, 236180_at
FIG. 5545: PRO86089
FIG. 5546: DNA329489, 338163.1, 236280_at
FIG. 5547: PRO85050
FIG. 5548: DNA257767, DNA257767, 236285_at
FIG. 5549: DNA288255, 236339.1, 236295_s_at
FIG. 5550: PRO70016
FIG. 5551: DNA331696, 215451.1, 236338_at
FIG. 5552: PRO86691
FIG. 5553: DNA329490, 267918.1, 236347_at
FIG. 5554: PRO85051
FIG. 5555: DNA330930, 407665.1, 236379_at
FIG. 5556: PRO86091
FIG. 5557: DNA331697, 342862.1, 236419_at
FIG. 5558: PRO86692
FIG. 5559: DNA330932, 231907.4, 236470_at
FIG. 5560: PRO86093
FIG. 5561: DNA330933, 407881.1, 236506_at
FIG. 5562: PRO86094
FIG. 5563: DNA330934, 406491.1, 236595_at
FIG. 5564: PRO86095
FIG. 5565: DNA330935, 229915.1, 236610_at
FIG. 5566: PRO86096
FIG. 5567A-C: DNA330936, 351043.1, 236641_at
FIG. 5568: PRO86097
FIG. 5569: DNA330937, 205124.1, 236645_at
FIG. 5570: PRO86098
FIG. 5571: DNA330938, 7688127.1, 236668_at
FIG. 5572: PRO86099
FIG. 5573: DNA259749, DNA259749, 236782_at
FIG. 5574: DNA329491, 211743.1, 236787_at
FIG. 5575: PRO85052
FIG. 5576: DNA330939, 214517.1, 236796_at
FIG. 5577: PRO86100
FIG. 5578: DNA330940, 211136.19, 236832_at
FIG. 5579: PRO86101
FIG. 5580: DNA330941, 399601.2, 236836_at
FIG. 5581: PRO86102
FIG. 5582: DNA330942, 337215.1, 236907_at
FIG. 5583: PRO86103
FIG. 5584: DNA330943, 1042935.2, 237009_at
FIG. 5585: PRO86104
FIG. 5586: DNA330944, 218030.2, 237180_at
FIG. 5587: PRO86105
FIG. 5588: DNA330945, 334209.1, 237181_at
FIG. 5589: PRO86106
FIG. 5590A-B: DNA226536, TFRC, 237215_s_at
FIG. 5591: PRO36999
FIG. 5592: DNA330946, 023719.1, 237626_at
FIG. 5593: PRO86107
FIG. 5594: DNA331698, 341647.1, 237741_at
FIG. 5595: PRO86693
FIG. 5596: DNA330948, 305319.1, 237746_at
FIG. 5597: PRO86109
FIG. 5598: DNA330949, 089765.7, 237759_at
FIG. 5599: PRO86110
FIG. 5600: DNA331699, 983684.2, 237953_at
FIG. 5601: PRO86694
FIG. 5602: DNA330951, 253376.3, 238012_at
FIG. 5603: PRO86112
FIG. 5604A-B: DNA330952, 333610.10,
238021_s_at
FIG. 5605: PRO86113
FIG. 5606: DNA330953, BC019600, 238063_at
FIG. 5607: DNA331700, 983399.1, 238082_at
FIG. 5608: PRO86695
FIG. 5609: DNA330955, 311471.1, 238303_at
FIG. 5610: PRO86115
FIG. 5611: DNA330956, 329762.1, 238311_at
FIG. 5612: PRO86116
FIG. 5613: DNA329493, 337072.5, 238423_at
FIG. 5614: PRO85054
FIG. 5615: DNA108695, DNA108695, 238508_at
FIG. 5616: PRO9743
FIG. 5617: DNA330957, 003538.1, 238509_at
FIG. 5618: PRO86117
FIG. 5619: DNA330958, 370339.1, 238541_at
FIG. 5620: PRO86118
FIG. 5621: DNA330959, 358161.17, 238545_at
FIG. 5622: PRO86119
FIG. 5623: DNA260158, DNA260158, 2238551_at
FIG. 5624: DNA329495, 1447201.1, 238581_at
FIG. 5625: PRO85056
FIG. 5626: DNA329496, AK056126, 238600_at
FIG. 5627: PRO85057
FIG. 5628: DNA329497, 232064.1, 238619_at
FIG. 5629: PRO85058
FIG. 5630: DNA330960, 001782.1, 238633_at
FIG. 5631: PRO86120
FIG. 5632: DNA330961, 903323.1, 238651_at
FIG. 5633: PRO86121
FIG. 5634A-B: DNA329499, 332504.1, 238778_at
FIG. 5635: PRO85060
FIG. 5636: DNA330962, 336371.2, 238893_at
FIG. 5637: PRO86122
FIG. 5638A-B: DNA330963, 241110.10, 238910_at
FIG. 5639: PRO86123
FIG. 5640: DNA331701, 337114.1, 238913_at
FIG. 5641: PRO86696
FIG. 5642: DNA330965, NP_443111.2, 238960_s_at
FIG. 5643: PRO86125
FIG. 5644: DNA330966, 213271.1, 238987_at
FIG. 5645: PRO86126
FIG. 5646: DNA330967, 1499563.1, 238996_x_at
FIG. 5647: PRO86127
FIG. 5648: DNA330968, 005876.1, 239002_at
FIG. 5649: PRO86128
FIG. 5650: DNA328194, 998827.1, 239049_at
FIG. 5651: PRO84097
FIG. 5652: DNA331702, 406864.3, 239062_at
FIG. 5653: PRO86697
FIG. 5654: DNA330970, 026912.1, 239081_at
FIG. 5655: PRO86130
FIG. 5656: DNA330971, 413731.1, 239096_at
FIG. 5657: PRO86131
FIG. 5658A-B: DNA330972, 141588.42, 239133_at
FIG. 5659: PRO86132
FIG. 5660: DNA330973, NP_003328.1, 239163_at
FIG. 5661: PRO10751
FIG. 5662: DNA330974, 348211.8, 239219_at
FIG. 5663: PRO86133
FIG. 5664: DNA330975, 207267.3, 239278_at
FIG. 5665: PRO86134
FIG. 5666: DNA330976, 023223.1, 239328_at
FIG. 5667: PRO86135
FIG. 5668: DNA329501, 205323.1, 239331_at
FIG. 5669: PRO85062
FIG. 5670: DNA330977, 996962.2, 239364_at
FIG. 5671: PRO86136
FIG. 5672: DNA330978, 039840.1, 239376_at
FIG. 5673: PRO86137
FIG. 5674: DNA330979, 407730.7, 239388_at
FIG. 5675: PRO86138
FIG. 5676: DNA330980, 1134871.2, 239401_at
FIG. 5677: PRO86139
FIG. 5678: DNA330981, 406409.1, 239404_at
FIG. 5679: PRO86140
FIG. 5680: DNA330982, 980479.1, 239413_at
FIG. 5681: PRO86141
FIG. 5682: DNA328200, 405394.1, 239442_at
FIG. 5683: PRO84103
FIG. 5684: DNA330983, 305289.1, 239448_at
FIG. 5685: PRO86142
FIG. 5686: DNA330984, 015432.1, 239476_at
FIG. 5687: PRO86143
FIG. 5688: DNA330985, 317098.1, 239494_at
FIG. 5689: PRO86144
FIG. 5690: DNA330986, 215812.1, 239533_at
FIG. 5691: PRO86145
FIG. 5692: DNA330987, 116344.1, 239655_at
FIG. 5693: PRO86146
FIG. 5694: DNA330988, 333476.1, 239680_at
FIG. 5695: PRO86147
FIG. 5696: DNA330989, 298825.1, 239695_at
FIG. 5697: PRO86148
FIG. 5698: DNA330990, 004652.1, 239721_at
FIG. 5699: PRO86149
FIG. 5700: DNA330991, 017874.1, 239757_at
FIG. 5701: PRO86150
FIG. 5702: DNA330992, AK027783, 239771_at
FIG. 5703: PRO86151
FIG. 5704: DNA330993, 1439890.1, 239803_at
FIG. 5705: PRO86152
FIG. 5706: DNA330994, NP_115730.1, 239824_s_at
FIG. 5707: PRO86153
FIG. 5708A-C: DNA330995, 233142.9, 239897_at
FIG. 5709: PRO84857
FIG. 5710: DNA258952, DNA258952, 239901_at
FIG. 5711: DNA257698, DNA257698, 240070_at
FIG. 5712: DNA328206, 1384214.3, 240277_at
FIG. 5713: PRO84109
FIG. 5714: DNA330996, 144353.1, 240347_at
FIG. 5715: PRO86154
FIG. 5716: DNA331703, 314831.10, 240452_at
FIG. 5717: PRO86698
FIG. 5718: DNA330998, 979930.1, 240665_at
FIG. 5719: PRO86156
FIG. 5720: DNA330999, 1454193.1, 240830_at
FIG. 5721: PRO86157
FIG. 5722: DNA331000, 7693121.3, 240890_at
FIG. 5723: PRO86158
FIG. 5724: DNA331704, CARS, 240983_s_at
FIG. 5725: PRO86699
FIG. 5726: DNA329504, 197187.1, 241365_at
FIG. 5727: PRO85065
FIG. 5728: DNA331001, 1499887.1, 241370_at
FIG. 5729: PRO86159
FIG. 5730: DNA331002, 231340.1, 241435_at
FIG. 5731: PRO86160
FIG. 5732: DNA331003, 391185.30, 241495_at
FIG. 5733: PRO86161
FIG. 5734: DNA331004, 314498.1, 241505_at
FIG. 5735: PRO86162
FIG. 5736: DNA331005, 197725.1, 241722_x_at
FIG. 5737: PRO86163
FIG. 5738: DNA329505, BC017102, 241734_at
FIG. 5739: DNA331006, 193718.1, 241740_at
FIG. 5740: PRO86164
FIG. 5741: DNA331007, 405858.1, 241756_at
FIG. 5742: PRO86165
FIG. 5743: DNA331705, 428179.1, 241775_at
FIG. 5744: PRO86700
FIG. 5745: DNA195721, DNA195721, 241819_at
FIG. 5746: DNA331009, 222011.1, 241824_at
FIG. 5747: PRO86167
FIG. 5748: DNA331010, 218800.1, 241843_at
FIG. 5749: PRO86168
FIG. 5750: DNA331011, 979953.1, 241859_at
FIG. 5751: PRO86169
FIG. 5752: DNA331012, 030070.1, 241869_at
FIG. 5753: PRO86170
FIG. 5754: DNA331013, 406509.1, 241924_at
FIG. 5755: PRO86171
FIG. 5756: DNA329506, NP_387510.1, 241937_s_at
FIG. 5757: PRO85067
FIG. 5758: DNA331014, 1447958.2, 241985_at
FIG. 5759: PRO86172
FIG. 5760: DNA331015, 109159.1, 242031_at
FIG. 5761: PRO86173
FIG. 5762: DNA331016, 229438.1, 242051_at
FIG. 5763: PRO86174
FIG. 5764: DNA328213, 419856.5, 242059_at
FIG. 5765: PRO84116
FIG. 5766: DNA331017, 409906.7, 242060_x_at
FIG. 5767: PRO86175
FIG. 5768: DNA331018, 355930.1, 242110_at
FIG. 5769: PRO86176
FIG. 5770: DNA331019, 234788.2, 242245_at
FIG. 5771: PRO86177
FIG. 5772: DNA331020, 403459.1, 242261_at
FIG. 5773: PRO86178
FIG. 5774: DNA331021, 017309.1, 242268_at
FIG. 5775: PRO86179
FIG. 5776: DNA331022, BC009627, 242304_at
FIG. 5777: DNA331023, 119753.1, 242362_at
FIG. 5778: PRO86181
FIG. 5779: DNA331024, 028992.1, 242388_x_at
FIG. 5780: PRO86182
FIG. 5781: DNA328220, 239839.1, 242405_at
FIG. 5782: PRO84123
FIG. 5783: DNA331025, 127891.1, 242457_at
FIG. 5784: PRO86183
FIG. 5785: DNA328221, 221374.1, 242471_at
FIG. 5786: PRO84124
FIG. 5787: DNA257874, DNA257874, 242517_at
FIG. 5788: DNA331026, 014632.1, 242518_at
FIG. 5789: PRO86184
FIG. 5790: DNA331027, 053796.1, 242560_at
FIG. 5791: PRO86185
FIG. 5792: DNA331028, 7693434.1, 242606_at
FIG. 5793: PRO86186
FIG. 5794: DNA331706, 351474.1, 242617_at
FIG. 5795: PRO86701
FIG. 5796: DNA331707, 330870.5, 242625_at
FIG. 5797: PRO86702
FIG. 5798: DNA331030, 407930.2, 242648_at
FIG. 5799: PRO86188
FIG. 5800: DNA331031, 405967.1, 242669_at
FIG. 5801: PRO86189
FIG. 5802A-C: DNA331708, NP_006258.2,
242712_x_at
FIG. 5803: PRO86703
FIG. 5804A-C: DNA331033, AF330045, 242722_at
FIG. 5805: PRO86191
FIG. 5806: DNA331034, 7689086.1, 242735_x_at
FIG. 5807: PRO86192
FIG. 5808: DNA331035, 210512.1, 242783_at
FIG. 5809: PRO86193
FIG. 5810: DNA331036, 360991.1, 242836_at
FIG. 5811: PRO86194
FIG. 5812: DNA328224, 028975.1, 242859_at
FIG. 5813: PRO84127
FIG. 5814: DNA331037, 206873.1, 242890_at
FIG. 5815: PRO86195
FIG. 5816: DNA331709, 017276.1, 242903_at
FIG. 5817: PRO86704
FIG. 5818: DNA331710, 227540.15, 242960_at
FIG. 5819: PRO86705
FIG. 5820: DNA331711, 427600.1, 243006_at
FIG. 5821: PRO86706
FIG. 5822: DNA331041, 982079.2, 243030_at
FIG. 5823: PRO86199
FIG. 5824: DNA331042, 019764.1, 243037_at
FIG. 5825: PRO86200
FIG. 5826: DNA331043, 005042.1, 243134_at
FIG. 5827: PRO86201
FIG. 5828: DNA331044, 226264.10, 243154_at
FIG. 5829: PRO86202
FIG. 5830: DNA331045, 066434.1, 243222_at
FIG. 5831: PRO86203
FIG. 5832: DNA331712, 005752.1, 243271_at
FIG. 5833: PRO86707
FIG. 5834: DNA331047, BC020624, 243362_s_at
FIG. 5835: DNA331048, 7688599.1, 243366_s_at
FIG. 5836: PRO86206
FIG. 5837: DNA331049, 402027.4, 243395_at
FIG. 5838: PRO86207
FIG. 5839: DNA331713, 982999.2, 243423_at
FIG. 5840: PRO86708
FIG. 5841: DNA331051, 306804.1, 243469_at
FIG. 5842: PRO86209
FIG. 5843: DNA331714, 332965.1, 243496_at
FIG. 5844: PRO86709
FIG. 5845: DNA331053, 243689.1, 243509_at
FIG. 5846: PRO86211
FIG. 5847: DNA331715, 7683458.1, 243514_at
FIG. 5848: PRO86710
FIG. 5849: DNA331055, 1512996.3, 243561_at
FIG. 5850: PRO86213
FIG. 5851: DNA258957, DNA258957, 243631_at
FIG. 5852: DNA331056, 218946.1, 243759_at
FIG. 5853: PRO86214
FIG. 5854: DNA194184, DNA194184, 243764_at
FIG. 5855: PRO23576
FIG. 5856: DNA331057, 031316.1, 243888_at
FIG. 5857: PRO86215
FIG. 5858: DNA331058, 400813.1, 243918_at
FIG. 5859: PRO86216
FIG. 5860: DNA331059, 035870.32, 243934_at
FIG. 5861: PRO86217
FIG. 5862: DNA210271, DNA210271, 243999_at
FIG. 5863: PRO33803
FIG. 5864A-B: DNA331060, 406931.1, 244008_at
FIG. 5865: PRO86218
FIG. 5866: DNA331061, 198683.4, 244026_at
FIG. 5867: PRO86219
FIG. 5868: DNA331062, BC021973, 244052_at
FIG. 5869: PRO23771
FIG. 5870: DNA331716, 212607.1, 244267_at
FIG. 5871: PRO86711
FIG. 5872: DNA331064, 006039.1, 244313_at
FIG. 5873: PRO86221
FIG. 5874: DNA108738, DNA108738, 244321_at
FIG. 5875: PRO9822
FIG. 5876: DNA331065, 341348.1, 244382_at
FIG. 5877: PRO86222
FIG. 5878: DNA331066, 207228.1, 244443_at
FIG. 5879: PRO86223
FIG. 5880: DNA328239, 331922.4, 244450_at
FIG. 5881: PRO84142
FIG. 5882: DNA331067, 164869.1, 244599_at
FIG. 5883: PRO86224
FIG. 5884: DNA331068, 337465.1, 244677_at
FIG. 5885: PRO86225
FIG. 5886: DNA329512, 336575.1, 244780_at
FIG. 5887: PRO85073
FIG. 5888: DNA331069, 008651.1, 244798_at
FIG. 5889: PRO86226
FIG. 5890: DNA331070, 393412.1, 244801_at
FIG. 5891: PRO86227
FIG. 5892: DNA331071, 343563.1, 244869_at
FIG. 5893: PRO86228
FIG. 5894A-B: DNA254566, BAA11502.1,
D80007_at
FIG. 5895: PRO49669
FIG. 5896: DNA328961, BC011049, DNA36995_at
FIG. 5897: PRO84667
FIG. 5898A-B: DNA331072, AB046821,
DNA53991_at
FIG. 5899: DNA327200, KSP37, DNA59602_at
FIG. 5900: PRO1065
FIG. 5901: DNA327205, GBP5, DNA61875_at
FIG. 5902: PRO83478
FIG. 5903: DNA331717, BC020203, DNA71289_at
FIG. 5904: PRO86712
FIG. 5905: DNA331718, AK024409, DNA92232_at
FIG. 5906: PRO86713
FIG. 5907: DNA96866, DNA96866, DNA96866_at
FIG. 5908: PRO6015
FIG. 5909: DNA331073, BC011775, DNA101926_at
FIG. 5910: PRO86229
FIG. 5911: DNA108670, DNA108670,
DNA108670_at
FIG. 5912: PRO7171
FIG. 5913: DNA304467, BC004535, DNA108688_at
FIG. 5914: PRO71043
FIG. 5915A-B: DNA108728, DNA108728,
DNA108728_at
FIG. 5916: PRO9741
FIG. 5917: DNA329215, ICOS, DNA108917_at
FIG. 5918: PRO7424
FIG. 5919: DNA331719, BC002424, DNA143288_at
FIG. 5920: PRO12705
FIG. 5921A-B: DNA150956, HUMORFKG1P,
DNA150956_at
FIG. 5922: DNA330417, APOL6, DNA164989_at
FIG. 5923: PRO21341
FIG. 5924: DNA329483, AF384857, DNA166819_at
FIG. 5925: PRO20110
FIG. 5926: DNA26842, DNA26842, P_Z64949_at
FIG. 5927: PRO180
FIG. 5928: DNA304468, NP_077300.1, P_Z93700_at
FIG. 5929: PRO71044
FIG. 5930: DNA39423, DNA39423, P_X52252_at
FIG. 5931: PRO271
FIG. 5932: DNA330262, GW112, P_Z64962_at
FIG. 5933: PRO85493
FIG. 5934: DNA331074, AF252257, P_A37030_at
FIG. 5935: DNA60764, DNA60764, P_A46906_at
FIG. 5936: PRO1265
FIG. 5937: DNA331720, AF289594, P_A37063_at
FIG. 5938: PRO86714
FIG. 5939: DNA331721, BC017876, P_A37079_at
FIG. 5940: PRO71045
FIG. 5941: DNA76401, DNA76401, P_A37126_at
FIG. 5942: PRO1575
FIG. 5943: DNA304475, NP_116246.1, P_A37128_at
FIG. 5944: PRO71049
FIG. 5945: DNA66480, HSAPO1, NM_000043_at
FIG. 5946: PRO1207
FIG. 5947: DNA88195, CD3G, NM_000073_at
FIG. 5948: PRO2693
FIG. 5949: DNA325712, CDK4, NM_000075_at
FIG. 5950: PRO82194
FIG. 5951: DNA329934, BC013083, NM_000099_at
FIG. 5952: PRO2721
FIG. 5953A-B: DNA331722, HUMFVA,
NM_000130_at
FIG. 5954: PRO36374
FIG. 5955: DNA331723, U66095, NM_000161_at
FIG. 5956: PRO86715
FIG. 5957: DNA227668, HUMGLYKINB,
NM_000167_at
FIG. 5958: PRO38131
FIG. 5959A-D: DNA331724, HSGLA,
NM_000169_at
FIG. 5960: DNA331725, BC006342, NM_000175_at
FIG. 5961: DNA150823, NP_000185.1,
NM_000194_at
FIG. 5962: PRO12810
FIG. 5963: DNA331726, HUMICAMA1A,
NM_000201_at
FIG. 5964: PRO86716
FIG. 5965A-B: DNA88419, HSINTA6R,
NM_000210_at
FIG. 5966: PRO2339
FIG. 5967: DNA88428, HUMLAP, NM_000211_at
FIG. 5968: PRO2787
FIG. 5969: DNA226014, NP_000230.1,
NM_000239_at
FIG. 5970: PRO36477
FIG. 5971: DNA97287, NP_000240.1,
NM_000249_at
FIG. 5972: PRO3634
FIG. 5973: DNA88554, NP_000241.1,
NM_000250_at
FIG. 5974: PRO2839
FIG. 5975: DNA331727, BC008015, NM_000269_at
FIG. 5976: PRO37534
FIG. 5977A-E: DNA331728, PTEN4, NM_000314_at
FIG. 5978: DNA83020, NP_000349.1,
NM_000358_at
FIG. 5979: PRO2561
FIG. 5980: DNA227081, EGR2, NM_000399_at
FIG. 5981: PRO37544
FIG. 5982: DNA76512, HSIL2REC, NM_000417_at
FIG. 5983: PRO2020
FIG. 5984: DNA76514, HSIL4R, NM_000418_at
FIG. 5985: PRO2540
FIG. 5986: DNA329522, NP_000433.2,
NM_000442_at
FIG. 5987: PRO85080
FIG. 5988: DNA188732, NP_000475.1,
NM_000484_at
FIG. 5989: PRO25302
FIG. 5990: DNA331729, AF281258, NM_000517_at
FIG. 5991: DNA331730, BC014514, NM_000527_at
FIG. 5992: PRO2915
FIG. 5993: DNA331731, HSASM2MR,
NM_000543_at
FIG. 5994: DNA76516, IL6R, NM_000565_at
FIG. 5995: PRO2022
FIG. 5996: DNA36718, HUMIL10, NM_000572_at
FIG. 5997: PRO73
FIG. 5998: DNA324158, NP_000567.1,
NM_000576_at
FIG. 5999: PRO65
FIG. 6000A-B: DNA331732, HSCCR5AB2,
NM_000579_at
FIG. 6001: PRO25194
FIG. 6002: DNA290585, NP_000573.1,
NM_000582_f_at
FIG. 6003: PRO70536
FIG. 6004: DNA216500, NP_000575.1,
NM_000584_at
FIG. 6005: PRO34252
FIG. 6006: DNA36712, HUMIL3, NM_000588_at
FIG. 6007: PRO67
FIG. 6008A-B: DNA331733, AF361105,
NM_000590_at
FIG. 6009: DNA331734, BC014081, NM_000593_at
FIG. 6010: PRO36996
FIG. 6011A-B: DNA331735, AY066019,
NM_000594_at
FIG. 6012A-B: DNA331736, AY070490,
NM_000595_at
FIG. 6013: DNA331737, BC009902, NM_000597_at
FIG. 6014: PRO2587
FIG. 6015: DNA217246, NP_000591.1,
NM_000600_at
FIG. 6016: PRO34288
FIG. 6017: DNA331075, NP_000601.2,
NM_000610_at
FIG. 6018: PRO86231
FIG. 6019A-C: DNA331738, AF375790,
NM_000619_at
FIG. 6020A-B: DNA220752, ITGAM,
NM_000632_at
FIG. 6021: PRO34730
FIG. 6022A-B: DNA97288, HUMBCL2C,
NM_000633_at
FIG. 6023: PRO3635
FIG. 6024: DNA331739, A12178, NM_000636_at
FIG. 6025: PRO86720
FIG. 6026: DNA331740, HUMHPC, NM_000639_at
FIG. 6027: PRO1208
FIG. 6028: DNA329000, HSU03905, NM_000647_at
FIG. 6029: PRO84690
FIG. 6030: DNA328253, NP_004029.1,
NM_000699_at
FIG. 6031: PRO84149
FIG. 6032: DNA89242, ANXA1, NM_000700_at
FIG. 6033: PRO2907
FIG. 6034: DNA88194, CD3E, NM_000733_at
FIG. 6035: PRO2220
FIG. 6036: DNA329975, PRO2325, NM_000791_at
FIG. 6037: DNA331741, BC003097, NM_000873_at
FIG. 6038: PRO86721
FIG. 6039: DNA331076, HSIFNABR,
NM_000874_at
FIG. 6040: PRO86232
FIG. 6041A-B: DNA83101, NP_000868.1,
NM_000877_at
FIG. 6042: PRO2590
FIG. 6043A-B: DNA76508, A07795, NM_000878_at
FIG. 6044: PRO2538
FIG. 6045: DNA36714, NP_000870.1,
NM_000879_at
FIG. 6046: PRO69
FIG. 6047A-B: DNA88417, HSINTAL4,
NM_000885_at
FIG. 6048: PRO2337
FIG. 6049: DNA88433, HUMINTB7A,
NM_000889_at
FIG. 6050: PRO2346
FIG. 6051: DNA226053, NP_000908.1,
NM_000917_at
FIG. 6052: PRO36516
FIG. 6053A-B: DNA331742, BC018127,
NM_000919_at
FIG. 6054: PRO86722
FIG. 6055: DNA227709, PTGER2, NM_000956_at
FIG. 6056: PRO38172
FIG. 6057: DNA226195, NP_000949.1,
NM_000958_at
FIG. 6058: PRO36658
FIG. 6059: DNA327639, TCN1, NM_001062_at
FIG. 6060: PRO83640
FIG. 6061A-B: DNA150748, ADCY7,
NM_001114_at
FIG. 6062: PRO12446
FIG. 6063: DNA171404, HSU45878, NM_001165_at
FIG. 6064: PRO20132
FIG. 6065: DNA331743, AAA19687.1,
NM_001168_at
FIG. 6066: PRO12242
FIG. 6067A-B: DNA325972, BC018739,
NM_001211_at
FIG. 6068: PRO82417
FIG. 6069: DNA287267, CCNA2, NM_001237_at
FIG. 6070: PRO37015
FIG. 6071: DNA196674, D86042, NM_001243_at
FIG. 6072: PRO2938
FIG. 6073: DNA325568, BC017575, NM_001274_at
FIG. 6074: PRO12187
FIG. 6075: DNA226177, CCR1, NM_001295_at
FIG. 6076: PRO36640
FIG. 6077: DNA331744, CTSW, NM_001335_at
FIG. 6078: PRO1574
FIG. 6079: DNA93466, HUMEDG, NM_001400_at
FIG. 6080: PRO4936
FIG. 6081: DNA331745, HSU77085, NM_001423_at
FIG. 6082: PRO12467
FIG. 6083A-C: DNA151167, HSABP280,
NM_001456_at
FIG. 6084: PRO12867
FIG. 6085A-C: DNA331746, AF043045,
NM_001457_at
FIG. 6086: PRO86723
FIG. 6087: DNA188346, FLT3LG, NM_001459_at
FIG. 6088: PRO21766
FIG. 6089: DNA227173, HSU93049, NM_001465_at
FIG. 6090: PRO37636
FIG. 6091A-B: DNA331747, GABBR1,
NM_001470_at
FIG. 6092: PRO86724
FIG. 6093A-B: DNA76503, IL10RA, NM_001558_at
FIG. 6094: PRO2536
FIG. 6095A-B: DNA227750, IL12RB2,
NM_001559_at
FIG. 6096: PRO38213
FIG. 6097: DNA76556, HSU03397, NM_001561_at
FIG. 6098: PRO2023
FIG. 6099: DNA82362, CXCL10, NM_001565_at
FIG. 6100: PRO1718
FIG. 6101: DNA227013, NP_001560.1,
NM_001569_at
FIG. 6102: PRO37476
FIG. 6103: DNA331748, BC009799, NM_001657_at
FIG. 6104: PRO46
FIG. 6105: DNA150716, HSZNFNPRA,
NM_001706_at
FIG. 6106: PRO12790
FIG. 6107: DNA331077, HUMBGPAB,
NM_001712_at
FIG. 6108: PRO86233
FIG. 6109: DNA150718, NP_001727.1,
NM_001736_at
FIG. 6110: PRO12435
FIG. 6111A-B: DNA226387, HSCYCLF,
NM_001761_at
FIG. 6112: PRO36850
FIG. 6113: DNA329002, CCT6A, NM_001762_at
FIG. 6114: PRO4912
FIG. 6115: DNA226380, HSCD37, NM_001774_at
FIG. 6116: PRO4695
FIG. 6117: DNA331749, D84277, NM_001775_at
FIG. 6118: PRO86725
FIG. 6119: DNA88199, HUMMEMGL1,
NM_001778_at
FIG. 6120: PRO2696
FIG. 6121: DNA226436, CD69, NM_001781_at
FIG. 6122: PRO36899
FIG. 6123: DNA331750, A23013, NM_001803_at
FIG. 6124: PRO2496
FIG. 6125: DNA151798, NP_001797.1,
NM_001806_at
FIG. 6126: PRO12186
FIG. 6127: DNA227232, SLC31A1, NM_001859_at
FIG. 6128: PRO37695
FIG. 6129: DNA331751, S68134, NM_001881_at
FIG. 6130: PRO86726
FIG. 6131: DNA331078, NP_001894.1,
NM_001903_at
FIG. 6132: PRO86234
FIG. 6133: DNA331752, BC010240, NM_001908_at
FIG. 6134: PRO86727
FIG. 6135: DNA225810, HSCATHL, NM_001912_at
FIG. 6136: PRO36273
FIG. 6137: DNA83048, DEFA4, NM_001925_at
FIG. 6138: PRO2057
FIG. 6139: DNA88215, NP_001919.1,
NM_001928_at
FIG. 6140: PRO2703
FIG. 6141: DNA196562, HSPCHDP7,
NM_001935_at
FIG. 6142: PRO25042
FIG. 6143: DNA226871, NP_001942.1,
NM_001951_at
FIG. 6144: PRO37334
FIG. 6145: DNA227332, NP_001943.1,
NM_001952_at
FIG. 6146: PRO37795
FIG. 6147: DNA225661, ECGF1, NM_001953_at
FIG. 6148: PRO36124
FIG. 6149: DNA273174, HSEF1DELA,
NM_00196_at
FIG. 6150: PRO61211
FIG. 6151: DNA150779, HUMETR103,
NM_001964_at
FIG. 6152: PRO12798
FIG. 6153: DNA331753, HUMENOG,
NM_001975_at
FIG. 6154: PRO38010
FIG. 6155: DNA331754, BC002464, NM_001992_at
FIG. 6156: PRO86728
FIG. 6157: DNA331755, D83920, NM_002003_at
FIG. 6158: PRO86729
FIG. 6159: DNA226881, HUMERGBFLI,
NM_002017_at
FIG. 6160: PRO37344
FIG. 6161: DNA88332, FVT1, NM_002035_at
FIG. 6162: PRO2753
FIG. 6163: DNA225979, G1P3, NM_002038_at
FIG. 6164: PRO36442
FIG. 6165: DNA331756, BC002666, NM_002053_at
FIG. 6166: PRO12478
FIG. 6167: DNA88374, GZMK, NM_002104_at
FIG. 6168: PRO2768
FIG. 6169: DNA228014, ICAM3, NM_002162_at
FIG. 6170: PRO38477
FIG. 6171: DNA331757, A17548, NM_002167_at
FIG. 6172: PRO86730
FIG. 6173: DNA76517, IL7R, NM_002185_at
FIG. 6174: PRO2541
FIG. 6175: DNA188271, NP_002179.1,
NM_002188_at
FIG. 6176: PRO21795
FIG. 6177: DNA226396, IL15RA, NM_002189_at
FIG. 6178: PRO36859
FIG. 6179: DNA227014, NP_002190.1,
NM_002199_at
FIG. 6180: PRO37477
FIG. 6181A-B: DNA88427, HSFNRB,
NM_002211_at
FIG. 6182: PRO2786
FIG. 6183: DNA103215, NP_002210.1,
NM_002219_at
FIG. 6184: PRO4545
FIG. 6185: DNA331758, S82269, NM_002222_at
FIG. 6186: PRO86731
FIG. 6187: DNA331759, BC002646, NM_002228_at
FIG. 6188: PRO4671
FIG. 6189: DNA331760, BC009466, NM_002229_at
FIG. 6190: PRO4650
FIG. 6191A-B: DNA331761, AF305731S5,
NM_002250_at
FIG. 6192: DNA150971, KLRB1, NM_002258_at
FIG. 6193: PRO12564
FIG. 6194: DNA326343, BC003572, NM_002265_at
FIG. 6195: PRO82739
FIG. 6196: DNA288243, LAG3, NM_002286_at
FIG. 6197: PRO36451
FIG. 6198A-B: DNA188301, LIF, NM_002309_at
FIG. 6199: PRO21834
FIG. 6200A-B: DNA331762, HUMLYTOXBB,
NM_002341_at
FIG. 6201: DNA88666, NP_002334.1,
NM_002343_at
FIG. 6202: PRO2892
FIG. 6203: DNA227150, LY6E, NM_002346_at
FIG. 6204: PRO37613
FIG. 6205: DNA327255, BC001061, NM_002394_at
FIG. 6206: PRO57298
FIG. 6207: DNA150937, HSU94352, NM_002405_at
FIG. 6208: PRO11598
FIG. 6209: DNA82376, CXCL9, NM_002416_at
FIG. 6210: PRO1723
FIG. 6211: DNA103283, MNDA, NM_002432_at
FIG. 6212: PRO4613
FIG. 6213: DNA103525, NP_002457.1,
NM_002466_at
FIG. 6214: PRO4852
FIG. 6215A-B: DNA331763, AF058696,
NM_002485_at
FIG. 6216: PRO36001
FIG. 6217: DNA103382, HSU49395, NM_002561_at
FIG. 6218: PRO4711
FIG. 6219A-B: DNA88331, HSFUR, NM_002569_at
FIG. 6220: PRO2752
FIG. 6221: DNA103488, PCNA, NM_002592_at
FIG. 6222: PRO4815
FIG. 6223: DNA328587, NP_002612.1,
NM_002621_at
FIG. 6224: PRO2854
FIG. 6225: DNA331764, NP_071438.1,
NM_002624_at
FIG. 6226: PRO86732
FIG. 6227: DNA227067, HSPKCB1A,
NM_002738_at
FIG. 6228: PRO37530
FIG. 6229: DNA227090, NP_002750.1,
NM_002759_at
FIG. 6230: PRO37553
FIG. 6231: DNA88626, HUMSAPABCD,
NM_002778_at
FIG. 6232: PRO2875
FIG. 6233: DNA329098, BC007897, NM_002808_at
FIG. 6234: PRO84749
FIG. 6235: DNA326853, NP_002818.1,
NM_002827_at
FIG. 6236: PRO38066
FIG. 6237: DNA88607, NP_002892.1,
NM_002901_at
FIG. 6238: PRO2863
FIG. 6239: DNA331765, AF294009, NM_002934_at
FIG. 6240: PRO2444
FIG. 6241: DNA331766, AF043339, NM_002983_at
FIG. 6242: DNA51778, HSHC21, NM_002984_at
FIG. 6243: PRO80
FIG. 6244: DNA330124, CCL22, NM_002990_at
FIG. 6245: PRO34107
FIG. 6246: DNA227788, NP_002995.1,
NM_003004_at
FIG. 6247: PRO38251
FIG. 6248: DNA329005, IISU [symbol] , NM_003037_at
FIG. 6249: PRO12612
FIG. 6250: DNA196489, HUMMCT, NM_003051_at
FIG. 6251: PRO24980
FIG. 6252A-B: DNA103542, HSLR11,
NM_003105_at
FIG. 6253: PRO4869
FIG. 6254: DNA331767, D78130, NM_003129_at
FIG. 6255: PRO37946
FIG. 6256: DNA328259, AF029311, NM_003150_at
FIG. 6257: DNA227447, HSTCF1C, NM_003202_at
FIG. 6258: PRO37910
FIG. 6259A-B: DNA226536, HSTRR,
NM_003234_at
FIG. 6260: PRO36999
FIG. 6261A-B: DNA83176, TGFBR3,
NM_003243_at
FIG. 6262: PRO2620
FIG. 6263: DNA103532, TM7SF1, NM_003272_at
FIG. 6264: PRO4859
FIG. 6265A-B: DNA103585, HUMTOPI,
NM_003286_at
FIG. 6266: PRO4909
FIG. 6267: DNA331768, BC007935, NM_003316_at
FIG. 6268: PRO22907
FIG. 6269: DNA331769, AF065241, NM_003329_at
FIG. 6270A-B: DNA331770, AF019563,
NM_003332_at
FIG. 6271: DNA331771, HSU76367, NM_003355_at
FIG. 6272: PRO86733
FIG. 6273: DNA151906, HSUNG, NM_003362_f_at
FIG. 6274: PRO12214
FIG. 6275: DNA103380, HUMVDAC1X,
NM_003374_at
FIG. 6276: PRO4710
FIG. 6277: DNA225992, NP_003374.1,
NM_003383_at
FIG. 6278: PRO36455
FIG. 6279: DNA227330, NP_003443.1,
NM_003452_at
FIG. 6280: PRO37793
FIG. 6281: DNA93449, AF025375, NM_003467_at
FIG. 6282: PRO4516
FIG. 6283: DNA331772, BC010022, NM_003504_at
FIG. 6284: PRO71058
FIG. 6285: DNA331773, AF123318, NM_003550_at
FIG. 6286: PRO86734
FIG. 6287: DNA331079, AF036342, NM_003650_at
FIG. 6288: PRO1191
FIG. 6289: DNA328260, AF305428, NM_003661_at
FIG. 6290: PRO84152
FIG. 6291: DNA151802, AB004066, NM_003670_at
FIG. 6292: PRO12890
FIG. 6293: DNA227213, NP_003671.1,
NM_003680_at
FIG. 6294: PRO37676
FIG. 6295: DNA331774, AK001769, NM_003730_at
FIG. 6296: PRO86735
FIG. 6297: DNA150433, AB005043, NM_003745_at
FIG. 6298: PRO12771
FIG. 6299: DNA328377, NP_003759.1,
NM_003768_at
FIG. 6300: PRO84232
FIG. 6301: DNA194746, HSM800355,
NM_003798_at
FIG. 6302: DNA196431, AF064090, NM_003807_at
FIG. 6303: PRO5810
FIG. 6304: DNA61870, HSU57059, NM_003810_at
FIG. 6305: PRO1096
FIG. 6306A-B: DNA200236, NP_003807.1,
NM_003816_at
FIG. 6307: PRO34137
FIG. 6308: DNA331775, BC000334, NM_003824_at
FIG. 6309: PRO4801
FIG. 6310: DNA331080, NP_003835.2,
NM_003844_at
FIG. 6311: PRO86235
FIG. 6312: DNA225550, IL18RAP, NM_003853_at
FIG. 6313: PRO36013
FIG. 6314: DNA103451, IL18R1, NM_003855_at
FIG. 6315: PRO4778
FIG. 6316: DNA151011, AF037989, NM_003877_at
FIG. 6317: PRO12839
FIG. 6318: DNA331776, IER3, NM_003897_at
FIG. 6319: PRO84760
FIG. 6320A-B: DNA150765, SLC7A6,
NM_003983_at
FIG. 6321: PRO12458
FIG. 6322: DNA88308, HUMFCREA,
NM_004106_at
FIG. 6323: PRO2739
FIG. 6324A-B: DNA331777, AF200219S2,
NM_004107_at
FIG. 6325: DNA227133, GBP2, NM_004120_at
FIG. 6326: PRO37596
FIG. 6327: DNA83091, HUMSP13E, NM_004131_at
FIG. 6328: PRO2081
FIG. 6329A-B: DNA151108, SREBF1,
NM_004176_at
FIG. 6330: PRO12105
FIG. 6331: DNA218676, AF125304, NM_004195_at
FIG. 6332: PRO34454
FIG. 6333: DNA103394, HSU81800, NM_004207_at
FIG. 6334: PRO4722
FIG. 6335: DNA329533, BC018782, NM_004221_at
FIG. 6336: PRO85085
FIG. 6337: DNA331778, AK027513, NM_004265_at
FIG. 6338: PRO86736
FIG. 6339: DNA151142, NP_004321.1,
NM_004330_at
FIG. 6340: PRO12110
FIG. 6341: DNA227303, NP_004322.1,
NM_004331_at
FIG. 6342: PRO37766
FIG. 6343: DNA287240, BST2, NM_004335_at
FIG. 6344: PRO29371
FIG. 6345: DNA225910, NP_004336.1,
NM_004345_at
FIG. 6346: PRO36373
FIG. 6347: DNA331779, CASP3, NM_004346_at
FIG. 6348: PRO12832
FIG. 6349A-B: DNA326191, NP_004351.1,
NM_004360_at
FIG. 6350: PRO2672
FIG. 6351A-C: DNA150729, HSU47741,
NM_004380_at
FIG. 6352: PRO12147
FIG. 6353A-B: DNA151420, S40832,
NM_004430_at
FIG. 6354: PRO12876
FIG. 6355A-B: DNA218283, EPHB6,
NM_004445_at
FIG. 6356: PRO34335
FIG. 6357: DNA331780, BC003110, NM_004512_at
FIG. 6358: PRO4843
FIG. 6359: DNA150935, NP_004547.1,
NM_004556_at
FIG. 6360: PRO12155
FIG. 6361A-B: DNA151831, NP_004564.1,
NM_004573_at
FIG. 6362: PRO12198
FIG. 6363: DNA328262, HSU57094, NM_004580_at
FIG. 6364: PRO84153
FIG. 6365: DNA331781, HSU77035, NM_004591_at
FIG. 6366: PRO1724
FIG. 6367: DNA331782, HUMVAIPR,
NM_004624_at
FIG. 6368: DNA329984, WRB, NM_004627_at
FIG. 6369: PRO11656
FIG. 6370: DNA329119, NP_004633.1,
NM_004642_at
FIG. 6371: PRO4550
FIG. 6372: DNA328578, NP_004656.2,
NM_004665_at
FIG. 6373: PRO7426
FIG. 6374: DNA331783, BC011726, NM_004706_at
FIG. 6375: PRO86737
FIG. 6376: DNA218284, AF053004, NM_004843_at
FIG. 6377: PRO34336
FIG. 6378: DNA151017, AB005047, NM_004844_at
FIG. 6379: PRO12841
FIG. 6380A-B: DNA150447, AB011098,
NM_004863_at
FIG. 6381: PRO12256
FIG. 6382: DNA88295, HUMERP72H,
NM_004911_at
FIG. 6383: PRO2733
FIG. 6384: DNA331784, AB001325, NM_004925_at
FIG. 6385: PRO38028
FIG. 6386A-B: DNA331785, DSC1, NM_004948_at
FIG. 6387: PRO36355
FIG. 6388: DNA227563, NP_004946.1,
NM_004955_at
FIG. 6389: PRO38026
FIG. 6390: DNA331786, HUMSTPK13,
NM_005030_at
FIG. 6391: PRO86738
FIG. 6392: DNA329011, BCL3, NM_005178_at
FIG. 6393: PRO4785
FIG. 6394: DNA331787, AF213050, NM_005192_at
FIG. 6395: PRO86739
FIG. 6396: DNA103330, HUMPOPSTK,
NM_005204_at
FIG. 6397: PRO4660
FIG. 6398: DNA331788, HUMIGCTL3,
NM_005214_at
FIG. 6399: DNA325060, NP_004075.1,
NM_005217_at
FIG. 6400: PRO2570
FIG. 6401: DNA331789, HSCFOS, NM_005252_at
FIG. 6402: DNA304668, HSPA1A, NM_005346_at
FIG. 6403: PRO71095
FIG. 6404: DNA331790, HUMCMYBA,
NM_005375_at
FIG. 6405: DNA227376, NP_005393.1,
NM_005402_at
FIG. 6406: PRO37839
FIG. 6407: DNA331791, BC005292, NM_005409_at
FIG. 6408: PRO19838
FIG. 6409: DNA329319, TOSO, NM_005449_at
FIG. 6410: PRO1607
FIG. 6411A-B: DNA189702, AF047348,
NM_005503_at
FIG. 6412: PRO22775
FIG. 6413: DNA150989, HSP27, NM_005532_at
FIG. 6414: PRO12569
FIG. 6415A-C: DNA331792, HUMOP18A,
NM_005563_at
FIG. 6416: DNA97285, LDHA, NM_005566_at
FIG. 6417: PRO3632
FIG. 6418: DNA225675, LMAN1, NM_005570_at
FIG. 6419: PRO36138
FIG. 6420: DNA331793, AF148645, NM_005614_at
FIG. 6421: PRO37938
FIG. 6422: DNA331794, BC001263, NM_005627_at
FIG. 6423: PRO86741
FIG. 6424: DNA226500, NP_005619.1,
NM_005628_at
FIG. 6425: PRO36963
FIG. 6426A-B: DNA227206, NP_005648.1,
NM_005657_at
FIG. 6427: PRO37669
FIG. 6428: DNA323937, NP_005689.2,
NM_005698_at
FIG. 6429: PRO80670
FIG. 6430: DNA331081, NP_005714.2,
NM_005723_at
FIG. 6431: PRO4845
FIG. 6432: DNA304459, PPIF, NM_005729_at
FIG. 6433: PRO37073
FIG. 6434A-B: DNA331082, AF057299,
NM_005732_at
FIG. 6435: PRO86236
FIG. 6436: DNA88541, PBEF, NM_005746_at
FIG. 6437: PRO2834
FIG. 6438: DNA329014, EBI3, NM_005755_at
FIG. 6439: PRO9998
FIG. 6440: DNA93548, NP_005758.1,
NM_005767_at
FIG. 6441: PRO4929
FIG. 6442: DNA331083, NP_005759.2,
NM_005768_at
FIG. 6443: PRO86237
FIG. 6444: DNA193866, AF081675, NM_005810_at
FIG. 6445: PRO23288
FIG. 6446: DNA75525, GPA33, NM_005814_at
FIG. 6447: PRO2524
FIG. 6448A-B: DNA88650, TACTILE,
NM_005816_at
FIG. 6449: PRO2460
FIG. 6450: DNA150959, NP_005813.1,
NM_005822_at
FIG. 6451: PRO11599
FIG. 6452: DNA329538, BC001731, NM_005898_at
FIG. 6453: PRO85088
FIG. 6454: DNA324110, MDH1, NM_005917_at
FIG. 6455: PRO4918
FIG. 6456: DNA328266, NP_005993.1,
NM_006002_at
FIG. 6457: PRO12125
FIG. 6458: DNA150941, NP_006012.1,
NM_006021_at
FIG. 6459: PRO12548
FIG. 6460: DNA227138, NP_006045.1,
NM_006054_at
FIG. 6461: PRO37601
FIG. 6462: DNA88614, HSRING6, NM_006120_at
FIG. 6463: PRO2867
FIG. 6464: DNA331795, NP_006129.2,
NM_006138_at
FIG. 6465: PRO81984
FIG. 6466: DNA331796, HUMCD284,
NM_006139_at
FIG. 6467: DNA330114, GPR19, NM_006143_at
FIG. 6468: PRO4946
FIG. 6469: DNA88372, HUMHFSP, NM_006144_at
FIG. 6470: PRO2312
FIG. 6471: DNA103526, HSU10485, NM_006152_at
FIG. 6472: PRO4853
FIG. 6473: DNA331797, BC020544, NM_006159_at
FIG. 6474: PRO2520
FIG. 6475: DNA151049, S74017, NM_006164_at
FIG. 6476: PRO12170
FIG. 6477A-B: DNA151841, HUMA20,
NM_006290_at
FIG. 6478: PRO12904
FIG. 6479: DNA331798, TSG101, NM_006292_at
FIG. 6480: PRO86742
FIG. 6481: DNA83109, HUMIIP, NM_006332_at
FIG. 6482: PRO2592
FIG. 6483: DNA331799, AY034481, NM_006372_at
FIG. 6484: PRO83688
FIG. 6485: DNA329540, UBD, NM_006398_at
FIG. 6486: PRO85090
FIG. 6487: DNA331800, BC007107, NM_006406_at
FIG. 6488: PRO12111
FIG. 6489: DNA331801, BC012589, NM_006419_at
FIG. 6490: PRO21708
FIG. 6491: DNA227795, CCT7, NM_006429_at
FIG. 6492: PRO38258
FIG. 6493: DNA329225, BC005926, NM_006495_at
FIG. 6494: PRO84833
FIG. 6495: DNA327702, AF074002, NM_006499_at
FIG. 6496: PRO83684
FIG. 6497: DNA151804, RELB, NM_006509_at
FIG. 6498: PRO12188
FIG. 6499A-B: DNA331802, AF012108,
NM_006534_at
FIG. 6500: PRO86743
FIG. 6501: DNA93439, HSY13248, NM_006564_at
FIG. 6502: PRO4515
FIG. 6503: DNA227751, NP_006557.1,
NM_006566_at
FIG. 6504: PRO38214
FIG. 6505: DNA227126, NP_006559.1,
NM_006568_at
FIG. 6506: PRO37589
FIG. 6507: DNA331803, AF116456, NM_006573_at
FIG. 6508: PRO738
FIG. 6509: DNA331804, BC001572, NM_006579_at
FIG. 6510: PRO12082
FIG. 6511: DNA324740, NP_006577.1,
NM_006586_at
FIG. 6512: PRO81365
FIG. 6513: DNA331805, HSM801976,
NM_006620_at
FIG. 6514: PRO86744
FIG. 6515: DNA328544, HSFIBLP, NM_006682_at
FIG. 6516: PRO84347
FIG. 6517: DNA227035, HUMHUMCM5,
NM_006739_at
FIG. 6518: PRO37498
FIG. 6519: DNA227512, NP_006736.1,
NM_006745_at
FIG. 6520: PRO37975
FIG. 6521A-B: DNA331806, AB005666,
NM_006747_at
FIG. 6522: PRO86745
FIG. 6523: DNA227416, NP_006745.1,
NM_006754_at
FIG. 6524: PRO37879
FIG. 6525: DNA331807, HSU30498, NM_006762_at
FIG. 6526: PRO86746
FIG. 6527: DNA227190, NP_006830.1,
NM_006839_at
FIG. 6528: PRO37653
FIG. 6529: DNA150812, RTVP1, NM_006851_at
FIG. 6530: PRO12481
FIG. 6531: DNA331808, HSU82278, NM_006866_at
FIG. 6532: PRO86747
FIG. 6533: DNA103221, NP_006866.1,
NM_006875_at
FIG. 6534: PRO4551
FIG. 6535: DNA328271, ZWINT, NM_007057_at
FIG. 6536: PRO81868
FIG. 6537: DNA331809, NP_009046.1,
NM_007115_at
FIG. 6538: PRO86748
FIG. 6539: DNA103587, HSMRL3R, NM_007208_at
FIG. 6540: PRO4911
FIG. 6541: DNA330180, TRC8, NM_007218_at
FIG. 6542: PRO85428
FIG. 6543: DNA331810, HSU64805, NM_007295_at
FIG. 6544: PRO23937
FIG. 6545: DNA331086, AB027467, NM_012112_at
FIG. 6546: PRO86239
FIG. 6547A-B: DNA226290, HSU28811,
NM_012201_at
FIG. 6548: PRO36753
FIG. 6549: DNA227143, NP_036400.1,
NM_012268_at
FIG. 6550: PRO37606
FIG. 6551A-B: DNA150955, NP_036420.1,
NM_012288_at
FIG. 6552: PRO12559
FIG. 6553: DNA331811, AF083247, NM_012328_at
FIG. 6554: PRO1471
FIG. 6555A-B: DNA227255, STAG3,
NM_012447_at
FIG. 6556: PRO37718
FIG. 6557: DNA304476, NP_036585.1,
NM_012453_at
FIG. 6558: PRO1125
FIG. 6559: DNA88510, HSNKG5, NM_012483_at
FIG. 6560: PRO2822
FIG. 6561: DNA103418, AF032862, NM_012484_at
FIG. 6562: PRO4746
FIG. 6563: DNA88189, HUMCD24B,
NM_013230_at
FIG. 6564: PRO2690
FIG. 6565: DNA331812, BC019883, NM_013269_at
FIG. 6566: PRO86749
FIG. 6567: DNA103481, HUMAUANTIG,
NM_013285_at
FIG. 6568: PRO4808
FIG. 6569: DNA196426, H963, NM_013308_at
FIG. 6570: PRO24924
FIG. 6571A-B: DNA329017, AF035947,
NM_013324_at
FIG. 6572: PRO84692
FIG. 6573: DNA150648, HIG2, NM_013332_at
FIG. 6574: PRO11576
FIG. 6575A-B: DNA331813, AF213467,
NM_013448_at
FIG. 6576: PRO86750
FIG. 6577: DNA304461, HSPC067, NM_014158_at
FIG. 6578: PRO71039
FIG. 6579: DNA331814, BC009642, NM_014164_at
FIG. 6580: PRO86751
FIG. 6581: DNA330374, ORMDL2, NM_014182_at
FIG. 6582: PRO23321
FIG. 6583: DNA88203, CD5, NM_014207_at
FIG. 6584: PRO2698
FIG. 6585A-B: DNA331815, AF135372,
NM_014232_at
FIG. 6586: DNA331816, BC003067, NM_014330_at
FIG. 6587: PRO12543
FIG. 6588: DNA331817, NP_055154.2,
NM_014339_at
FIG. 6589: PRO86240
FIG. 6590: DNA227233, NP_055157.1,
NM_014342_at
FIG. 6591: PRO37696
FIG. 6592: DNA227351, AF191020, NM_014367_at
FIG. 6593: PRO37814
FIG. 6594: DNA331088, NP_055252.2,
NM_014437_at
FIG. 6595: PRO80674
FIG. 6596: DNA330084, SIT, NM_014450_at
FIG. 6597: PRO9895
FIG. 6598: DNA324198, NP_055400.1,
NM_014585_at
FIG. 6599: PRO37675
FIG. 6600A-B: DNA151879, NP_055463.1,
NM_014648_at
FIG. 6601: PRO12743
FIG. 6602: DNA194805, NP_055500.1,
NM_014685_at
FIG. 6603: PRO24075
FIG. 6604A-B: DNA150467, AB018335,
NM_014698_at
FIG. 6605: PRO12272
FIG. 6606A-B: DNA194778, KIAA0152,
NM_014730_at
FIG. 6607: PRO24056
FIG. 6608A-B: DNA277809, KIAA0275,
NM_14767_at
FIG. 6609: PRO64556
FIG. 6610A-B: DNA227353, SEC24D,
NM_014822_at
FIG. 6611: PRO37816
FIG. 6612: DNA93507, NP_055694.1,
NM_014879_at
FIG. 6613: PRO4948
FIG. 6614A-B: DNA150954, KIAA0022,
NM_014880_at
FIG. 6615: PRO12558
FIG. 6616A-B: DNA227293, DNA227293,
NM_014883_at
FIG. 6617: PRO37756
FIG. 6618: DNA150805, FAM3C, NM_014888_at
FIG. 6619: PRO11583
FIG. 6620A-B: DNA194837, NP_055714.1,
NM_014899_at
FIG. 6621: PRO24100
FIG. 6622A-B: DNA304464, CHSY1,
NM_014918_at
FIG. 6623: PRO71042
FIG. 6624: DNA330103, MD-2, NM_015364_at
FIG. 6625: PRO19671
FIG. 6626: DNA150872, NP_56202.1,
NM_015387_at
FIG. 6627: PRO12814
FIG. 6628: DNA328590, BC001232, NM_015864_at
FIG. 6629: PRO84375
FIG. 6630: DNA196569, NP_056957.1,
NM_015873_at
FIG. 6631: PRO19859
FIG. 6632: DNA150865, LOC51596, NM_015921_at
FIG. 6633: PRO11587
FIG. 6634: DNA150832, NP_057019.2,
NM_015935_at
FIG. 6635: PRO12491
FIG. 6636: DNA331089, NP_057143.1,
NM_016059_at
FIG. 6637: PRO4984
FIG. 6638: DNA331818, AF151899, NM_016072_at
FIG. 6639: PRO793
FIG. 6640: DNA328663, AK001280, NM_016073_at
FIG. 6641: PRO36183
FIG. 6642: DNA331819, BC006807, NM_016077_at
FIG. 6643: PRO38080
FIG. 6644: DNA150661, LOC51030, NM_016078_at
FIG. 6645: PRO12398
FIG. 6646: DNA329292, AF085360, NM_016101_at
FIG. 6647: PRO84882
FIG. 6648: DNA329923, HSPC035, NM_016127_at
FIG. 6649: PRO85237
FIG. 6650: DNA304832, NP_057327.1,
NM_016243_at
FIG. 6651: PRO71239
FIG. 6652: DNA328831, AF126780, NM_016245_at
FIG. 6653: PRO233
FIG. 6654: DNA328513, AF151895, NM_016283_at
FIG. 6655: PRO37815
FIG. 6656: DNA304781, LOC51184, NM_016301_at
FIG. 6657: PRO71191
FIG. 6658: DNA331820, BC001144, NM_016306_at
FIG. 6659: PRO1080
FIG. 6660: DNA331821, AK023410, NM_016354_at
FIG. 6661: PRO86752
FIG. 6662: DNA330390, AF178985, NM_016546_at
FIG. 6663: PRO85599
FIG. 6664: DNA331822, AF318357, NM_016553_at
FIG. 6665: PRO86753
FIG. 6666: DNA227298, NP_057649.1,
NM_016565_at
FIG. 6667: PRO37761
FIG. 6668: DNA327869, NRN1, NM_016588_at
FIG. 6669: PRO1898
FIG. 6670: DNA331823, AK027682, NM_017424_at
FIG. 6671: PRO86754
FIG. 6672: DNA225694, FLJ20005, NM_017617_at
FIG. 6673: PRO36157
FIG. 6674: DNA326385, NP_060117.2,
NM_017647_at
FIG. 6675: PRO82778
FIG. 6676: DNA287206, FLJ20073, NM_017654_at
FIG. 6677: PRO69488
FIG. 6678: DNA227294, FLJ20303, NM_017755_at
FIG. 6679: PRO37757
FIG. 6680: DNA226646, NP_060352.1,
NM_017882_at
FIG. 6681: PRO37109
FIG. 6682: DNA331824, BC010907, NM_017906_at
FIG. 6683: PRO86755
FIG. 6684: DNA330537, HELLS, NM_018063_at
FIG. 6685: PRO81892
FIG. 6686: DNA328628, BC011983, NM_018072_at
FIG. 6687: PRO84406
FIG. 6688: DNA328841, BC003082, NM_018087_at
FIG. 6689: PRO84575

[symbol]

FIG. 6730: DNA327199, DJ971N18.2,
NM_021156_at
FIG. 6731: PRO83475
FIG. 6732: DNA227276, NP_005702.1,
NM_021618_at
FIG. 6733: PRO37739
FIG. 6734A-B: DNA331832, AF051850,
NM_021738_at
FIG. 6735: PRO86758
FIG. 6736: DNA331833, AF269133, NM_021798_at
FIG. 6737: PRO86759

[symbol]

HUMKG1BB_at
FIG. 6776: PRO86762
FIG. 6777A-B: DNA331842, BC004375,
AF261758_at
FIG. 6778: PRO38492
FIG. 6779: DNA331095, NP_005216.1, HUME2F_at
FIG. 6780: PRO86245
FIG. 6781: DNA331843, AF202723, AB014568_at
FIG. 6782: DNA159542, DNA159542,
HUMMAC30X_at
FIG. 6783: DNA331844, BC009267,
HUMLAMBBA_at
FIG. 6784: PRO82888
FIG. 6785: DNA331096, S75881, P_V84330_at
FIG. 6786: PRO86246
FIG. 6787: DNA287239, AF212242, AK024843_at
FIG. 6788: PRO38497
FIG. 6789: DNA154390, DNA154390,
HUMP13KIN_at
FIG. 6790: DNA151247, DNA151247, P_V43601_at
FIG. 6791: PRO11643
FIG. 6792: DNA329950, MGC5576, P_V43613_at
FIG. 6793: PRO11558
FIG. 6794: DNA161927, DNA161927, P_Z29229_at
FIG. 6795: DNA155316, DNA155316, P_A09058_at
FIG. 6796: DNA329026, AF230200, AK021966_at
FIG. 6797A-B: DNA228052, DNA228052,
AB006624_at
FIG. 6798: PRO38515
FIG. 6799: DNA161913, DNA161913,
HSM800208_at
FIG. 6800: DNA331845, AK027432,
HSM800284_at
FIG. 6801: PRO86763
FIG. 6802: DNA329430, SPPL2A, AX027882_at
FIG. 6803: PRO38524
FIG. 6804: DNA151422, DNA151422, P_X04312_at
FIG. 6805: PRO11792
FIG. 6806: DNA228066, NP_079431.1,
AK021910_at
FIG. 6807: PRO38529
FIG. 6808A-C: DNA330360, FYCO1, AK023397_at
FIG. 6809: PRO85576
FIG. 6810: DNA287185, DNA287185, P_V84564_at
FIG. 6811: PRO37492
FIG. 6812: DNA331846, AF272741,
HUMTCBYY_at
FIG. 6813: DNA331097, AK027322, AX041977_at
FIG. 6814: PRO86247
FIG. 6815: DNA151756, DNA151756, P_X84947_at
FIG. 6816: PRO12037
FIG. 6817: DNA151761, DNA151761, P_X84970_at
FIG. 6818: PRO12039
FIG. 6819: DNA331847, BC008330, AK026632_at
FIG. 6820: PRO38556
FIG. 6821: DNA326258, MGC2941, AK026537_at
FIG. 6822: PRO82665
FIG. 6823A-B: DNA287330, AB032991,
AB032991_at
FIG. 6824: DNA331848, 1510819.1, P_X99863_at
FIG. 6825: PRO86764
FIG. 6826: DNA331849, HSINSP4BP,
HSINSP4BP_at
FIG. 6827: PRO66285
FIG. 6828A-B: DNA331850, HSA237724,
HSA237724_at
FIG. 6829: DNA328049, 981676.1, HSM800856_at
FIG. 6830: PRO83963
FIG. 6831: DNA331851, AK027334, P_A51904_at
FIG. 6832: PRO23392
FIG. 6833: DNA193996, DNA193996, P_A40502_at
FIG. 6834: PRO23400
FIG. 6835: DNA194019, DNA194019, AK000004_at
FIG. 6836: PRO23421
FIG. 6837: DNA194063, DNA194063, P_V84608_at
FIG. 6838: PRO23460
FIG. 6839: DNA83046, NP_000565.1, P_X30170_at
FIG. 6840: PRO2569
FIG. 6841: DNA331852, 985629.1, P_Z59467_at
FIG. 6842: PRO86765
FIG. 6843: DNA195915, DNA195915, P_X85020_at
FIG. 6844: DNA331853, BC001305, AK027031_at
FIG. 6845: PRO23769
FIG. 6846A-B: DNA328720, NP_078800.2,
P_X35729_at
FIG. 6847: PRO84476
FIG. 6848: DNA194679, BAA05062.1,
HUMORFT_at
FIG. 6849: PRO23989
FIG. 6850: DNA331854, AF244129, AF244129_at
FIG. 6851: PRO86766
FIG. 6852: DNA194766, DJ434O14.3,
HS434O143_at
FIG. 6853: PRO24046
FIG. 6854: DNA331855, BLP1, P_Z98236_at
FIG. 6855: PRO85742
FIG. 6856: DNA330358, BC008904, AX011617_at
FIG. 6857: PRO85574
FIG. 6858: DNA330380, FLJ12436, AK022498_at
FIG. 6859: PRO85592
FIG. 6860: DNA328288, NP_073591.1,
AK022938_at
FIG. 6861: PRO69876
FIG. 6862: DNA196036, DNA196036,
AI471699_RC_at
FIG. 6863: DNA331098, AY052405, AX047348_at
FIG. 6864: PRO86248
FIG. 6865A-B: DNA331099, AB058685,
AX048187_at
FIG. 6866: DNA331100, BC021238, P_X84987_at
FIG. 6867: PRO86249
FIG. 6868: DNA331101, NP_114143.1, 250446.2_at
FIG. 6869: PRO86250
FIG. 6870: DNA331856, BC022522, 237658.8_at
FIG. 6871: PRO71209
FIG. 6872: DNA106360, DNA106360, 164869.1_at
FIG. 6873: DNA155526, DNA155526, 251178.1_at
FIG. 6874: DNA331102, NP_116052.1, 481267.1_at
FIG. 6875: PRO86251
FIG. 6876: DNA196289, DNA196289, 230230.2_at
FIG. 6877: DNA323696, BC015160, 428335.22_at
FIG. 6878: DNA326749, NP_116101.1,
DNA167237_at
FIG. 6879: PRO23238
FIG. 6880: DNA210391, DNA210391, P_X85039_at
FIG. 6881: PRO34886
FIG. 6882: DNA331103, NP_009125.1,
NM_007194_at
FIG. 6883: PRO34956
FIG. 6884: DNA331857, SIRPB2, NM_018556_at
FIG. 6885: PRO86767
FIG. 6886: DNA254406, NP_060854.1,
NM_018384_at
FIG. 6887: PRO49516
FIG. 6888A-B: DNA331858, ABCA7,
NM_019112_at
FIG. 6889: PRO86768
FIG. 6890: DNA255704, NP_057570.1,
NM_016486_at
FIG. 6891: PRO50764
FIG. 6892: DNA331859, AF267245, NM_016523_at
FIG. 6893: PRO86769
FIG. 6894: DNA254470, HSU11050, NM_002497_at
FIG. 6895: PRO49578
FIG. 6896A-B: DNA256461, HSAJ6266,
NM_007086_at
FIG. 6897: PRO51498
FIG. 6898: DNA330480, FLJ10808, NM_018227_at
FIG. 6899: PRO85677
FIG. 6900: DNA328669, NP_005882.1,
NM_005891_at
FIG. 6901: PRO84441
FIG. 6902: DNA254777, CORO1C, NM_014325_at
FIG. 6903: PRO49875
FIG. 6904A-B: DNA329991, TIMELESS,
NM_003920_at
FIG. 6905: PRO85284
FIG. 6906: DNA255161, IFRG28, NM_022147_at
FIG. 6907: PRO50241
FIG. 6908: DNA327812, IFI44, NM_006417_at
FIG. 6909: PRO83773
FIG. 6910: DNA304716, CDKN1A, NM_000389_at
FIG. 6911: PRO71142
FIG. 6912: DNA328431, HSCKSHS1,
NM_001826_at
FIG. 6913: PRO45093
FIG. 6914: DNA269926, HSCDC2R, NM_001786_at
FIG. 6915: PRO58324
FIG. 6916: DNA331104, NP_066280.1,
NM_021000_f_at
FIG. 6917: PRO86252
FIG. 6918A-B: DNA274893, NP_006282.1,
NM_006291_at
FIG. 6919: PRO62634
FIG. 6920: DNA271455, LOC51339, NM_016651_at
FIG. 6921: PRO59751
FIG. 6922: DNA329172, GFI1, NM_005263_at
FIG. 6923: PRO84796
FIG. 6924: DNA331860, BC010940, NM_004833_at
FIG. 6925: PRO86770
FIG. 6926: DNA329274, AF187064, NM_014380_at
FIG. 6927: PRO84870
FIG. 6928A-B: DNA328535, NP_009147.2,
NM_007216_at
FIG. 6929: PRO60044
FIG. 6930: DNA331861, MAP2K6, NM_002758_at
FIG. 6931: PRO86771
FIG. 6932: DNA331105, NP_009012.1,
NM_007081_at
FIG. 6933: PRO86253
FIG. 6934: DNA256257, LAMP3, NM_014398_at
FIG. 6935: PRO51301
FIG. 6936: DNA256033, NP_060164.1,
NM_017694_at
FIG. 6937: PRO51081
FIG. 6938A-B: DNA331106, NP_065107.1,
NM_020374_at
FIG. 6939: PRO86254
FIG. 6940A-B: DNA254789, LOC51696,
NM_016217_at
FIG. 6941: PRO49887
FIG. 6942A-B: DNA254376, AB023180,
NM_014963_at
FIG. 6943: PRO49486
FIG. 6944: DNA254129, ARMET, NM_006010_at
FIG. 6945: PRO49244
FIG. 6946: DNA331862, AX008892, NM_005484_at
FIG. 6947: PRO86772
FIG. 6948: DNA256407, HSA249248,
NM_014373_at
FIG. 6949: PRO51448
FIG. 6950A-C: DNA256495, HSU33841,
NM_000051_at
FIG. 6951: PRO51531
FIG. 6952: DNA330384, FLJ20647, NM_017918_at
FIG. 6953: PRO51129
FIG. 6954: DNA331863, AK000318, NM_017760_at
FIG. 6955: PRO86773
FIG. 6956: DNA256533, NP_006105.1,
NM_006114_at
FIG. 6957: PRO51565
FIG. 6958A-B: DNA287273, AF092563,
NM_006444_at
FIG. 6959: PRO69545
FIG. 6960: DNA256295, DNA256295,
NM_002319_at
FIG. 6961: PRO51339
FIG. 6962A-C: DNA331864, HSA303086,
NM_002266_at
FIG. 6963: DNA254350, AF002697, NM_004052_at
FIG. 6964: PRO49461
FIG. 6965: DNA255010, NP_061869.1,
NM_018996_at
FIG. 6966: PRO50099
FIG. 6967: DNA329900, HUMA1SBU,
NM_002914_at
FIG. 6968: PRO81549
FIG. 6969: DNA323838, CDKN2C, NM_001262_at
FIG. 6970: PRO59546
FIG. 6971: DNA271093, CNK, NM_004073_at
FIG. 6972: PRO59417
FIG. 6973: DNA331108, NP_005265.1,
NM_005274_at
FIG. 6974: PRO10780
FIG. 6975: DNA290234, RGS2, NM_002923_at
FIG. 6976: PRO70333
FIG. 6977: DNA275015, HSU31383, NM_004125_at
FIG. 6978: PRO62743
FIG. 6979: DNA256854, HSU76638, NM_000465_at
FIG. 6980: PRO51785
FIG. 6981: DNA331865, IRF7, NM_004031_at
FIG. 6982: PRO86774
FIG. 6983: DNA255271, EMR2, NM_013447_at
FIG. 6984: PRO50348
FIG. 6985: DNA331866, AB020970, NM_022154_at
FIG. 6986: DNA331867, AF058762, NM_003857_at
FIG. 6987A-B: DNA331109, NP_005155.1,
NM_005164_at
FIG. 6988: PRO50662
FIG. 6989: DNA331110, NP_057563.3,
NM_016479_at
FIG. 6990: PRO86256
FIG. 6991: DNA254276, HSPC154, NM_014177_at
FIG. 6992: PRO49387
FIG. 6993: DNA287241, LAP3, NM_015907_at
FIG. 6994: PRO69516
FIG. 6995A-B: DNA331111, NP_004229.1,
NM_004238_at
FIG. 6996: PRO86257
FIG. 6997: DNA255261, NP_060262.1,
NM_017792_at
FIG. 6998: PRO50338
FIG. 6999A-B: DNA331868, HSM802180,
NM_017631_at
FIG. 7000: DNA331869, NP_067024.1,
NM_021201_at
FIG. 7001: PRO50191
FIG. 7002A-B: DNA255846, NP_057424.1,
NM_016340_at
FIG. 7003: PRO50900
FIG. 7004: DNA331870, AK001274, NM_017613_at
FIG. 7005: PRO86776
FIG. 7006: DNA287221, LOC51191, NM_016323_at
FIG. 7007: PRO69500
FIG. 7008: DNA331871, BC017842, NM_004851_at
FIG. 7009: PRO86777
FIG. 7010: DNA260982, NP_060819.1,
NM_018349_at
FIG. 7011: PRO54728
FIG. 7012: DNA329033, NP_005375.1,
NM_005384_at
FIG. 7013: PRO84700
FIG. 7014: DNA271095, AF091433, NM_004702_at
FIG. 7015: PRO59418
FIG. 7016: DNA297387, NP_003494.1,
NM_003503_at
FIG. 7017: PRO58394
FIG. 7018: DNA331872, AF099644, NM_001255_at
FIG. 7019: PRO86778
FIG. 7020: DNA331873, PTPN7, NM_002832_at
FIG. 7021: PRO69609
FIG. 7022: DNA269750, NP_002919.1,
NM_002928_at
FIG. 7023: PRO58159
FIG. 7024: DNA268036, AB023416, NM_013258_at
FIG. 7025: PRO57311
FIG. 7026: DNA270522, NP_006013.1,
NM_006022_at
FIG. 7027: PRO58899
FIG. 7028: DNA330057, MT1G, NM_005950_at
FIG. 7029: PRO85337
FIG. 7030A-B: DNA331113, NP_005914.1,
NM_005923_at
FIG. 7031: PRO60244
FIG. 7032: DNA331874, BC002847, NM_016103_at
FIG. 7033: PRO84581
FIG. 7034: DNA269791, NP_001168.1,
NM_001177_at
FIG. 7035: PRO58197
FIG. 7036: DNA331114, AF291719, NM_007182_at
FIG. 7037: PRO86258
FIG. 7038: DNA329580, HSU78170, NM_005825_at
FIG. 7039: PRO85114
FIG. 7040: DNA281436, NP_003286.1,
NM_017627_at
FIG. 7041: PRO66275
FIG. 7042: DNA331875, CDC25B, NM_021874_at
FIG. 7043: PRO83123
FIG. 7044: DNA331876, BC005912, NM_002001_at
FIG. 7045: PRO2280
FIG. 7046: DNA256737, FLJ20406, NM_017806_at
FIG. 7047: PRO51671
FIG. 7048: DNA255432, NP_060283.1,
NM_017813_at
FIG. 7049: PRO50499
FIG. 7050A-B: DNA254262, NP_055197.1,
NM_014382_at
FIG. 7051: PRO49373
FIG. 7052: DNA331877, HUMHMGAB,
NM_000859_at
FIG. 7053: DNA328901, BC002748, NM_017866_at
FIG. 7054: PRO84622
FIG. 7055: DNA254416, NP_060915.1,
NM_018445_at
FIG. 7056: PRO49526
FIG. 7057: DNA331115, AF221521, NM_020524_at
FIG. 7058: PRO86259
FIG. 7059: DNA255326, AF055993, NM_003864_at
FIG. 7060: PRO50396
FIG. 7061A-C: DNA328498, AF285167,
NM_005502_at
FIG. 7062: PRO84320
FIG. 7063: DNA331878, AF246240, NM_018480_at
FIG. 7064: PRO86779
FIG. 7065: DNA331879, AK021999, NM_022765_at
FIG. 7066: PRO86780
FIG. 7067: DNA255135, AB016068, NM_005857_at
FIG. 7068: PRO50216
FIG. 7069: DNA331116, NP_060656.1,
NM_018186_at
FIG. 7070: PRO86260
FIG. 7071: DNA237817, HSU33286, NM_001316_at
FIG. 7072: PRO38923
FIG. 7073A-B: DNA329904, AF203032,
NM_021076_at
FIG. 7074: PRO85221
FIG. 7075: DNA329583, AD24, NM_022451_at
FIG. 7076: PRO85117
FIG. 7077: DNA331117, NP_065170.1,
NM_020437_at
FIG. 7078: PRO86261
FIG. 7079: DNA254710, FLJ20637, NM_017912_at
FIG. 7080: PRO49810
FIG. 7081: DNA331880, HSIFI56R, NM_001548_at
FIG. 7082: PRO59911
FIG. 7083A-B: DNA270323, HSU34605,
NM_012420_at
FIG. 7084: PRO58710
FIG. 7085: DNA287224, ISG15, NM_005101_at
FIG. 7086: PRO69503
FIG. 7087: DNA269922, HSISG20GN,
NM_002201_at
FIG. 7088: PRO58320
FIG. 7089: DNA331118, NP_201569.1,
NM_003672_at
FIG. 7090: PRO86262
FIG. 7091: DNA272655, HSCKSHS2,
NM_001827_at
FIG. 7092: PRO60781
FIG. 7093A-B: DNA329160, NP_002821.1,
NM_002830_at
FIG. 7094: PRO84789
FIG. 7095: DNA270415, GNA15, NM_002068_at
FIG. 7096: PRO58796
FIG. 7097: DNA331881, AK023223, NM_016131_at
FIG. 7098: PRO10928
FIG. 7099: DNA270059, NP_003920.1,
NM_003929_at
FIG. 7100: PRO58452
FIG. 7101: DNA273487, RAB33A, NM_004794_at
FIG. 7102: PRO61470
FIG. 7103: DNA326306, NP_066960.1,
NM_021137_at
FIG. 7104: PRO62566
FIG. 7105: DNA287378, AF244135, NM_018428_at
FIG. 7106: PRO69637
FIG. 7107: DNA327879, MDA5, NM_022168_at
FIG. 7108: PRO83818
FIG. 7109A-B: DNA327674, NP_002739.1,
NM_002748_at
FIG. 7110: PRO83661
FIG. 7111: DNA331882, AB030251, NM_013277_at
FIG. 7112: PRO86781
FIG. 7113: DNA254518, LOC51713, NM_016270_at
FIG. 7114: PRO49625
FIG. 7115: DNA256561, CRTAM, NM_019604_at
FIG. 7116: PRO51592
FIG. 7117: DNA331883, AF096290, NM_003645_at
FIG. 7118: PRO51139
FIG. 7119: DNA255215, AF207600, NM_018638_at
FIG. 7120: PRO50294
FIG. 7121A-B: DNA256807, FAM8A1,
NM_016255_at
FIG. 7122: PRO51738
FIG. 7123: DNA260974, TRIM22, NM_006074_at
FIG. 7124: PRO54720
FIG. 7125: DNA330443, PRO2037, NM_018616_at
FIG. 7126: PRO2037
FIG. 7127: DNA331119, NP_005433.2,
NM_005442_at
FIG. 7128: PRO50745
FIG. 7129: DNA254274, NP_073573.1,
NM_022736_at
FIG. 7130: PRO49385
FIG. 7131: DNA255088, HUMTK, NM_003258_at
FIG. 7132: PRO50174
FIG. 7133: DNA255113, FLJ22693, NM_022750_at
FIG. 7134: PRO50195
FIG. 7135: DNA331884, BC008870, NM_017606_at
FIG. 7136: PRO49604
FIG. 7137: DNA331885, MRPL35, NM_016622_at
FIG. 7138: PRO86782
FIG. 7139: DNA272245, NP_055301.1,
NM_0144861_f_at
FIG. 7140: PRO60507
FIG. 7141A-D: DNA331886, AF051160,
NM_003463_at
FIG. 7142: DNA330877, HKE2, NM_014260_at
FIG. 7143: PRO86040
FIG. 7144: DNA295327, IL21, NM_021803_at
FIG. 7145: PRO70773
FIG. 7146: DNA287178, HSU52513, NM_001549_at
FIG. 7147: PRO69467
FIG. 7148: DNA327661, HUMIFI16A,
NM_005531_at
FIG. 7149: PRO83652
FIG. 7150A-B: DNA329036, HSU63738,
NM_002460_at
FIG. 7151: PRO84703
FIG. 7152: DNA273523, NP_002154.1,
NM_002163_at
FIG. 7153: PRO61504
FIG. 7154: DNA331887, AF002822, NM_004701_at
FIG. 7155: PRO82442
FIG. 7156: DNA331888, AF022109, NM_001254_at
FIG. 7157: PRO60595
FIG. 7158: DNA273535, AB011421, NM_004226_at
FIG. 7159: PRO61515
FIG. 7160: DNA275012, NMI, NM_004688_at
FIG. 7161: PRO62740
FIG. 7162: DNA331120, NP_008976.1,
NM_007045_at
FIG. 7163: PRO86263
FIG. 7164: DNA331889, AF182076, NM_015710_at
FIG. 7165: PRO84173
FIG. 7166: DNA331890, AF095287,
NM_004219_f_at
FIG. 7167: PRO81319
FIG. 7168: DNA331121, AF175306, NM_014288_at
FIG. 7169: PRO86264
FIG. 7170: DNA327858, AF120334, NM_012341_at
FIG. 7171: PRO83800
FIG. 7172: DNA331122, NR_005728.2,
NM_005737_at
FIG. 7173: PRO86265
FIG. 7174: DNA289528, ARL3, NM_004311_at
FIG. 7175: PRO70286
FIG. 7176: DNA270526, HUMLYGDI,
NM_001175_at
FIG. 7177: PRO58903
FIG. 7178: DNA271931, NP_005745.1,
NM_005754_at
FIG. 7179: PRO60207
FIG. 7180: DNA329123, RANBP1, NM_002882_at
FIG. 7181: PRO84765
FIG. 7182A-B: DNA331891, HSM800983,
NM_014922_at
FIG. 7183: DNA331892, BC019255, NM_006452_at
FIG. 7184: PRO84240
FIG. 7185: DNA329587, AF192466, NM_012124_at
FIG. 7186: PRO85121
FIG. 7187A-B: DNA329248, A2B002359,
AB002359_at
FIG. 7188: DNA254668, AB002437, AB002437_at
FIG. 7189: DNA256233, DNA256233,
AB017268_f_at
FIG. 7190: PRO51278
FIG. 7191A-B: DNA255448, BAA92554.1,
AB037737_at
FIG. 7192: PRO50515
FIG. 7193A-B: DNA255619, AF054589,
AF054589_at
FIG. 7194: PRO50682
FIG. 7195: DNA331123, AF062649, AF062649_f_at
FIG. 7196: PRO86266
FIG. 7197A-B: DNA331893, AB058697,
AK001581_at
FIG. 7198: DNA331894, HSM802273, AB032963_at
FIG. 7199: DNA331895, HUMTLEIV, AB033087_at
FIG. 7200: PRO86785
FIG. 7201A-B: DNA329039, DORFIN,
AK027070_at
FIG. 7202: PRO84706
FIG. 7203: DNA255040, CAB55998.1,
HSM801103_at
FIG. 7204: PRO50128
FIG. 7205: DNA328509, NP_006739.1,
HSU44403_at
FIG. 7206: PRO57996
FIG. 7207: DNA254338, HUMPLT, HUMPLT_at
FIG. 7208: PRO49449
FIG. 7209: DNA331896, AF067008, NM_001363_at
FIG. 7210: PRO49881
FIG. 7211: DNA331897, BC008843, AB007915_at
FIG. 7212: PRO86786
FIG. 7213: DNA331124, NP_079430.1,
AB018353_at
FIG. 7214: PRO86267
FIG. 7215A-B: DNA330736, AB033044,
AB033044_at
FIG. 7216A-B: DNA331125, AB037815,
AB037815_at
FIG. 7217A-B: DNA331898, AF058925,
AF058925_at
FIG. 7218: PRO86787
FIG. 7219: DNA331126, AF078867, AF078866_at
FIG. 7220: PRO86269
FIG. 7221: DNA254836, BAA91233.1,
AK000529_at
FIG. 7222: PRO49931
FIG. 7223: DNA88277, NP_006721.1, AK027197_at
FIG. 7224: PRO2724
FIG. 7225: DNA331899, 1399286.1,
AW290940_RC_at
FIG. 7226: PRO86788
FIG. 7227: DNA256872, HSM801990,
HSM801990_at
FIG. 7228A-B: DNA254192, HUMKIAAK,
HUMKIAAK_at
FIG. 7229: DNA331900, BIN2, NM_016293_at
FIG. 7230: PRO86789
FIG. 7231A-B: DNA256731, BAA83028.1,
AB028999_at
FIG. 7232: PRO51665
FIG. 7233: DNA331901, HSM801036, AB029015_at
FIG. 7234A-B: DNA331127, BAA86477.1,
AB032989_at
FIG. 7235: PRO86270
FIG. 7236A-B: DNA254672, BAA92652.1,
AB037835_at
FIG. 7237: PRO49773
FIG. 7238A-C: DNA331128, NP_065892.1,
AB040884_at
FIG. 7239: PRO84841
FIG. 7240: DNA269976, AAC14260.1,
AF039023_at
FIG. 7241: PRO58372
FIG. 7242: DNA331129, HSA227869,
HSA227869_r_at
FIG. 7243: DNA256422, HSA227900,
HSA227900_at
FIG. 7244: DNA331902, BC014522,
HSSOM172M_at
FIG. 7245: PRO86790
FIG. 7246: DNA329040, BC001356, HSU72882_at
FIG. 7247: PRO84707
FIG. 7248: DNA331130, AAK50430.1,
HUMTI227HC_at
FIG. 7249: PRO86272
FIG. 7250A-B: DNA331131, HSA223948,
AY013288_at
FIG. 7251: DNA326056, NP_072088.1,
AY007810_at
FIG. 7252: PRO82491
FIG. 7253: DNA329041, HSM800399, AF132199_at
FIG. 7254: DNA255780, AK022209, AK022209_at
FIG. 7255: PRO50835
FIG. 7256: DNA254922, AK022604, AK022604_at
FIG. 7257: PRO50012
FIG. 7258: DNA330432, FLJ23235, AK026888_at
FIG. 7259: PRO85636
FIG. 7260A-B: DNA256299, AB051489,
AB051489_at
FIG. 7261: DNA331903, BC015380, HSM801707_at
FIG. 7262: DNA255626, HSM802849,
HSM802849_at
FIG. 7263: PRO50690
FIG. 7264: DNA331132, NP_115524.1,
HSM801796_at
FIG. 7265: PRO86273
FIG. 7266: DNA255964, NP_079113.1,
AK025125_at
FIG. 7267: PRO51015
FIG. 7268: DNA255465, AK024313, AK024313_at
FIG. 7269: PRO50532
FIG. 7270: DNA329597, AK022178, AK022178_at
FIG. 7271: PRO85129
FIG. 7272: DNA254228, NP_079236.1,
AK021791_at
FIG. 7273: PRO49340
FIG. 7274: DNA331904, AK023431, AF298880_at
FIG. 7275: PRO86791
FIG. 7276: DNA329078, AF214006, HSM801679_at
FIG. 7277: PRO23253
FIG. 7278: DNA256784, FLJ22104, AK025757_at
FIG. 7279: PRO51716
FIG. 7280: DNA331905, AK001823,
HSM801648_at
FIG. 7281: PRO86792
FIG. 7282: DNA329044, NP_064562.1,
AK025265_at
FIG. 7283: PRO84709
FIG. 7284: DNA331906, HSA227916,
NM_001530_at
FIG. 7285: DNA330023, GADD45A, NM_001924_at
FIG. 7286: PRO85308
FIG. 7287A-B: DNA272191, RSN, NM_002956_at
FIG. 7288: PRO60456
FIG. 7289: DNA328418, HUMG0S24A,
NM_003407_at
FIG. 7290: PRO84261
FIG. 7291: DNA331133, HSU63830, NM_004180_at
FIG. 7292: PRO86274
FIG. 7293: DNA271310, DUSP8, NM_004420_at
FIG. 7294: PRO59617
FIG. 7295: DNA331907, AKAP7, NM_004842_at
FIG. 7296: PRO63228
FIG. 7297: DNA287203, NP_006182.1,
NM_006191_at
FIG. 7298: PRO69487
FIG. 7299: DNA274783, HSU26424, NM_006281_at
FIG. 7300: PRO62549
FIG. 7301A-B: DNA255281, NP_006380.1,
NM_006389_at
FIG. 7302: PRO50357
FIG. 7303: DNA328712, NP_006501.1,
NM_006510_at
FIG. 7304: PRO84469
FIG. 7305: DNA331908, AF161440, NM_012111_at
FIG. 7306: DNA330065, STK18, NM_014264_at
FIG. 7307: PRO85345
FIG. 7308: DNA152148, DNA152148,
HSP1CDC21_at
FIG. 7309: PRO10290
FIG. 7310: DNA329925, HSBP1, NM_001537_at
FIG. 7311: PRO85239
FIG. 7312: DNA331909, HSCFANT, NM_002964_at
FIG. 7313: PRO86795
FIG. 7314: DNA329139, NP_003893.2,
NM_003902_at
FIG. 7315: PRO84774
FIG. 7316: DNA331910, HSSEC232, NM_006363_at
FIG. 7317: PRO86796
FIG. 7318: DNA329047, BATF, NM_006399_at
FIG. 7319: PRO58425
FIG. 7320: DNA274167, AF026166, NM_006431_at
FIG. 7321: PRO62097
FIG. 7322: DNA254572, NP_006576.1,
NM_006585_at
FIG. 7323: PRO49675
FIG. 7324A-B: DNA331911, AB003334,
NM_006644_at
FIG. 7325: PRO86797
FIG. 7326: DNA331912, BC009405, NM_013411_at
FIG. 7327: PRO86798
FIG. 7328: DNA255289, MELK, NM_014791_at
FIG. 7329: PRO50363
FIG. 7330A-B: DNA331913, BAB21784.1,
NM_015383_at
FIG. 7331: PRO86799
FIG. 7332: DNA329148, LOC51042, NM_015871_at
FIG. 7333: PRO84782
FIG. 7334: DNA326221, AF125098, NM_016095_at
FIG. 7335: PRO82634
FIG. 7336: DNA331914, BC009398,
HUMP1CDC47_at
FIG. 7337: PRO86800
FIG. 7338A-B: DNA328312, HUMAREB6,
HUMAREB6_at
FIG. 7339: PRO84180
FIG. 7340: DNA325941, HSPCA, HSHSP90R_at
FIG. 7341: PRO82388
FIG. 7342: DNA328483, VIT1, NM_000179_at
FIG. 7343: PRO84309
FIG. 7344: DNA271847, HUMDNAJHOM,
NM_001539_at
FIG. 7345: PRO60127
FIG. 7346: DNA331915, BC001786, NM_002014_at
FIG. 7347: PRO59262
FIG. 7348: DNA331916, HUMMIF, NM_002415_at
FIG. 7349: DNA331917, PHF1, NM_002636_at
FIG. 7350: PRO86802
FIG. 7351: DNA329604, SRP54, NM_003136_at
FIG. 7352: PRO85134
FIG. 7353A-B: DNA331134, NP_003381.1,
NM_003390_at
FIG. 7354: PRO86275
FIG. 7355A-B: DNA290265, ZNF91,
NM_003430_f_at
FIG. 7356: PRO70395
FIG. 7357A-C: DNA331918, AF009425,
NM_004338_at
FIG. 7358: PRO86803
FIG. 7359: DNA254582, NP_004626.1,
NM_004635_at
FIG. 7360: PRO49685
FIG. 7361A-B: DNA275334, NP_112162.1,
NM_004749_at
FIG. 7362: PRO63009
FIG. 7363: DNA254157, HSU13045, NM_005254_at
FIG. 7364: PRO49271
FIG. 7365A-B: DNA124122, RBL2, NM_005611_at
FIG. 7366: PRO6323
FIG. 7367: DNA330776, TOB1, NM_005749_at
FIG. 7368: PRO58014
FIG. 7369: DNA326980, AF140598, NM_014248_at
FIG. 7370: PRO83289
FIG. 7371: DNA271608, HUMRSC419,
NM_014670_at
FIG. 7372: PRO59895
FIG. 7373: DNA272928, HUMORFKG1F,
NM_014764_at
FIG. 7374: PRO61012
FIG. 7375: DNA290235, NP_057121.1,
NM_016037_at
FIG. 7376: PRO70335
FIG. 7377: DNA331135, HUMKG1DD,
HUMKG1DD_at
FIG. 7378A-B: DNA330119, AF226044,
HUMKIAAQ_at
FIG. 7379: PRO85381
FIG. 7380: DNA331137, HS24P52,
HUMHSP70H_at
FIG. 7381: PRO86278
FIG. 7382A-B: DNA269805, NP_001263.1,
NM_001272_at
FIG. 7383: PRO58209
FIG. 7384: DNA270689, HSGATA3R,
NM_002051_at
FIG. 7385: PRO59053
FIG. 7386: DNA331919, HUMCFA, NM_002965_at
FIG. 7387: PRO80648
FIG. 7388A-B: DNA304800, NP_004146.1,
NM_004155_at
FIG. 7389: PRO69458
FIG. 7390: DNA273418, AAG01157.1,
NM_004301_at
FIG. 7391: PRO61417
FIG. 7392: DNA330066, MLLT3, NM_004529_at
FIG. 7393: PRO85346
FIG. 7394: DNA270733, S46622, NM_005605_at
FIG. 7395: PRO59094
FIG. 7396: DNA331138, NP_005997.2,
NM_006006_at
FIG. 7397: PRO86279
FIG. 7398: DNA331139, NP_006865.1,
NM_006874_at
FIG. 7399: PRO81172
FIG. 7400: DNA331920, AF090950, NM_015675_at
FIG. 7401: PRO84384
FIG. 7402: DNA329050, MRPS17, NM_015969_at
FIG. 7403: PRO84712
FIG. 7404A-B: DNA329122, GS3955,
NM_021643_at
FIG. 7405: PRO84764
FIG. 7406: DNA331921, 244055.1, AF320911_at
FIG. 7407: PRO86804
FIG. 7408: DNA331922, AK026275, AK026275_at
FIG. 7409: PRO86805
FIG. 7410A-B: DNA254516, AF288399,
AF288399_at
FIG. 7411: PRO49623
FIG. 7412: DNA328313, NP_115579.1,
AK025076_at
FIG. 7413: PRO84181
FIG. 7414: DNA327865, NP_079105.1,
AK026315_at
FIG. 7415: PRO83806
FIG. 7416: DNA294813, NP_444283.1, P_T67134_at
FIG. 7417: PRO70763
FIG. 7418A-B: DNA254706, AB046851,
AB046851_at
FIG. 7419: DNA329052, NP_078801.1,
AK026237_at
FIG. 7420: PRO84714
FIG. 7421: DNA256890, BC008988, P_Z00467_at
FIG. 7422: PRO51824
FIG. 7423: DNA256291, FLJ21032, AK024685_f_at
FIG. 7424: PRO51335
FIG. 7425: DNA331923, HSUCP2X12, P_C69111_at
FIG. 7426: DNA213665, DNA213665, P_X30166_at
FIG. 7427: PRO35126
FIG. 7428: DNA331140, 332752.10, AK023798_at
FIG. 7429: PRO86280
FIG. 7430A-B: DNA331141, BAB13420.1,
AB046814_at
FIG. 7431: PRO86281
FIG. 7432: DNA331924, BC004932, AK024551_at
FIG. 7433: PRO21434
FIG. 7434A-B: DNA256267, AB046838,
AB046838_at
FIG. 7435: DNA327954, BAL, P_D00629_at
FIG. 7436: PRO83879
FIG. 7437: DNA255798, FLJ12377, AK022439_at
FIG. 7438: PRO50853
FIG. 7439: DNA330389, FLJ12888, AK022950_at
FIG. 7440: PRO85598
FIG. 7441: DNA330086, FLJ12973, AK023035_at
FIG. 7442: PRO85360
FIG. 7443: DNA331142, NP_116325.1, P_Z98137_at
FIG. 7444: PRO51781
FIG. 7445: DNA329384, BC008502, P_Z33372_at
FIG. 7446: PRO84960
FIG. 7447A-B: DNA331143, NP_149075.2,
AK022613_at
FIG. 7448: PRO86282
FIG. 7449: DNA331925, 424693.10, AK022231_at
FIG. 7450: PRO86806
FIG. 7451: DNA331144, NP_078834.1,
AK023982_at
FIG. 7452: PRO86283
FIG. 7453A-B: DNA331926, BAB13449.1,
AB046843_at
FIG. 7454: PRO51258
FIG. 7455: DNA255197, DNA255197, P_Z50392_at
FIG. 7456: PRO50276
FIG. 7457: DNA328010, NP_149016.1,
HSM801092_at
FIG. 7458: PRO83928
FIG. 7459: DNA262805, DNA262805,
HSM800425_at
FIG. 7460: DNA331146, 1400830.1,
HUMJNLTRA_at
FIG. 7461: PRO86284
FIG. 7462: DNA328317, cig5, AF026941_at
FIG. 7463: PRO69493
FIG. 7464: DNA331147, NP_079104.1,
AF131768_at
FIG. 7465: PRO86285
FIG. 7466: DNA255770, DNA255770, AK022106_at
FIG. 7467A-C: DNA254412, EVI5, AF008915_at
FIG. 7468: PRO49522
FIG. 7469: DNA331148, 978273.10, AK023244_at
FIG. 7470: PRO86286
FIG. 7471: DNA330532, AK026279, AK026279_at
FIG. 7472: PRO85719
FIG. 7473: DNA330388, FLJ23468, AK027121_at
FIG. 7474: PRO85597
FIG. 7475: DNA331927, AK026969, AK026969_at
FIG. 7476: PRO86807
FIG. 7477: DNA330447, FLJ22757, AK026410_at
FIG. 7478: PRO85648
FIG. 7479: DNA324984, FLJ12298, AK022360_at
FIG. 7480: PRO81578
FIG. 7481: DNA331149, 7697327.1, HSM802839_at
FIG. 7482: PRO86287
FIG. 7483A-B: DNA256267, DNA256267,
AK023113_at
FIG. 7484: PRO51311
FIG. 7485: DNA331150, BC017725, 1387341.2_at
FIG. 7486: PRO86288
FIG. 7487: DNA257606, DNA257606, 428093.1_at
FIG. 7488: DNA258375, AF283301, 413231.5_at
FIG. 7489: PRO52516
FIG. 7490: DNA331928, AK027419, 154551.10_at
FIG. 7491: PRO86808
FIG. 7492: DNA328319, BC019562, 411364.2_at
FIG. 7493: DNA290812, DNA290812,
220495.3_CON_at
FIG. 7494: PRO70559
FIG. 7495: DNA304799, BC022410, 337588.1_at
FIG. 7496: PRO52633
FIG. 7497: DNA257403, DNA257403, 012814.1_at
FIG. 7498: DNA304820, NP_115940.1, 317557.1_at
FIG. 7499: PRO47351
FIG. 7500: DNA331929, BC019246,
441855.8_CON_at
FIG. 7501: PRO83338
FIG. 7502: DNA260581, DNA260581, 127987.6_at
FIG. 7503: PRO54507
FIG. 7504: DNA257576, DNA257576, 334945.2_at
FIG. 7505: DNA304819, BC004398, 202113.2_at
FIG. 7506: DNA304794, FBXO30, 222128.1_at
FIG. 7507: PRO71206
FIG. 7508: DNA259323, DNA259323, 022997.1_at
FIG. 7509: PRO53256
FIG. 7510: DNA304796, MED8, 237428.13_at
FIG. 7511: PRO71208
FIG. 7512: DNA259615, DNA259615, 1000203.1_at
FIG. 7513: DNA304805, AK027628, 475113.7_at
FIG. 7514: PRO69531
FIG. 7515: DNA304793, GBP4, 206425.2_at
FIG. 7516: PRO71205
FIG. 7517: DNA331151, 018033.1,
018033.1_CON_at
FIG. 7518: PRO86289
FIG. 7519: DNA304068, AK057631, 1091656.1_at
FIG. 7520: PRO71035
FIG. 7521: DNA257714, EPSTI1, 337352.17_at
FIG. 7522: PRO52268
FIG. 7523: DNA304798, NP_443097.1, 246119.7_at
FIG. 7524: PRO71210
FIG. 7525: DNA258721, DNA258721, 197627.1_at
FIG. 7526A-B: DNA257461, NP_113607.1,
086533.1_at
FIG. 7527: PRO52040
FIG. 7528: DNA331152, 1042156.3, 1042156.3_at
FIG. 7529: PRO86290
FIG. 7530: DNA331153, 004052.1, 004052.1_at
FIG. 7531: PRO86291
FIG. 7532: DNA331930, AK054582, 978231.1_at
FIG. 7533: PRO86809
FIG. 7534: DNA259587, DNA259587, 220866.1_at
FIG. 7535: DNA106195, DNA106195, 359193.13_at
FIG. 7536: DNA331154, 212376.1, 212376.1_at
FIG. 7537: PRO86292
FIG. 7538: DNA331155, 112652.1, 112652.1_at
FIG. 7539: PRO86293
FIG. 7540: DNA304806, BC019022, 983343.1_at
FIG. 7541: PRO71215
FIG. 7542: DNA262708, DNA262708,
118516.1_RC_at
FIG. 7543: DNA259475, DNA259475, 239162.1_at
FIG. 7544: DNA269148, DNA269148, 411192.2_at
FIG. 7545: DNA304817, BC015532, 211436.3_at
FIG. 7546: PRO71224
FIG. 7547: DNA260313, DNA260313, 1098929.1_at
FIG. 7548: PRO54242
FIG. 7549A-B: DNA328325, NP_061142.1,
445188.4_at
FIG. 7550: PRO84190
FIG. 7551A-B: DNA304800, SERPINB9,
354740.1_at
FIG. 7552: PRO69458
FIG. 7553: DNA331156, 118180.1, 118180.1_at
FIG. 7554: PRO86294
FIG. 7555: DNA287659, AK027790, 406833.1_at
FIG. 7556: PRO69903
FIG. 7557: DNA331931, 092555.3, 092555.4_at
FIG. 7558: PRO86810
FIG. 7559: DNA331157, NP_439896.1, 022541.5_at
FIG. 7560: PRO86295
FIG. 7561: DNA260573, DNA260573, 899597.1_at
FIG. 7562: PRO54499
FIG. 7563: DNA260157, DNA260157, 236833.1_at
FIG. 7564: PRO54086
FIG. 7565: DNA174145, DNA174145, 100083.2_at
FIG. 7566: PRO35770
FIG. 7567: DNA260167, DNA260167, 264556.1_at
FIG. 7568A-B: DNA331932, 239260.1, 239260.1_at
FIG. 7569: PRO86811
FIG. 7570: DNA260031, DNA260031, 161526.1_at
FIG. 7571: DNA258907, DNA258907, 347940.2_at
FIG. 7572: PRO52840
FIG. 7573: DNA257455, DNA257455, 977723.3_at
FIG. 7574: PRO52035
FIG. 7575: DNA304807, BC014978, 005415.2_at
FIG. 7576: PRO71216
FIG. 7577: DNA258864, DNA258864, 331965.1_at
FIG. 7578: DNA304811, 428051.2, 428051.2_at
FIG. 7579: PRO71220
FIG. 7580: DNA257389, FLJ14906, 987098.1_at
FIG. 7581: PRO51974
FIG. 7582: DNA331158, 130352.1, 130352.1_at
FIG. 7583: PRO86296
FIG. 7584: DNA258951, DNA258951, 222361.1_at
FIG. 7585: DNA331159, NP_077291.1,
411426.29_at
FIG. 7586: PRO86297
FIG. 7587: DNA257784, DNA257784, 481853.1_at
FIG. 7588: DNA331933, AF272148, 074299.1_at
FIG. 7589: PRO86812

### BRIEF DESCRIPTION OF THE DRAWINGS

In the list of figures for the present application, specific cDNA sequences which are differentially expressed in differentiated macrophages as compared to normal undifferentiated monocytes are individually identified with a specific alphanumerical designation. These cDNA sequences are differentially expressed in monocytes that are specifically treated as described in Example 1 below. If start and/or stop codons have been identified in a cDNA sequence shown in the attached figures, they are shown in bold and underlined font, and the encoded polypeptide is shown in the next consecutive figure.

The FIGS. 1-7589 show the nucleic acids of the invention and their encoded PRO polypeptides. Also included, for convenience is a List of Figures, which gives the figure number and the corresponding DNA or PRO number.

### DETAILED DESCRIPTION OF THE PREFERRED EMBODIMENTS

I. Definitions

The terms “PRO polypeptide” and “PRO” as used herein and when immediately followed by a numerical designation refer to various polypeptides, wherein the complete designation (i.e., PRO/number) refers to specific polypeptide sequences as described herein. The terms “PRO/number polypeptide” and “PRO/number” wherein the term “number” is provided as an actual numerical designation as used herein encompass native sequence polypeptides and polypeptide variants (which are further defined herein). The PRO polypeptides described herein may be isolated from a variety of sources, such as from human tissue types or from another source, or prepared by recombinant or synthetic methods. The term “PRO polypeptide” refers to each individual PRO/number polypeptide disclosed herein. All disclosures in this specification which refer to the “PRO polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “PRO polypeptide” also includes variants of the PRO/number polypeptides disclosed herein.

A “native sequence PRO polypeptide” comprises a polypeptide having the same amino acid sequence as the corresponding PRO polypeptide derived from nature. Such native sequence PRO polypeptides can be isolated from nature or can be produced by recombinant or synthetic means. The term “native sequence PRO polypeptide” specifically encompasses naturally-occurring truncated or secreted forms of the specific PRO polypeptide (e.g., an extracellular domain sequence), naturally-occurring variant forms (e.g., alternatively spliced forms) and naturally-occurring allelic variants of the polypeptide. In various embodiments of the invention, the native sequence PRO polypeptides disclosed herein are mature or full-length native sequence polypeptides comprising the full-length amino acids sequences shown in the accompanying figures. Start and stop codons are shown in bold font and underlined in the figures. However, while the PRO polypeptide disclosed in the accompanying figures are shown to begin with methionine residues designated herein as amino acid position 1 in the figures, it is conceivable and possible that other methionine residues located either upstream or downstream from the amino acid position 1 in the figures may be employed as the starting amino acid residue for the PRO polypeptides.

The PRO polypeptide “extracellular domain” or “ECD” refers to a form of the PRO polypeptide which is essentially free of the transmembrane and cytoplasmic domains. Ordinarily, a PRO polypeptide ECD will have less than 1% of such transmembrane and/or cytoplasmic domains and preferably, will have less than 0.5% of such domains. It will be understood that any transmembrane domains identified for the PRO polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a PRO polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal peptides” of the various PRO polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO polypeptide variant” means an active PRO polypeptide as defined above or below having at least about 80% amino acid sequence identity with a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Such PRO polypeptide variants include, for instance, PRO polypeptides wherein one or more amino acid residues are added, or deleted, at the N- or C-terminus of the full-length native amino acid sequence. Ordinarily, a PRO polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other specifically defined fragment of a full-length PRO polypeptide sequence as disclosed herein. Ordinarily, PRO variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

“Percent (%) amino acid sequence identity” with respect to the PRO polypeptide sequences identified herein is defined as the percentage of amino acid residues in a candidate sequence that are identical with the amino acid residues in the specific PRO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“PRO variant polynucleotide” or “PRO variant nucleic acid sequence” means a nucleic acid molecule which encodes an active PRO polypeptide as defined below and which has at least about 80% nucleic acid sequence identity with a nucleotide acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Ordinarily, a PRO variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with a nucleic acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal sequence, as disclosed herein or any other fragment of a full-length PRO polypeptide sequence as disclosed herein. Variants do not encompass the native nucleotide sequence.

Ordinarily, PRO variant polynucleotides are at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 450 nucleotides in length, alternatively at least about 600 nucleotides in length, alternatively at least about 900 nucleotides in length, or more.

“Percent (%) nucleic acid sequence identity” with respect to PRO-encoding nucleic acid sequences identified herein is defined as the percentage of nucleotides in a candidate sequence that are identical with the nucleotides in the PRO nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides.

Unless specifically stated otherwise, all % nucleic acid sequence identity values used herein are obtained as described in the immediately preceding paragraph using the ALIGN-2 computer program. However, % nucleic acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, PRO variant polynucleotides are nucleic acid molecules that encode an active PRO polypeptide and which are capable of hybridizing, preferably under stringent hybridization and wash conditions, to nucleotide sequences encoding a full-length PRO polypeptide as disclosed herein. PRO variant polypeptides may be those that are encoded by a PRO variant polynucleotide.

“Isolated,” when used to describe the various polypeptides disclosed herein, means polypeptide that has been identified and separated and/or recovered from a component of its natural environment Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO polypeptide natural environment will not be present Ordinarily, however, isolated polypeptide will be prepared by at least one purification step.

An “isolated” PRO polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PRO monoclonal antibodies (including agonist, antagonist, and neutralizing antibodies), anti-PRO antibody compositions with polyepitopic specificity, single chain anti-PRO antibodies, and fragments of anti-PRO antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50M sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of a PRO polypeptide which retain a biological and/or an immunological activity of native or naturally-occurring PRO, wherein “biological” activity refers to a biological function (either inhibitory or stimulatory) caused by a native or naturally-occurring PRO other than the ability to induce the production of an antibody against an antigenic epitope possessed by a native or naturally-occurring PRO and an “immunological” activity refers to the ability to induce the production of an antibody against an antigenic epitope possessed by a native or naturally-occurring PRO.

The term “antagonist” is used in the broadest sense, and includes any molecule that partially or fully blocks, inhibits, or neutralizes a biological activity of a native PRO polypeptide disclosed herein. In a similar manner, the term “agonist” is used in the broadest sense and includes any molecule that mimics a biological activity of a native PRO polypeptide disclosed herein. Suitable agonist or antagonist molecules specifically include agonist or antagonist antibodies or antibody fragments, fragments or amino acid sequence variants of native PRO polypeptides, peptides, antisense oligonucleotides, small organic molecules, etc. Methods for identifying agonists or antagonists of a PRO polypeptide may comprise contacting a PRO polypeptide with a candidate agonist or antagonist molecule and measuring a detectable change in one or more biological activities normally associated with the PRO polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and B, herpes, etc., bacterial infections, fungal infections, protozoal infections and parasitic infections.

The term “effective amount” is a concentration or amount of a PRO polypeptide and/or agonist/antagonist which results in achieving a particular stated purpose. An “effective amount” of a PRO polypeptide or agonist or antagonist thereof may be determined empirically. Furthermore, a “therapeutically effective amount” is a concentration or amount of a PRO polypeptide and/or agonist/antagonist which is effective for achieving a stated therapeutic effect. This amount may also be determined empirically.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhône-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), melphalan and other related nitrogen mustards. Also included in this definition are hormonal agents that act to regulate or inhibit hormone action on tumors such as tamoxifen and onapristone.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ, colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; a tumor necrosis factor such as TNF-α or TNF-β; and other polypeptide factors including LIP and kit ligand (KL). As used herein, the term cytokine includes proteins from natural sources or from recombinant cell culture and biologically active equivalents of the native sequence cytokines.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

As used herein, the term “inflammatory cells” designates cells that enhance the inflammatory response such as mononuclear cells, eosinophils, macrophages, and polymorphonuclear neutrophils (PMN).

[symbol]

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleatides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

II. Compositions and Methods of the Invention

A. Full-Length PRO Polypeptides

The present invention provides newly identified and isolated nucleotide sequences encoding polypeptides referred to in the present application as PRO polypeptides. In particular, cDNAs encoding various PRO polypeptides have been identified and isolated, as disclosed in further detail in the Examples below. However, for sake of simplicity, in the present specification the protein encoded by the full length native nucleic acid molecules disclosed herein as well as all further native homologues and variants included in the foregoing definition of PRO, will be referred to as “PRO/number”, regardless of their origin or mode of preparation.

As disclosed in the Examples below, various cDNA clones have been disclosed. The predicted amino acid sequence can be determined from the nucleotide sequence using routine skill. For the PRO polypeptides and encoding nucleic acids described herein, Applicants have identified what is believed to be the reading frame best identifiable with the sequence information available at the time.

B. PRO Polypeptide Variants

In addition to the full-length native sequence PRO polypeptides described herein, it is contemplated that PRO variants can be prepared. PRO variants can be prepared by introducing appropriate nucleotide changes into the PRO DNA, and/or by synthesis of the desired PRO polypeptide. Those skilled in the art will appreciate that amino acid changes may alter post-translational processes of the PRO, such as changing the number or position of glycosylation sites or altering the membrane anchoring characteristics.

Variations in the native full-length sequence PRO or in various domains of the PRO described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO that results in a change in the amino acid sequence of the PRO as compared with the native sequence PRO. Optionally, the variation is by substitution of at least one amino acid with any other amino acid in one or more of the domains of the PRO. Guidance in determining which amino acid residue may be inserted, substituted or deleted without adversely affecting the desired activity may be found by comparing the sequence of the PRO with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO polypeptide.

PRO fragments may be prepared by any of a number of conventional techniques. Desired peptide fragments may be chemically synthesized. An alternative approach involves generating PRO fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO polypeptide fragments share at least one biological and/or immunological activity with the native PRO polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | |
| | norleucine | leu |
| Leu (L) | norleucine; ile; val; | |
| | met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | |
| | ala; norleucine | leu |

Substantial modifications in function or immunological identity of the PRO polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res. 13:4331 (1986); Zoller et al., Nucl. Acids Res., 10:6487 (1987)], cassette mutagenesis [Wells et al., Gene. 34:315 (1985)], restriction selection mutagenesis [Wells et al., Philos. Trans. R. Soc. London SerA, 317:415 (1986)] or other known techniques can be performed on the cloned DNA to produce the PRO variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol. 150:1 (1976)]. If alanine substitution does not yield adequate amounts of variant, an isoteric amino acid can be used.

C. Modifications of PRO

Covalent modifications of PRO are included within the scope of this invention. One type of covalent modification includes reacting targeted amino acid residues of a PRO polypeptide with an organic derivatizing agent that is capable of reacting with selected side chains or the N- or C-terminal residues of the PRO. Derivatization with bifunctional agents is useful, for instance, for crosslinking PRO to a water-insoluble support matrix or surface for use in the method for purifying anti-PRO antibodies, and vice-versa Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO polypeptide included within the scope of this invention comprises altering the native glycosylation pattern of the polypeptide. “Altering the native glycosylation pattern” is intended for purposes herein to mean deleting one or more carbohydrate moieties found in native sequence PRO (either by removing the underlying glycosylation site or by deleting the glycosylation by chemical and/or enzymatic means), and/or adding one or more glycosylation sites that are not present in the native sequence PRO. In addition, the phrase includes qualitative changes in the glycosylation of the native proteins, involving a change in the nature and proportions of the various carbohydrate moieties present.

Addition of glycosylation sites to the PRO polypeptide may be accomplished by altering the amino acid sequence. The alteration may be made, for example, by the addition of, or substitution by, one or more serine or threonine residues to the native sequence PRO (for O-linked glycosylation sites). The PRO amino acid sequence may optionally be altered through changes at the DNA level, particularly by mutating the DNA encoding the PRO polypeptide at preselected bases such that codons are generated that will translate into the desired amino acids.

Another means of increasing the number of carbohydrate moieties on the PRO polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO comprises linking the PRO polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337.

The PRO of the present invention may also be modified in a way to form a chimeric molecule comprising PRO fused to another, heterologous polypeptide or amino acid sequence.

In one embodiment, such a chimeric molecule comprises a fusion of the PRO with a tag polypeptide which provides an epitope to which an anti-tag antibody can selectively bind. The epitope tag is generally placed at the amino- or carboxyl-terminus of the PRO. The presence of such epitope-tagged forms of the PRO can be detected using an antibody against the tag polypeptide. Also, provision of the epitope tag enables the PRO to be readily purified by affinity purification using an anti-tag antibody or another type of affinity matrix that binds to the epitope tag. Various tag polypeptides and their respective antibodies are well known in the art Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto (Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the PRO with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of PRO

The description below relates primarily to production of PRO by culturing cells transformed or transfected with a vector containing PRO nucleic acid. It is, of course, contemplated that alternative methods, which are well known in the art, may be employed to prepare PRO. For instance, the PRO sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO may be chemically synthesized separately and combined using chemical or enzymatic methods to produce the full-length PRO.

1. Isolation of DNA Encoding PRO

DNA encoding PRO may be obtained from a cDNA library prepared from tissue believed to possess the PRO mRNA and to express it at a detectable level. Accordingly, human PRO DNA can be conveniently obtained from a cDNA library prepared from human tissue, such as described in the Examples. The PRO-encoding gene may also be obtained from a genomic library or by known synthetic procedures (e.g., automated nucleic acid synthesis).

Libraries can be screened with probes (such as antibodies to the PRO or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO-encoding vectors. Saccharomyces cerevisiae is a commonly used lower eukaryotic host microorganism. Others include Schizosaccharomyces pombe (Beach and Nurse, Nature, 290: 140 (1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PRO are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/−DHFR (CHO, Urlaub and Chasin, Proc. Natl. Acad. Sci. USA, 77:4216 (1980)); mouse sertoli cells (TM4, Mather, Biol. Retrod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding PRO may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO-encoding nucleic acid, such as DHFR or thymidine kinase. An appropriate host cell when wild-type DHFR is employed is the CHO cell line deficient in DHPR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PRO-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature. 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg. 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO.

Still other methods, vectors, and host cells suitable for adaptation to the synthesis of PRO in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO polypeptide or against a synthetic peptide based on the DNA sequences provided herein or against exogenous sequence fused to PRO DNA and encoding a specific antibody epitope.

5. Purification of Polypeptide

Forms of PRO may be recovered from culture medium or from host cell lysates. If membrane-bound, it can be released from the membrane using a suitable detergent solution (e.g. Triton-X 100) or by enzymatic cleavage. Cells employed in expression of PRO can be disrupted by various physical or chemical means, such as freeze-thaw cycling, sonication, mechanical disruption, or cell lysing agents.

It may be desired to purify PRO from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PRO produced.

E. Tissue Distribution

The location of tissues expressing the PRO can be identified by determining mRNA expression in various human tissues. The location of such genes provides information about which tissues are most likely to be affected by the stimulating and inhibiting activities of the PRO polypeptides. The location of a gene in a specific tissue also provides sample tissue for the activity blocking assays discussed below.

As noted before, gene expression in various tissues may be measured by conventional Southern blotting, Northern blotting to quantitate the transcription of mRNA (Thomas, Proc. Natl. Acad. Sci. USA, 77:5201-5205 [1980]), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes.

Gene expression in various tissues, alternatively, may be measured by immunological methods, such as immunohistochemical staining of tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence of a PRO polypeptide or against a synthetic peptide based on the DNA sequences encoding the PRO polypeptide or against an exogenous sequence fused to a DNA encoding a PRO polypeptide and encoding a specific antibody epitope. General techniques for generating antibodies, and special protocols for Northern blotting and in situ hybridization are provided below.

F. Antibody Binding Studies

The activity of the PRO polypeptides can be further verified by antibody binding studies, in which the ability of anti-PRO antibodies to inhibit the effect of the PRO polypeptides, respectively, on tissue cells is tested. Exemplary antibodies include polyclonal, monoclonal, humanized, bispecific, and heteroconjugate antibodies, the preparation of which will be described hereinbelow.

Antibody binding studies may be carried out in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tissue sample may be fresh or frozen or may be embedded in paraffin and fixed with a preservative such as formalin, for example.

G. Cell-Based Assays

Cell-based assays and animal models for immune related diseases can be used to further understand the relationship between the genes and polypeptides identified herein and the development and pathogenesis of immune related disease.

In a different approach, cells of a cell type known to be involved in a particular immune related disease are transfected with the cDNAs described herein, and the ability of these cDNAs to stimulate or inhibit immune function is analyzed. Suitable cells can be transfected with the desired gene, and monitored for immune function activity. Such transfected cell lines can then be used to test the ability of poly- or monoclonal antibodies or antibody compositions to inhibit or stimulate immune function, for example to modulate T-cell proliferation or inflammatory cell infiltration. Cells transfected with the coding sequences of the genes identified herein can further be used to identify drug candidates for the treatment of immune related diseases.

In addition, primary cultures derived from transgenic animals (as described below) can be used in the cell-based assays herein, although stable cell lines are preferred. Techniques to derive continuous cell lines from transgenic animals are well known in the art (see, e.g., Small et al., Mol. Cell. Biol. 5: 642-648 [1985]).

One suitable cell based assay is the mixed lymphocyte reaction (MLR). Current Protocols in Immunology, unit 3.12; edited by J B Coligan, A M Kruisbeek, D H Marglies, B M Shevach, W Strober, National Institutes of Health, Published by John Wiley & Sons, Inc. In this assay, the ability of a test compound to stimulate or inhibit the proliferation of activated T cells is assayed. A suspension of responder T cells is cultured with allogeneic stimulator cells and the proliferation of T cells is measured by uptake of tritiated thymidine. This assay is a general measure of T cell reactivity. Since the majority of T cells respond to and produce IL-2 upon activation, differences in responsiveness in this assay in part reflect differences in IL-2 production by the responding cells. The MLR results can be verified by a standard lymphokine (IL-2) detection assay. Current Protocols in Immunology, above, 3.15, 6.3.

A proliferative T cell response in an MLR assay may be due to direct mitogenic properties of an assayed molecule or to external antigen induced activation. Additional verification of the T cell stimulatory activity of the PRO polypeptides can be obtained by a costimulation assay. T cell activation requires an antigen specific signal mediated through the T-cell receptor (TCR) and a costimulatory signal mediated through a second ligand binding interaction, for example, the B7 (CD80, CD86)/CD28 binding interaction. CD28 crosslinking increases lymphokine secretion by activated T cells. T cell activation has both negative and positive controls through the binding of ligands which have a negative or positive effect CD28 and CTLA-4 are related glycoproteins in the Ig superfamily which bind to B7. CD28 binding to B7 has a positive costimulation effect of T cell activation; conversely, CTLA4 binding to B7 has a T cell deactivating effect Chambers, C. A. and Allison, J. P., Curr. Opin. Immunol. (1997) 9:396. Schwartz, R. H., Cell (1992) 71:1065; Linsey, P. S. and Ledbetter, J. A., Annu. Rev. Immunol. (1993) 11:191; June, C. H. et al, Immunol. Today (1994) 15:321; Jenkins, M. K., Immunity (1994) 1:405. In a costimulation assay, the PRO polypeptides are assayed for T cell costimulatory or inhibitory activity.

Direct use of a stimulating compound as in the invention has been validated in experiments with 4-1BB glycoprotein, a member of the tumor necrosis factor receptor family, which binds to a ligand (4-IBBL) expressed on primed T cells and signals T cell activation and growth. Alderson, M. E. et al., J. Immunol. (1994) 24:2219.

The use of an agonist stimulating compound has also been validated experimentally. Activation of 4-BB by treatment with an agonist anti-4-1BB antibody enhances eradication of tumors. Hellstrom, I. and Hellstrom, K. E., Crit. Rev. Immunol. (1998) 18:1. Immunoadjuvant therapy for treatment of tumors, described in more detail below, is another example of the use of the stimulating compounds of the invention.

Alternatively, an immune stimulating or enhancing effect can also be achieved by administration of a PRO which has vascular permeability enhancing properties. Enhanced vascular permeability would be beneficial to disorders which can be attenuated by local infiltration of immune cells (e.g., monocytes, eosinophils, PMNs) and inflammation.

On the other hand, PRO polypeptides, as well as other compounds of the invention, which are direct inhibitors of T cell proliferation/activation, lymphokine secretion, and/or vascular permeability can be directly used to suppress the immune response. These compounds are useful to reduce the degree of the immune response and to treat immune related diseases characterized by a hyperactive, superoptimal, or autoimmune response. This use of the compounds of the invention has been validated by the experiments described above in which CTLA4 binding to receptor B7 deactivates T cells. The direct inhibitory compounds of the invention function in an analogous manner. The use of compound which suppress vascular permeability would be expected to reduce inflammation. Such uses would be beneficial in treating conditions associated with excessive inflammation.

Alternatively, compounds, e.g., antibodies, which bind to stimulating PRO polypeptides and block the stimulating effect of these molecules produce a net inhibitory effect and can be used to suppress the T cell mediated immune response by inhibiting T cell proliferation/activation and/or lymphokine secretion. Blocking the stimulating effect of the polypeptides suppresses the immune response of the mammal. This use has been validated in experiments using an anti-IL2 antibody. In these experiments, the antibody binds to IL2 and blocks binding of IL2 to its receptor thereby achieving a T cell inhibitory effect.

H. Animal Models

The results of the cell based in vitro assays can be further verified using in vivo animal models and assays for T-cell function. A variety of well known animal models can be used to further understand the role of the genes identified herein in the development and pathogenesis of immune related disease, and to test the efficacy of candidate therapeutic agents, including antibodies, and other antagonists of the native polypeptides, including small molecule antagonists. The in vivo nature of such models makes them predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g., subcutaneous injection, tail vein injection, spleen implantation, intraperitoneal implantation, implantation under the renal capsule, etc.

Graft-versus-host disease occurs when immunocompetent cells are transplanted into immunosuppressed or tolerant patients. The donor cells recognize and respond to host antigens. The response can vary from life threatening severe inflammation to mild cases of diarrhea and weight loss. Graft-versus-host disease models provide a means of assessing T cell reactivity against MHC antigens and minor transplant antigens. A suitable procedure is described in detail in Current Protocols in Immunology, above, unit 4.3.

An animal model for skin allograft rejection is a means of testing the ability of T cells to mediate in vivo tissue destruction and a measure of their role in transplant rejection. The most common and accepted models use murine tail-skin grafts. Repeated experiments have shown that skin allograft rejection is mediated by T cells, helper T cells and killer-effector T cells, and not antibodies. Auchincloss, H. Jr. and Sachs, D. H., Fundamental Immunology, 2nd ed., W. E. Paul ed., Raven Press, NY, 1989, 889-992. A suitable procedure is described in detail in Current Protocols in Immunology, above, unit 4.4. Other transplant rejection models which can be used to test the compounds of the invention are the allogeneic heart transplant models described by Tanabe, M. et al, Transplantation (1994) 58:23 and Tinubu, S. A. et al, J. Immunol. (1994) 4330-4338.

Animal models for delayed type hypersensitivity provides an assay of cell mediated immune function as well. Delayed type hypersensitivity reactions are a T cell mediated in vivo immune response characterized by inflammation which does not reach a peak until after a period of time has elapsed after challenge with an antigen. These reactions also occur in tissue specific autoimmune diseases such as multiple sclerosis (MS) and experimental autoimmune encephalomyelitis (EAE, a model for MS). A suitable procedure is described in detail in Current Protocols in Immunology, above, unit 4.5.

EAE is a T cell mediated autoimmune disease characterized by T cell and mononuclear cell inflammation and subsequent demyelination of axons in the central nervous system. FAB is generally considered to be a relevant animal model for MS in humans. Bolton, C., Multiple Sclerosis (1995) 1:143. Both acute and relapsing-remitting models have been developed. The compounds of the invention can be tested for T cell stimulatory or inhibitory activity against immune mediated demyelinating disease using the protocol described in Current Protocols in Immunology, above, units 15.1 and 15.2. See also the models for myelin disease in which oligodendrocytes or Schwann cells are grafted into the central nervous system as described in Duncan, I. D. et al, Molec. Med. Today (1997) 554-561.

Contact hypersensitivity is a simple delayed type hypersensitivity in vivo assay of cell mediated immune function. In this procedure, cutaneous exposure to exogenous haptens which gives rise to a delayed type hypersensitivity reaction which is measured and quantitated. Contact sensitivity involves an initial sensitizing phase followed by an elicitation phase. The elicitation phase occurs when the T lymphocytes encounter an antigen to which they have had previous contact. Swelling and inflammation occur, making this an excellent model of human allergic contact dermatitis. A suitable procedure is described in detail in Current Protocols in Immunology, Eds. J. E. Cologan, A. M. Kruisbeek, D. H. Margulies, E. M. Shevach and W. Strober, John Wiley & Sons, Inc., 1994, unit 4.2. See also Grabbe, S. and Schwarz, T, Immun. Today 19 (1): 3744 (1998).

An animal model for arthritis is collagen-induced arthritis. This model shares clinical, histological and immunological characteristics of human autoimmune rheumatoid arthritis and is an acceptable model for human autoimmune arthritis. Mouse and rat models are characterized by synovitis, erosion of cartilage and subchondral bone. The compounds of the invention can be tested for activity against autoimmune arthritis using the protocols described in Current Protocols in Immunology, above, units 15.5. See also the model using a monoclonal antibody to CD18 and VLA-4 integrins described in Issekutz, A. C. et al., Immunology (1996) 88:569.

A model of asthma has been described in which antigen-induced airway hyper-reactivity, pulmonary eosinophilia and inflammation are induced by sensitizing an animal with ovalbumin and then challenging the animal with the same protein delivered by aerosol. Several animal models (guinea pig, rat, non-human primate) show symptoms similar to atopic asthma in humans upon challenge with aerosol antigens. Murine models have many of the features of human asthma. Suitable procedures to test the compounds of the invention for activity and effectiveness in the treatment of asthma are described by Wolyniec, W. W. et al, Am. J. Respir. Cell Mol. Biol. (1998) 18:777 and the references cited therein.

Additionally, the compounds of the invention can be tested on animal models for psoriasis like diseases. Evidence suggests a T cell pathogenesis for psoriasis. The compounds of the invention can be tested in the scid/scid mouse model described by Schon, M. P. et al, Nat. Med. (1997) 3:183, in which the mice demonstrate histopathologic skin lesions resembling psoriasis. Another suitable model is the human skin/scid mouse chimera prepared as described by Nickoloff, B. J. et al, Am. J. Path. (1995) 146:580.

Recombinant (transgenic) animal models can be engineered by introducing the coding portion of the genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA 82, 6148-615 [1985]); gene targeting in embryonic stem cells (Thompson et al., Cell 5, 313-321 [1989]); electroporation of embryos (Lo, Mol. Cel. Biol. 3, 1803-1814 [19831); sperm-mediated gene transfer (Lavitrano et al., Cell 57, 717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA 89, 6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry.

The animals may be further examined for signs of immune disease pathology, for example by histological examination to determine infiltration of immune cells into specific tissues. Blocking experiments can also be performed in which the transgenic animals are treated with the compounds of the invention to determine the extent of the T cell proliferation stimulation or inhibition of the compounds. In these experiments, blocking antibodies which bind to the PRO polypeptide, prepared as described above, are administered to the animal and the effect on immune function is determined.

Alternatively, “knock out” animals can be constructed which have a defective or altered gene encoding a polypeptide identified herein, as a result of homologous recombination between the endogenous gene encoding the polypeptide and altered genomic DNA encoding the same polypeptide introduced into an embryonic cell of the animal. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the polypeptide.

I. ImmunoAdjuvant Therapy

In one embodiment, the immunostimulating compounds of the invention can be used in immunoadjuvant therapy for the treatment of tumors (cancer). It is now well established that T cells recognize human tumor specific antigens. One group of tumor antigens, encoded by the MAGE, BAGE and GAGE families of genes, are silent in all adult normal tissues, but are expressed in significant amounts in tumors, such as melanomas, lung tumors, head and neck tumors, and bladder carcinomas. DeSmet, C. et al., (1996) Proc. Natl. Acad. Sci. USA, 93:7149. It has been shown that costimulation of T cells induces tumor regression and an antitumor response both in vitro and in vivo. Melero, I. et al., Nature Medicine (1997) 3:682; Kwon, E. D. et al., Proc. Natl. Acad. Sci. USA (1997) 94: 8099; Lynch, D. H. et al, Nature Medicine (1997) 3:625; Finn, O. J. and Lotze, M. T., J. Immunol. (1998) 21:114. The stimulatory compounds of the invention can be administered as adjuvants, alone or together with a growth regulating agent, cytotoxic agent or chemotherapeutic agent, to stimulate T cell proliferation/activation and an antitumor response to tumor antigens. The growth regulating, cytotoxic, or chemotherapeutic agent may be administered in conventional amounts using known administration regimes. Immunostimulating activity by the compounds of the invention allows reduced amounts of the growth regulating, cytotoxic, or chemotherapeutic agents thereby potentially lowering the toxicity to the patient

J. Screening Assays for Drug Candidates

Screening assays for drug candidates are designed to identify compounds that bind to or complex with the polypeptides encoded by the genes identified herein or a biologically active fragment thereof, or otherwise interfere with the interaction of the encoded polypeptides with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art. All assays are common in that they call for contacting the drug candidate with a polypeptide encoded by a nucleic acid identified herein under conditions and for a time sufficient to allow these two components to interact.

In binding assays, the interaction is binding and the complex formed can be isolated or detected in the reaction mixture. In a particular embodiment, the polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labelled antibody specifically binding the immobilized complex.

If the candidate compound interacts with but does not bind to a particular protein encoded by a gene identified herein, its interaction with that protein can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system described by Fields and co-workers [Fields and Song, Nature (London) 340, 245-246 (1989); Chien et al., Proc. Natl. Acad. Sci. USA 88, 9578-9582 (1991)] as disclosed by Chevray and Nathans, Proc. Natl. Acad. Sci. USA a 5789-5793 (1991). Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

In order to find compounds that interfere with the interaction of a gene identified herein and other intra- or extracellular components can be tested, a reaction mixture is usually prepared containing the product of the gene and the intra- or extracellular component under conditions and for a time allowing for the interaction and binding of the two products. To test the ability of a test compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described above. The formation of a complex in the control reaction(s) but not in the reaction mixture containing the test compound indicates that the test compound interferes with the interaction of the test compound and its reaction partner.

K. Compositions and Methods for the Treatment of Immune Related Diseases

The compositions useful in the treatment of immune related diseases include, without limitation, proteins, antibodies, small organic molecules, peptides, phosphopeptides, antisense and ribozyme molecules, triple helix molecules, etc. that inhibit or stimulate immune function, for example, T cell proliferation/activation, lymphokine release, or immune cell infiltration.

For example, antisense RNA and RNA molecules act to directly block the translation of mRNA by hybridizing to targeted mRNA and preventing protein translation. When antisense DNA is used, oligodeoxyribonucleotides derived from the translation initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology 4, 469-471 (1994), and PCT publication No. WO 97/33551 (published Sep. 18, 1997).

Nucleic acid molecules in triple helix formation used to inhibit transcription should be single-stranded and composed of deoxynucleotides. The base composition of these oligonucleotides is designed such that it promotes triple helix formation via Hoogsteen base pairing rules, which generally require sizeable stretches of purines or pyrimidines on one strand of a duplex. For further details see, e.g., PCT publication No. WO 97/33551, supra.

These molecules can be identified by any or any combination of the screening assays discussed above and/or by any other screening techniques well known for those skilled in the art.

L. Anti-PRO Antibodies

The present invention further provides anti-PRO antibodies. Exemplary antibodies include polyclonal, monoclonal, humanized, bispecific, and heteroconjugate antibodies.

1. Polyclonal Antibodies

The anti-PRO antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the PRO polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-PRO antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the PRO polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against PRO. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art

3. Human and Humanized Antibodies

The anti-PRO antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

4. Bispecific Antibodies

Bispecific antibodies are monoclonal, preferably human or humanized, antibodies that have binding specificities for at least two different antigens. In the present case, one of the binding specificities is for the PRO, the other one is for any other antigen, and preferably for a cell-surface protein or receptor or receptor subunit.

Methods for making bispecific antibodies are known in the art Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolyticaly cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies may bind to two different epitopes on a given PRO polypeptide herein. Alternatively, an anti-PRO polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular PRO polypeptide. Bispecific antibodies may also be used to localize cytotoxic agents to cells which express a particular PRO polypeptide. These antibodies possess a PRO-binding arm and an arm which binds a cytotoxic agent or a radionuclide chelator, such as EOTUBE, DPTA, DOTA, or TETA. Another bispecific antibody of interest binds the PRO polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al., Anti-Cancer Drug Design. 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

M. Pharmaceutical Compositions

The active PRO molecules of the invention (e.g., PRO polypeptides, anti-PRO antibodies, and/or variants of each) as well as other molecules identified by the screening assays disclosed above, can be administered for the treatment of immune related diseases, in the form of pharmaceutical compositions.

Therapeutic formulations of the active PRO molecule, preferably a polypeptide or antibody of the invention, are prepared for storage by mixing the active molecule having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Compounds identified by the screening assays disclosed herein can be formulated in an analogous manner, using standard techniques well known in the art.

Lipofections or liposomes can also be used to deliver the PRO molecule into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Natl. Acad. Sci. USA 90, 7889-7893 [1993]).

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent Such molecules are suitably present in combination in amounts that are effective for the purpose intended.

The active PRO molecules may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations or the PRO molecules may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

N. Methods of Treatment

It is contemplated that the polypeptides, antibodies and other active compounds of the present invention may be used to treat various immune related diseases and conditions, such as T cell mediated diseases, including those characterized by infiltration of inflammatory cells into a tissue, stimulation of T-cell proliferation, inhibition of T-cell proliferation, increased or decreased vascular permeability or the inhibition thereof.

Exemplary conditions or disorders to be treated with the polypeptides, antibodies and other compounds of the invention, include, but are not limited to systemic lupus erythematosis, rheumatoid arthritis, juvenile chronic arthritis, osteoarthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, EB and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In systemic lupus erythematosus, the central mediator of disease is the production of auto-reactive antibodies to self proteins/tissues and the subsequent generation of immune-mediated inflammation. Antibodies either directly or indirectly mediate tissue injury. Though T lymphocytes have not been shown to be directly involved in tissue damage, T lymphocytes are required for the development of auto-reactive antibodies. The genesis of the disease is thus T lymphocyte dependent. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, intestitial pneumonitis with pulmonary fibrosis, keratoconjunctivitis sicca, and rhematoid nodules.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjögren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type I diabetes mellitus or insulin-dependent diabetes is the autoimmune destruction of pancreatic islet β cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including Multiple Sclerosis; idiopathic demyelinating polyneuropathy or Guillain-Barré syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple Sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+ T lymphocytes are the predominant cell type at lesions. The mechanism of oligodendrocyte cell death and subsequent demyelination is not known but is likely T lymphocyte driven.

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Autoimmune or Immune-mediated Skin Disease including Bullous Skin Diseases, Erythema Multiforme, and Contact Dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent These diseases are predominantly mediated by T lymphocyte induced inflammation, IgE mediated-inflammation or a combination of both.

Transplantation associated diseases, including Graft rejection and Graft-Versus-Host-Disease (GVHD) are T lymphocyte-dependent; inhibition of T lymphocyte function is ameliorative.

Other diseases in which intervention of the immune and/or inflammatory response have benefit are infectious disease including but not limited to viral infection (including but not limited to AIDS, hepatitis A, B, C, D, E and herpes) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (i.e., as from chemotherapy) immunodeficiency, and neoplasia.

It has been demonstrated that some human cancer patients develop an antibody and/or T lymphocyte response to antigens on neoplastic cells. It has also been shown in animal models of neoplasia that enhancement of the immune response can result in rejection or regression of that particular neoplasm. Molecules that enhance the T lymphocyte response in the MLR have utility in vivo in enhancing the immune response against neoplasia Molecules which enhance the T lymphocyte proliferative response in the MLR (or small molecule agonists or antibodies that affected the same receptor in an agonistic fashion) can be used therapeutically to treat cancer. Molecules that inhibit the lymphocyte response in the MLR also function in vivo during neoplasia to suppress the immune response to a neoplasm; such molecules can either be expressed by the neoplastic cells themselves or their expression can be induced by the neoplasm in other cells. Antagonism of such inhibitory molecules (either with antibody, small molecule antagonists or other means) enhances immune-mediated tumor rejection.

Additionally, inhibition of molecules with proinflammatory properties may have therapeutic benefit in reperfusion injury; stroke; myocardial infarction; atherosclerosis; acute lung injury; hemorrhagic shock; burn; sepsis/septic shock; acute tubular necrosis; endometriosis; degenerative joint disease and pancreatis.

The compounds of the present invention, e.g., polypeptides or antibodies, are administered to a mammal, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation (intranasal, intrapulmonary) routes. Intravenous or inhaled administration of polypeptides and antibodies is preferred.

In immunoadjuvant therapy, other therapeutic regimens, such administration of an anti-cancer agent, may be combined with the administration of the proteins, antibodies or compounds of the instant invention. For example, the patient to be treated with a the immunoadjuvant of the invention may also receive an anti-cancer agent (chemotherapeutic agent) or radiation therapy. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the immunoadjuvant or may be given simultaneously therewith. Additionally, an anti-estrogen compound such as tamoxifen or an anti-progesterone such as onapristone (see, EP 616812) may be given in dosages known for such molecules.

It may be desirable to also administer antibodies against other immune disease associated or tumor associated antigens, such as antibodies which bind to CD20, CD11a, CD18, ErbB2, EGFR, ErbB3, ErbB4, or vascular endothelial factor (VEGF). Alternatively, or in addition, two or more antibodies binding the same or two or more different antigens disclosed herein may be coadministered to the patient. Sometimes, it may be beneficial to also administer one or more cytokines to the patient. In one embodiment, the PRO polypeptides are coadministered with a growth inhibitory agent. For example, the growth inhibitory agent may be administered first, followed by a PRO polypeptide. However, simultaneous administration or administration first is also contemplated. Suitable dosages for the growth inhibitory agent are those presently used and may be lowered due to the combined action (synergy) of the growth inhibitory agent and the PRO polypeptide.

For the treatment or reduction in the severity of immune related disease, the appropriate dosage of an a compound of the invention will depend on the type of disease to be treated, as defined above, the severity and course of the disease, whether the agent is administered for preventive or therapeutic purposes, previous therapy, the patient's clinical history and response to the compound, and the discretion of the attending physician. The compound is suitably administered to the patient at one time or over a series of treatments.

For example, depending on the type and severity of the disease, about 1 μg/kg to 15 mg/kg (e.g., 0.1-20 mg/kg) of polypeptide or antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

O. Articles of Manufacture

In another embodiment of the invention, an article of manufacture containing materials (e.g., comprising a PRO molecule) useful for the diagnosis or treatment of the disorders described above is provided. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is usually a polypeptide or an antibody of the invention. An instruction or label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

P. Diagnosis and Prognosis of Immune Related Disease

Cell surface proteins, such as proteins which are overexpressed in certain immune related diseases, are excellent targets for drug candidates or disease treatment. The same proteins along with secreted proteins encoded by the genes amplified in immune related disease states find additional use in the diagnosis and prognosis of these diseases. For example, antibodies directed against the protein products of genes amplified in multiple sclerosis, rheumatoid arthritis, or another immune related disease, can be used as diagnostics or prognostics.

For example, antibodies, including antibody fragments, can be used to qualitatively or quantitatively detect the expression of proteins encoded by amplified or overexpressed genes (“marker gene products”). The antibody preferably is equipped with a detectable, e.g., fluorescent label, and binding can be monitored by light microscopy, flow cytometry, fluorimetry, or other techniques known in the art. These techniques are particularly suitable, if the overexpressed gene encodes a cell surface protein Such binding assays are performed essentially as described above.

In situ detection of antibody binding to the marker gene products can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Microarray Analysis of Stimulated T-Cells

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support Hybridization of a labeled probe with a particular array member indicates that the sample from which the probe was derived expresses that gene. If the hybridization signal of a probe from a test (in this instance, activated CD4+ T cells) sample is greater than hybridization signal of a probe from a control (in this instance, non-stimulated CD4+ T cells) sample, the gene or genes overexpressed in the test tissue are identified. The implication of this result is that an overexpressed protein in a test tissue is useful not only as a diagnostic marker for the presence of the disease condition, but also as a therapeutic target for treatment of the disease condition.

The methodology of hybridization of nucleic acids and microarray technology is well known in the art In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

In this experiment, CD4+ T cells were purified from a single donor using the RossetteSep™ protocol from (Stem Cell Technologies, Vancouver BC) which contains anti-CD8, anti-CD16, anti-CD19, anti-CD36 and anti-CD56 antibodies used to produce a population of isolated CD4+ T cells. Isolated CD4+ T cells were activated with an anti-CD3 antibody (used at a concentration that does not stimulate proliferation) together with either ICAM-1 or anti-CD28 antibody. At 24 or 72 hours cells were harvested, RNA extracted and analysis run on Affimax (Affymetrix Inc. Santa Clara, Calif.) microarray chips. Non-stimulated (resting) cells were harvested immediately after purification, and subjected to the same analysis. Genes were compared whose expression was upregulated at either of the two timepoints in activated vs. resting cells.

Below are the results of these experiments, demonstrating that various PRO polypeptides of the present invention are differentially expressed in isolated CD4+ T cells activated by anti-CD3/ICAM-1 or anti-CD3/anti-CD28 as compared to isolated resting CD4+ T cells. As described above, these data demonstrate that the PRO polypeptides of the present invention are useful not only as diagnostic markers for the presence of one or more immune disorders, but also serve as therapeutic targets for the treatment of those immune disorders.

The results of this experment are FIGS. 1-7589 show increase or decrease in expression upon stimulation with anti-CD3/ICAM1 and also show increase or decrease in expression upon stimulation with anti-CD3/anti-CD28. The nucleic acids and encoded proteins of FIG. 946, FIG. 1520, FIG. 1574, FIG. 1622, FIG. 1816, FIG. 2433, FIG. 2986, FIG. 3220, FIG. 4120 and FIG. 5421 are significantly overexpressed in isolated CD4+ T cells activated by anti-CD3/ICAM-1 or anti-CD3/anti-CD28 as compared to isolated resting CD4+ T cells.

### Example 2

### Use of PRO as a Hybridization Probe

The following method describes use of a nucleotide sequence encoding PRO as a hybridization probe.

DNA comprising the coding sequence of full-length or mature PRO as disclosed herein is employed as a probe to screen for homologous DNAs (such as those encoding naturally-occurring variants of PRO) in human tissue cDNA libraries or human tissue genomic libraries.

Hybridization and washing of filters containing either library DNAs is performed under the following high stringency conditions. Hybridization of radiolabeled PRO-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence PRO can then be identified using standard techniques known in the art.

### Example 3

### Expression of PRO in E. coli

This example illustrates preparation of an unglycosylated form of PRO by recombinant expression in E. coli.

The DNA sequence encoding PRO is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO protein can then be purified using a metal chelating column under conditions that allow tight binding of the protein.

PRO may be expressed in E. coli in a poly-His tagged form, using the following procedure. The DNA encoding PRO is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate•2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

Many of the PRO polypeptides disclosed herein were successfully expressed as described above.

### Example 4

### Expression of PRO in Mammalian Cells

This example illustrates preparation of a potentially glycosylated form of PRO by recombinant expression in mammalian cells.

The vector, pRK5 (see EP 307,247, published Mar. 15, 1989), is employed as the expression vector. Optionally, the PRO DNA is ligated into pRK5 with selected restriction enzymes to allow insertion of the PRO DNA using ligation methods such as described in Sambrook et al., supra. The resulting vector is called pRK5-PRO.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO DNA is mixed with about 1 μg DNA encoding the VA RNA gene [Thimmappaya et al., Cell. 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO polypeptide. The cultures containing transfected cells may undergo further incubation (in serum free medium) and the medium is tested in selected bioassays.

In an alternative technique, PRO may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO can then be concentrated and purified by any selected method, such as dialysis and/or column chromatography.

In another embodiment, PRO can be expressed in CHO cells. The pRK5-PRO can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO polypeptide, the culture medium may be replaced with serum free medium. Preferably, the cultures are incubated for about 6 days, and then the conditioned medium is harvested. The medium containing the expressed PRO can then be concentrated and purified by any selected method.

Epitope-tagged PRO may also be expressed in host CHO cells. The PRO may be subcloned out of the pRK5 vector. The subclone insert can undergo PCR to fuse in frame with a selected epitope tag such as a poly-his tag into a Baculovirus expression vector. The poly-his tagged PRO insert can then be subcloned into a SV40 promoter/enhancer containing vector containing a selection marker such as DHFR for selection of stable clones. Finally, the CHO cells can be transfected (as described above) with the SV40 promoter/enhancer containing vector. Labeling may be performed, as described above, to verify expression. The culture medium containing the expressed poly-His tagged PRO can then be concentrated and purified by any selected method, such as by Ni²⁺-chelate affinity chromatography.

PRO may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mL of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3 L production spinner is seeded at 1.2×10⁶ cells/mL. On day 0, pH is determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μl of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Many of the PRO polypeptides disclosed herein were successfully expressed as described above.

### Example 5

### Expression of PRO in Yeast

The following method describes recombinant expression of PRO in yeast.

First, yeast expression vectors are constructed for intracellular production or secretion of PRO from the ADH2/GAPDH promoter. DNA encoding PRO and the promoter is inserted into suitable restriction enzyme sites in the selected plasmid to direct intracellular expression of PRO. For secretion, DNA encoding PRO can be cloned into the selected plasmid, together with DNA encoding the ADH2/GAPDH promoter, a native PRO signal peptide or other mammalian signal peptide, or, for example, a yeast alpha-factor or invertase secretory signal/leader sequence, and linker sequences (if needed) for expression of PRO.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO can subsequently be isolated and purified by removing the yeast cells from the fermentation medium by centrifugation and then concentrating the medium using selected cartridge filters. The concentrate containing PRO may further be purified using selected column chromatography resins.

Many of the PRO polypeptides disclosed herein were successfully expressed as described above.

### Example 6

### Expression of PRO in Baculovirus-Infected Insect Cells

The following method describes recombinant expression of PRO in Baculovirus-infected insect cells.

The sequence coding for PRO is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding PRO or the desired portion of the coding sequence of PRO such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO are pooled and dialyzed against loading buffer.

Alternatively, purification of the IgG tagged (or Fc tagged) PRO can be performed using known chromatography techniques, including for instance, Protein A or protein G column chromatography.

Many of the PRO polypeptides disclosed herein were successfully expressed as described above.

### Example 7

### Preparation of Antibodies that Bind PRO

This example illustrates preparation of monoclonal antibodies which can specifically bind PRO.

Techniques for producing the monoclonal antibodies are known in the art and are described, for instance, in Goding, supra Immunogens that may be employed include purified PRO, fusion proteins containing PRO, and cells expressing recombinant PRO on the cell surface. Selection of the immunogen can be made by the skilled artisan without undue experimentation.

Mice, such as Balb/c, are immunized with the PRO immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO antibodies.

After a suitable antibody titer has been detected, the animals “positive” for antibodies can be injected with a final intravenous injection of PRO. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO. Determination of “positive” hybridoma cells secreting the desired monoclonal antibodies against PRO is within the skill in the art.

The positive hybridoma cells can be injected intraperitoneally into syngeneic Balb/c mice to produce ascites containing the anti-PRO monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 8

### Purification of PRO Polypeptides Using Specific Antibodies

Native or recombinant PRO polypeptides may be purified by a variety of standard techniques in the art of protein purification. For example, pro-PRO polypeptide, mature PRO polypeptide, or pre-PRO polypeptide is purified by immunoaffinity chromatography using antibodies specific for the PRO polypeptide of interest. In general, an immunoaffinity column is constructed by covalently coupling the anti-PRO polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PRO polypeptide by preparing a fraction from cells containing PRO polypeptide in a soluble form. This preparation is derived by solubilization of the whole cell or of a subcellular fraction obtained via differential centrifugation by the addition of detergent or by other methods well known in the art Alternatively, soluble PRO polypeptide containing a signal sequence may be secreted in useful quantity into the medium in which the cells are grown.

A soluble PRO polypeptide-containing preparation is passed over the immunoaffinity column, and the column is washed under conditions that allow the preferential absorbance of PRO polypeptide (e.g., high ionic strength buffers in the presence of detergent). Then, the column is eluted under conditions that disrupt antibody/PRO polypeptide binding (e.g., a low pH buffer such as approximately pH 2-3, or a high concentration of a chaotrope such as urea or thiocyanate ion), and PRO polypeptide is collected.

### Example 9

### Drug Screening

This invention is particularly useful for screening compounds by using PRO polypeptides or binding fragment thereof in any of a variety of drug screening techniques. The PRO polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO polypeptide or fragment. Drugs are screened against such transformed cells in competitive binding assays. Such cells, either in viable or fixed form, can be used for standard binding assays. One may measure, for example, the formation of complexes between PRO polypeptide or a fragment and the agent being tested. Alternatively, one can examine the diminution in complex formation between the PRO polypeptide and its target cell or target receptors caused by the agent being tested.

Thus, the present invention provides methods of screening for drugs or any other agents which can affect a PRO polypeptide-associated disease or disorder. These methods comprise contacting such an agent with an PRO polypeptide or fragment thereof and assaying (I) for the presence of a complex between the agent and the PRO polypeptide or fragment, or (ii) for the presence of a complex between the PRO polypeptide or fragment and the cell, by methods well known in the art. In such competitive binding assays, the PRO polypeptide or fragment is typically labeled. After suitable incubation, free PRO polypeptide or fragment is separated from that present in bound form, and the amount of free or uncomplexed label is a measure of the ability of the particular agent to bind to PRO polypeptide or to interfere with the PRO polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO polypeptide, the peptide test compounds are reacted with PRO polypeptide and washed. Bound PRO polypeptide is detected by methods well known in the art. Purified PRO polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO polypeptide specifically compete with a test compound for binding to PRO polypeptide or fragments thereof. In this manner, the antibodies can be used to detect the presence of any peptide which shares one or more antigenic determinants with PRO polypeptide.

### Example 10

### Rational Drug Design

The goal of rational drug design is to produce structural analogs of biologically active polypeptide of interest (i.e., a PRO polypeptide) or of small molecules with which they interact, e.g., agonists, antagonists, or inhibitors. Any of these examples can be used to fashion drugs which are more active or stable forms of the PRO polypeptide or which enhance or interfere with the function of the PRO polypeptide in vivo (c.f., Hodgson, Bio/Technology, 9: 19-21 (1991)).

In one approach, the three-dimensional structure of the PRO polypeptide, or of a PRO polypeptide-inhibitor complex, is determined by x-ray crystallography, by computer modeling or, most typically, by a combination of the two approaches. Both the shape and charges of the PRO polypeptide must be ascertained to elucidate the structure and to determine active site(s) of the molecule. Less often, useful information regarding the structure of the PRO polypeptide may be gained by modeling based on the structure of homologous proteins. In both cases, relevant structural information is used to design analogous PRO polypeptide-like molecules or to identify efficient inhibitors. Useful examples of rational drug design may include molecules which have improved activity or stability as shown by Braxton and Wells, Biochemistry, 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO polypeptide may be made available to perform such analytical studies as X-ray crystallography. In addition, knowledge of the PRO polypeptide amino acid sequence provided herein will provide guidance to those employing computer modeling techniques in place of or in addition to x-ray crystallography.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed:

1. Isolated nucleic acid having at least 80% nucleic acid sequence identity to a nucleotide sequence identity to:
(a) the nucleotide sequence shown in any one of the FIGS. 1-7589 (SEQ ID NOS: 1-7589); or
(b) the nucleotide sequence encoding the polypeptide shown in any one of the FIGS. 1-7589 (SEQ ID NOS: 1-7589).

2. A vector comprising the nucleic acid of claim 1.

3. The vector of claim 2 operably linked to control sequences recognized by a host cell transformed with the vector.

4. A host cell comprising the vector of claim 2.

5. The host cell of claim 4, wherein said cell is a CHO cell, an E. coli cell or a yeast cell.

6. A process for producing a PRO polypeptide comprising culturing the host cell of claim 5 under conditions suitable for expression of said PRO polypeptide and recovering said PRO polypeptide from the cell culture.

7. An isolated polypeptide having at least 80% amino acid sequence identity to:
(a) a polypeptide shown in any one of FIGS. 1-7589 (SEQ ID NOS: 1-7589); or
(b) a polypeptide encoded by the full length coding region of the nucleotide sequence shown in any one of FIGS. 1-7589 (SEQ ID NOS: 1-7589).

8. A chimeric molecule comprising a polypeptide according to claim 7 fused to a heterologous amino acid sequence.

9. The chimeric molecule of claim 8, wherein said heterologous amino acid sequence is an epitope tag sequence or an Fc region of an immunoglobulin.

10. An antibody which specifically binds to a polypeptide according to claim 7.

11. The antibody of claim 10, wherein said antibody is a monoclonal antibody, a humanized antibody or a single-chain antibody.

12. A composition of matter comprising (a) a polypeptide of claim 7, (b) an agonist of said polypeptide, (c) an antagonist of said polypeptide, or (d) an antibody that binds to said polypeptide, in combination with a carrier.

13. The composition of matter of claim 12, wherein said carrier is a pharmaceutically acceptable carrier.

14. The composition of matter of claim 13 comprising a therapeutically effective amount of (a), (b), (c) or (d).

15. An article of manufacture, comprising:
a container;
a label on said container; and
a composition of matter comprising (a) a polypeptide of claim 7, (b) an agonist of said polypeptide, (c) an antagonist of said polypeptide, or (d) an antibody that binds to said polypeptide, contained within said container, wherein label on said container indicates that said composition of matter can be used for treating an immune related disease.

16. A method of treating an immune related disorder in a mammal in need thereof comprising administering to said mammal a therapeutically effective amount of (a) a polypeptide of claim 7, (b) an agonist of said polypeptide, (c) an antagonist of said polypeptide, or (d) an antibody that binds to said polypeptide.

17. The method of claim 16, wherein the immune related disorder is systemic lupus erythematosis, rheumatoid arthritis, osteoarthritis, juvenile chronic arthritis, a spondyloarthropathy, systemic sclerosis, an idiopathic inflammatory myopathy, Sjögren's syndrome, systemic vasculitis, sarcoidosis, autoimmune hemolytic anemia, autoimmune thrombocytopenia, thyroiditis, diabetes mellitus, immune-mediated renal disease, a demyelinating disease of the central or peripheral nervous system, idiopathic demyelinating polyneuropathy, Guillain-Barré syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease, gluten-sensitive enteropathy, Whipple's disease, an autoimmune or immune-mediated skin disease, a bullous skin disease, erythema multiforme, contact dermatitis, psoriasis, an allergic disease, asthma, allergic rhinitis, atopic dermatitis, food hypersensitivity, urticaria, an immunologic disease of the lung, eosinophilic pneumonias, idiopathic pulmonary fibrosis, hypersensitivity pneumonitis, a transplantation associated disease, graft rejection or graft-versus-host-disease.

18. A method for determining the presence of a PRO polypeptide of the invention as described in FIGS. 1-7589 (SEQ ID NOS: 1-7589), in a sample suspected of containing said polypeptide, said method comprising exposing said sample to an anti-PRO antibody, where the and determining binding of said antibody to a component of said sample.

19. A method of diagnosing an immune related disease in a mammal, said method comprising detecting the level of expression of a gene encoding a PRO polypeptide of the invention as described in FIGS. 1-7589 (SEQ ID NOS: 1-7589), (a) in a test sample of tissue cells obtained from the mammal, and (b) in a control sample of known normal tissue cells of the same cell type, wherein a higher or lower level of expression of said gene in the test sample as compared to the control sample is indicative of the presence of an immune related disease in the mammal from which the test tissue cells were obtained.

20. A method of diagnosing an immune related disease in a mammal, said method comprising (a) contacting a PRO polypeptide of the invention as described in FIGS. 1-7589 (SEQ ID NOS: 1-7589), anti-PRO antibody with a test sample of tissue cells obtained from said mammal and (b) detecting the formation of a complex between the antibody and the polypeptide in the test sample, wherein formation of said complex is indicative of the presence of an immune related disease in the mammal from which the test tissue cells were obtained.

21. A method of identifying a compound that inhibits the activity of a PRO polypeptide of the invention as described in FIGS. 1-7589 (SEQ ID NOS: 1-7589), said method comprising contacting cells which normally respond to said polypeptide with (a) said polypeptide and (b) a candidate compound, and determining the lack responsiveness by said cell to (a).

22. A method of identifying a compound that inhibits the expression of a gene encoding a PRO polypeptide of the invention as described in FIGS. 1-7589 (SEQ ID NOS: 1-7589), said method comprising contacting cells which normally express said polypeptide with a candidate compound, and determining the lack of expression said gene.

23. The method of claim 22, wherein said candidate compound is an antisense nucleic acid.

24. A method of identifying a compound that mimics the activity of a PRO polypeptide of the invention as described in any one of FIGS. 1-7589 (SEQ ID NOS: 1-7589), said method comprising contacting cells which normally respond to said polypeptide with a candidate compound, and determining the responsiveness by said cell to said candidate compound.

25. A method of stimulating the immune response in a mammal, said method comprising administering to said mammal an effective amount of a PRO polypeptide of the invention as described in any one of FIGS. 1-7589 (SEQ ID NOS: 1-7589), antagonist, wherein said immune response is stimulated.

26. A method of diagnosing an inflammatory immune response in a mammal, said method comprising detecting the level of expression of a gene encoding a PRO polypeptide of the invention as described in any one of FIGS. 1-7589 (SEQ ID NOS: 1-7589), (a) in a test sample of tissue cells obtained from the mammal, and (b) in a control sample of known normal tissue cells of the same cell type, wherein a higher or lower level of expression of said gene in the test sample as compared to the control sample is indicative of the presence of an inflammatory immune response in the mammal from which the test tissue cells were obtained.
